(19) United States
(12) Patent Application Publication
(10) Pub. No.: US 2026/0199403 A1
(43) Pub. Date: Jul. 16, 2026

(54) SYSTEMS FOR MODULATING MICROBIOME TARGET CELLS, METHODS AND COMPOSITIONS THEREOF

(71) Applicant: TROBIX BIO LTD, Netanya (IL)

(72) Inventors: Adi ELKELES, Tel Mond (IL); Ziv LIFSHITZ, Kfar Yona (IL); Yaron FELDHEIM, Herzeliya (IL); Shmuel TUVIA, Netanya (IL)

(21) Appl. No.: 18/998,106

(22) PCT Filed: Jul. 25, 2023
(86) PCT No.: PCT/IL2023/050774

§ 371 Date: Jan. 23, 2025

Related U.S. Application Data
(60) Provisional application No. 63/369,336, filed on Jul. 25, 2022.

(51) Int. Cl. A61K 35/74; A61K 35/00; A61K 48/00; A61P 29/00; C07K 14/54; C07K 14/55; C07K 14/605; C12N 1/20; C12N 7/04; C12N 9/22; C12N 9/52; C12N 9/64; C12N 15/113

(52) U.S. Cl. CPC A61K 35/74; A61K 48/00; A61P 29/00; C07K 14/5428; C07K 14/55; C07K 14/605; C12N 1/20; C12N 7/045; C12N 9/222; C12N 9/52; C12N 9/6472; C12N 15/113; C12Y 304/21026; C12Y 304/21062; A61K 2035/11; C12N 2310/20; C12N 2795/00043

(57) ABSTRACT

The present disclosure provides systems for modulating bacterial populations in the microbiome, specifically in the gut microbiome, by modifying target cell/s and/or population of cells comprising the target cell/s. The disclosed systems comprise the following components: (a) at least one modulating component comprising at least one nucleic acid sequence encoding and/or modulating at least one product of interest, at least one cas gene and at least one Clustered, Regularly Interspaced Short Palindromic Repeat (CRISPR) array; and (b) at least one selective component comprising at least one protospacer. The at least one protospacer is targeted by at least one spacer of the CRISPR array of (a), thereby inactivating the selective component. The present disclosure further provides compositions and methods using the disclosed systems and any components thereof in modulation of the microbiome and in therapeutic applications.

The Sequence Listing .xml file, entitled 2026-03-27-SeqLst-23464.665US1, created on Mar. 26, 2026, comprising 179,819 bytes, submitted on Mar. 27, 2026, is hereby incorporated herein by reference.

### FIELD OF THE INVENTION

The present disclosure relates to the field of manipulation of the microbiome. More specifically, the present disclosure relates to modulation of microbiome population/s for targeted production of a product of interest by target cells residing in the gut microbiome.

### BACKGROUND ART

References considered to be relevant as background to the presently disclosed subject matter are listed below:
WO 2016/084088
WO 2018/002940

Acknowledgement of the above references herein is not to be inferred as meaning that these are in any way relevant to the patentability of the presently disclosed subject matter.

### BACKGROUND OF THE INVENTION

The gut microbiome, a highly complex and dynamic ecosystem, comprises up to 2 Kg of bacteria in adult humans. The gut microbiome within an individual is established early in life and remains relatively stable during life-time, but its composition and/or function may be influenced by a range of factors such as diet, probiotics, and drugs, especially antibiotics.

This aggregate of trillions of microorganisms is now known to play a critical role in human health and predisposition to disease. It also performs various essential biological functions such as synthesis of vitamins, development and modulation of the immune system, bacterial defense, the intestinal response to epithelial cell injury and nutrient metabolism. In addition, the gut microbiota can produce a number of neurotransmitters, including serotonin, dopamine and noradrenaline. Researchers are now referring to the gut microbiota as the “second brain” or “second genome” due to its profound physiological role.

As noted above, the mammalian gut is a highly dense bacterial ecosystem, in fact, the highest among any known ecosystems. The human gut microbiota, for example, is populated predominantly by two phyla of bacteria, the Firmicutes (Gram positive aerobic and anaerobic cocci and rod shaped bacteria, e.g. Streptococcus and Heliobacteria, respectively) and the Bacteroidetes (Gram negative anaerobic motile and non-motile bacilli, e.g. Acidophilus), both of which constitute about 85-90% of the core gut microbiota. The rest consists of Proteobacteria (about 9%, e.g. Escherichia coli) and Actinobacteria (about 5% predominantly in the large intestine, e.g. Bifidobacterium). In addition, there are temporarily stable fungi populations dominated by various strains of Candida. Estimates of the number of bacterial species present in the human gut suggest that the collective human gut microbiome (i.e. a collective number of microbiota genomes) reaches over 35,000 bacterial species.

The gut microbiota regulates development and repair of the intestinal mucosal barrier. Intactness and functionality of the intestinal mucosal barrier provides the first-line defense for preservation of a body homeostasis to allow selective passage of nutrients, while preventing the passage of antigens, bacterial toxins and pathogens. Apart from serving as a physical barrier, the gut mucosa also serves an immunological sentinel that signals to resident innate immune cells in the mucosa to recruit and ultimately regulate the function of innate and adaptive immune system.

It has been suggested that the gut microbiota may further impact on the entire immune system of an organism thereby influencing various pathological conditions, including chronic liver disease, certain forms of heart disease and metabolic syndrome.

Therefore, modifying target cells in the gut microbiome may provide a powerful tool for the management of diseases and health processes and conditions. It is therefore important to specifically manipulate particular target cells within the microbiome to produce desired products.

Targeted delivery vehicles and expression systems have been recently developed. WO 2016/084088 relates to kits, systems and methods for interfering with horizontal transfer of a pathogenic gene between bacteria and for preventing a pathologic condition in a mammalian subject caused by a bacterial infection.

WO 2018/002940 relates to a platform for the preparation of improved nucleic acid delivery vehicles, specifically, vehicles having an extended host recognition ability, compositions and uses thereof.

There is therefore a need for effective systems for targeted manipulation of the microbiota in mammalian subjects to produce products having potential metabolic, immunological and/or neurological impact on the subject's health and development. These unmet needs are addressed by the present disclosure.

### SUMMARY OF THE INVENTION

A first aspect of the present disclosure relates to a system for modifying target bacterial cell/s and/or target bacterial cell population to produce a product of interest. More specifically, the disclosed system comprises at least one modulatory component and at least one selective component, specifically, the following components:
First (a), at least one microbiome modulating component comprising: (i) at least one nucleic acid sequence encoding and/or modulating said at least one product of interest and/or at least one element participating in the production and/or metabolism of said at least one product of interest; and (ii) at least one cas gene and at least one Clustered, Regularly Interspaced Short Palindromic Repeat (CRISPR) array, comprising at least one spacer; and
The second component (b), at least one selective component comprising at least one protospacer, wherein said at least one protospacer is targeted by at least one spacer of the CRISPR array of (a), thereby inactivating said selective component.

A further aspect of the present disclosure relates to a method for modifying target bacterial cell/s and/or target bacterial cell population to produce a product of interest. More specifically, the method comprising at least one of:
First (a), contacting the cell/s or any cell population comprising the cell/s with at least one microbiome modulating component comprising: (i) at least one nucleic acid sequence encoding and/or modulating at least one product of interest and/or at least one element participating in the production and/or metabolism of the at least one product of interest; and (ii) at least one cas gene and at least one CRISPR array comprising at least one spacer. The next step (b), involves contacting the cells or any cell population comprising the cell/s with at least one selective component comprising at least one protospacer. In some embodiments, the at least one protospacer is targeted by at least one spacer of the CRISPR array of (a), such that the selective component is inactivated.

Thereby, modifying target bacterial cell/s and/or target bacterial cell population to produce the product of interest.

A further aspect of the present disclosure relates to at least one bacterial cell and/or a population of the bacterial cells, or any composition or product thereof, producing at least one product of interest. In some embodiments, the cell and/or cell population is prepared by a method comprising at least one of: First (a), contacting the cell/s or any cell population comprising the cell/s with at least one microbiome modulating component comprising: (i) at least one nucleic acid sequence encoding and/or modulating at least one said product of interest and/or at least one element participating in the production and/or metabolism of said at least one product of interest; and (ii) at least one cas gene and at least one CRISPR array comprising at least one spacer; and The next step (b), contacting the cells or any cell population comprising the cell/s with at least one selective component comprising at least one protospacer. In some embodiments, the at least one protospacer is targeted by at least one spacer of the CRISPR array of (a), such that the selective component is inactivated; thereby modifying target bacterial cell/s and/or target bacterial cell population to produce the product of interest.

A further aspect of the present disclosure relates to a method for modifying and/or manipulating target bacterial cell/s and/or target bacterial cell population to produce a product of interest in a subject in need thereof. More specifically, the method comprising the step of administering to the subject an effective amount of at least one of: (a), at least one microbiome modulating component comprising: (i) at least one nucleic acid sequence encoding and/or modulating at least one the product of interest and/or at least one element participating in the production and/or metabolism of the at least one product of interest; and (ii), at least one cas gene and at least one Clustered, Regularly Interspaced Short Palindromic Repeat (CRISPR) array, comprising at least one spacer; and
(b) at least one selective component comprising at least one protospacer, wherein the at least one protospacer is targeted by at least one spacer of the CRISPR array of (a), such that the selective component is inactivated; (c) at least one composition, kit or system comprising at least one of (a) and (b); and (d) any combination of the at least one of (a), (b) and (c) with the at least one drug.

A further aspect of the present disclosure relates to a method for treating, preventing, ameliorating, reducing or delaying the onset of at least one pathologic disorder in a subject in need thereof. More specifically, the method comprising the step of administering to said subject an effective amount of at least one of:
(I) (a) at least one microbiome modulating component comprising: (i) at least one nucleic acid sequence encoding and/or modulating the at least one product of interest and/or at least one element participating in the production and/or metabolism of the at least one product of interest; and (ii) at least one cas gene and at least one CRISPR array, comprising at least one spacer; and
(b) at least one selective component comprising at least one protospacer. It should be noted that the at least one protospacer is targeted by at least one spacer of the CRISPR array of (a), thereby inactivating the selective component;
(II) at least one bacterial cell and/or a population of the bacterial cells, or any composition or product thereof, producing the product of interest;
(III) at least one composition, kit or system comprising at least one of (I) and (II); and
(IV) any combination of the at least one of (I), (II) and (III) with at least one therapeutic agent.

A further aspect of the present disclosure relates to a method for modulating at least one of an immune-related process, a metabolic process and a neuronal and/or neuro-behavioral processes in a subject in need thereof. According to some embodiments, the method comprising the step of administering to the subject an effective amount of at least one of:
(I) (a) at least one microbiome modulating component comprising:
(i) at least one nucleic acid sequence encoding and/or modulating at least one the product of interest and/or at least one element participating in the production and/or metabolism of the at least one product of interest; and
(ii) at least one cas gene and at least one CRISPR array, comprising at least one spacer; and
(b) at least one selective component comprising at least one protospacer, wherein the at least one protospacer is targeted by at least one spacer of the CRISPR array of (a), thereby inactivating the selective component;
(II) at least one bacterial cell and/or a population of the bacterial cells, or any composition or product thereof, producing the product of interest;
(III) at least one composition, kit or system comprising at least one of (I) and (II); and
(IV) any combination of the at least one of (I), (II) and (III) with at least one therapeutic agent.

A further aspect of the present disclosure relates to a pharmaceutical composition or kit comprising at least one of: (I) (a) at least one microbiome modulating component comprising: (i) at least one nucleic acid sequence encoding and/or modulating the at least one product of interest and/or at least one element participating in the production and/or metabolism of the at least one product of interest; and (ii), at least one cas gene and at least one CRISPR array, comprising at least one spacer; and (b) at least one selective component comprising at least one protospacer. The at least one protospacer is targeted by at least one spacer of the CRISPR array of (a), thereby inactivating the selective component. The disclosed composition or kit may additionally or alternatively comprise in some embodiments, (II) at least one bacterial cell and/or a population of the bacterial cells producing the product of interest, or any product thereof. In yet some alternative or additional embodiments, the disclosed compositions or kits may comprise (III), any combination of the at least one of (I) and (II), optionally, with at least one therapeutic agent. Still further, the composition or kit of the present disclosure may optionally further comprise at least one of pharmaceutically acceptable carrier/s, diluent/s, excipient/s and additive/s.

These and other aspects of the present disclosure will become apparent by the hand of the following description.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1. The selective component

Ori-origin of replication; Toxin—the gene encodes toxin that kills bacteria; Proto-spacer-the region on the selective vector that is targeted by the cognate spacer resides in the vector; PS-phage packaging signal.

FIG. 2A-2B. Microbiome modulating component for cytokine production

FIG. 2A: Microbiome modulating component for IL-2 production.

Ori-origin of replication; IL-2-nucleic acid sequence encoding IL-2; Cas genes, CRISPR array-comprise alternating conserved repeats and spacers; spacer that target the selective vector; PS-phage packaging signal.

FIG. 2B: Microbiome modulating component for IL-10 production.

Ori-origin of replication; IL-10-nucleic acid sequence encoding IL-10; Cas genes, CRISPR array-comprise alternating conserved repeats and spacers; spacer that target the selective vector; PS-phage packaging signal.

FIG. 3A-3F. Schematic illustration of the six different constructs of Interleukin-10

Six different constructs of Interleukin-10 (SEQ ID NO: 12 to 17, that contain nucleic acid sequences of control elements, and variants of human IL-10 encoding sequences as denoted by SEQ ID NO: 25 to 30) were designed and evaluated. J23113-bacterial promoter (SEQ ID NO: 18); Cm-Chloramphenicol antibiotic resistance cassette; YebF-bacterial secretion protein fused to the hIL construct (SEQ ID NO: 2); pLPP-the LPP gene bacterial promoter (SEQ ID NO: 19); Lightning signs mark the location and the nature of the mutations in the hIL-10 amino acid sequence.

FIG. 3A. 323113 hIL-10 cut construct (SEQ ID NO: 12), that comprise the variant nucleic acid sequence encoding IL-10, as denoted by SEQ ID NO: 25.

FIG. 3B. 323113 hIL-10 mod construct (SEQ ID NO: 13), that comprise the variant nucleic acid sequence encoding IL-10, as denoted by SEQ ID NO: 26.

FIG. 3C. pLpp hIL-10 cut construct (SEQ ID NO: 14), that comprise the variant nucleic acid sequence encoding IL-10, as denoted by SEQ ID NO: 27.

FIG. 3D. pLpp Hil-10 mod construct (SEQ ID NO: 15), that comprise the variant nucleic acid sequence encoding IL-10, as denoted by SEQ ID NO: 28.

FIG. 3E. 323113 hIL-10 mut cut construct (SEQ ID NO: 16), that comprise the variant nucleic acid sequence encoding IL-10, as denoted by SEQ ID NO: 29.

FIG. 3F. 323113 hIL-10 mut mod construct (SEQ ID NO: 17), that comprise the variant nucleic acid sequence encoding IL-10, as denoted by SEQ ID NO: 30.

FIG. 4A-4F. Schematic illustration of the final IL-10 constructs Schematic illustration of the final constructs used in the IL-10 secretion assay and the IL-10 biological activity assay. The CRISPR-Cas3 system encode for the Cas genes and CRISPR array that target the Selective phages. Ori-origin of replication, PS-phage packaging signal.

FIG. 4A. 323113 hIL-10 cut construct (SEQ ID NO: 12).

FIG. 4B. 323113 hIL-10 mod construct (SEQ ID NO: 13).

FIG. 4C. pLpp hIL-10 cut construct (SEQ ID NO: 14).

FIG. 4D. pLpp hIL-10 mod construct (SEQ ID NO: 15).

FIG. 4E. 323113 hIL-10 mut cut construct (SEQ ID NO: 16).

FIG. 4F. 323113 hIL-10 mut mod construct (SEQ ID NO: 17).

FIG. 5. Microbiome modulating component for metabolic polypeptide production Ori-origin of replication; GLP-1-nucleic acid sequence encoding GLP-1 (SEQ ID NO: 20), Cas genes; CRISPR array-comprise alternating conserved repeats and spacers; spacer that target the selective vector; PS-phage packaging signal.

FIG. 6. Microbiome modulating component for gliadins-degrading enzymes production

Ori-origin of replication; GDE1,2,3-nucleic acid sequence encoding three different gliadin degrading enzymes: 1-prolyl endopeptidases (Myxococcus xanthus) (SEQ ID NO: 9), 2-cysteine proteinase (Hordeum vulgare) (SEQ ID NO: 10) and 3—Subtilisin (Rothia mucilaginosa) (SEQ ID NO: 11); Cas genes; CRISPR array-comprise alternating conserved repeats and spacers; spacer that target the selective vector; PS-phage packaging signal.

FIG. 7. Expression of Human GLP-1 protein using the microbiome modulating component

Human GLP-1 protein was secreted from bacteria containing the Microbiome modulating payload. E. coli BW25113 (DSM 27469) bacterial cultures containing the Microbiome modulating GLP-1 payloads or empty vector were grown in LB to reach stationary growth phase. The concentrations of the secreted GLP-1 protein were determined from samples taken from the culture's supernatant, using ELISA (Abcam) and according to the manufacturer's instructions. Samples were also plated on LB plates and CFU/ml were determined to normalize the protein concentrations to the concentration of bacteria in the cultures. Data represents the average of three independent studies using triplicates.

FIG. 8A-8B. Expression of cytokines using the microbiome modulating component

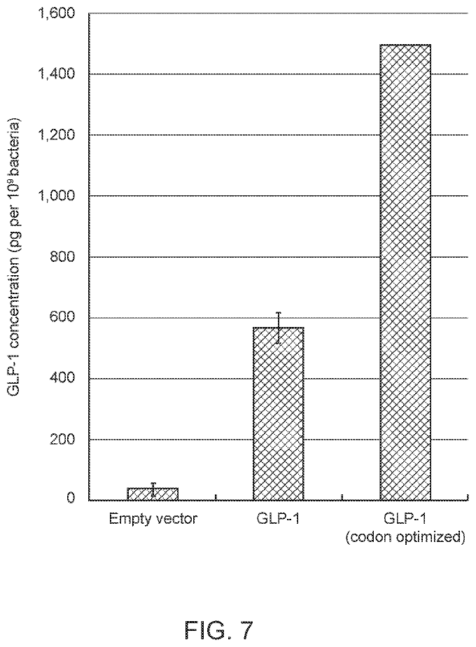

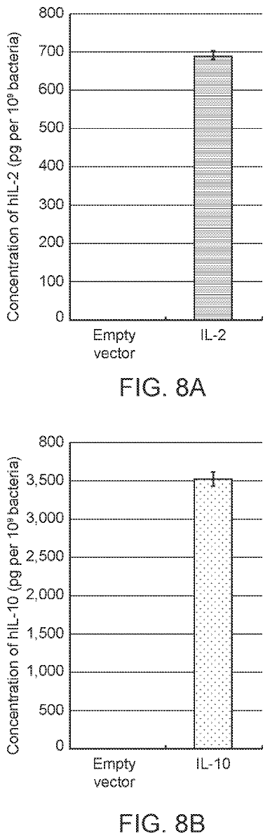

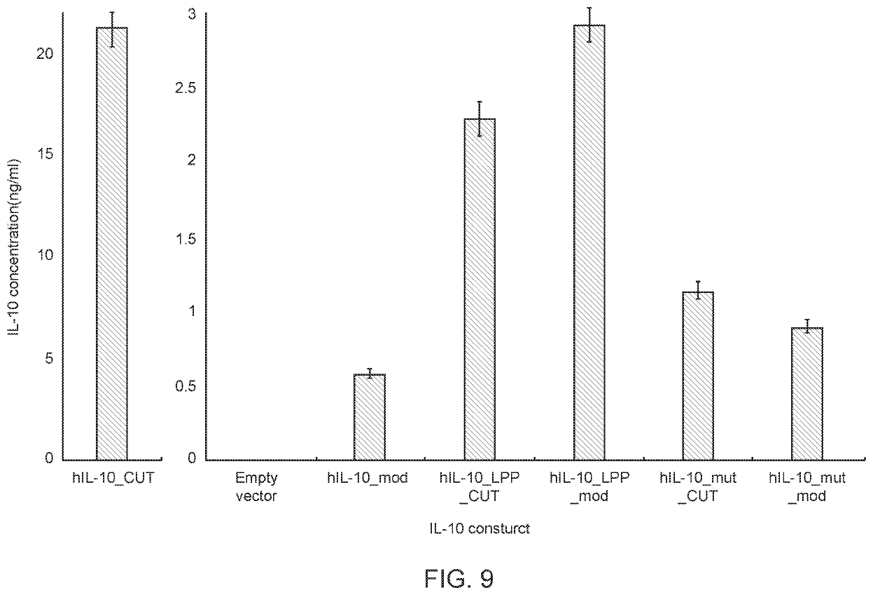

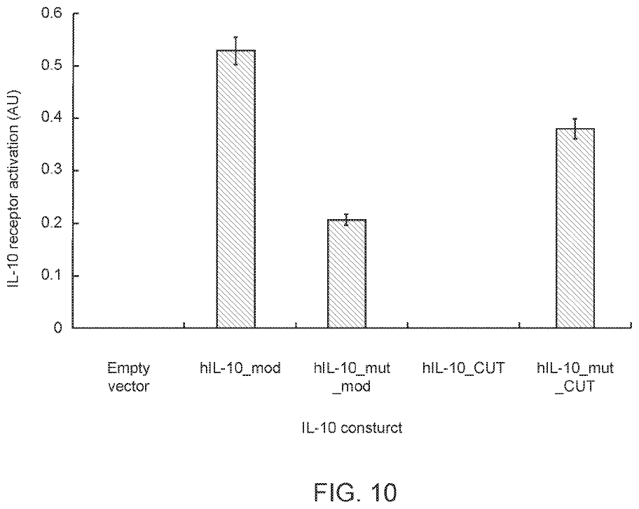

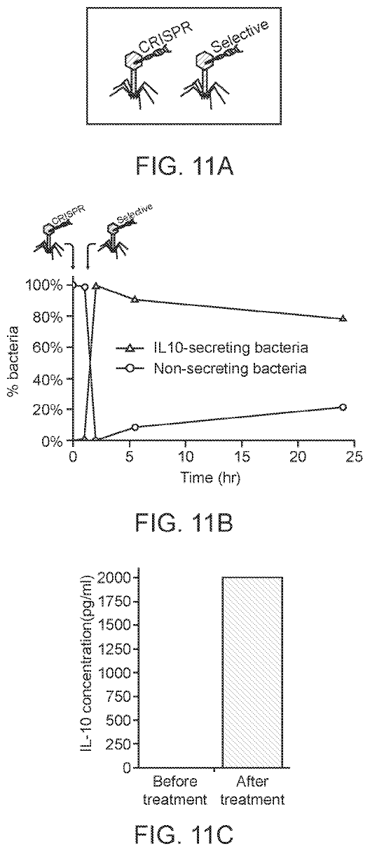

Human IL-2 and IL-10 proteins were secreted from bacteria containing the corresponding microbiome modulating component comprising nucleic acids sequence encoding each of these cytokines, as a payload. The concentrations of the human IL-2 (FIG. 8A) or IL-10 (FIG. 8B) were determined as described in FIG. 7, using ELISA and according to manufacture instructions (#ab229441, Abcam for IL-2 and #DY217B, R&D systems for IL-10). Data represents the average of three independent studies using triplicates.

FIG. 9. Determination of hIL-10 concentration secreted from bacteria

E. coli BW25113 bacterial cultures, each containing one of the six tested constructs that encode for hIL-10 or empty vector, were grown in LB to reach stationary growth phase. The cultures were centrifugated and the concentrations of the hIL-10 were determined from samples taken from the supernatant, using ELISA and according to manufacture instructions (#DY217B, R&D systems). The cultures were also plated on LB plates and CFU/ml were determined to normalize the protein concentrations to the concentration of bacteria in the cultures. Data represents the average of three independent studies using triplicates.

FIG. 10. Bioactivity of the secreted hIL-10

The ability of the variant secreted hIL-10 to activate IL-10 receptor was determined using the HEK-blue cellular system, in which the binding to membrane bound IL-10 receptors leads to the secretion of Secreted alkaline phospbaase (SEAP) measured by absorbance at OD₆₃₀. Samples of the different hIL-10 variants or empty vector were incubated overnight with HEK-Blue IL-10 cells and the SEAP activity was assessed by measuring the absorbance OD630 nm. Data represent the average of two measurements (duplicates)±range of the observations.

FIG. 11A-11C. In-vitro assay to modify and enrich bacterial population with hIL-10-producing bacteria

FIG. 11A. The system is composed of two phages, that have an identical structure, target the same bacteria, but differ in their payloads: left-the “CRISPR” phage that packs the hIL-10-encoding construct; Right-the “Selective” phage that packs a modified genome of non-replicating T7 lytic phage. In some embodiments, these phages act sequentially.

FIG. 11B. Bacterial culture of E. coli BW25113 isolate was grown until reached mid-exponential growth phase (107 CFU/ml). At time point 0 hr, “CRISPR” phages were used to infect the culture at an MOI of ~0.1. Two hours later the “Selective” phages were used to infect the culture at an MOI of ~10. The proportion of hIL-10-producing (blue) and non-hIL-10-producing bacteria (orange) from the total bacterial population (% bacteria) was determined. Phages with arrows indicate the timepoint of infection. Data are representative of three independent experiments.

FIG. 11C. Samples from the bacterial culture were taken at timepoint 0 hr (before the addition of the “CRISPR” phages) and at timepoint 48 hr and the concentration of hIL-10 was measured.

### DETAILED DESCRIPTION OF THE INVENTION

A first aspect of the present disclosure relates to a system for modifying target bacterial cell/s and/or target bacterial cell population to produce a product of interest. the disclosed systems comprise at least one modulatory component and at least one selective component. More specifically, the disclosed systems comprise the following components.

First (a), at least one microbiome modulating component comprising: (i) at least one nucleic acid sequence encoding and/or modulating the at least one product of interest and/or at least one element participating in the production and/or metabolism of the at least one product of interest; and (ii) at least one cas gene and at least one Clustered, Regularly Interspaced Short Palindromic Repeat (CRISPR) array, comprising at least one spacer; and

The second component (b) of the disclosed systems comprise at least one selective component comprising at least one protospacer. It should be noted that at least one protospacer is targeted by at least one spacer of the CRISPR array of (a) (i), thereby inactivating said selective component. Thus, a cell that comprises the microbiome modulating component of (a), is capable of specifically inactivating the selective component of (b).

In some embodiments, the nucleic acid sequence of the microbiome modulating component of the systems disclosed herein encodes the at least one product of interest. In yet some further embodiments, and specifically in cases where the nucleic acid sequence of the microbiome modulating component of the systems disclosed herein modulates at least one product of interest and/or at least one element participating in the production and/or metabolism of the product of interest, such nucleic acid sequence may be any modulating nucleic acid sequence. Examples for modulating nucleic acid sequences may include but are not limited to siRNAs, miRNA, dsRNA, antisense oligo, gRNA that recruits nucleases (e.g., Cas/CRISPR system) and the like, that may enhance, reduce, or modify directly or indirectly at least one element participating in metabolism and/or production of the desired product of interest. In some embodiments, the CRISPR system is further used not only to provide an absolute linkage between the selective component and the modulating component, but also to directly modulate the target element participating in metabolism of the target drug, e.g., either to eliminate (knockdown) the target element, or to enhance and elevate this component (e.g., where the CRISPR system requires activators, for example transcription activators). Thus, in such particular and no-limiting embodiments, the at least one nucleic acid sequence encoding or modulating at least one element participating in metabolism of the desired product of interest is comprised within the CRISPR array (e.g., spacers that specifically target the target element participating in metabolism of the desired product of interest).

In some embodiments of the disclosed systems, the selective component/s may comprise at least one protospacer targeted by at least one spacer of the microbiome modulating component. In such case, the selective component is specifically inactivated by the CRISPR array of the microbiome modulating component.

Still further, in some embodiments of the system disclosed herein, the microbiome modulating component is comprised within at least one delivery vehicle that specifically targets the cell/s and/or the cells in a bacterial cell population.

In some embodiments, the selective component is comprised within at least one delivery vehicle. In yet some particular and non-limiting embodiments, the delivery vehicle specifically targets the cell/s and/or the cells in a bacterial cell population.

In some embodiments, the at least one delivery vehicle used for the microbiome modulating component and/or the selective component is, or comprises at least one genetic element. In some non-limiting embodiments, the genetic element may be at least one of: at least one transducing particle, at least one bacteriophage or any fragments or parts thereof, at least one bacteriophage-like particle, at least one chimeric and/or recombinant bacteriophage or transducing particle, at least one hybrid bacteriophage or transducing particle, at least one vector, at least one plasmid, at least one phagemid and/or or any mixture or cocktail thereof.

In yet some further embodiments, the selective component of the system of the present disclosure is comprised within at least one delivery vehicle that comprise at least one agent that affects cell viability and/or activity.

Thus, in some embodiments, the at least one delivery vehicle used as the selective component is, or comprises at least one genetic element. In some non-limiting embodiments, the genetic element may be at least one of: at least one transducing particle, at least one lytic bacteriophage or any fragments or parts thereof, at least one vector, plasmid and/or construct that comprises and/or encodes at least one toxic element that kill bacterial cells or disrupt, attenuate, and/or inhibit bacterial growth and any combinations or cocktails thereof. As indicated above, the selective component of the disclosed systems comprises at least one protospacer targeted by at least one spacer comprised within the modulating component. In some particular embodiments, such protospacer may be comprised within a nucleic acid sequence comprising and/or encoding at least one toxic element, a toxin that kill bacterial cells or disrupt, attenuate, and/or inhibit bacterial growth.

In yet some further embodiments, the delivery vehicle used for the modulatory component and/or the selective component/s of the system of the is at least one bacteriophage or any bacteriophage-like transducing particle.

In some specific embodiments, the selective component of the system disclosed herein comprising at least one nucleic acid sequence encoding at least one factor affecting element/s essential for growth of the target cell. The selective component comprises at least one protospacer targeted by at least one spacer of the microbiome modulating component such that the selective component is specifically inactivated by the CRISPR array of the microbiome modulating component.

In some embodiments, the delivery vehicle is at least one bacteriophage or any bacteriophage-like, or bacteriophage-derived transducing particle. Thus, cells that carry the modulatory component of the disclosed systems are capable of inactivating the selective component. The selective component therefore acts in some embodiments to destroy, kill, attenuate growth, inactivate and/or affect the function of cells that were not transduced by and/or do not contain the modulatory component of the disclosed systems.

“Vehicles” or “delivery Vehicles” as used herein encompass vectors or any transducing particles such as bacteriophage, bacteriophage-like particles, plasmids, phagemides, viruses, integratable DNA fragments, hybrid bacteriophages, recombinant bacteriophages, and other vehicles, which enable the transfer, introduction, and/or transduction of nucleic acid molecules into a desired target cell, and in some further embodiments, leads to expression of the transduced nucleic acid molecule in the target cell. A transducing particle or element as used herein refers to any vector or vehicle capable of transducing and inserting nucleic acid molecule or any cargo into a target cell, or an artificial cellular system.

Vectors are typically self-replicating DNA or RNA constructs containing the desired nucleic acid sequences, and operably linked genetic control elements that are recognized in a suitable cell and effect the translation of the desired gene. Generally, the genetic control elements can include a prokaryotic promoter system. Such system typically includes a transcriptional promoter, transcription enhancers to elevate the level of RNA expression. Vectors usually contain an origin of replication that allows the vector to replicate independently of the cell.

Accordingly, the term control and regulatory elements includes promoters, terminators and other expression control elements. Such regulatory elements are described in Goeddel; [Goeddel., et al., Gene Expression Technology: Methods in Enzymology 185, Academic Press, San Diego, Calif. (1990)]. For instance, any of a wide variety of expression control sequences that control the expression of a DNA sequence when operatively linked to it may be used in these vectors to express DNA sequences encoding any desired protein using the method of this invention.

A vector or delivery vehicle may additionally include appropriate restriction sites, antibiotic resistance or other markers for selection of vector-containing cells. Plasmids are the most commonly used form of vector but other forms of vectors which serve an equivalent function and which are, or become, known in the art are suitable for use herein. See, e.g., Pouwels et al., Cloning Vectors: a Laboratory Manual (1985 and supplements), Elsevier, N.Y.; and Rodriquez, et al. (eds.) Vectors: a Survey of Molecular Cloning Vectors and their Uses, Buttersworth, Boston, Mass (1988). It is to be understood that this definition of delivery vehicle/s is relevant to any system, method or composition as described in the present disclosure.

Under the term “bacteriophage” it is meant a virus that infects, replicates and assembles within prokaryotes, such as bacteria. It should be noted that the term “bacteriophage” is synonymous with the term “phage”. Phages are composed of proteins that encapsulate a DNA or RNA genome, which may encode only a few or hundreds of genes thereby producing virions with relatively simple or elaborate structures. Phages are classified according to the International Committee on Taxonomy of Viruses (ICTV) considering morphology and the type of nucleic acid (DNA or RNA, single- or double-stranded, linear or circular). About 19 phage families have been recognized so far that infect bacteria and/or archaea (a prokaryotic domain previously classified as archaebacteria). Many bacteriophages are specific to a particular genus or species or strain of cell. It should be appreciated that any suitable phage may be used as the delivery vehicle by the methods, systems and compositions of the present disclosure.

In some non-limiting embodiments the bacteriophage of the presently disclosed subject matter belongs to the order Caudovirales (for example to the family of Podoviridae, Myoviridae or Siphoviridae) or to the order of Ligamenvirales (for example to the family of Lipothrixviridae or Rudivirus). Phages from other families are also encompassed by the present disclosure, for example Ampullaviridae, Bicaudaviridae, and Clavaviridae to name but few.

In other embodiments the bacteriophage according to the present disclosure is one of (but not limited to) the bacteriophage family Podoviridae, Myoviridae or Siphoviridae, Lipothrixviridae or Rudivirus.

In some embodiments, the bacteriophage or bacteriophage-like transducing particle and/or transducing particle is of the T7-like viruses, the T7-like viruses, or any phage-like particles or transducing particles thereof. In some embodiments, the bacteriophage or bacteriophage-like transducing particle is of the T4-like viruses, the T4-like viruses, or any phage-like particles or transducing particles thereof. In yet some further embodiments, the bacteriophage used as a delivery and the Escherichia virus Lambda. It should be however noted that in some embodiments, any bacteriophage, bacteriophage particles or any hybrid composed of part of sequences of at least one bacteriophage, may be used herein as a suitable delivery vehicle for the at least one microbiome modulating component of the system of the invention and/or the at least one selective component of the system of the invention. It should be further appreciated that the present disclosure further encompasses the option of using cocktails of various delivery vehicles (e.g., transducing particles, bacteriophage-like particles, bacteriophages and the like), that are directed at and are capable of transducing the modulating component and/or the selective component to several, optionally, different target cells in a cell population.

In certain specific embodiments, the bacteriophage according to the present disclosure is at least one of T7 like-virus or T4 like-virus.

In further specific embodiments, the phage used as the delivery vehicle by the methods of the invention, systems and compositions of the disclosure described herein after, may be a T7-like-virus, specifically, Enterobacteria phage T7. Bacteriophage T7 are DNA viruses having a lytic life cycle.

More specifically, the phage according to the present disclosure may be Escherichia coli phage T7 (a member of the Podoviridae family of the Caudovirales (tailed phages) order, as detailed above). T7 is composed of an icosahedral capsid with a 20-nm short tail at one of the vertices. The capsid is formed by the shell protein gene product (gp) 10 and encloses a DNA of 40 kb. A cylindrical structure composed of gp14, gp15, and gp16 is present inside the capsid, attached to the special vertex formed by the connector, a circular dodecamer of gp8 (8, 10). The proteins gp11 and gp12 form the tail; gp13, gp6.7, and gp7.3 have also been shown to be part of the virion and to be necessary for infection, although their location has not been established. The main portion of the tail is composed of gp12, a large protein of which six copies are present; the small gp 11 protein is also located in the tail. Attached to the tail are six fibers, each containing three copies of the gp17 protein.

Phages used as the delivery vehicle by the methods, systems and compositions of the present disclosure may include other groups members of the family Podoviridae, for example but not limited to T3 phages, Φ29, P22, P-SPP7, N4, ε15, K1E, K1-5 and P37.

In some specific embodiments, phages used as the delivery vehicle by the methods, systems and compositions of the present disclosure may include, but are not limited to Enterobacteria phage T7, Enterobacteria phage 13a, Yersinia phage YpsP-G, Enterobacteria phage T3, Yersinia phage YpP-R, Salmonella phage phiSG-JL2, Salmonella phage Vi06, Pseudomonas phage gh-1, Klebsiella phage K11, Enterobacter phage phiEap-1, Enterobacter phage E-2, Klebsiella phage KP32, Klebsiella phage KP34, Klebsiella phage vB_KpnP_KpV289 and Pseudomonas phage phiKMV.

By way of another example, the bacteriophage/s include, but are not limited to, any bacteriophage capable of infecting a bacterium including but not limited to any one of the proteobacteria, Firmicutes and Bacterioidetes phyla.

To name but few, these bacteriophages, may include but are not limited to bacteriophages specific for Staphylococcus aureus, specifically, at least one of vB_Sau. My D1, vB_Sau My 1140, vB_SauM 142, Sb-1, vB_SauM 232, vB_SauS 175, vB_SauM 50, vB_Sau 51/18, vB_Sau.M. 1, vB_Sau.M. 2, vB_Sau.S. 3, vB_Sau.M. 4, vB_Sau.S. 5, vB_Sau.S. 6, vB_Sau.M.7, vB_Sau.S.8, vB_Sau.S.9, vB_Sau.M.10, vB_Sau.M.11. In yet some further embodiments, bacteriophages specific for Klebsiella pneumoniae, may be also applicable for the present invention. In more specific embodiments, these phages may include vB_Klp 1, vB_Klp 2, vB_Klp. M.1, vB_Klp. M.2, vB_Klp. P.3, vB_Klp. M.4, vB_Klp. M.5, vB_Klp. M.6, vB_Klp. 7, vB_Klp. M.8, vB_Klp. M.9, vB_Klp. M.10, vB_Klp. P.11, vB_Klp. P.12, vB_Klp. 13, vB_Klp. P.14, vB_Klp. 15, vB_Klp. M.16. Still further, in certain embodiments, bacteriophages specific for Pseudomonas aeruginosa, may be applicable as the delivery vehicles of the invention or alternatively, as a source for heterologous host recognition elements for a suitable delivery vehicle. Non-limiting examples for such bacteriophages include but are not limited to vB_Psa.Shis 1, vB_PsaM PAT5, vB_PsaP PAT14, vB_PsaM PAT13, vB_PsaM ST-1, vB_Psa CT 27, vB_Psa CT 44 K, vB_Psa CT 44 M, vB_Psa 16, vB_Psa Ps-1, vB_Psa 8-40, vB_Psa 35 K, vB_Psa 44, vB_Psa 1, vB_Psa 9, vB_Psa 6-131 M, vB_Psa CT 37, vB_Psa CT 45 S, vB_Psa CT 45 M, vB_Psa CT 16 MU, vB_Psa CT 41, vB_Psa CT 44 MU, vB_Psa CT 43, vB_Psa CT 11 K, vB_Psa 1638, vB_Psa Ps-2, vB_Psa 35 CT, vB_Psa 35 M, vB_Psa S.Ch.L, vB_Psa R1, vB_Psa SAN, vB_Psa L24, vB_Psa F8, vB_Psa BT-4, vB_Psa BT-2(8), vB_Psa BT-1(10), vB_Psa BT-4-16, vB_Psa BT-5, vB_Psa F-2, vB_Psa B-CF, vB_Psa Ph7/32, vB_Psa Ph7/63, vB_Psa Ph5/32, vB_Psa Ph8/16, vB_Psa Ph 1/1, vB_Psa, vB_Psa 3, vB_Psa 4, vB_Psa 5, vB_Psa 6, vB_Psa 7, vB_Psa.P. 15, vB_Psa.17, vB_Psa.M. 18, vB_Psa. 28, vB_Psa.M 0.2, vB_Psa.M 3, vB_Psa.23, vB_Psa.P. 8, vB_Psa.M. PST7, vB_Psa.M .C5, vB_Psa.M D1038. In further embodiments, bacteriophages specific for Acinetobacter baumanii, may be applicable for the present invention. Such lytuic or temperate phages may include any one of vB_Aba B37, vB_Aba G865, vB_Aba G866, vB_Aba U7, vB_Aba U8, vB_Acb 1, vB_Acb 2. In yet some further embodiments, bacteriophages specific for Enterobacter, may be applicable as the delivery vehicles of the invention or alternatively, as a source for heterologous host recognition elements for a suitable delivery vehicle, specifically, any one of vB_Eb 1, vB_Eb 2, vB_Eb 3, vB_Eb 4 bacteriophages. In yet some further embodiments, Enterococcus faecalis specific bacteriophages may be used. Several non-limiting examples include any one of, vB_Ec 1, vB_Ec 2, vB_Enf.S.4, vB_Enf.S.5 bacteriophages.

In yet some further embodiments, bacteriophages that specifically infect Bacillus anthracis, for example, vB_BaK1, vB_BaK2, vB_BaK6, vB_BaK7, vB_BaK9, vB_BaK10, vB_BaK11, vB_BaK12, vB_BaGa4, vB_BaGa5, vB_BaGa6, may be also applicable for the present invention. Still further, bacteriophages specific for Brucella abortus for example, Tb, vB_BraP IV, vB_BraP V, vB_BraP VI, vB_BraP VII, vB_BraP VIII, vB_BraP IX, vB_BraP X, vB_BraP XII, vB_BraP 12(b), vB_BraP BA, vB_BraP 544, vB_BraP 141, vB_BraP 141m, vB_BraP 19x, vB_BraP 19m, vB_BraP 9, bacteriophages specific for Brucella canis, specifically, vB_BrcP 1066, bacteriophages specific for Clostridium perfigenes A.B.C.D.E, for example, vB_CpPI, vB_CpII, vB_CpIII, vB_CpIV, bacteriophages specific for Desulfovibrio vulgaris, specifically, vB_DvRCH1/M1, vB_DvH/P15, vB_DvH/M15, those specific for Enterococcus faecalis, specifically, vB_Ec 1, vB_Ec 2, vB_Enf S.4, vB_Enf.S.5, bacteriophages specific for Escherichia coli, specifically, vB_Eschc.pod 9, vB_Eschc.Pod 4, vB_Eschc.Shis 7, vB_Eschc.Shis 14, vB_Eschc.Shis 5, vB_Eschc.My 2, PhI-1, PhI-2, PhI3, PhI4, PhI5, T2, T4, T5, DDII, DDVI, DDVII, vB_Eschc.Shis 7/20, vB_Eschc.Shis 1161, vB_Eschc.Shis 8963, vB_Esche 4, vB_Esche 11/24, vB_Eschc.Shis 18, vB_Shis 3/14, vB_Sau A, vB_Shis G, vB_Eschc.Shis W, vB_Shis GE25, vB_Eschc.Shis 8962, vB_Esche 90/25, vB_Esche 5/25, vB_Esche 12/25, vB_Esche H, T3, T6, T7, vB_Esche 4, vB_Esche 121, vB_Esche BaK2, vB_Esche L7-2, vB_Esche L7-3, vB_Esche L7-7, vB_Esche L7-8, vB_Esche L7-9, vB_Esche L7-10, vB_Esche D8, vB_Eschc.Shis 20, vB_Eschc.Shis 25, vB_Eschc.Shis 27, vB_Eschc.Shis MY, vB_Eschc 11, vB_Eschc 12, vB_Eschc 13, vB_Eschc 17, vB_Eschc 18, vB_Eschc 19, vB_Eschc 20, vB_Eschc 21, vB_Eschc 22, vB_Eschc 23, vB_Eschc 24, vB_Eschc 25, vB_Eschc 26, vB_Eschc 27, vB_Eschc 28, vB_Eschc 29, vB_Eschc 30, vB_Eschc 31, vB_Eschc 32, vB_Eschc 34, vB_Eschc 35, vB_Eschc 37, vB_Eschc 38, vB_Eschc 39, vB_Eschc 44, vB_Eschc 45, vB_Eschc 46, vB_E. coli.M. 1, vB_E. coli.M. 2, vB_E. coli. P.3, vB_E. coli. P.4, vB_E. coli. P.5, vB_E. coli. P.6, vB_E. coli. P.7, vB_E. coli. P.8, phages specific for Salmonella paratyphi, specifically, vB_SPB Diag 1, vB_SPB Diag 2, vB_SPB Diag 3, vB_SPB Diag 3b, vB_SPB Diag Jersey, vB_SPB Diag Beecles, vB_SPB Diag Taunton, vB_SPB DiagB.A.O.R, vB_SPB Diag Dundee, vB_SPBDiagWorksop, vB_SPB Diag E, vB_SPB Diag D, vB_SPB Diag F, vB_SPB Diag H, specific for Salmonella typhi abdominalis vB_Sta Diag A, vB_Sta Diag B1, vB_Sta Diag B2, vB_Sta Diag C1, vB_Sta Diag C2, vB_Sta Diag C3, vB_Sta Diag C4, vB_Sta Diag C5, vB_Sta Diag C6, vB_Sta Diag C7, vB_Sta Diag D1, vB_Sta Diag D2, vB_Sta Diag D4, vB_Sta Diag D5, vB_Sta Diag D6, vB_Sta Diag D7, vB_Sta Diag D8, vB_Sta Diag E1, vB_Sta Diag E2, vB_Sta Diag E5, vB_Sta Diag E10, vB_Sta Diag F1, vB_Sta Diag F2, vB_Sta Diag F5, vB_Sta Diag G, vB_Sta Diag H, vB_Sta Diag J1, vB_Sta Diag J2, vB_Sta Diag K, vB_Sta Diag L1, vB_Sta Diag L2, vB_Sta Diag M1, vB_Sta Diag M2, vB_Sta Diag N, vB_Sta Diag 0, vB_Sta Diag T, vB_Sta Diag Vi1, vB_Sta Diag27, vB_Sta Diag 28, vB_Sta Diag 38, vB_Sta Diag 39, vB_Sta Diag 40, vB_Sta Diag 42, vB_Sta Diag 46, Salmonella typhimurium, specifically, vB_Stm.My 11, vB_Stm.My 28, vB_Stm.Shis 13, vB_Stm.My 760, vB_Stm.Shis 1, IRA, vB_Stm 16, vB_Stm 17, vB_Stm 18, vB_Stm 19, vB_Stm 20, vB_Stm 21, vB_Stm 29, vB_Stm 512, vB_Stm Diag I, vB_Stm Diag II, vB_Stm Diag III, vB_Stm Diag IV, vB_Stm Diag V, vB_Stm Diag VI, vB_Stm Diag VII, vB_Stm Diag VIII, vB_Stm Diag IX, vB_Stm Diag X, vB_Stm Diag XI, vB_Stm Diag XII, vB_Stm Diag XIII, vB_Stm Diag XIV, vB_Stm Diag XV, vB_Stm Diag XVI, vB_Stm Diag XVII, vB_Stm Diag XVIII, vB_Stm Diag XIX, vB_Stm Diag XX, vB_Stm Diag XXI, vB_Stm Diag 1, vB_Stm Diag 2, vB_Stm Diag 3, vB_Stm Diag 4, vB_Stm Diag 5, vB_Stm Diag 6, vB_Stm Diag 7, vB_Stm Diag 8, vB_Stm Diag 9, vB_Stm Diag 10, vB_Stm Diag 11, vB_Stm Diag 12, vB_Stm Diag 13. vB_Stm Diag 14. vB_Stm Diag 15. vB_Stm Diag 16, vB_Stm Diag 17, vB_Stm Diag 18, vB_Stm Diag 19, vB_Stm Diag 20, vB_Stm Diag 21, vB_Stm Diag 22, vB_Stm Diag 23, vB_Stm Diag 24, vB_Stm Diag 25, vB_Stm Diag 26, vB_Stm Diag 27, vB_Stm Diag 28, vB_Stm Diag 29, vB_Stm Diag 30, vB_Stm Diag 31, vB_Stm Diag 32, vB_Stm Diag 33, vB_Stm Diag 34, vB_Stm Diag 35, vB_Stm Diag 36, vB_Stm Diag 37, vB_Stm Diag 38, vB_Stm Diag 39, vB_Stm Diag 40, vB_Stm Diag 41, vB_Stm Diag 42, vB_Stm Diag 43, vB_Stm Diag 44, vB_Stm Diag 45, vB_Stm Diag 46, vB_Stm Diag 47, vB_Stm Diag 48, vB_Stm Diag 49, vB_Stm Diag 50, vB_Stm Diag 51, vB_Stm Diag 52, vB_Stm Diag 53, vB_Stm Diag 54, vB_Stm Diag 55, vB_Stm Diag 56, vB_Stm Diag 57, vB_Stm Diag 58, vB_Stm Diag 59, vB_Stm Diag 60, vB_Stm Diag 61, vB_Stm Diag 62, vB_Stm Diag 63, vB_Stm Diag 64, vB_Stm Diag 65, vB_Stm. P. 1, vB_Stm. P. 2, vB_Stm. P. 3, vB_Stm. P. 4, Shigella sonnei, specifically, vB_Shs.Pod 3, vB_Eschc.Shis 7/20, vB_Eschc.Shis 1161, vB_Eschc.Shis 8963, vB_Eschc.Shis 8962, vB_Shis GE25, vB_Eschc.Shis W, vB_Shis G, vB_Shis 3/14, vB_Eschc.Shis 18, vB_Shis 1188, vB_Shis 1188 Γ, vB_Shis 1188 Y, vB_Shis 1188 X, vB_Shis 5514, vB_Shis L7-2, vB_Shis L7-4, vB_Shis L7-5, vB_Shis L7-11, vB_Shis K3, vB_Shis TuI A, vB_Shis Ox2, vB_Shis SCL, vB_Shis Bak C2, vB_Shis 4/1188, vB_Shis 8962, vB_Shis 8963, vB_Shis XIV, vB_Shis 116, vB_Shis 106/8, vB_Shis 20, vB_Shis 90/25, vB_Shis 87/25, vB_Shis 16/25, vB_Shs 7, vB_Shs 38, vB_Shs 92, vB_Shs 1391, vB_Shs. P. 1, vB_Shs. P. 2, vB_Shs. P. 3.

It should be appreciated that in some embodiments, the invention encompasses the use of any of the bacteriophages listed herein and disclosed herein, as well as any variants or hybrids thereof, as delivery vehicles to the components of the present disclosure, specifically, the microbiome modulating component (also indicated herein as modulating component, or modulatory component) and/or the selective component of the disclosed systems, as well as the compositions, kits and methods disclosed herein after.

Still further, it should be appreciated that any delivery vehicles may be used, specifically and phage-based delivery vehicle an any variants thereof. More specifically, “delivery vehicle variants” as herein, may differ one from the other by the content of their nucleic acid sequences and/or by the amino acid sequences of their proteins. Delivery vehicle variants may comprise at least one host recognition element (HRE) that may be homologous or heterologous, hybrid, native, mutated or any combinations thereof. Such HRE may be derived in some embodiments, from HREs two or more of any of any of the bacteriophages disclosed by the present disclosure. Specifically, these variants may carry a desirable host recognition element that is compatible with any target cell of interest. In yet some particular embodiments, useful HRE used in the delivery vehicles (transducing particles) of components of the systems of the present disclosure may comprise for example, at least one of the TC1 (as denoted by SEQ ID NO: 5), and/or TC20 (as denoted by SEQ ID NO: 6, or any variants thereof).

As indicated herein, the delivery vehicle applicable for transducing the modulatory component and/or the selective component as disclosed herein of the disclosed system, are compatible with a target cell, for example, a specific microbiome microorganism, more specifically, gut microbiome bacterial cells. It should be therefore understood that in case phage-based delivery vehicles are used, any native, variant, hybrid, mutated or modified bacteriophage may be used. Such bacteriophage may carry a modified, hybrid or native host recognition element that is compatible with the host cell. In some embodiments the host recognition element may comprise at least one protein, at least two proteins, at least three proteins or more, specifically, structural bacteriophage protein/s, either native or modified, that interact with the host receptor. In some specific embodiments, such structural bacteriophage protein may be a protein/s residing in the tail region of a bacteriophage. As known in the art, in bacteriophages the tail is a protein complex present in the majority of the phages and is involved in host recognition and genome delivery. Two main features are shared by tail structures: tails have a central tubular structure that forms the channel for DNA ejection, which is surrounded by fibers or spikes that are essential in the initial steps of host recognition. For example, the tail of T7 phage is assembled from a dodecamer (i.e. 12 copies) of gp 11 (the adaptor) and a hexamer (i.e. 6 copies) of gp12 (the nozzle), onto which six trimers of gp17 attach. T7's six tail fibers attach at the interface between the adaptor and nozzle, thus making contacts with both proteins. The adaptor ring is responsible for the attachment of the preformed tail to the prohead via interactions with the portal composed of 12 subunits of gp8. Bacteriophage components localized at the tail-end of the bacteriophage may be classified as “tail proteins” or “tail-tube proteins” (e.g. referring to gp 11 and gp12) and tail fiber (e.g. referring to gp17). As noted above, the host recognition element of the bacteriophage-based delivery vehicle may comprise at least one of these proteins, derived from any of the bacteriophages disclosed by the present disclosure.

Thus bacteriophage components localized at the tail-end of the bacteriophage may be classified as tail proteins (e.g. referring to gp11 and gp12) and tail fiber (e.g. referring to gp17). In specific embodiments the host-recognition element of the phage-based delivery vehicle used in the present disclosure may comprise at least one tail fiber or at least one tail protein.

In some embodiments the at least one protein residing in the tail region of such bacteriophage may be at least one of a tail protein and a fiber protein.

In specific embodiments, the host-recognition element herein described may comprise at least one of gp11, gp12 and gp17, or any combinations thereof. In some specific and non-limiting embodiments, these proteins may be, but not limited to, T7 gp17, gp 11 or gp12, any mutant thereof as described herein of or any native or mutated heterologous variants as explained below, or any combination thereof.

Any protein residing in the tail region of any naturally occurring bacteriophage that infects target cells as herein defined is encompassed by the present disclosure, specifically, any host recognition elements compatible with the desired target cell, as well as any combinations thereof.

It should be understood that the delivery vehicle that comprises, or used as, the modulatory or the selective component of the present disclosure may be modified bacteriophages, that may be also referred to herein as “modified particles”, “transducing particles”, “programed transducing particles”, “transducing vehicles”, “vehicles of the invention”, “bacteriophage or phage particles”, “delivery vehicles” and the like.

It should be understood that in some embodiments, where a bacteriophage-based transducing particles (e.g., bacteriophages, or hybrid phages or modified phages), such component may further comprise at least one packaging signal and any other suitable element that facilitates packaging in the delivery vehicle. As noted above, the nucleic acid sequence provided by the invention comprises a packaging signal. The term “packaging signal” as herein defined refers to a nucleotide sequence in e.g. a viral or bacteriophage genome that directs the packaging the of viral or bacteriophage genome into preformed capsids (envelops) during the infectious cycle.

In some embodiments, the systems of the present disclosure target microbiome organism/s in the microbiome cell population. For example, bacterial cells residing within the microbiome.

In yet some further embodiments, the target bacterial cell/s and/or cell population is, or comprised within gut microbiome cell population. In more specific embodiments, the cell population comprises at least one bacteria of the Proteobacteria, Firmicutes, Bacteroidetes, Actinobacteria, and p/al, or any mutant, variant or isolate or any combination thereof.

Prokaryotic cells according to the present disclosure encompass bacteria cells. The term “bacteria” (in singular a “bacterium”) in this context refers to any type of a single celled microbe. Herein the terms “bacterium” and “microbe” are interchangeable. This term encompasses herein bacteria belonging to general classes according to their basic shapes, namely spherical (cocci), rod (bacilli), spiral (spirilla), comma (vibrios) or corkscrew (spirochaetes), as well as bacteria that exist as single cells, in pairs, chains or clusters.

It should be noted that the term “bacteria” as used herein refers to any of the prokaryotic microorganisms that exist as a single cell or in a cluster or aggregate of single cells. In more specific embodiments, the term “bacteria” specifically refers to Gram positive, Gram negative or Acid-fast organisms. The Gram-positive bacteria can be recognized as retaining the crystal violet stain used in the Gram staining method of bacterial differentiation, and therefore appear to be purple-colored under a microscope. The Gram-negative bacteria do not retain the crystal violet, making positive identification possible. In other words, the term ‘bacteria’ applies herein to bacteria with a thicker peptidoglycan layer in the cell wall outside the cell membrane (Gram-positive), and to bacteria with a thin peptidoglycan layer of their cell wall that is sandwiched between an inner cytoplasmic cell membrane and a bacterial outer membrane (Gram-negative). This term further applies to some bacteria, such as Deinococcus, which stain Gram-positive due to the presence of a thick peptidoglycan layer, but also possess an outer cell membrane, and thus suggested as intermediates in the transition between monoderm (Gram-positive) and diderm (Gram-negative) bacteria. Acid fast organisms like Mycobacterium contain large amounts of lipid substances within their cell walls called mycolic acids that resist staining by conventional methods such as a Gram stain.

It should be however understood that when referring to “cells”, the present disclosure further encompasses in add-on to any of the prokaryotic cells exemplified and disclosed by the invention, in some specific embodiments other systems that imitate or mimic cells, artificial cells, vesicles and the like.

In some specific embodiments, the methods of the invention may be used for manipulating, editing and changing the microbiome of a subject in need.

The term “microbiome”, as used herein, refers to the ecological community of commensal, symbiotic, or pathogenic microorganisms in a sample. Examples of microbiomes that can be used with the present disclosure include but are not limited to skin microbiome, umbilical microbiome, vaginal microbiome, conjunctival microbiome, intestinal microbiome, stomach microbiome, gut microbiome and oral microbiome, nasal microbiome, gastrointestinal tract microbiome, and urogenital tract microbiome.

In some embodiments, the methods of the invention may be applicable in manipulating the gut microbiome in a subject. The term ‘gut microbiome’ (in the colloquial ‘gut flora’) encompasses a complex community of microorganism species that live in the digestive tracts of animals (in this case mammals). In this context gut is synonymous with intestinal and flora with microbiota and microflora. The gut microbiome refers to the genomes of the gut microbiota. Although the mammalian host can most probably survive without the gut flora, the relationship between the two is not merely commensal (a non-harmful coexistence), but rather mutualistic. The mammalian gut microflora fulfill a variety of useful functions, including digestion of unutilized energy substrates, stimulating cell growth, repressing the growth of harmful microorganisms, training the immune system to respond only to pathogens and defending against some diseases. In certain conditions, however, some species are capable of causing disease by producing infection or increasing risk for cancer, and other pathologies. Thus, by targeting specific subpopulation of the gut microbiome, the invention provides a therapeutic tailor-made tool for modulating conditions caused by certain microorganisms that are part of the gut microbiome, and/or product/s of interest that have a modulatory effect on the host and may be expressed by any target cell in the gut microbiome.

Composition and/or content of the mammalian gut microbiome consists predominantly of bacteria, for the most part anaerobic Gram positive and Gram negative strains, and to a lesser extent of fungi, protozoa, and archaea. Populations of bacterial species vary widely among different individuals, but are relatively constant within an individual over time, some alterations, however, may occur with changes in lifestyle, diet and age. Common evolutionary patterns in the composition of gut microbiome have been observed during life-time of human individuals. Gut microbiome composition and content can change following a long-term diet; and also depends on a geographic origin.

More specifically, when referring to composition or content of the human microbiome, or microbiota, is meant a composition with respect to the four predominant phyla of bacteria, namely Firmicutes, Bacteroidetes, Actinobacteria and Proteobacteria, or alternatively with respect to the predominant bacterial genera, namely Bacteroides, Clostridium, Fusobacterium, Eubacterium, Ruminococcus, Peptococcus, Peptostreptococcus and Bifidobacterium. Particularly the Bacteroides, which are the most predominant, may be important for host functioning. Other genera, such as Escherichia and Lactobacillus, although present to a lesser extent, were shown to contribute to host functioning.

Further, of particular relevance to the human gut microbiome is the enterotype classification basing on bacteriological ecosystem, which is independent of age, gender, body weight, or national divisions. There are three human enterotypes: Type 1 is characterized by high levels of Bacteroides (Gram negative); Type 2 has few Bacteroides, but Prevotella (Gram negative) are common; and Type 3 has high levels of Ruminococcus (Gram positive). Enterotypes, however, can be influenced by a long-term diet, for example, people having a high protein and fat diet are predominantly enterotype Type 1 and if changing their dietary patterns to a high carbohydrates diet—in the long-term become enterotype Type 2.

Thus, methods of the present disclosure pertain to the entire range of bacterial species constituting the mammalian gut microbiome, including qualitative as well as quantitative aspects thereof. They further pertain to less ubiquitous microbiome components, such as of fungi, the known genera include Candida, Saccharomyces, Aspergillus and Penicillium, as well as microorganisms belonging to the domain of Archaea (also Archaebacteria), and further yet unclassified species that cannot be cultured.

As indicate above, the disclosed systems comprise at least two components, the selective component and the modulating component.

The “Selective component” as used herein, refers to an element or component of the system of the invention that enables, facilitates, leads to and acts on selecting, choosing, electing or enriching a specific population of bacterial cells, specifically, a population of cells that carry the cas-CRISPR system of the invention, more specifically, a population of bacterial cells that carry the microbiome modulating component of the present disclosure. The selective component provides selective advantage to the desired population, for example by imposing conditions that enable and allow only the survival, and/or growth, and/or functioning of the selected desired population. In specific embodiments, any population or cells that carry the modulating component of the present disclosure.

As indicated herein, in some embodiments, the selective component of the present disclosure comprises a toxic element that kill, inhibit, or reduces the target cells (e.g., bacterial cells) growth, viability and/or function. It should be appreciated that the terms “inhibition”, “moderation”, “reduction”, “decrease” or “attenuation”, “prevention”, “suppression”, “repression”, “elimination” as referred to herein, relate to the retardation, restraining or reduction of a process (e.g., growth, viability and/or function) by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more. With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively. In some specific and non-limiting embodiments, a selective component may be a lytic bacteriophage. In some further embodiments, the lytic page may be in some embodiments, a T7 bacteriophage. In more specific embodiments, such bacteriophage may comprise the nucleic acid sequence as denoted by SEQ ID NO: 22, or any homologs or variants thereof. Still further, in some alternative embodiments, the selective component used in the disclosed systems may comprise a transducing particle, or any phage-like particle comprising at least one toxic element that attenuates, and/or inhibits bacterial growth or even in some embodiments, kill bacteria. In some embodiments such element may comprise nucleic acid sequences encoding any lytic protein, for example, bacteriophage lytic proteins, such as the Gp0.4 protein, for example, as denoted by SEQ ID NO: 23. In yet some further additional or alternative embodiments, the toxic protein may be the Gp0.6 protein, for example, as denoted by SEQ ID NO: 24. In yet some further alternative embodiments, a selective component useful in the systems of the present disclosure may comprise the nucleic acid sequence as denoted by SEQ ID NO: 7, or any variants or derivatives thereof. It should be understood that the disclosed selective components comprise at least one protospacer (e.g., within essential genes and/or control elements) that are targeted by the spacers comprised within the microbiome modulating component of the disclosed systems.

“Modulating component”, “microbiome modulating component” or “metabolism modulating component” as used herein, refers to an element or component of the system of the present disclosure that enables, facilitates, leads to, and/or acts on modulating, a specific population of bacterial cells, specifically in the microbiome to increase, initiate, attenuate and/or cease the production of a product of interest. Moreover, the modulating component comprise a cas-CRISPR system that prevents from the selective component to eliminate the desired population, specifically any population or cells that carry the drug microbiome or metabolism modulating component of the present disclosure. In yet some further embodiments, the modulating component of the present disclosure leads to the expression, activation, functionalization, or alternatively, elimination or attenuation of at least one product of interest that may affect either directly or indirectly various systems in the host that carry the bacterial population manipulated by the disclosed systems (e.g., immune-system, metabolism). As such, in some embodiments, the modulatory component of the present disclosure may be considered as a modulatory component, at the level of the host that carry the target bacterial population.

It should be understood that any of the modulating components and/or selective components disclosed by the systems of the present disclosure, are also applicable for any of the methods, compositions and kits disclosed herein after. As indicated above, one of the two major elements of the modulating component of the systems of the present disclosure is the CRISPR array that enables the enrichment of target cells that are transduced by and carry the modulating component. Moreover, at least one spacer of the CRISPR array of the invention may be sufficiently complementary to a nucleic acid sequence (or a proto-spacer) comprised as the nucleic acid sequence of interest within the selective component of the invention or any systems thereof, so as to target and inactivate the selective component, where “inactivate” means delay, decrease, inhibit, eliminate, attenuate or stop the activity of the selective component. It should be noted that such inactivation renders a bacterium comprising the microbiome modulating component insensitive and resistant to the selective component of the invention or systems thereof. It should be appreciated that sufficient complementarity as used herein reflects any complementarity of between about 10% to 100%, more specifically, complementarity of about 10%, 15%, 20%, 25%, 30%, 35%, 40%, 45%, 50%, 65%, 70%, 75%, 80%, 85%, 90%, 95%, 96%, 97%, 98%, 99% and 100%.

In certain embodiments, “Complementarity” refers to a relationship between two structures each following the lock-and-key principle. In nature complementarity is the base principle of DNA replication and transcription as it is a property shared between two DNA or RNA sequences, such that when they are aligned antiparallel to each other, the nucleotide bases at each position in the sequences will be complementary (e.g., A and T or U, C and G).

Still further, in certain embodiments, the microbiome modulating component of the present disclosure may comprise as a nucleic acid sequence of interest, at least one CRISPR spacer that targets at least one nucleic acid sequence comprised within said selective component (that may be in some embodiment, a bacteriophage encoding toxin that comprise the protospacer, or essential genes of a lytic bacteriophage) and at least one CRISPR spacer that targets a nucleic acid sequence comprised within said at least one pathogenic or undesired gene. In such way the microbiome modulating component of the present disclosure may target and/or inactivate both, the lytic phage/s that serve as the selective component and the element participating in metabolism and/or the production of at least one product of interest.

As used herein, CRISPR arrays also known as SPIDRs (Spacer Interspersed Direct Repeats) constitute a family of DNA loci that are usually specific to a particular bacterial species. The CRISPR array is a distinct class of interspersed short sequence repeats (SSRs) that were first recognized in E. coli. In subsequent years, similar CRISPR arrays were found in Mycobacterium tuberculosis, Haloferax mediterranei, Methanocaldococcus jannaschii, Thermotoga maritima and other bacteria and archaea. It should be understood that the present disclosure contemplates the use of any of the known CRISPR systems, particularly and of the CRISPR systems disclosed herein after. More specifically, the Clustered Regularly Interspaced Short Palindromic Repeats (CRISPR) system is a bacterial immune system that has been modified for genome engineering. CRISPR-Cas systems fall into two classes. Class 1 systems use a complex of multiple Cas proteins to degrade foreign nucleic acids. Class 2 systems use a single large Cas protein for the same purpose. More specifically, Class 1 may be divided into types I, III, and IV and class 2 may be divided into types II, V, and VI. The CRISPR-Cas system has evolved in prokaryotes to protect against phage attack and undesired plasmid replication by targeting foreign DNA or RNA. In bacterial immunity, the CRISPR-Cas system, targets DNA molecules based on short homologous DNA sequences, called spacers that have previously been extracted by the bacterium from the foreign pathogen sequence and inserted between repeats as a memory system. These spacers are transcribed and processed and this RNA, named crRNA or guide-RNA (gRNA), guides CRISPR-associated (Cas) proteins to matching (and/or complementary) sequences within the foreign DNA, called proto-spacers, which are subsequently cleaved. The spacers, or other suitable constructs or RNAs can be rationally designed and produced to target any DNA sequence. Moreover, this recognition element may be designed separately to recognize and target any desired target.

As used herein, the phrase “CRISPR array polynucleotide” refers to a DNA or RNA segment which comprises sufficient CRISPR repeats such that it is capable of down regulating (e.g. eliminating, targeting) a complementary gene.

According to some embodiments, the CRISPR array polynucleotide comprised in the modulating component of the disclosed systems may comprise at least two repeats with one spacer between them. In yet some further embodiments, the CRISPR array of the microbiome modulating component of the present disclosure may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more, specifically, 110, 120, 130, 140, 150, 160, 170, 180, 190, 200 or more spacers. It should be further understood that the spacers of the microbiome modulating component of the present disclosure may be either identical or different spacers. In more embodiments, these spacers may target either an identical or different target undesired gene/s. In yet some other embodiments, such spacer may target at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more undesired gene/s.

In an exemplary embodiment, the CRISPR array polynucleotide comprises all of the CRISPR repeats, starting with the first nucleotide of the first CRISPR repeat and ending with the last nucleotide of the last (terminal) repeat.

It should be further understood that the CRISPR array used in the modulating components of the disclosed systems may be used not only to target and/or inactivate the selective component of the disclosed systems, but also to target and manipulate at least one product of interest that has modulatory activity on the host. For example, to inactivate or alternatively activate an element that directly or indirectly the product of interest or any target that affects the product of interest.

The disclosed CRISPR array comprises at least one spacer. As used herein, the term “spacer” refers to a non-repetitive spacer sequence that is found between multiple short direct repeats (i.e., CRISPR repeats) of CRISPR arrays. In some embodiments, CRISPR spacers are located in between two identical CRISPR repeats.

In some embodiments, CRISPR spacer is naturally present in between two identical, short direct repeats that are palindromic. It should be noted that the spacers of the invention may be located or present between two identical or not identical repeats, and moreover, these spacers encode crRNA that targets the proto-spacer within the selective component and/or protospacers within a target nucleic acid sequence that affect directly or indirectly the product of interest. In yet some alternative embodiments, the modulatory component of the disclosed systems may comprise at least one spacer, or in other words, at least one nucleic acid sequence encoding at least one gRNA that targets at least one protospacer within the selective component, and optionally in addition, at least one spacer, or in other words, at least one nucleic acid sequence encoding at least one gRNA that targets a target sequence that affect either directly or indirectly the production, stability, activity, affinity of the at least one product of interest.

Still further, in addition to the CRISPR array, specifically, at least one spacer, or in other words, at least one nucleic acid sequence encoding at least one gRNA, in some specific and non-limiting embodiments, the disclosed microbiome modulating component may further comprise at least one nucleic acid sequence that encodes at least one cas protein. In some embodiments, the modulatory component is indicated herein as comprising at least one cas gene. As used herein, the term “cas gene” refers to the genes that are generally coupled, associated or close to or in the vicinity of flanking CRISPR arrays that encode at least one Cas protein.

As indicated above, it should be understood that the present disclosure contemplates the use of any of the known CRISPR systems, particularly and of the CRISPR systems disclosed herein.

In some embodiments, a CRISPR system useful in the modulating component of the present disclosure may be of Class 1, for example, a Type I system. Thus, in some embodiments, the modulating component of the disclosed systems may comprise a Cas-CRISPR type I system, specifically, CRISPR array and at least one nucleic acid sequence encoding at least one Cas protein of Type I. More specifically, Type I CRISPR-Cas systems contain the cas3 gene, which encodes a large protein with separate helicase and DNase activities, in addition to genes encoding proteins that probably form Cascade-like complexes with different compositions. These complexes contain numerous proteins that have been included in the repeat-associated mysterious proteins (RAMPs), which form a large superfamily of Cas proteins, and contain at least one RNA recognition motif (RRM; also known as a ferredoxin-fold domain) and a characteristic glycine-rich loop. RAMP superfamily encompasses the large Cas5 and Cas6 families on the basis of extensive sequence and structure comparisons. Furthermore, the Cas7 (COG1857) proteins represent another distinct, large family within the RAMP superfamily.

The type I CRISPR-Cas systems seem to target DNA where the target cleavage is catalyzed by the HD nuclease domains of Cas3. As the RecB nuclease domain of Cas4 is fused to Cas1 in several type I CRISPR-Cas systems, Cas4 could potentially play a part in spacer acquisition instead. It should be noted that any type I CRISPR-Cas systems may be applicable in the present invention, specifically, any one of type I-A, B, C, D, E, and F.

In some specific embodiment, the CRISPR-Cas proteins used in microbiome modulating component of the systems of the present disclosure may be of a CRISPR Class 2 system. In yet some further particular embodiments, such class 2 system may be any one of CRISPR type II, and type V systems. In certain embodiments, the Cas applicable in the present disclosure may be any Cas protein of the CRISPR type II system. The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cas1 and Cas2. Cas9 contains at least two nuclease domains, a RuvC-like nuclease domain near the amino terminus and the HNH (or McrA-like) nuclease domain in the middle of the protein, but the function of these domains remains to be elucidated. However, as the HNH nuclease domain is abundant in restriction enzymes and possesses endonuclease activity, it is likely to be responsible for target cleavage.

Type II systems cleave the pre-crRNA through an unusual mechanism that involves duplex formation between a tracrRNA and part of the repeat in the pre-crRNA; the first cleavage in the pre-crRNA processing pathway subsequently occurs in this repeat region. This cleavage is catalyzed by the housekeeping, double-stranded RNA-specific RNase III in the presence of Cas9. Still further, type II system comprise at least one of cas9, cas1, cas2 csn2, and cas4 genes. It should be appreciated that any type II CRISPR-Cas systems may be applicable in the present invention, specifically, any one of type II-A or B.

The type III CRISPR-Cas systems contain polymerase and RAMP modules in which at least some of the RAMPs seem to be involved in the processing of the spacer-repeat transcripts, analogous to the Cascade complex. Type III systems can be further divided into sub-types III-A (also known as Mtube or CASS6) and III-B (also known as the polymerase-RAMP module). Subtype III-A systems can target plasmids, as has been demonstrated in vivo for S. epidermidis, and it seems plausible that the HD domain of the polymerase-like protein encoded in this subtype (COG1353) might be involved in the cleavage of target DNA. There is strong evidence that, at least in vitro, the type III-B CRISPR-Cas systems can target RNA, as shown for a subtype III-B system from furiosus. It should be appreciated that any cas gene that belongs to the type III CRISPR system may be used for the purpose of the invention, for example, any one of cas6, cas10, csm2, csm3, csm4, csm5, csm6, cmr1, cmr3, cmr4, cmr5, cmr6, cas1 and cas2. Still further, any one of typeIII-A or typeIII-B systems may be used for the systems, components and method of the invention.

In yet some further particular embodiments, the class 2 system in accordance with the invention, may be a CRISPR type V system. In a more specific embodiment, the RNA guided DNA binding protein nuclease may be CRISPR-associated endonuclease X (CasX) system or CRISPR-associated endonuclease 14 (Cas14) system or CRISPR-associated endonuclease F (CasF, also known as Cas12j) system. The type V CRISPR-Cas systems are distinguished by a single RNA-guided RuvC domain-containing nuclease. As with type II CRISPR-Cas systems, CRISPR type V system as used herein requires the inclusion of two essential components: a gRNA and a CRISPR-associated endonuclease (CasX/Cas14/CasF). The gRNA is a short synthetic RNA composed of a “scaffold” sequence necessary for CasX/Cas14/CasF-binding and about 20 nucleotide long “spacer” or “targeting” sequence, which defines the genomic target to be modified.

In some particular embodiments, the at least one cas gene in the CRISPR-Cas system used in the modulating component of the disclosed systems, may be at least one cas gene of type I-E CRISPR system. The “type-IE CRISPR” system refers to native to K-type Escherichia coli. It has been shown to inhibit phage infection, cure plasmids, prevent conjugal element transfer and kill cells. This CRISPR machinery can be used to degrade specific intracellular DNA in an inducible and targeted manner, leaving the remainder DNA intact. In yet some specific embodiments, the CRISPR-Cas system used in the systems, specifically in the microbiome modulating component of the systems of the present disclosure my be the type-IE system of E. coli. Still further in some embodiments, the modulating component of the present disclosure may comprise at least one nucleic acid sequence encoding any cas gene required for a functional type-I-E system. In some embodiments, the modulating component may comprise at least one nucleic acid sequence encoding the cas3 nuclease. Still further, in some embodiments, the cas3 may be the E. coli cas3. In yet some further embodiments, the nucleic acid sequence encoding cas3 used in the modulatory component of the present disclosure may comprise the nucleic acid sequence as denoted by SEQ ID NO: 31, or any variants, homologs and derivatives thereof. In yet some further embodiments, the modulating component may further comprise at least one nucleic acid sequence encoding at least one additional E. coli Type-I-E system, in addition to cas3, specifically, at least one of CasA (also known as cse1), CasB (also known as (cse2), CasC (also known as cas4), CasD (also known as cas5), CasE (also known as cas6e) and the like. In some specific embodiments, the additional case proteins may be encoded by the nucleic acid sequence as denoted by at least one of SEQ ID NO: 32, 33, 34, 35 and 36, respectively.

As indicated above, any CRISPR/Cas proteins may be used by the modulating component of the systems of the present disclosure. Still further, in some embodiments of the present disclosure, the cas protein encoded by the nucleic acid sequence comprised within the modulating component may be at least one of Cas9, CasX, Cas12, Cas13, Cas14, Cas6, Cpf1, CMS1 protein, or any variant thereof that is derived or expressed from Methanococcus maripaludis C7, Corynebacterium diphtheria, Corynebacterium efficiens YS-314, Corynebacterium glutamicum (ATCC 13032), Corynebacterium glutamicum (ATCC 13032), Corynebacterium glutamicum R, Corynebacterium kroppenstedtii (DSM 44385), Mycobacterium abscessus (ATCC 19977), Nocardia farcinica IFM10152, Rhodococcus erythropolis PR4, Rhodococcus jostii RFIA1, Rhodococcus opacus B4 (uid36573), Acidothermus cellulolyticus 11 B, Arthrobacter chlorophenolicus A6, Kribbella flavida (DSM 17836), Thermomonospora curvata (DSM43183), Bifidobacterium dentium Bdl, Bifidobacterium longum DJO10A, Slackia heliotrinireducens (DSM 20476), Persephonella marina EX H 1, Bacteroides fragilis NCTC 9434, Capnocytophaga ochracea (DSM 7271), Flavobacterium psychrophilum JIP02 86, Akkermansia muciniphila (ATCC BAA 835), Roseiflexus castenholzii (DSM 13941), Roseiflexus RS1, Synechocystis PCC6803, Elusimicrobium minutum Pei191, uncultured Termite group 1 bacterium phylotype Rs D17, Fibrobacter succinogenes S85, Bacillus cereus (ATCC 10987), Listeria innocua, Lactobacillus casei, Lactobacillus rhamnosus GG, Lactobacillus salivarius UCC118, Streptococcus agalactiae-5-A909, Streptococcus agalactiae NEM316, Streptococcus agalactiae 2603, Streptococcus dysgalactiae equisimilis GGS 124, Streptococcus equi zooepidemicus MGCS10565, Streptococcus gallolyticus UCN34 (uid46061), Streptococcus gordonii Challis subst CH1, Streptococcus mutans NN2025 (uid46353), Streptococcus mutans, Streptococcus pyogenes M1 GAS, Streptococcus pyogenes MGAS5005, Streptococcus pyogenes MGAS2096, Streptococcus pyogenes MGAS9429, Streptococcus pyogenes MGAS 10270, Streptococcus pyogenes MGAS6180, Streptococcus pyogenes MGAS315, Streptococcus pyogenes SSI-1, Streptococcus pyogenes MGAS10750, Streptococcus pyogenes NZ131, Streptococcus thermophiles CNRZ1066, Streptococcus thermophiles LMD-9, Streptococcus thermophiles LMG 18311, Clostridium botulinum A3 Loch Maree, Clostridium botulinum B Eklund 17B, Clostridium botulinum Ba4 657, Clostridium botulinum F Langeland, Clostridium cellulolyticum H10, Finegoldia magna (ATCC 29328), Eubacterium rectale (ATCC 33656), Mycoplasma gallisepticum, Mycoplasma mobile 163K, Mycoplasma penetrans, Mycoplasma synoviae 53, Streptobacillus, moniliformis (DSM 12112), Bradyrhizobium BTAil, Nitrobacter hamburgensis X14, Rhodopseudomonas palustris BisB18, Rhodopseudomonas palustris BisB5, Parvibaculum lavamentivorans DS-1, Dinoroseobacter shibae. DFL 12, Gluconacetobacter diazotrophicus Pal 5 FAPERJ, Gluconacetobacter diazotrophicus Pal 5 JGI, Azospirillum B510 (uid46085), Rhodospirillum rubrum (ATCC 11170), Diaphorobacter TPSY (uid29975), Verminephrobacter eiseniae EF01-2, Neisseria meningitides 053442, Neisseria meningitides alpha14, Neisseria meningitides Z2491, Desulfovibrio salexigens DSM 2638, Campylobacter jejuni doylei 269 97, Campylobacter jejuni 81116, Campylobacter jejuni, Campylobacter lari RM2100, Helicobacter hepaticus, Wolinella succinogenes, Tolumonas auensis DSM 9187, Pseudoalteromonas atlantica T6c, Shewanella pealeana (ATCC 700345), Legionella pneumophila Paris, Actinobacillus succinogenes 130Z, Pasteurella multocida, Francisella tularensis novicida U 112, Francisella tularensis holarctica, Francisella tularensis FSC 198, Francisella tularensis, Francisella tularensis WY96-3418, or Treponema denticola (ATCC 35405).

Still further, the present disclosure may further encompass in some embodiments, the use of at least one defective cas protein, specifically, where target sequences that modulate the product of interested are also targeted by the CRISPR system of the modulatory component. A defective cas protein (e.g., a defective mutant, variant or fragment) may relate to an enzyme that displays an activity reduced in about 1%, 2%, 3%, 4%, 5% to about 100%, specifically, about 5% to about 10%, about 10% to about 15%, about 15% to about 20%, about 20% to about 25%, about 25% to about 30%, about 35% to about 40%, about 40% to about 45%, about 45% to about 50%, about 50% to about 55%, about 55% to about 60%, about 65% to about 70%, about 75% to about 80%, about 80% to about 85%, about 85% to about 90%, about 90% to about 95%, about 95% to about 99.9%, more specifically, reduced activity of about 98% to about 100%, as compared to the wild type active nuclease. More specifically, an enzyme that displays an activity reduced in about 1%, 2%, 3%, 4%, 5%, 6%, 7%, 8%, 9%, 10%, 11%, 12%, 13%, 14%, 1%, 6%, 17%, %18%, %19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 99.99%, 99.999%, 99.9999% or about 100%, as compared to the wild type active nuclease.

In some embodiments, the microbiome metabolism modulating component of the disclosed system affects and/or modulates, and/or changes at least one of the expression, production, activity, bioavailability, clearance, stability, toxicity, and absorption of the product of interest. As will be detailed herein after, in some embodiments, the product of interest has a modulatory effect on the host itself.

Still further, in some embodiments, the modulatory component modulates, for example, increases, elevates, enhances, or alternatively decrease, attenuate or inhibit, at least one of the expression, production, activity, bioavailability, clearance, stability, toxicity, and absorption of the product of interest. More specifically, the terms “increase”, “elevation”, “enlarge”, “elevate” or “expend”, “improve”, “as referred to herein, relate to the elevation and increase of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more. In the same manner, it should be appreciated that the terms “inhibition”, “moderation”, “reduction”, “decrease” or “attenuation”, “prevention”, “suppression”, “repression”, “elimination” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

More specifically, Expression or gene expression, as used herein, refers to the process by which information from a gene is used in the synthesis of a functional gene product that enables it to produce proteins or non-coding RNA, and ultimately affect a phenotype obtained by the product of interest. These gene products are often proteins, but in non-protein-coding genes such as transfer RNA (tRNA) and small nuclear RNA (snRNA), the product is a functional non-coding RNA. It should be further understood that this term encompasses all steps in the gene expression process that may be modulated and/or regulated, including the transcription, RNA splicing, translation, and post-translational modification of a protein. Regulation of gene expression gives control over the timing, location, and amount of a given gene product (protein or ncRNA) present in a cell and can have a profound effect on the cellular structure and function. Still further, in some embodiments, the modulating component may modulate the production of the product of interest. Production, refers herein to the is the process of generating a specific product of interest. It is typically achieved by the manipulation of gene expression in an organism such that it expresses large amounts of the gene product. This includes the transcription of the desired DNA to messenger RNA (mRNA), the translation of mRNA into polypeptide chains, which are ultimately folded into functional proteins and may be targeted to specific subcellular or extracellular location.

Still further, in some embodiments, the modulating component may modulate the activity of the product of interest. Activity refers herein to the functional capability and/or effectiveness of a substance in carrying out its specific biochemical or biology function.

Still further, in some embodiments, the modulating component may modulate the bioavailability of the product of interest. Bioavailability as used herein is a subcategory of absorption and is the fraction (%) of a desired product that reaches the systemic circulation, and is available for performing its biological action. Bioavailability of a desired product is generally affected by intestinal endothelium absorption and first-pass metabolism.

In some embodiments, the modulating component may modulate the clearance of the product of interest. Clearance is a pharmacokinetic measurement of the volume of plasma from which a product of interest is completely removed per unit time. Usually, clearance is measured in L/h or mL/min. The quantity reflects the rate of product of interest elimination divided by plasma concentration. Excretion, on the other hand, is a measurement of the amount of a substance removed from the body per unit time (e.g., mg/min, g/min, etc.).

A product of interest in the body can be cleared by various organs, including the kidneys, liver, lungs, etc. Thus, total body clearance is equal to the sum clearance of the substance by each organ (e.g., renal clearance+hepatic clearance+lung clearance=total body clearance). For many products, however, clearance is solely a function of renal excretion.

Still further, in some embodiments, the modulating component may modulate the stability of the product of interest. Stability is used herein is the ability of a product to retain its chemical, physical, properties and/or activity within specified time limits.

Still further, in some embodiments, the modulating component may modulate the toxicity of the product of interest. Toxicity is the degree to which a product of interest or a particular mixture of substances can damage an organism. Toxicity can refer to the effect on a whole organism, such as an animal, bacterium, or plant, as well as the effect on a substructure of the organism, such as a cell (cytotoxicity) or an organ such as the liver (hepatotoxicity).

In some embodiments, the modulating component may modulate the absorption of the product of interest. Absorption is the journey of a product of interest travelling from the site of administration to the site of action.

In some embodiments, the product of interest may be at least one of: a product affecting and/or modulating immune-related processes and/or conditions, a metabolic product and/or a product affecting and/or modulating a metabolic process or condition/s, a product affecting and/or modulating neurological or neuronal and/or neuro-behavioral processes or conditions, a therapeutic product, a cosmetic product and a nutritional product.

In some embodiments, the product is a protein or polypeptide product encoded by a sequence comprised within at least one nucleic acid sequence of said microbiome modulating component of (a)(i). Alternatively, or additionally, the product may be produced directly or indirectly by an element encoded by the nucleic acid sequence of said microbiome modulating component of (a)(i).

Still further, in some embodiments, the product of interest is a product affecting and/or modulating immune-related processes and/or conditions. In yet some further embodiments, the product is a protein or polypeptide product encoded by a sequence comprised within at least one nucleic acid sequence of the microbiome modulating component of the system as define by (a)(i).

Immune-related processes and/or conditions, “Immune-related disorder” or “Immune-mediated disorder”, as used herein encompasses any condition, or process that is associated with the immune system of a subject. More specifically, any process or condition acting through inhibition or the activation of the immune system, or that can be treated, prevented, or ameliorated by reducing degradation of a certain component of the immune response in a subject, such as the adaptive or innate immune response. More specifically, an ‘immune-related disorder’, as meant herein, encompasses a range of dysfunctions of the innate and adaptive immune systems. In more specific terms, immune-related disorder can be characterized, for example, (1) by the component(s) of the immune system; (2) by whether the immune system is overactive or underactive; (3) by whether the condition is congenital or acquired, as will be specified herein after.

Accordingly, in some embodiments, the immune-related condition is an inflammatory condition. The terms “inflammatory condition” or “inflammatory-associated condition” refers to any disease or pathologically condition which can benefit from the reduction of at least one inflammatory parameter, for example, induction of an inflammatory cytokine such as IFN-γ and reduction in IL-6, IL-4, and/or IL-10 levels. The condition may be caused (primarily) from inflammation, or inflammation may be one of the manifestations of the diseases caused by another physiological cause.

In yet some further embodiments, the product of interest encoded and/or modulated by the microbiome modulating component of the system of the present disclosure is at least one cytokine. Cytokines are a broad and loose category of small proteins (typically ~5-25 kDa) important in cell signaling. Due to their size, cytokines cannot cross the lipid bilayer of cells to enter the cytoplasm and therefore typically exert their functions by interacting with specific cytokine receptors on the target cell surface. Cytokines have been shown to be involved in autocrine, paracrine and endocrine signaling as immunomodulating agents. Cytokines include chemokines, interferons, interleukins, lymphokines, and tumour necrosis factors, but generally not hormones or growth factors (despite some overlap in the terminology). Cytokines are produced by a broad range of cells, including immune cells like macrophages, B lymphocytes, T lymphocytes and mast cells, as well as endothelial cells, fibroblasts, and various stromal cells; a given cytokine may be produced by more than one type of cell. They act through cell surface receptors and are especially important in the immune system; cytokines modulate the balance between humoral and cell-based immune responses, and they regulate the maturation, growth, and responsiveness of particular cell populations. Some cytokines enhance or inhibit the action of other cytokines in complex ways. Cytokines are important in health and disease, specifically in host immune responses to infection, inflammation, trauma, sepsis, cancer, and reproduction. In some embodiments, a cytokine used as the product of interest by the present disclosure may be any cytokine known in the art. In some embodiments, the cytokines may be anti-inflammatory cytokines. More specifically, at least one of IL-10, IL-2, IL-17, IL-4, IL-11, and IL-13. Leukemia inhibitory factor, interferon-alpha, IL-6, and transforming growth factor (TGF)-β, IL-19, IL-20, IL-22, IL-24 (Mda-7), IL-26, IL-28A, IL-28B, IL-29 and IL-35. In yet some further embodiments, proinflammatory may be used herein as the product of interest, for example, interleukin-I (IL-1), IL-6, IL-12, and IL-18, tumor necrosis factor alpha (TNF-α), interferon gamma (IFNγ), and granulocyte-macrophage colony stimulating factor (GM-CSF). In more specific embodiments, the cytokine, used as a product of interest may be at least one of Interleukine-10 (IL-10) Interleukine-2 (IL-2), and Interleukine-17 (IL-17).

Interleukin 10 (IL-10), also known as human cytokine synthesis inhibitory factor (CSIF), is an anti-inflammatory cytokine. In humans, interleukin 10 is encoded by the IL10 gene. IL-10 signals through a receptor complex consisting of two IL-10 receptor-1 and two IL-10 receptor-2 proteins. Consequently, the functional receptor consists of four IL-10 receptor molecules. IL-10 binding induces STAT3 signalling via the phosphorylation of the cytoplasmic tails of IL-10 receptor 1 and IL-10 receptor 2 by JAKI and Tyk2 respectively. IL-10 is classified as a class-2 cytokine, a set of cytokines including IL-19, IL-20, IL-22, IL-24 (Mda-7), IL-26 and interferons type-I (IFN-alpha, -beta, -epsilon, -kappa, -omega), type-II (IFN-gamma) and type-III (IFN-lambda, also known as IL-28A, IL-28B, and IL-29). IL-10 is a cytokine with multiple, pleiotropic, effects in immunoregulation and inflammation. For examples, it downregulates the expression of Th1 cytokines, MHC class II antigens, and co-stimulatory molecules on macrophages. It also enhances B cell survival, proliferation, and antibody production. IL-10 can block NF-κB activity, and is involved in the regulation of the JAK-STAT signaling pathway.

Still further, Interleukin-2 (IL-2) is an interleukin, a type of cytokine signaling molecule in the immune system, which stimulate the growth of helper, cytotoxic and regulatory T cells. It is a 15.5-16 kDa protein that regulates the activities of white blood cells (leukocytes, often lymphocytes) that are responsible for immunity. IL-2 is a member of a cytokine family, each member of which has a four alpha helix bundle; the family also includes IL-4, IL-7, IL-9, IL-15 and IL-21. IL-2 signals through the IL-2 receptor, a complex consisting of three chains, termed alpha (CD25), beta (CD122) and gamma (CD132). The gamma chain is shared by all family members. IL-2 is part of the body's natural response to microbial infection, and in discriminating between foreign (“non-self”) and “self”. IL-2 mediates its effects by binding to IL-2 receptors, which are expressed by lymphocytes. The major sources of IL-2 are activated CD4⁺ T cells and activated CD8⁺ T cells.

Interleukin 17 family (IL17 family) is a family of pro-inflammatory cystine knot cytokines. They are produced by a group of T helper cell known as T helper 17 cell in response to their stimulation with IL-23. The protein encoded by IL17A is a founding member of IL-17 family. In rodents, IL-17A is often referred to as CTLA8. The biologically active IL-17 interacts with type I cell surface receptor IL-17R. In turn, there are at least three variants of IL-17R referred to as IL17RA, IL17RB, and IL17RC. After binding to the receptor, IL-17 activates several signaling cascades that, in turn, lead to the induction of chemokines. Acting as chemoattractant, these chemokines recruit the immune cells, such as monocytes and neutrophils to the site of inflammation. Typically, the signaling events mentioned above follow an invasion of the body by pathogens. Promoting the inflammation, IL-17 acts in concert with tumor necrosis factor and interleukin-1. Moreover, an activation of IL-17 signaling is often observed in the pathogenesis of various autoimmune disorders, such as psoriasis. Numerous immune regulatory functions have been reported for the IL-17 family of cytokines, presumably due to their induction of many immune signaling molecules. The most notable role of IL-17 is its involvement in inducing and mediating proinflammatory responses.

More specifically, in some embodiments, product of interest may be IL-10.

Still further, in some embodiments, the IL-10 as indicated by the present disclosure refers to the human IL-10. In some embodiments, the human IL-10 having the Gene ID: 3586 may be used as the product of interest in accordance with the present disclosure.

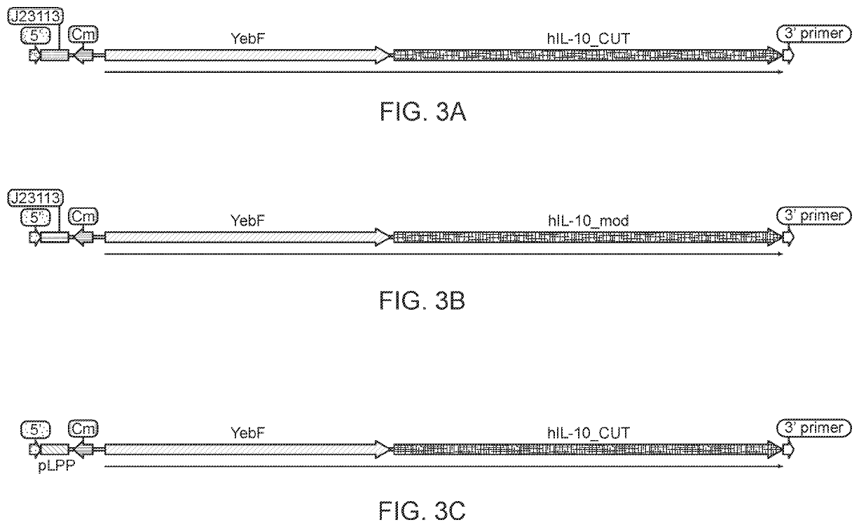

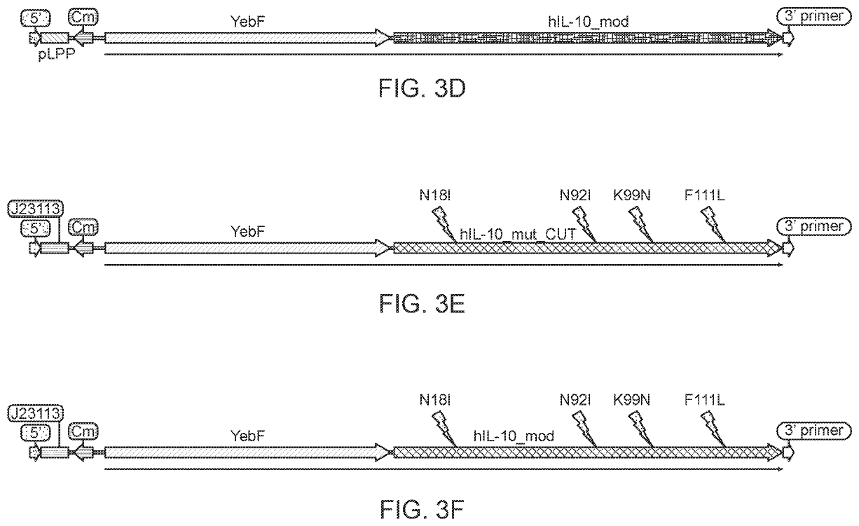

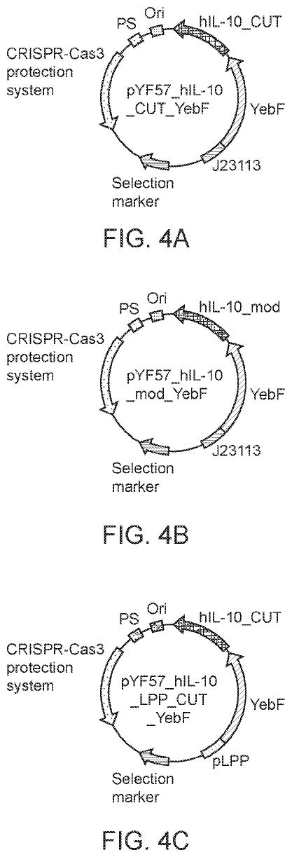

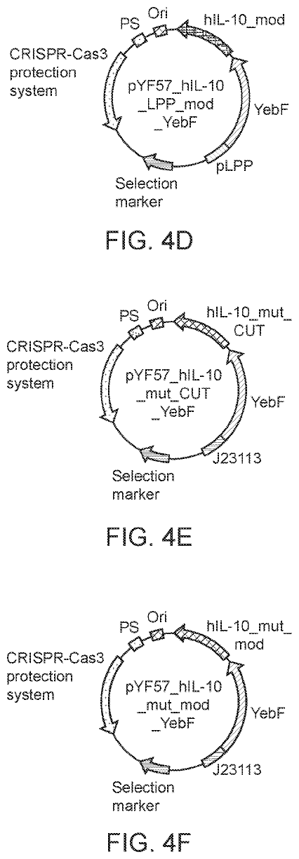

Still further, in some embodiments, the human IL-10 used in the modulating component may be encoded by the nucleic acid sequence as denoted by SEQ ID NO: 4, or any variants or derivatives thereof. Still further, in some embodiments, the human IL-10 used in the modulating component of the present disclosure may be the human IL-10 encoded by a nucleic acid sequence comprising SEQ ID NO: 25, or any variants or derivatives thereof (also referred to herein as hIL-10_CUT). In some embodiments, this IL-10 variant is provided in the modulatory component of the present disclosure comprised within a construct comprising the nucleic acid sequence as denoted by SEQ ID NO: 12, or any variants or derivatives thereof (as presented by FIGS. 3A and 4A).

Still further, in some embodiments, the human IL-10 used in the modulating component of the present disclosure may be the human IL-10 encoded by a nucleic acid sequence comprising SEQ ID NO: 26, or any variants or derivatives thereof (also referred to herein as hIL-10_mod). In some embodiments, this IL-10 variant is provided in the modulatory component of the present disclosure comprised within a construct comprising the nucleic acid sequence as denoted by SEQ ID NO: 13, or any variants or derivatives thereof (as presented by FIGS. 3B and 4B).

In some embodiments, the human IL-10 used in the modulating component of the present disclosure may be the human IL-10 encoded by a nucleic acid sequence comprising SEQ ID NO: 27, or any variants or derivatives thereof (also referred to herein as hIL-10_LPP_CUT). In some embodiments, this IL-10 variant is provided in the modulatory component of the present disclosure comprised within a construct comprising the nucleic acid sequence as denoted by SEQ ID NO: 14, or any variants or derivatives thereof (as presented by FIGS. 3C and 4C).

In some embodiments, the human IL-10 used in the modulating component of the present disclosure may be the human IL-10 encoded by a nucleic acid sequence comprising SEQ ID NO: 28, or any variants or derivatives thereof (also referred to herein as hIL-10_LPP_mod). In some embodiments, this IL-10 variant is provided in the modulatory component of the present disclosure comprised within a construct comprising the nucleic acid sequence as denoted by SEQ ID NO: 15, or any variants or derivatives thereof (as presented by FIGS. 3D and 4D).

In some embodiments, the human IL-10 used in the modulating component of the present disclosure may be the human IL-10 encoded by a nucleic acid sequence comprising SEQ ID NO: 29, or any variants or derivatives thereof (also referred to herein as hIL-10_mut_CUT). In some embodiments, this IL-10 variant is provided in the modulatory component of the present disclosure comprised within a construct comprising the nucleic acid sequence as denoted by SEQ ID NO: 16, or any variants or derivatives thereof (as presented by FIGS. 3E and 4E).

In some embodiments, the human IL-10 used in the modulating component of the present disclosure may be the human IL-10 encoded by a nucleic acid sequence comprising SEQ ID NO: 30, or any variants or derivatives thereof (also referred to herein as hIL-10_mut_mod). In some embodiments, this IL-10 variant is provided in the modulatory component of the present disclosure comprised within a construct comprising the nucleic acid sequence as denoted by SEQ ID NO: 17, or any variants or derivatives thereof (as presented by FIGS. 3F and 4F).

It should be noted that in some embodiments, a system of the present disclosure that comprises at least one modulatory component expressing IL-10 as the product of interest, and at least one selective component, is referred to herein as the IL-10-modulatory system or IL-10-modulatory system of the present disclosure.

Still further, in some embodiments, the product of interest may be the Interleukin-2 (IL-2). In more specific embodiments, the product of interest may be the human IL-2. Still further, in some embodiments, the product of interest may be IL-2, as denoted by Gene ID: 3586. Still further, in some embodiments, the product of interest in accordance with the systems and methods of the present disclosure may be IL-2, that is encoded by the nucleic acid sequence that comprise SEQ ID NO: 3, or any homologs, variants or derivatives thereof.

According to such embodiments, the inflammatory condition may be any one of rheumatoid arthritis (RA), Crohn's disease, colitis, and psoriasis.

Rheumatoid arthritis, or RA, is an autoimmune and inflammatory disease, wherein the immune system mistakenly attacks healthy cells in the body, causing inflammation (painful swelling) in the affected parts of the body. RA mainly attacks the joints, usually many joints at once. RA commonly affects joints in the hands, wrists, and knees. In a joint with RA, the lining of the joint becomes inflamed, causing damage to joint tissue. This tissue damage can cause long-lasting or chronic pain, unsteadiness (lack of balance), and deformity (misshapenness). RA can also affect other tissues throughout the body and cause problems in organs such as the lungs, heart, and eyes.

Crohn's disease is a type of inflammatory bowel disease (IBD) that may affect any segment of the gastrointestinal tract. Symptoms often include abdominal pain, diarrhea (which may be bloody if inflammation is severe), fever, abdominal distension, and weight loss. Complications outside of the gastrointestinal tract may include anemia, skin rashes, arthritis, inflammation of the eye, and fatigue. The skin rashes may be due to infections as well as pyoderma gangrenosum or erythema nodosum. Bowel obstruction may occur as a complication of chronic inflammation, and those with the disease are at greater risk of colon cancer and small bowel cancer. Although the precise causes of Crohn's disease (CD) are unknown, it is believed to be caused by a combination of environmental, immune, and bacterial factors in genetically susceptible individuals. It results in a chronic inflammatory disorder, in which the body's immune system defends the gastrointestinal tract, possibly targeting microbial antigens. The exact underlying immune problem is not clear. However, it may be an immunodeficiency state. About half of the overall risk is related to genetics, involvement of various genes. It often begins after gastroenteritis. Still further, Irritable bowel syndrome (IBS) is a “disorder of gut-brain interaction” characterized by a group of symptoms that commonly include abdominal pain, abdominal bloating and changes in the consistency of bowel movements. These symptoms may occur over a long time, sometimes for years. Disorders such as anxiety, major depression, and chronic fatigue syndrome are common among people with IBS. The causes of IBS may well be multi-factorial. Theories include combinations of “gut-brain axis” problems, alterations in gut motility, visceral hypersensitivity, infections including small intestinal bacterial overgrowth, neurotransmitters, genetic factors, and food sensitivity. Onset may be triggered by an intestinal infection (“post-infectious irritable bowel syndrome”) or a stressful life event.

Ulcerative colitis (UC) is a chronic inflammatory bowel disease of unknown etiology affecting the colon and rectum. Multiple factors, such as genetic background, environmental and luminal factors, and mucosal immune dysregulation, have been suggested to contribute to UC pathogenesis. UC has evolved into a global burden given its high incidence in developed countries and the substantial increase in incidence in developing countries. Despite recent improvements in patient care, a substantial proportion of patients, for example, those who are refractory to medical treatment or those who develop colitis-associated colorectal dysplasia or cancer, still require restorative proctocolectomy.

Still further, Psoriasis (from Greek, meaning “itching condition” or “being itchy”) is a long-lasting, noncontagious autoimmune disease characterized by raised areas of abnormal skin. These areas are red, pink, or purple, dry, itchy, and scaly. Psoriasis varies in severity from small, localized patches to complete body coverage. Injury to the skin can trigger psoriatic skin changes at that spot, which is known as the Koebner phenomenon. The five main types of psoriasis are plaque, guttate, inverse, pustular, and erythrodermic. Plaque psoriasis, also known as psoriasis vulgaris, makes up about 90% of cases. It typically presents as red patches with white scales on top. Areas of the body most commonly affected are the back of the forearms, shins, navel area, and scalp. Guttate psoriasis has drop-shaped lesions. Pustular psoriasis presents as small, noninfectious, pus-filled blisters. Inverse psoriasis forms red patches in skin folds. Erythrodermic psoriasis occurs when the rash becomes very widespread, and can develop from any of the other types. Fingernails and toenails are affected in most people with psoriasis at some point in time. This may include pits in the nails or changes in nail color. Psoriasis is generally thought to be a genetic disease that is triggered by environmental factors. If one twin has psoriasis, the other twin is three times more likely to be affected if the twins are identical than if they are nonidentical. This suggests that genetic factors predispose to psoriasis. The underlying mechanism involves the immune system reacting to skin cells. Diagnosis is typically based on the signs and symptoms. Psoriasis is associated with an increased risk of psoriatic arthritis, lymphomas, cardiovascular disease, Crohn's disease, and depression. Psoriatic arthritis affects up to 30% of individuals with psoriasis.

As indicated above, the product of interest is a product affecting and/or modulating at least one immune-related condition. In some further alternative or additional specific embodiments, the product of interest may be at least one Gliadin degrading enzyme. In yet some further embodiments, the immune-related condition is an inflammatory condition, specifically, at least one Gluten-related disorder.

In more specific embodiments, the Gluten-related disorder includes celiac disease (CD), wheat allergy, baker's asthma and non-celiac gluten sensitivity. Thus, the system of the present disclosure may provide a tool for modulating an immune-related condition.

In more specific embodiments, the at least one Gliadin degrading enzyme is at least one of at least one prolyl endopeptidase, at least one cysteine protease and at least one subtilisin.

Gluten is the common name for proteins present in all grains of the Triticeae grass tribe in which the major industrially relevant crops are barley, wheat and rye. A high content of proline and glutamine residues is found in gluten proteins, which makes them resistant to gastrointestinal track and potentiates their deamidation by tissue transglutaminase.

Gluten-related disorders including celiac disease (CD), wheat allergy, baker's asthma and non-celiac gluten sensitivity, which have gradually emerged as an epidemiologically significant phenomenon with a relatively high global prevalence (5-10% of the population). Celiac disease develops in genetically susceptible individuals and is triggered by the exposure to partially digested gluten proteins. To date, the only available therapy for CD is the complete avoidance of dietary gluten. However, sustaining a strictly gluten-free diet (GFD) is very challenging. Although gluten levels in individual products have been determined, the amount of gluten in a “gluten-free” diet as consumed by people with CD remains unknown.

Therefore, targeted modification of microbiome cells to produce enzymes that cleave the digestion-.resistant gluten peptides in vivo, may be used as a therapeutic approach for CD patients. Gliadin (a type of prolamin), which is a component of gluten and is found in products such as wheat flour, is essential for giving bread the ability to rise properly during baking. Gliadins and glutenins are the two main components of the gluten fraction of the wheat seed. Gluten is split about evenly between the gliadins and glutenins, although there are variations found in different sources. There are three main types of gliadin (α, γ, and ω), to which the body is intolerant in coeliac (or celiac) disease.

Gluten degrading enzymes also referred to as gliadin degrading enzymes are any animal, microbial or plant enzyme, including but not limited to mammalian, bacterial, fungal enzymes, which had the ability to degrade either completely or partially gluten proteins, specifically gliadin.

Gliadin degrading enzymes include enzymes such as prolyl endopeptidases (PEPs, also known as prolyl oligopeptidases) which may constitute therapeutic keys for the treatment of CD, since they are especially effective in the hydrolysis of peptide bonds on the carboxyl side of internal proline residues in gluten-derived oligopeptides. PEPs belong to serine protease family (clan SC, family S9), which is a group of peptidases can hydrolyze peptides smaller than 30 residues. PEPs are structurally and functionally closely related to that of the dipeptidyl peptidase IV (DPP-IV), oligo peptidase B and acyl-aminoacyl peptidase sub-families.

Cysteine protease, also known as thiol proteases, are hydrolase enzymes that degrade proteins. Cysteine proteases are commonly encountered in fruits including the papaya, pineapple, fig and kiwifruit as well as other plants such as barely. Cysteine proteases was shown to degrade gluten proteins, specifically Gliadin, and therefore may also be considered as a therapeutic candidate for CD.

Subtilisins, additional potential therapeutic candidates, belong to subtilases, a group of serine proteases that like all serine proteases initiate the nucleophilic attack on the peptide (amide) bond through a serine residue at the active site. Subtilisins typically have molecular weights 27 kDa. They can be obtained from certain types of soil bacteria, for example, Bacillus amyloliquefaciens from which they are secreted in large amounts as well as from Bacillus subtilis. In yet some further embodiments, the product of interest is at least one agonist of the guanylyl cyclase receptor guanylate cyclase 2C (GC-C). Accordingly, in some embodiments, the immune-related condition is an inflammatory condition. In yet some further specific embodiments, the inflammatory condition is irritable bowel syndrome.

Still further, in some embodiments, the GC-C agonist is at least one peptide of the Guanylin peptides (GPs) family, or any mimetics thereof. More specifically, Guanylate cyclase 2C, also known as guanylyl cyclase C (GC-C), intestinal guanylate cyclase, guanylate cyclase-C receptor, or the heat-stable enterotoxin receptor (hSTAR) is an enzyme found in the luminal aspect of intestinal epithelium and dopamine neurons in the brain. The receptor has an extracellular ligand-binding domain, a single transmembrane region, a region with sequence similar to that of protein kinases, and a C-terminal guanylate cyclase domain. Tyrosine kinase activity mediates the GC-C signaling pathway within the cell. GC-C is a key receptor for heat-stable enterotoxins that are responsible for acute secretory diarrhea. Heat-stable enterotoxins are produced by pathogens such as Escherichia coli. Knockout mice deficient in the GC-C gene do not show secretory diarrhea on infection with E. coli, though they do with cholera toxin. This demonstrates the specificity of the GC-C receptor. Guanylate cyclase 2C is the target of linaclotide and plecanatide, oligopeptide agonists used for the treatment of chronic constipation.

In yet some further embodiments, the Guanylin peptides (GPs) family includes guanylin (GN), uroguanylin (UGN), lymphoguanylin, and recently discovered renoguanylin and mimetics Linaclotide. The guanylin peptide (GPs) family has several subclasses of peptides, including guanylin and uroguanylin. The sources of these peptides are the intestine and the kidneys but also other epithelia. Guanylin mRNA has been detected mainly in the gastrointestinal tract in numerous cell types and in the kidney. Uroguanylin mRNA is, for example, present in the gut, kidney, heart, reproductive system, and brain. The expression pattern of both peptides is differential in the intestine: the highest expression of guanylin occurs in the ileum to the proximal colon, and of uroguanylin in the jejunum. As in the gut, guanylin and uroguanylin are not equally expressed along the different nephron segments. Guanylin is more pronounced in the collecting duct, and uroguanylin is present mostly in the proximal tubule. In the classical point of view., guanylin and proguanylin are primarily thought to participate in the regulation of salt and water homeostasis in the intestine and the kidney. More specifically, Guanylin is a 15 amino acid peptide that is secreted by goblet cells in the colon. Guanylin acts as an agonist of the guanylyl cyclase receptor GC-C and regulates electrolyte and water transport in intestinal and renal epithelia. Upon receptor binding, guanylin increases the intracellular concentration of cGMP, induces chloride secretion and decreases intestinal fluid absorption, ultimately causing diarrhea. The peptide stimulates the enzyme through the same receptor binding region as the heat-stable enterotoxins.

In some alternative embodiments, the product of interest is a metabolic product and/or a product affecting and/or modulating a metabolic process or condition/s. Accordingly, in some embodiments, the product is a protein or polypeptide product encoded by at least one nucleic acid sequence of the microbiome modulating component of (a)(i).

In some embodiments, the protein or polypeptide product increases insulin secretion and/or production. Accordingly, such product affects and/or modulates a metabolic process and/or condition/s, for example, at least one of diabetes and metabolic syndrome and associated conditions. More specifically, Insulin is a peptide hormone produced by beta cells of the pancreatic islets encoded in humans by the INS gene. It is considered to be the main anabolic hormone of the body. It regulates the metabolism of carbohydrates, fats and protein by promoting the absorption of glucose from the blood into liver, fat and skeletal muscle cells. In these tissues the absorbed glucose is converted into either glycogen via glycogenesis or fats (triglycerides) via lipogenesis, or, in the case of the liver, into both. Glucose production and secretion by the liver is strongly inhibited by high concentrations of insulin in the blood. Circulating insulin also affects the synthesis of proteins in a wide variety of tissues. It is therefore an anabolic hormone, promoting the conversion of small molecules in the blood into large molecules inside the cells. Low insulin levels in the blood have the opposite effect by promoting widespread catabolism, especially of reserve body fat.

In yet some further embodiments, the protein or polypeptide product is glucagon-like peptide-1 (GLP-1) or any mimetics and derivatives thereof. Glucagon-like peptide-1 (GLP-1) is a 30- or 31-amino-acid-long peptide hormone deriving from the tissue-specific posttranslational processing of the proglucagon peptide. It is produced and secreted by intestinal enteroendocrine L-cells and certain neurons within the nucleus of the solitary tract in the brainstem upon food consumption. The initial product GLP-1 (1-37) is susceptible to amidation and proteolytic cleavage, which gives rise to the two truncated and equipotent biologically active forms, GLP-1 (7-36) amide and GLP-1 (7-37). Active GLP-1 protein secondary structure includes two α-helices from amino acid position 13-20 and 24-35 separated by a linker region. Alongside glucose-dependent insulinotropic peptide (GIP), GLP-1 is an incretin. Thus, it has the ability to decrease blood sugar levels in a glucose-dependent manner by enhancing the secretion of insulin. Beside the insulinotropic effects, GLP-1 has been associated with numerous regulatory and protective effects.

Still further, in some additional embodiments, the product of interest encoded and/or modulated by the modulatory component of the system of the invention is a product affecting and/or modulating neurological and/or neuro-behavioral processes or conditions. In more specific embodiments, such product is a protein or polypeptide product encoded by at least one nucleic acid sequence of the microbiome modulating component of (a)(i), or a product produced directly or indirectly by an element encoded by the nucleic acid sequence of said microbiome modulating component of (a)(i).

In some embodiments, the product of interest is a hormone peptide encoded by at least one nucleic acid sequence of said microbiome modulating component of (a)(i).

Hormone peptides or peptide hormones, as used herein, are hormones whose molecules are peptide. Peptide hormones have shorter amino acid chain lengths than protein hormones. These hormones have an effect on the endocrine system of animals, including humans. Most hormones can be classified as either amino acid-based hormones (amine, peptide, or protein) or steroid hormones. The former are water-soluble and act on the surface of target cells via second messengers; the latter, being lipid-soluble, move through the plasma membranes of target cells (both cytoplasmic and nuclear) to act within their nuclei. Like all peptides, peptide hormones are synthesized in cells from amino acids according to mRNA transcripts, which are synthesized from DNA templates inside the cell nucleus. Preprohormones, peptide hormone precursors, are then processed in several stages, typically in the endoplasmic reticulum, including removal of the N-terminal signal sequence and sometimes glycosylation, resulting in prohormones. The prohormones are then packaged into membrane-bound secretory vesicles, which can be secreted from the cell by exocytosis in response to specific stimuli (e.g. an increase in Ca2+ and cAMP concentration in cytoplasm). Peptide hormones in humans include, but are not limited to oxytocin, adrenocorticotropic hormone (ACTH), adropin, amylin, angiotensin, atrial natriuretic peptide (ANP), calcitonin, cholecystokinin (CCK), gastrin, ghrelin, glucagon, growth hormone, follicle-stimulating hormone (FSH), insulin, leptin, luteinizing, ormone (LH), melanocyte-stimulating hormone (MSH), parathyroid hormone (PTH), prolactin, renin, somatostatin, thyroid-stimulating hormone (TSH), thyrotropin-releasing, ormone (TRH), vasopressin, also called arginine vasopressin (AVP) or anti-diuretic hormone (ADH), vasoactive intestinal peptide (VIP).

Still further, a hormone is a class of signaling molecules in multicellular organisms that are sent to distant organs by complex biological processes to regulate physiology and behavior. Hormones are required for the correct development of animals, plants and fungi. As a signaling molecule that exerts its effects far from its site of production, numerous kinds of molecules can be classified as hormones. Among the substances that can be considered hormones, are eicosanoids (e.g. prostaglandins and thromboxanes), steroids (e.g. oestrogen), amino acid derivatives (e.g. epinephrine and auxin), protein or peptides (e.g. insulin and CLE peptides), and gases (e.g. ethylene and nitric oxide). In vertebrates, hormones are responsible for regulating a variety of physiological processes and behavioral activities such as digestion, metabolism, respiration, sensory perception, sleep, excretion, lactation, stress induction, growth and development movement, reproduction, and mood manipulation. Hormones affect distant cells by binding to specific receptor proteins in the target cell, resulting in a change in cell function. When a hormone binds to the receptor, it results in the activation of a signal transduction pathway that typically activates gene transcription, resulting in increased expression of target proteins. Hormones can also act in non-genomic pathways that synergize with genomic effects. Water-soluble hormones (such as peptides and amines) generally act on the surface of target cells via second messengers. Lipid soluble hormones, (such as steroids) generally pass through the plasma membranes of target cells (both cytoplasmic and nuclear) to act within their nuclei.

Accordingly, the product affects and/or modulates neuro-behavioral processes or condition is at least one of bonding and dyadic relations, and stress-related conditions.

In more specific embodiments, the hormone peptide is Oxytocin. More specifically, Oxytocin (Oxt or OT) is a peptide hormone and neuropeptide normally produced in the hypothalamus and released by the posterior pituitary. Present in animals since early stages of evolution, in humans it plays roles in behavior that include social bonding, reproduction, childbirth, and the period after childbirth. Oxytocin is released into the bloodstream as a hormone in response to sexual activity and during labour. It is also available in pharmaceutical form. In either form, oxytocin stimulates uterine contractions to speed up the process of childbirth. In its natural form, it also plays a role in bonding with the baby and milk production. Production and secretion of oxytocin is controlled by a positive feedback mechanism, where its initial release stimulates production and release of further oxytocin. For example, when oxytocin is released during a contraction of the uterus at the start of childbirth, this stimulates production and release of more oxytocin and an increase in the intensity and frequency of contractions. This process compounds in intensity and frequency and continues until the triggering activity ceases. A similar process takes place during lactation and during sexual activity. Oxytocin is derived by enzymatic splitting from the peptide precursor encoded by the human OXT gene.

In some further embodiments, the product of interest is at least one hormone produced by an element encoded by the nucleic acid sequence of the microbiome modulating component of (a)(i). In yet some further embodiments, such element is the tryptophan hydroxylase. Tryptophan hydroxylase (TPH) is an enzyme involved in the synthesis of the neurotransmitter serotonin. It is responsible for addition of the —OH group (hydroxylation) to the 5 position to form the amino acid 5-hydroxytryptophan (5-HTP), which is the initial and rate-limiting step in the synthesis of the neurotransmitter serotonin. It is also the first enzyme in the synthesis of melatonin. Tryptophan hydroxylase (TPH), tyrosine hydroxylase (TH) and phenylalanine hydroxylase (PAH) are members of a superfamily of biopterin-dependent aromatic amino acid hydroxylases, catalyzing key steps in important metabolic pathways. Analogously to phenylalanine hydroxylase and tyrosine hydroxylase, this enzyme uses (6R)-L-erythro-5,6,7,8-tetrahydrobiopterin (BH4) and dioxygen as cofactors.

Still further, the product affects and/or modulates a neuronal processes that may be at least one of neuronal differentiation and migration, axonal outgrowth, myelination and synapse formation.

In more specific embodiments, such product is Serotonin. Serotonin or 5-hydroxytryptamine (5-HT) is a monoamine neurotransmitter. Its biological function is complex and multifaceted, modulating mood, cognition, reward, learning, memory, and numerous physiological processes such as vomiting and vasoconstriction. Approximately 90% of the serotonin the human body produces is in the gastrointestinal tract's enterochromaffin cells, where it regulates intestinal movements. Serotonin is also produced in the central nervous system (CNS), specifically in the brainstem's raphe nuclei, the skin's Merkel cells, pulmonary neuroendocrine cells and the tongue's taste receptor cells. Additionally, it is stored in blood platelets and is released during agitation and vasoconstriction, where it then acts as an agonist to other platelets. About 8% is found in platelets and 1-2% in the CNS. The serotonin is secreted luminally and basolaterally, which leads to increased serotonin uptake by circulating platelets and activation after stimulation, which gives increased stimulation of myenteric neurons and gastrointestinal motility. The remainder is synthesized in serotonergic neurons of the CNS, where it has various functions, including the regulation of mood, appetite, and sleep.

As indicated above, the disclosed systems comprise at least two components, the modulating component and the selective component. In some embodiments, at least one of these components comprise at least one nucleic acid sequence. The term ‘polynucleotide’ or a ‘nucleic acid sequence’ or ‘nucleic acid molecule’ refer herein to a polymer of nucleic acids, such as deoxyribonucleic acid (DNA) or ribonucleic acid (RNA). As used herein, ‘nucleic acid/s’ (also or nucleic acid molecule or nucleotide) refers to any DNA or RNA polynucleotides, oligonucleotides, fragments generated by the polymerase chain reaction (PCR) and fragments generated by any of ligation, scission, endonuclease action, and exonuclease action, either single- or double-stranded. Nucleic acid molecules can be composed of monomers that are naturally-occurring nucleotides (such as DNA and RNA), or analogs of naturally-occurring nucleotides (e.g., alpha-enantiomeric forms of naturally-occurring nucleotides), or modified nucleotides or any combination thereof. Herein this term also encompasses a cDNA, i.e. complementary or copy DNA produced from an RNA template by the action of reverse transcriptase (RNA-dependent DNA polymerase).

A further aspect of the present disclosure relates to a method for modifying and/or manipulating, and/or modulating target bacterial cell/s and/or target bacterial cell population to produce a product of interest. More specifically, the method comprising at least one of:
First (a), contacting the cell/s or any cell population comprising the cell/s with at least one microbiome modulating component comprising: (i) at least one nucleic acid sequence encoding and/or modulating at least one product of interest and/or at least one element participating in the production and/or metabolism of the at least one product of interest; and (ii) at least one cas gene and at least one CRISPR array comprising at least one spacer. The next step (b), involves contacting the cells or any cell population comprising the cell/s with at least one selective component comprising at least one protospacer. In some embodiments, the at least one protospacer in the selective component of (b), is targeted by at least one spacer of the CRISPR array of (a), such that the selective component is inactivated.

Thereby, modifying target bacterial cell/s and/or target bacterial cell population to produce the product of interest.

It should be understood that in some embodiments, the term “contacting” includes the in vivo as well as the in vitro exposure of cells to the components or compositions disclosed herein. Thus, in some embodiments, “contacting” includes administering the at least one system and/or any components thereof, specifically, the modulatory component and/or the selective component as disclosed herein, or any delivery vehicle comprising these components, or any cells or population of cells (e.g., bacterial cells) or any composition thereof, to an individual.

In some embodiments, the at least one nucleic acid sequence encoding or modulating at least one product of interest comprised within the CRISPR array.

In some embodiments, the step of contacting the target cells with the systems of the present disclosure or with any components thereof, as provided by the present disclosure, may in some embodiments involve transformation and/or transduction of the nucleic acid sequences of the components of the system of the present disclosure to the target cell, for example, the target bacterial cell. “Transformation” and “transfection” means the introduction of a nucleic acid, e.g., naked DNA or the delivery vehicle as herein defined, into a recipient cell/s by nucleic acid-mediated gene transfer.

“Transduction” refers to a process by which foreign DNA is introduced into a cell by a virus or viral vector. An example is the viral transfer of DNA from one bacterium to another and hence an example of horizontal gene transfer.

In yet some further embodiments, the modulating component used by the methods disclosed herein, is comprised within at least one delivery vehicle that specifically targets the cell/s and/or the cells in a bacterial cell population.

In some embodiments, the selective component comprises at least one protospacer targeted by at least one spacer of the microbiome modulating component, such that the selective component is specifically inactivated by the CRISPR array of the microbiome modulating component.

In yet some further embodiments, the selective component is comprised within at least one delivery vehicle.

In some embodiments, the microbiome modulating component is comprised within at least one delivery vehicle that specifically targets the cell/s and/or said cells in a bacterial cell population.

In some embodiments, the delivery vehicle is, or comprises at least one genetic element. More specifically, in some embodiments, such genetic element is at least one of at least one transducing particle, at least one bacteriophage or any fragments or parts thereof, any bacteriophage-like transducing particle, or any mixture or cocktail thereof.

In some embodiments, the selective component is comprised within at least one delivery vehicle that is, or comprises at least one genetic element. In some embodiments, the genetic element is at least one of: at least one transducing particle, at least one lytic bacteriophage or any fragments or parts thereof, at least one plasmid that comprises and/or encodes at least one toxic element or factor that kill bacterial cells or affects, disrupt, attenuate, and/or inhibit bacterial cell growth.

In yet some further embodiments, the delivery vehicle is at least one bacteriophage or any bacteriophage-like transducing particle. It should be understood that delivery vehicle/s applicable in the present aspect may comprise any of the delivery vehicles disclosed herein above, in connection with other aspects of the present disclosure.

Still further, in some embodiments, the bacterial cell/s and/or cell population is, or comprised within gut microbiome cell population. More specifically, the cell population comprises at least one bacterium of the Proteobacteria, Firmicutes, Bacteroidetes, Actinobacteria, and phyla, or any mutant, variant or isolate or any combination thereof.

In some embodiments, the microbiome metabolism modulating component used by the disclosed methods affects and/or modulates at least one of the expression, production, activity, bioavailability, clearance, stability, toxicity, and absorption of the product of interest.

In yet some further embodiments, the product of interest may be at least one of: a product affecting and/or modulating immune-related processes and/or conditions, a metabolic product and/or a product affecting and/or modulating a metabolic process or condition/s, a product affecting and/or modulating neurological, neuronal and/or neuro-behavioral processes or conditions, a therapeutic product, a cosmetic product and a nutritional product.

In some embodiments, the product of interest encoded and/or modulated by the elements of the microbiome modulating component used by the methods of the present invention is a product affecting and/or modulating immune-related processes and/or conditions. In some embodiments, the product is a protein or polypeptide product encoded by at least one nucleic acid sequence of the microbiome modulating component of (a)(i).

In yet some further embodiments, the product of interest encoded and/or modulated by the elements of the microbiome modulating component used by the methods of the present invention is at least one cytokine.

In more specific embodiments o the disclosed methods, the cytokine is at least one of Interleukine-2 (IL-2), Interleukine-10 (IL-10) and Interleukine-17 (IL-17).

In some specific embodiments, the product of interest of the modulatory component of the system used by the methods of the present disclosure is IL-10.

Accordingly, in some embodiments, the immune-related condition is an inflammatory condition.

In yet some further embodiments, the inflammatory condition is any one of rheumatoid arthritis (RA), Crohn's disease, colitis, and psoriasis.

Still further, in some embodiments, the product of interest is at least one Gliadin degrading enzyme. In yet some further embodiments, the immune-related condition is an inflammatory condition, specifically, at least one Gluten-related disorder.

In certain embodiments, the Gliadin degrading enzyme include celiac disease (CD), wheat allergy, baker's asthma and non-celiac gluten sensitivity.

In yet some further embodiments, the at least one Gliadin degrading enzyme is at least one of at least one prolyl endopeptidase, at least one cysteine protease and at least one subtilisin.

In some embodiments, the product of interest is at least one agonist of the guanylyl cyclase receptor guanylate cyclase 2C (GC-C). Accordingly, the immune-related condition is an inflammatory condition. In more specific embodiments, the inflammatory condition is irritable bowel syndrome.

Still further, in some embodiments, the GC-C agonist is at least one peptide of the Guanylin peptides (GPs) family, or any mimetics thereof.

In more specific embodiments, Guanylin peptides (GPs) family includes guanylin (GN), uroguanylin (UGN), lymphoguanylin, and recently discovered renoguanylin and mimetics Linaclotide.

Still further, in some embodiments, the product of interest encoded and/or modulated by the elements of the microbiome modulating component used by the methods of the present disclosure is a metabolic product and/or a product affecting and/or modulating a metabolic process or condition/s. In some embodiment, the product is a protein or polypeptide product encoded by at least one nucleic acid sequence of the microbiome modulating component of (a)(i), used by the methods of the disclosure.

In some embodiments, the protein or polypeptide product increases insulin secretion and/or production. Accordingly, the metabolic condition/s is at least one of diabetes and metabolic syndrome and associated conditions.

Still further, in some embodiments of the disclosed methods, the protein or polypeptide product is glucagon-like peptide-1 (GLP-1) or any mimetics and derivatives thereof. In some embodiments, the GLP-1 is the human GLP-1. Still further, in some embodiments, the GLP-1 is encoded by the nucleic acid sequence as denoted by SEQ ID NO: 20, or any variants thereof.

In yet some further embodiments, the product of interest encoded and/or modulated by the elements of the microbiome modulating component used by the methods of the present invention is a product affecting and/or modulating neurological and/or neuro-behavioral processes or conditions. More specifically, such product is a protein or polypeptide product encoded by at least one nucleic acid sequence of the microbiome modulating component of (a)(i), or a product produced directly or indirectly by an element encoded by the nucleic acid sequence of the microbiome modulating component of (a)(i).

In some specific embodiments, the product of interest is a hormone peptide encoded by at least one nucleic acid sequence of said microbiome modulating component of (a)(i). Accordingly, the neuro-behavioral processes or condition is at least one of bonding and dyadic relations, and stress-related conditions.

In some specific embodiments, the hormone peptide is Oxytocin.

In some embodiments, the product of interest is at least one hormone produced by an element encoded by the nucleic acid sequence of the microbiome modulating component of (a)(i). In some embodiments, the element is the tryptophan hydroxylase. More specifically, the neuronal processes may be at least one of neuronal differentiation and migration, axonal outgrowth, myelination and synapse formation.

In et some further embodiments, the product is Serotonin.

A further aspect of the present disclosure relates to at least one bacterial cell and/or a population of the bacterial cells, or any composition or product thereof, manipulated for producing at least one product of interest. In some embodiments, the cell and/or cell population is prepared by a method comprising at least one of: First (a), contacting the cell/s or any cell population comprising the cell/s with at least one microbiome modulating component comprising: (i) at least one nucleic acid sequence encoding and/or modulating at least one said product of interest and/or at least one element participating in the production and/or metabolism of said at least one product of interest; and (ii) at least one cas gene and at least one CRISPR array comprising at least one spacer; and The next step (b), contacting the cells or any cell population comprising the cell/s with at least one selective component comprising at least one protospacer. In some embodiments, the at least one protospacer is targeted by at least one spacer of the CRISPR array of (a), such that the selective component is inactivated; thereby modifying target bacterial cell/s and/or target bacterial cell population to produce the product of interest.

In some embodiments of the bacterial cell and/or a population, the cell or population is prepared by a method as defined by the present disclosure herein above.

In some embodiments, the product of interest produced by the bacterial cell and/or a population of the present disclosure is a product affecting and/or modulating immune-related processes and/or conditions. Such product is a protein or polypeptide product encoded by a sequence comprised within at least one nucleic acid sequence of the microbiome modulating component of (a)(i).

In some further embodiments, the product of interest is a product affecting and/or modulating at least one immune-related condition, said immune-related condition is an inflammatory condition.

In more specific embodiments, the product of interest is at least one cytokine.

More specifically, in some embodiments, the cytokine is at least one of IL-10, IL-2, and IL-17.

In more specific embodiments of the disclosed bacterial cell and/or a population, the cytokine is IL-10.

In some embodiments, the IL-10 is useful for an inflammatory condition, specifically, is any one of Crohn's disease, colitis, rheumatoid arthritis (RA) and psoriasis.

A further aspect of the present disclosure relates to a method for modifying target bacterial cell/s and/or target bacterial cell population to produce a product of interest in a subject in need thereof. More specifically, the method comprising the step of administering to the subject an effective amount of at least one of: (a), at least one microbiome modulating component comprising: (i) at least one nucleic acid sequence encoding and/or modulating at least one the product of interest and/or at least one element participating in the production and/or metabolism of the at least one product of interest; and (ii), at least one cas gene and at least one Clustered, Regularly Interspaced Short Palindromic Repeat (CRISPR) array, comprising at least one spacer; and
(b) at least one selective component comprising at least one protospacer, wherein said at least one protospacer is targeted by at least one spacer of the CRISPR array of (a), such that the selective component is inactivated; (c) at least one composition, kit or system comprising at least one of (a) and (b); and (d) any combination of the at least one of (a), (b) and (c) with the at least one drug.

In some embodiments of the disclosed methods, the system is as defined by the present disclosure, the cell or cell population is as defined by the present disclosure.

In some embodiments, the product of interest produced by the disclosed method is a product affecting and/or modulating immune-related processes and/or conditions. Such product is a protein or polypeptide product encoded by a sequence comprised within at least one nucleic acid sequence of the microbiome modulating component of (a)(i).

In some further embodiments, the product of interest is a product affecting and/or modulating at least one immune-related condition, said immune-related condition is an inflammatory condition.

In more specific embodiments, the product of interest is at least one cytokine.

More specifically, in some embodiments, the cytokine is at least one of IL-10, IL-2, and IL-17.

In more specific embodiments of the disclosed bacterial cell and/or a population, the cytokine is IL-10. In some embodiments, the IL-10 is useful for an inflammatory condition, specifically, is any one of Crohn's disease, colitis, rheumatoid arthritis (RA) and psoriasis.

In yet some further embodiments, the subject administered and modulated by the disclosed methods is a subject suffering from an immune-related condition, a metabolic condition and a neurological condition and/or a subject undergoing/experiencing an immune-related process, a metabolic process and a neuronal and/or neuro-behavioral processes.

A further aspect of the present disclosure relates to a method for treating, preventing, ameliorating, reducing or delaying the onset of at least one pathologic disorder in a subject in need thereof. More specifically, the method comprising the step of administering to the subject an effective amount of at least one of:
(I) (a) at least one microbiome modulating component comprising: (i) at least one nucleic acid sequence encoding and/or modulating at least one the product of interest and/or at least one element participating in the production and/or metabolism of said at least one product of interest; and (ii) at least one cas gene and at least one CRISPR array, comprising at least one spacer; and
(b) at least one selective component comprising at least one protospacer. It should be noted that the at least one protospacer is targeted by at least one spacer of the CRISPR array of (a), thereby inactivating the selective component;
(II) at least one bacterial cell and/or a population of the bacterial cells, or any composition or product thereof, producing the product of interest;
(III) at least one composition, kit or system comprising at least one of (I) and (II); and
(IV) any combination of the at least one of (I), (II) and (III) with at least one therapeutic agent.

In some embodiments, the systems used by the therapeutic methods disclosed herein is as defined by the present disclosure, and the cell or cell population is defined by the various embodiments of the present disclosure.

Still further, in some embodiments, the methods of the present disclosure may be applicable for a pathologic disorder such as, at least one of an inflammatory disorder, a metabolic disorder, a neuronal condition, an autoimmune disorder and a neoplastic disorder.

Still further, in some embodiments the inflammatory disorder applicable for the methods of the invention is at least one of RA, Crohn's disease, colitis, and psoriasis. In such case, the product of interest is at least one cytokine. Thus, the immune-related condition is an inflammatory condition. More specifically, in some embodiments, the cytokine is at least one of IL-10, IL-2, and IL-17.

In more specific embodiments of the disclosed bacterial cell and/or a population, the cytokine is IL-10. In some embodiments, the IL-10 is useful for an inflammatory condition, specifically, is any one of Crohn's disease, colitis, rheumatoid arthritis (RA) and psoriasis.

In some embodiments, the inflammatory disorder is at least one Gluten-related disorder.

In more specific embodiments, one Gluten-related disorder may be any one of celiac disease (CD), wheat allergy, baker's asthma and non-celiac gluten sensitivity.

Still further, in some embodiments, the product of interest is at least one Gliadin degrading enzyme.

In some specific embodiments, Gliadin degrading enzyme is at least one of at least one prolyl endopeptidase, at least one cysteine protease and at least one subtilisin.

In some embodiments, the inflammatory condition treatable by the disclosed methods is irritable bowel syndrome. In some embodiments, the product of interest is at least one agonist of the guanylyl cyclase receptor guanylate cyclase 2C (GC-C).

In yet some further embodiments, the metabolic condition/s treatable by the disclosed methods is at least one of diabetes and metabolic syndrome and associated conditions. In such case, the product of interest is GLP-1 or any mimetics and derivatives thereof. In some embodiments, the GLP-1 is encoded by a sequence comprising the nucleic acid sequence as denoted by SEQ ID NO: 20.

A further aspect of the present disclosure relates to a method for modulating at least one of an immune-related process, a metabolic process and a neuronal and/or neuro-behavioral processes in a subject in need thereof. According to some embodiments, the method comprising the step of administering to the subject an effective amount of at least one of:
(I) (a) at least one microbiome modulating component comprising:
(i) at least one nucleic acid sequence encoding and/or modulating at least one said product of interest and/or at least one element participating in the production and/or metabolism of said at least one product of interest; and
(ii) at least one cas gene and at least one CRISPR array, comprising at least one spacer; and
(b) at least one selective component comprising at least one protospacer, wherein said at least one protospacer is targeted by at least one spacer of the CRISPR array of (a), thereby inactivating the selective component;
(II) at least one bacterial cell and/or a population of the bacterial cells, or any composition or product thereof, producing the product of interest;
(III) at least one composition, kit or system comprising at least one of (I) and (II); and
(IV) any combination of said at least one of (I), (II) and (III) with at least one therapeutic agent.

In some embodiments, the system used by the disclosed methods is as defined by the invention, and the cell or cell population is as defined by the present disclosure.

In some embodiments, the product of interest produced by the disclosed method is a product affecting and/or modulating immune-related processes and/or conditions. Such product is a protein or polypeptide product encoded by a sequence comprised within at least one nucleic acid sequence of the microbiome modulating component of (a)(i).

In some further embodiments, the product of interest is a product affecting and/or modulating at least one immune-related condition, said immune-related condition is an inflammatory condition. In more specific embodiments, the product of interest is at least one cytokine.

More specifically, in some embodiments, the cytokine is at least one of IL-10, IL-2, and IL-17.

In more specific embodiments of the disclosed bacterial cell and/or a population, the cytokine is IL-10. In some embodiments, the IL-10 is useful for an inflammatory condition, specifically, is any one of Crohn's disease, colitis, rheumatoid arthritis (RA) and psoriasis.

In some embodiments, the neuro-behavioral processes or condition is at least one of bonding and dyadic relations, and stress-related conditions. In such case, the product of interest is Oxytocin.

Still further, in some embodiments, the neuronal processes may be at least one of neuronal differentiation and migration, axonal outgrowth, myelination and synapse formation. In such case, the product of interest is Serotonin.

It should be understood however that the instant invention also pertains to animal health, particularly mammalian health conditions, as covered by veterinary sciences.

Still further, inflammatory conditions and specifically, IBD and colitis can be caused by bacterial infections, viruses, and autoimmune disorders. However, these disorders may be also a side effect of some cancer treatments regiment that may comprise in some embodiments, immunotherapy medications and/or chemotherapeutic agents. Therefore, in some aspects thereof, the preset disclosure further provides uses of the disclosed systems in methods for treating cancer and any proliferative disorder. In some embodiments, such treatment may be combined with any first, second or third line of treatment regimen.

Thus, in some embodiments, the present disclosure provides a method for treating, preventing, ameliorating, reducing or delaying the onset of at least one proliferative disorder in a subject in need thereof. In some embodiments the method comprising the step of administering to the subject an effective amount of at least one of:
(I) the systems of the present disclosure, specifically, (a) at least one microbiome modulating component comprising:
(i) at least one nucleic acid sequence encoding and/or modulating at least one said product of interest and/or at least one element participating in the production and/or metabolism of said at least one product of interest; and
(ii) at least one cas gene and at least one CRISPR array, comprising at least one spacer; and
(b) at least one selective component comprising at least one protospacer, wherein the at least one protospacer is targeted by at least one spacer of the CRISPR array of (a), thereby inactivating the selective component; and
(II) at least one bacterial cell and/or a population of the bacterial cells, or any composition or product thereof, producing said product of interest;
(III) at least one composition, kit or system comprising at least one of (I) and (II); and
(IV) any combination of said at least one of (I), (II) and (III) with at least one therapeutic agent, specifically, any agent suitable for the treatment of proliferative disorders.

In some specific embodiments, the modulating component of the systems used in the present method may comprise at least one nucleic acid sequence encoding at least one cytokine. In yet some further embodiments, such cytokines may be IL-10.

In some particular embodiments, the disclosed methods may be applicable for patients subjected to immuno-therapy, for example, treatment with immune-check-point inhibitors (ICIs). As disclosed in Example 12, a clinical study using the IL-10 modulatory system of the present disclosure is used for treating patients undergoing ICI treatment.

Immune checkpoint inhibitors (ICI), as used herein, are a type of immunotherapy that works by blocking certain molecules on immune cells, such as T cells, to enhance the immune response against cancer cells. These drugs have shown promising results in the treatment of various cancers and are often used in combination with other treatments, such as chemotherapy or radiation therapy. Key indications for ICI treatment include melanoma, non-small cell lung cancer, renal cell carcinoma, and bladder cancer, among others [Pauken K E, et al., 511-523, s.l.: Trends Immunol., Vol. June; 40(6) (2019)].

The use of ICIs has been steadily increasing in recent years as clinical trials continue to demonstrate their efficacy in treating a range of cancers and more indications are approved for marketing. There are several types of ICIs that target different checkpoint molecules on immune cells. Currently, the only inhibitory targets approved by the FDA are cytotoxic T-lymphocyte-associated protein 4 (CTLA-4) and programmed cell death protein 1 (PD-1) or its ligand (PD-L1). Non-limiting embodiments for these molecules and the ICI drugs that target them are detailed herein. In some embodiments, the ICI may comprise CTLA-4 inhibitors. CTLA-4 is a protein that regulates T-cell activation and can inhibit immune responses. CTLA-4 inhibitors, such as ipilimumab, block the interaction between CTLA-4 and its ligands, thereby enhancing T-cell activation [Korman A J, et al., 509-528, s.l.: Nat Rev Drug Discov., Vol. July; 21(7) (2022)].

In some embodiments, the ICI may comprise PD-1 inhibitors. PD-1 is another checkpoint protein that can suppress T-cell activation. PD-1 inhibitors, such as nivolumab and pembrolizumab, block the interaction between PD-1 and its ligands, thereby enhancing T-cell activation [Korman A J, et al., 509-528, s.l.: Nat Rev Drug Discov., Vol. July; 21(7) (2022)].

In some embodiments, the ICI may comprise PD-L1 inhibitors. PD-L1 is a protein that can be expressed on tumor cells and other cells in the tumor microenvironment and can interact with PD-1 to inhibit T-cell activation. PD-L1 inhibitors, such as atezolizumab and durvalumab, block the interaction between PD-L1 and PD-1, thereby enhancing T-cell activation [Korman A J, et al., ibid]. It should be noted that despite the promising benefits of ICI therapy, patients frequently develop autoimmune-like effects, termed immune-related adverse events. Although the underlying mechanism of ICI-induced adverse effects remains unclear, it is thought that it is related to removal of key peripheral tolerance pathways normally maintained by immune checkpoints, resulting in inflammation in several non-tumor tissues.

The treatment for ICI-induced adverse effects usually involves the use of high-dose corticosteroids as the first line for managing. Cases where the ICI-induced adverse effects are refractory to high-dose corticosteroids are managed with add-on immunosuppressive therapy, such as Infliximab [Conroy M, Naidoo J. 392, s.l.: Nat Commun. Vol. January 19; 13(1) (2022)]. Critically, an immunosuppressive therapy is not desirable as it may counteract the antitumor activity of the ICI therapy. Still further, one of the most frequently occurring adverse reactions of ICI therapy, and the most common in the cases of anti-CTLA-4 and combination therapies, is known as ICI-induced colitis (ICI-IC), an inflammation of the colon caused by an overactive immune response that results in significant morbidity with symptoms such as diarrhea, abdominal pain and rectal bleeding [Martins F, et al., 563-580, s.l.: Nat Rev Clin Oncol., Vol. September; 16(9) (2019); Nicolaides S, Boussioutas A. 691, Cancers: s.n., Vol. January 23; 15(3) (2023)]. The reported incidence of ICI-IC varies; The rate of all-grade ICI-IC ranges from 1% to 44.2%, and it depends on different risk factors, such as the treatment regimen and the health condition of the patients, which are further detailed below [Weingarden A R, et al., 772-798, s.l.: World J Gastrointest Oncol., Vol. August 15; 13(8) (2021)].

Severe ICI-associated adverse effects that lead to ICI discontinuation affect approximately 15%, 10%, and 5% of patients receiving anti-CTLA-4, anti-PD-1 or anti-PD-L1 agents, respectively, and 50% of patients receiving a combination of nivolumab (anti-PD-1) and ipilimumab (anti-CTLA-4). Moreover, a systematic review focused on the fatal toxicities of all ICI therapies using the WHO database found that, between the years 2009-2028, 198 deaths were reported due to CTLA-4 treatment, and ICI-IC was the cause in 135 cases (70%). In patients receiving combination therapies, ICI-related deaths were mainly attributed to colitis (37%).

Thus, in some embodiments, the systems of the present disclosure may be used to re-program the microbiome to produce mature human Interleukin 10 (IL-10), that once released to the gut, interfere with inflammatory pathways in adjacent to the site of inflammation and alleviate the immune response in patients suffer from ICI-IC.

The present disclosure thus provides effective and safe treatment for attenuating immune response, that is particularly applicable in the treatment and prevention of colitis as a local impact without harming the anti-cancer impact of the ICI as in steroids or inifliximab.

In some embodiments, the present disclosure provides the use of at least one of the IL-10 microbiome modulating components and systems disclosed herein that may comprise the nucleic acid sequence as denoted by any one of SEQ ID NO: 12 to 17, and/or the IL-10 encoding variants of at least one of SEQ ID NO: 25 to 30, or any variants or derivatives thereof, together in a combined treatment with at least one ICI.

The present disclosure therefore provides in some embodiments thereof a combined therapy, specifically, methods and compositions combining the IL-10 modulatory systems of the present disclosure (comprising the selective component and the modulatory component for expressing IL-10 as the desired product), with at least one ICI, for example, anti-CTLA-4, anti-PD1 and/or anti-PD1-L. Still further, in some embodiments, the present disclosure provides combined compositions comprising at least one of the IL-10 microbiome modulating components and/or systems disclosed herein that may comprise the nucleic acid sequence as denoted by any one of SEQ ID NO: 12 to 17, and/or the IL-10 encoding variants of at least one of SEQ ID NO: 25 to 30, or any variants or derivatives thereof, together with at least one ICI.

The present disclosure thus provides therapeutic methods for treating neoplastic disorders or cancer.

As used herein to describe the present invention, “proliferative disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods, compositions and systems of the present invention may be applicable for a patient suffering from any one of non-solid and solid tumors.

Malignancy, as contemplated in the present invention may be any one of carcinomas, melanomas, lymphomas, leukemia, myeloma and sarcomas. Therefore, in some embodiments any of the methods of the invention (specifically, therapeutic, prognostic and non-therapeutic methods), systems and compositions disclosed herein, may be applicable for any of the malignancies disclosed by the present disclosure.

More specifically, carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges.

Melanoma as used herein, is a malignant tumor of melanocytes. Melanocytes are cells that produce the dark pigment, melanin, which is responsible for the color of skin. They predominantly occur in skin but are also found in other parts of the body, including the bowel and the eye. Melanoma can occur in any part of the body that contains melanocytes.

Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas.

Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

In some embodiments, the methods of the present disclosure may be applicable for any solid tumor. In more specific embodiments, the methods disclosed herein may be applicable for any malignancy that may affect any organ or tissue in any body cavity, for example, the peritoneal cavity (e.g., liposarcoma), the pleural cavity (e.g., mesothelioma, invading lung), any tumor in distinct organs, for example, the urinary bladder, ovary carcinomas, and tumors of the brain meninges. Particular and non-limiting embodiments of tumors applicable in the methods, compositions and system of the present disclosure may include but are not limited to at least one of ovarian cancer, liver carcinoma, colorectal carcinoma, breast cancer, pancreatic cancer, brain tumors and any related conditions, as well as any metastatic condition, tissue or organ thereof.

It should be understood that the methods, compositions and systems of the present disclosure are applicable for any type and/or stage and/or grade of any of the malignant disorders discussed herein or any metastasis thereof. Still further, it must be appreciated that the methods, compositions and systems of the invention may be applicable for invasive as well as non-invasive cancers. When referring to “non-invasive” cancer it should be noted as a cancer that do not grow into or invade normal tissues within or beyond the primary location. When referring to “invasive cancers” it should be noted as cancer that invades and grows in normal, healthy adjacent tissues.

Still further, in some embodiments, the methods, compositions and systems of the present disclosure are applicable for any type and/or stage and/or grade of any metastasis, metastatic cancer or status of any of the cancerous conditions disclosed herein.

As used herein the term “metastatic cancer” or “metastatic status” refers to a cancer that has spread from the place where it first started (primary cancer) to another place in the body. A tumor formed by metastatic cancer cells originated from primary tumors or other metastatic tumors, that spread using the blood and/or lymph systems, is referred to herein as a metastatic tumor or a metastasis. Further malignancies that may find utility in the present invention can comprise but are not limited to hematological malignancies (including lymphoma, leukemia, myeloproliferative disorders, Acute lymphoblastic leukemia; Acute myeloid leukemia), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. The invention may be applicable as well for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma, Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrom; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma—see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenstrom macroglobulinemia and Wilms tumor (kidney cancer).

By “patient” or “subject in need” it is meant any organism who may be in need of at least one drug and to whom the system/s and methods herein described is desired, including humans, domestic and non-domestic mammals such as canine and feline subjects, bovine, simian, equine and murine subjects, rodents, domestic birds, aquaculture, fish and exotic aquarium fish. It should be appreciated that the treated subject may be also any reptile or zoo animal. More specifically, the system/s and method/s of the invention are intended for mammals. It should be noted that specifically in cases of non-human subjects, the method of the invention may be performed using administration via injection, drinking water, feed, spraying, oral gavage and directly into the digestive tract of subjects in need thereof.

It is to be understood that the terms “treat”, “treating”, “treatment” or forms thereof, as used herein, mean preventing, ameliorating or delaying the onset of one or more clinical indications of disease activity in a subject having a pathologic disorder. Treatment refers to therapeutic treatment. Those in need of treatment are subjects suffering from a pathologic disorder. Specifically, providing a “preventive treatment” (to prevent) or a “prophylactic treatment” is acting in a protective manner, to defend against or prevent something, especially a condition or disease. The term “treatment or prevention” as used herein, refers to the complete range of therapeutically positive effects of administrating to a subject including inhibition, reduction of, alleviation of, and relief from, pathologic disorder involved with at least one short term cellular stress condition/process and any associated condition, illness, symptoms, undesired side effects or related disorders. More specifically, treatment or prevention of relapse or recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction”, “decrease” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with the disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described herein.

The terms “delay”, “delaying the onset”, “retard” and all variations thereof are intended to encompass the slowing of the progress and/or exacerbation of a disorder associated with the at least one short term cellular stress condition/process and their symptoms, slowing their progress, further exacerbation or development, so as to appear later than in the absence of the treatment according to the invention.

As indicated above, the methods and compositions provided by the present invention may be used for the treatment of a “pathological disorder”, i.e., pathologic disorder or condition involved with at least one short term cellular stress condition/process, which refers to a condition, in which there is a disturbance of normal functioning, any abnormal condition of the body or mind that causes discomfort, dysfunction, or distress to the person affected or those in contact with that person. It should be noted that the terms “disease”, “disorder”, “condition” and “illness”, are equally used herein.

It should be appreciated that any of the methods, systems and compositions described by the invention may be applicable for treating and/or ameliorating any of the disorders disclosed herein or any condition associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

A further aspect of the present disclosure relates to a pharmaceutical composition or kit comprising at least one of: (I) (a) at least one microbiome modulating component comprising: (i) at least one nucleic acid sequence encoding and/or modulating the at least one product of interest and/or at least one element participating in the production and/or metabolism of the at least one product of interest; and (ii), at least one cas gene and at least one CRISPR array, comprising at least one spacer; and (b) at least one selective component comprising at least one protospacer. The at least one protospacer is targeted by at least one spacer of the CRISPR array of (a), thereby inactivating the selective component. The disclosed composition or kit may additionally or alternatively comprise in some embodiments, (II) at least one bacterial cell and/or a population of the bacterial cells producing the product of interest, or any product thereof. In yet some alternative or additional embodiments, the disclosed compositions or kits may comprise (III), any combination of the at least one of (I) and (II), optionally, with at least one therapeutic agent. Still further, the composition or kit of the present disclosure may optionally further comprise at least one of pharmaceutically acceptable carrier/s, diluent/s, excipient/s and additive/s.

In some embodiments, the system used by the disclosed methods is as defined by the invention, and the cell or cell population is as defined by the present disclosure.

In some embodiments, the product of interest produced by the disclosed compositions and/or kits is a product affecting and/or modulating immune-related processes and/or conditions. Such product is a protein or polypeptide product encoded by a sequence comprised within at least one nucleic acid sequence of the microbiome modulating component of (a)(i).

In some further embodiments, the product of interest is a product affecting and/or modulating at least one immune-related condition, said immune-related condition is an inflammatory condition.

In more specific embodiments, the product of interest is at least one cytokine.

More specifically, in some embodiments, the cytokine is at least one of IL-10, IL-2, and IL-17.

In more specific embodiments of the disclosed bacterial cell and/or a population, the cytokine is IL-10. In some embodiments, the IL-10 is useful for an inflammatory condition. Thus in some embodiments, the disclosed compositions and/or kits may be applicable for treating any one of Crohn's disease, colitis, rheumatoid arthritis (RA) and psoriasis.

More specifically, pharmaceutical compositions according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients, specifically, the nucleic acid delivery vehicle of the invention with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations.

In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. The active agent may be systemic after administration or may be localized by the use of regional administration, intramural administration, or use of an implant that acts to retain the active dose at the site of implantation. The active agent may be formulated for immediate activity or it may be formulated for sustained release. Illustrative and non-limiting embodiments for the formulation of the microbiome modulatory systems of the present disclosure, and/or any components thereof, specifically, the microbiome modulating component and/or the selective component, are disclosed in Example 11. Still further, it should be understood that any of the drugs disclosed in the present disclosure in connection with other aspects of the invention, is also applicable in the present aspect as well.

In yet some alternative embodiments, the composition of the present disclosure, as well as the disclosed systems and any components thereof, specifically, the modulatory component and/or the selective component as disclosed herein, or any cells or population of cells (e.g., bacterial cells) comprising the same, may be formulated as an oral dosage form. In yet some further embodiments, the composition disclosed herein may be formulated in an oral dosage unit form.

In some embodiments, the oral dosage form may be administered orally, for example, as a solution (e.g., syrup), or as a powder, tablet, capsule, and the like. In certain embodiments the compositions and/or systems and/or kits of the invention may be formulated in a formulation adapted for add-on to a solid, semi-solid or liquid food, beverage, food additive, food supplement, medical food, botanical drug, drug and/or any type of pharmaceutical compound.

In some embodiments, the add-on composition according to the present disclosure that comprises the disclosed systems and/or any components thereof, specifically, the modulatory component and/or the selective component as disclosed herein, or any delivery vehicle comprising these components, or any cells or population of cells (e.g., bacterial cells) comprising the same, or any and/or systems and/or composition and/or kits thereof, may be formulated as a food additive, food supplement or medical food. In other embodiment, such add-on composition of the invention may be further added or combined with drugs or any type of pharmaceutical products. The term ‘add-on’ as used herein is meant a composition or dosage unit form of the at least one systems and/or any components thereof, specifically, the modulatory component and/or the selective component as disclosed herein, or any delivery vehicle comprising these components, or any cells or population of cells (e.g., bacterial cells) comprising the same of the present disclosure that may be added to existing compound, composition or material (e.g., food or beverage), enhancing or modulating desired properties thereof or alternatively, adding specific desired property to an existing compound, composition, food or beverage.

More specifically, in certain embodiments, the at least one system and/or any components thereof, specifically, the modulatory component and/or the selective component as disclosed herein, or any delivery vehicle comprising these components, or any cells or population of cells (e.g., bacterial cells) of the present disclosure, or any dosage form or composition thereof may be an add-on to a food supplement, or alternatively, may be used as a food supplement. A food supplement, the term coined by the European Commission for Food and Feed Safety, or a dietary supplement, an analogous term adopted by the FDA, relates to any kind of substances, natural or synthetic, with a nutritional or physiological effect whose purpose is to supplement normal or restricted diet. In this sense, this term also encompasses food additives and dietary ingredients. Further, under the Dietary Supplement Health and Education Act of 1994 (DSHEA), a statute of US Federal legislation, the term dietary supplement is defined as a product intended to supplement the diet that bears or contains one or more of the following dietary ingredients: a vitamin, a mineral, an herb or other botanical, a dietary substance for use by a subject to supplement the diet by increasing the total dietary intake, or a concentrate, metabolite, constituent, extract, or combination of any of the aforementioned ingredients Food or dietary supplements are marketed a form of pills, capsules, powders, drinks, and energy bars and other dose forms. Unlike drugs, however, they are mainly unregulated, i.e., marketed without proof of effectiveness or safety. Therefore, the European and the US laws regulate dietary supplements under a different set of regulations than those covering “conventional” foods and drug products. According thereto, a dietary supplement must be labeled as such and be intended for ingestion and must not be represented for use as conventional food or as a sole item of a meal or a diet. However, the add-on dosage form or composition that comprise the systems and/or any components thereof, specifically, the modulatory component and/or the selective component as disclosed herein, or any delivery vehicle comprising these components, or any cells or population of cells (e.g., bacterial cells) provided herein, may be added to a meal or beverage consumed by the subject.

In yet some further embodiments, the systems and/or any components thereof, specifically, the modulatory component and/or the selective component as disclosed herein, or any delivery vehicle comprising these components, or any cells or population of cells (e.g., bacterial cells) or any composition thereof, in accordance with the present disclosure may be an add-on to medical foods or may be consumed as a medical food. Further in this connection should be mentioned medical foods, which are foods that are specially formulated and intended for the dietary management of a disease that has distinctive nutritional needs that cannot be met by normal diet alone. Of particular interest are add-on formulations of the disclosed components, delivery vehicles comprising the disclosed components (the modulatory and/or the selective components) systems and/or cells, that may be formulated as add-o formulations to a drug, specifically, any of the drugs disclosed by the present disclosure (e.g., in Table 1). More specifically, as a add-on formulation to Irinotecan, or any derivatives and biosimilars thereof.

More specifically, pharmaceutical compositions according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients, specifically, the nucleic acid delivery vehicle/s of the present disclosure (e.g., modulatory component and/or the selective component of the disclosed system) with liquid carriers or finely divided solid carriers or both, and then, if necessary, shaping the product. The compositions may be formulated into any of many possible dosage forms such as, but not limited to, tablets, pills, capsules, liquid syrups, soft gels, sprays, matrixes, suppositories and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations.

In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. The active agent may be systemic after administration or may be localized by the use of regional administration, intramural administration, or use of an implant that acts to retain the active dose at the site of implantation. The active agent (e.g., the modulatory and/or selective components and systems thereof, may be formulated for immediate activity or it may be formulated for sustained release.

It should be appreciated that the compositions and/or kits of the disclosed systems and/or any components thereof, specifically, the modulatory component and/or the selective component as disclosed herein, or any delivery vehicle comprising these components, or any cells or population of cells (e.g., bacterial cells), may be formulated in any appropriate formulation.

As used interchangeably herein, “dosage units”, “dosage forms”, “oral or injectable dosage units”, “dosage unit forms”, “oral or injectable dosage unit forms” and the like refer to both, solid dosage forms as known in the art, or to a liquid dosage form. The dosage forms are intended for peroral use, i.e., to be swallowed (ingested), or even injected or applicated in any other means, either by a subject in need thereof, or for administration by a medical practitioner. The terms “active substance” or “active ingredient”, used herein interchangeably, refer to a therapeutically or physiologically active substance, specifically, the systems and/or any components thereof, specifically, the modulatory component and/or the selective component as disclosed herein, or any delivery vehicle comprising these components, or any cells or population of cells (e.g., bacterial cells) disclosed herein, that provides a therapeutic/physiological effect to a patient, and can also refer to a mixture of at least two thereof.

All definitions, as defined and used herein, should be understood to control over dictionary definitions, definitions in documents incorporated by reference, and/or ordinary meanings of the defined terms.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to ±10%.

The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should understood to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

### Experimental Procedures

### Plasmids:

pYF57—a plasmid based on p15A replicon, constructed by the present inventors, comprising the nucleic acid sequence as denoted by SEQ ID NO: 1.

### Antibodies:

IL-10 antibodies ELISA kit, Cat no. #DY217B, R&D systems.

Il-2 antibodies ELISA kit, Cat no. #ab229441, Abcam.

### Cells:

HEK-Blue™ IL-10 cells were engineered from the human embryonic kidney HEK293 cell line to detect bioactive human interleukin 10 (IL-10), Cat. No. #hkb-il10, InvivoGen.

### Production of Phage-Based Particles Lysate:

Phage-based particles are designed to target enteric E. coli bacterial population.

The Escherichia coli K12 BW25113 strain (CP009273) was used as the host cell for the propagation of all types of phage-based particles.

The host cells harbor plasmid that encodes the tail genes (TC1 as denoted by SEQ ID NO: 5 or TC20 as denoted by SEQ ID NO: 6) to facilitate the propagation of intact active phage-based particles.

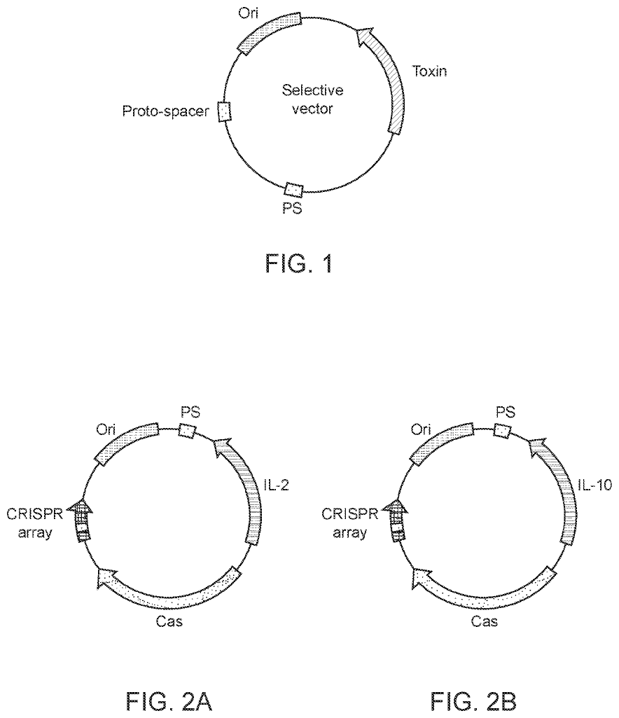

Two different host cells were used to produce two different types of phage-based particles:
i) For the production of particles containing the nucleic acid sequence encoding the desired product of interest-expressing vector, the host cells also contain a plasmid with a packaging signal and desired product encoding sequence that is packed into the particles. This plasmid (expression vector) is transduced into the targeted bacteria by the engineered phage-based transducing particles.
ii) For the production of particles containing the selective vector (nucleic acid sequence as denoted by SEQ ID NO: 7), the host cells also contain plasmid with packaging signal and bacterial toxin encoding sequence that are packed into the particles (FIG. 1). This plasmid (selective vector) is transduced into the targeted bacteria by the engineered phage-based particles.

Host cells culture (harboring the plasmid that encodes the tail genes and either the expression vector or the selective vector) was grown over night in LB supplemented with the appropriate antibiotic/s at 37° C. and 220 RPM agitation. On the next day, the cells were refreshed, and the culture grow until reach OD₆₀₀ of 0.6. The culture was then infected with T7 helper-phage (nucleic acid sequence as denoted by SEQ ID NO: 8) at a multiplicity of infection (MOI) of approximately 1. The used genome of the T7 helper-phage lacks the three essential structural genes encoding the phage tail module. Three hours post infection chloroform was added to the culture followed by brief vortex.

The lysate titer was determined using the transducing forming units (TFU) assay as previously described elsewhere (WO 2018/002940).

### TFU Assay

The TFU assay is performed to test the ability of phages to transduce the modulating component into target bacteria. The assay is performed as follows:

A lysate of phages is prepared by propagating phages in BW25113 which harbors one the pYF plasmid described here. The lysate is then used to infect mid-exponential culture of target bacteria at a MOI of ~1.2 hours later, the culture is 10-fold serially diluted and plated on selective agar LB plates. The plates are incubated over-night and CFU is counted to determine the concentration of bacteria that were transduced by the phages.

### IL-2 Bioassay:

The biological assay of YebF-hIL2 is based on a published method using the IL-2-dependent T-lymphocyte cell line CTLL-2 [Lei, Gene Therapy, 707-713 (2000)]. Briefly, the YebF-hIL2-expressing E. coli culture medium is used to supplement CTLL growth media at different ratios. Cell proliferation is measured using the MTT (3-(4,5-dimethylthiazol-2-yl)-2,5-diphenyl tetrazolium bromide) assay.

### GLP-1 Bioassay

To test if the GLP-1 expressing bacteria could induce insulin secretion in human epithelial cells, Caco-2 cells were cultured with cell-free medium from overnight cultures of GLP-1 expressing bacteria without glucose. 16 hours later, the Caco-2 cells were challenged with glucose and samples are taken and analyzed for insulin secretion. In order to determine the amount of secreted insulin, samples are assayed using standard ELISA procedures with capture (E86802M at 5p g/mL) and biotinylated detection (E86306B at 1 μg/mL) antibodies.

### Example 1

### Production and Design of Microbiome Selective Components

As a proof of concept, the gut microbiome bacterial cells were targeted for production of various desired products, for demonstrating the ability of the present disclosure to manipulate bacterial populations and thereby to modulate bacterial populations, for modulation of pathologic conditions or various biological processes. The inventors first designed selective components. More specifically, for constructing the selective component, the investors used the genome of the well-known T7 phage (ATCC #BAA-1025-B2, 39936 bp). Still further, in some alternative embodiments, the selective component may comprise a transducing particle, that comprises a toxic element and a protospacer. Non-limiting example for such selective component is illustrated by FIG. 1 and various modulating components are shown in FIGS. 2, 3, 4, 5 and 6.

As described in Example 2, various desired products were designed for modifying gut microbiome bacterial populations to express products of interest that can be used as therapeutic proteins or proteins that participate in cancer, metabolic or autoimmune conditions.

Still further, in some embodiments, the lytic phage T7 was used as a selective component. It should be understood that in some embodiments, the lytic phage used as the selective component may comprise the nucleic acid sequence as denoted b SEQ ID NO: 22, or any variants and derivatives thereof. It should be further understood that in some embodiments, the protospacers within the lytic bacteriophage used as the selective components recognized by the CRISPR array of the modulating component of the system of the present disclosure may be comprised within at least one of the gp1 bacteriophage gene, with the gp6 bacteriophage gene and/or within any key regulatory control sequence.

More specifically, for preparing the selective component that comprises the T7 lytic bacteriophage, BW25113 bacterial culture was grown until reached mid-exponential growth phase, followed by infection of the culture with the selective phages T7 at a multicity of infection (MOI) of 0.001. About thirty minutes later, once the culture was clear and the lysis was complete, the culture was centrifuged for two min at maximum speed, and chloroform (0.01%) was added. The lysate is then stored at refrigerator (2-8c). The titer of the lysate is determined according to the TFU assay described in the experimental procedures.

The aforementioned lysate contains T7 phages used as the selective phages in the Examples described below. As indicated above, the CRISPR system on the modulating component contains spacers that target their corresponding proto-spacers which are located at essential genetic element on the genome of the T7 (such as gp1 which is the T7 RNA polymerase and key T7 promoters). Targeting and eliminating these elements by the CRISPR protects the bacteria containing the modulating component and drives the conversion.

In yet some further additional and/or alternative embodiments, a selective component has been construed as illustrated generally in FIG. 1. This construct is the based on the ColE1 replicon, harboring streptomycin selection marker, phage packaging signal and also comprises two genes derived from the lytic phage T7, that are known to inhibit bacterial growth: gp0.4 (GeneID:1261053) which inhibit the essential bacterial cell-division FtsZ protein (also denoted by SEQ ID NO: 23), and gp0.6 (GeneID:1261071), which inhibit the essential cytoskeleton MreB bacterial protein (also denoted by SEQ ID NO: 24). The selective construct as generally illustrated by FIG. 1, also comprises at least one protospacer recognized and targeted by at least one spacer in the CRISPR array comprised within the modulating component of the disclosed system. In some non-limiting embodiments, the selective component plasmid comprises the nucleic acid sequence as denoted by SEQ ID NO: 7, or any variants or derivatives thereof.

### Example 2

### Production and Design of Microbiome Modulating Component for Cytokine Production

The inventors designed a microbiome modulating component suitable for the expression of various interleukins, that can be used as modulating agents for treating immune-related disorders such as cancer, and inflammatory conditions.

### Bacterial Enteric Production of Human Interleukin-2

### Construction of the IL2—Expressing Vector:

As illustrated by FIG. 2, a vector encoding a microbiome modulating component for expressing IL-2 was designed and constructed as follows:

The pYF57 backbone (as denoted by SEQ ID NO: 1) was digested with the restriction enzymes XbaI and PsiI and purified from gel using commercial gel-extraction kit.

In order to produce human IL-2 in E. coli which is also secreted to the culture media, the protein was fused to the carboxyl end of a bacterial carrier protein, YebF (SEQ ID NO: 2), that is a secreted endogenous bacterial protein [(Guijin Zhang, Nature Biotechnology, 100-104 (2006)], thereby facilitating the secretion of IL-2.

The nucleic acid sequence encoding human Interleukin-2 (SEQ ID NO: 3), fused to the bacterial protein YebF (SEQ ID NO: 2), flanked with the XbaI and PsiI restriction sites, was synthesized and its sequence was verified.

The abovementioned nucleic acid sequence was next digested with XbaI and PsiI, purified from gel and ligated into the pYF57 backbone using standard laboratory ligation procedures to form the final construct pYF57-YebF-hIL2.

Positive clones were screened by colony-PCR and correct clones were verified by sequencing.

Detection of IL-2 in the bacterial culture was performed by human IL-2 ELISA Kit (#ab229441, Abcam). Functional assay for confirming that the produced IL-2 is active was performed using the IL-2 bioassay as described in Experimental procedures.

### Bacterial Enteric Production of Human INTERLEUKIN-10

### Construction of the IL10-Expressing Vector:

As illustrated by FIG. 2B, vector encodes microbiome modulating component for expressing IL-10 was designed and constructed as follows:

The pYF57 backbone (as denoted by SEQ ID NO: 1) was digested with the restriction enzymes XbaI and PsiI and purified from gel using commercial gel-extraction kit.

The nucleic acid sequence encoding human Interleukin-10 (SEQ ID NO: 4), flanked with the XbaI and PsiI restriction sites, was synthesized and its sequence was verified.

The abovementioned nucleic acid sequence was digested with XbaI and PsiI, purified from gel and ligated into the pYF57 backbone using standard laboratory ligation procedures to form the final construct pYF57-hIL10.

Positive clones are screened by colony-PCR and correct clones are verified by sequencing.

Detection of IL-10 in the bacterial culture was performed by ELISA according to manufacture instructions (Cat no. #DY217B, R&D systems).

### Example 3

### Production and Design of Microbiome Modulating Component for Interleukin-10 Production

### Construction of the IL10-Expressing Vector:

Six different nucleic acid sequences, which encode for the expression and secretion of six variants of human IL-10, were designed and constructed as follows:
a) Recombinant human IL-10 encoding an mRNA sequence that is optimized to the expression and translation in E. coli using standard, available codon-usage tables (CUT) TWIST BIOSCIENCE, USA) (hIL-10_CUT) (FIG. 3A (as denoted by SEQ ID NO: 12), comprising the IL-10 encoding sequence as denoted by SEQ ID NO: 25.
b) Recombinant human IL-10 encoding an mRNA sequence that is optimized to the expression and translation in E. coli using a NCRF++ 1 model that was trained with BW25113 E. coli proteins, to infer from amino acids and corresponding mRNA folding the appropriate codons. Then, hIL-10 amino-acid sequence and its predicted mRNA folding were fed into the model, resulting with an E. coli-optimized mRNA sequence encoding for hIL-10 (hIL-10_mod) (FIG. 3B) (as denoted by SEQ ID NO: 13), comprising the IL-10 encoding sequence as denoted by SEQ ID NO: 26.
c) Recombinant human IL-10 encoding an optimized mRNA sequence as (a). In this construct, the bacterial promoter upstream to the IL-10 encoding gene (J23113) was replaced with the bacterial Lipoprotein (LPP) promoter, which is a strong constitutive promoter of E. coli and successfully express recombinant proteins in E. coli. (hIL-10_LPP_CUT) (FIG. 3C) (as denoted by SEQ ID NO: 14), comprising the IL-10 encoding sequence as denoted by SEQ ID NO: 27.
d) Recombinant human IL-10 encoding an optimized mRNA sequence as (b) with the LPP promoter as (c) (hIL-10_LPP_mod) (FIG. 3D) (as denoted by SEQ ID NO: 15), comprising the IL-10 encoding sequence as denoted by SEQ ID NO: 28.
e) Recombinant human IL-10 encoding an optimized mRNA sequence as (a). The sequence of this sequence is mutated to alter the protein amino acid sequence in four positions which are known to enhance the affinity of the cytokine to the receptor presented on the target human cells: N18I, N92I, K99N and F111L (Gorby C, 2020) (hIL-10_mut_CUT) (FIG. 3E) (as denoted by SEQ ID NO: 16), comprising the IL-10 encoding sequence as denoted by SEQ ID NO: 29.
f) Recombinant human IL-10 encoding an optimized mRNA sequence as (b), mutated in the same four positions as (e) (hIL-10_mut_mod) (FIG. 3F) (as denoted by SEQ ID NO: 17), comprising the IL-10 encoding sequence as denoted by SEQ ID NO: 30.

In order to produce human IL-10 in E. coli which is also secreted to the culture media, each of the abovementioned recombinant hIL-10 sequences, flanked with the XbaI and PsiI restriction sites, were fused to the carboxyl end of a bacterial carrier protein, YebF, to form the constructs hIL-10_CUT_YebF, hIL-10_mod_YebF, hIL-10_LPP_CUT_YebF, hIL-10_LPP_mod_YebF, hIL-10_mut_CUT_YebF and hIL-10_mut_mod_YebF [(Guijin Zhang, Nature Biotechnology, 100-104 (2006)].

The pYF57 backbone (as denoted by SEQ ID NO: 1) was digested with the restriction enzymes XbaI and PsiI and purified from gel using commercial gel-extraction kit.

The nucleic acid sequences were digested with XbaI and PsiI, purified from gel and ligated into the pYF57 backbone using standard laboratory ligation procedures to form the final constructs pYF_hIL-10_CUT_YebF, pYF_hIL-10_mod_YebF, pYF_hIL-10_LPP_CUT_YebF, pYF_hIL-10_LPP_mod_YebF, pYF_hIL-10_mut_CUT_YebF and pYF_hIL-10_mut_mod_YebF (as illustrated by FIG. 4A to 4F, and the nucleic acid sequences as denoted by SEQ ID NO: 12 to 17).Positive clones were screened by colony-PCR and correct clones were verified by sequencing.

### Example 4

### Production and Design of Microbiome Modulating Component for Metabolic Polypeptide Production

The inventors next designed a microbiome modulating component suitable for the expression of the metabolic peptides, such as the glucagon-like peptide-1 analog, that can be used for treating metabolic conditions such as metabolic syndrome, diabetes and obesity.

### Construction of the GLP-1-Expressing Vector:

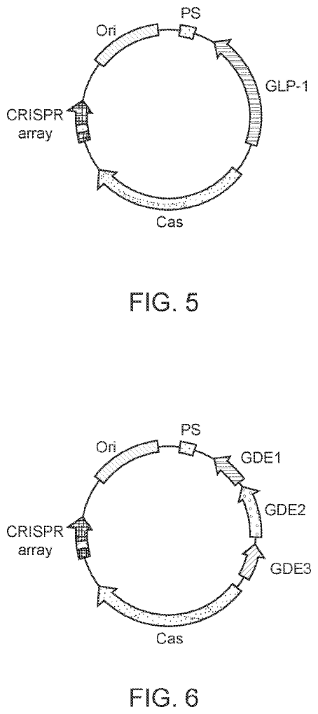

As illustrated by FIG. 5, a vector encoding a microbiome modulating component for expressing GLP-1 was designed and constructed as follows:

The pYF57 backbone (as denoted by SEQ ID NO: 1) was digested with the restriction enzymes XbaI and PsiI and purified from gel using commercial gel-extraction kit.

The nucleic acid sequence encoding GLP-1 (the human GLP-1, as denoted by SEQ ID NO: 20), fused to components of the bacterial protein FliC, flanked with the XbaI and PsiI restriction sites, was synthesized and its sequence was verified.

The abovementioned nucleic acid sequence was digested with XbaI and PsiI, purified from gel and ligated into the pYF57 backbone using standard laboratory ligation procedures to form the final construct pYF57-GLP-1.

Positive clones were screened by colony-PCR and correct clones were verified by sequencing.

Detection of GLP-1 in the bacterial culture was performed by immunodetection as previously described using anti-GLP-1 antibodies.

### Example 5

### Production and Design of Microbiome Modulating Component for Gliadin Degrading Enzymes Production

The inventors next designed a microbiome modulating component suitable for the expression of various Gliadin degrading enzymes, that can be used for Celiac treatment and management.

As illustrated by FIG. 6, vector encoding the microbiome modulating component specific for expressing various Gliadin degrading enzymes, prolyl endopeptidases, cysteine proteinase and Subtilisins, as denoted by SEQ ID NOs: 9, 10 and 11, respectively, was designed and constructed as follows:

The pYF57 backbone (as denoted by SEQ ID NO: 1) was digested with the restriction enzymes XbaI and PsiI and purified from gel using commercial gel-extraction kit.

The nucleic acid sequence encoding the three Gliadin-degrading enzymes, flanked with the XbaI and PsiI restriction sites, was synthesized and its sequence was verified.

The abovementioned nucleic acid sequence is digested with XbaI and PsiI, purified from gel and ligated into the pYF57 backbone using standard laboratory ligation procedures to form the final construct pYF57-GDE.

Positive clones are screened by colony-PCR and correct clones are verified by sequencing.

### Example 6

### Production and Design of Microbiome Modulating Component for Metabolic Polypeptide Production

The first step in the development of the microbiome modulating component, was the design and engineering of innovative payload/s to facilitate the expression of the target protein in the bacterial hosts and allow its secretion from the cells to achieve therapeutic effect.

To demonstrate the microbiome modulating component system feasibility, the human hormone GLP-1 was first selected as an example target protein. A validated oral drug, (e.g., RYBELSUS®) of this peptide was recently demonstrated as having a clear clinical benefit. This target was also shown to be successfully produced in E. coli bacterial cells [Xu F, et al., s.l.: PLoS One. Vol. July 27; 12(7):e0181939 (2017)]. Thus, providing means for continuous in situ production of this peptide may be advantageous.

For this aim, the inventors designed and constructed a microbiome modulating component payload that harbored the GLP-1 protein which was under consensus bacterial promoter and was conjugated to a known bacterial secretion system (Example 4). The payload also contained the CRISPR system, that allows enrichment of the GLP-1-producing bacterial population, and a packaging signal that facilitates the payload packaging into the phages-based transducing particles used as a delivery vehicle to the disclosed microbiome modulating component.

This payload was introduced into target bacteria, the bacteria were grown until reached stationary growth phase, and the concentration of the secreted GLP-1 protein was determined from the bacteria culture supernatant using ELISA and according to manufacture instructions (#ab229413, Abcam). As shown by FIG. 7, two payloads, containing two versions of the GLP-1 encoding sequence, were constructed and tested: unmodified and codon-optimized, which was aimed to increase production yields. The use of the payload containing the codon optimized GLP-1 protein resulted in accumulation of more GLP-1 protein extracellularly, in comparison to the non-optimized GLP-1 construct. Interestingly, theoretical calculation led to the conclusion that the concentration of secreted GLP-1 that were obtained in the current study have reached the same levels that the therapeutic GLP-1 is expected to reach in the human gut.

This study demonstrated the feasibility of the microbiome modulating component payloads to express and secrete optimized human protein from target bacteria out to the extracellular matrix, reaching therapeutic concentration levels.

### Example 7

### Production and Design of Microbiome Modulating Component for Cytokine Production

Inflammatory bowel disease (IBD) are chronic conditions affecting the digestive tract, characterized by periods of exacerbation and remission. These diseases can significantly impair the patients' quality of life if not treated.

Microbiome modulating component are developed as an orally available treatment that enables the production of biotherapeutics in targeted gut microbiome bacteria.

Inflammatory bowel disease (IBD) are chronic conditions affecting the digestive tract, characterized by periods of exacerbation and remission. These diseases occur due to a dis-regulated immune response towards gut microbes in genetically predisposed patients. Eventually, inflammation occurs due to a dis-equilibrium between pro-inflammatory pathways and cytokines (such as Tumor Necrosis Factor α (TNFα)) and anti-inflammatory cytokines (such as Interleukin 10 (IL-10)). Prolonged inflammation results in damage to the GI tract. These diseases can significantly impair the patients' quality of life if not treated [Levin A D, et al., J. Crohns Colitis, Vol. 10(8), pp. 989-97 (2016)].

A microbiome modulating component was developed as an orally available treatment that enables the production of biotherapeutics in targeted gut microbiome bacteria. Once released from the bacteria, the therapeutic proteins interfere with inflammatory pathways in adjacent to the site of inflammation and attenuate immune response to improve treatment outcome.

These oral treatments for IBD patients act locally offering a safe and effective disease management care:

anti-TNFα antibody—TNFα is a 26 kDa homotrimeric transmembrane protein that is expressed on the surface of macrophages, T-lymphocytes, natural killer cells, smooth muscle cells, and fibroblasts. TNFα is an important proinflammatory cytokine and has been implicated in the pathogenesis of many inflammatory and autoimmune diseases including rheumatoid arthritis, IBD, Crohn's disease, colitis, and more.

Since high levels of TNFα are thought to be part of the pathophysiology of many chronic inflammatory disorders, anti-TNFα antibody drugs were developed to inhibit TNFα and alleviate its role in the abovementioned diseases. Anti-TNFα drugs, such as Remicade (infliximab), Enbrel (etanercept), Humira (adalimumab), and Simponi (golimumab) have emerged as important agents in the treatment of many inflammatory diseases and were shown to have therapeutic value in the clinic (Ali T, 2013) [Levin A D. (2016) ibid.]. Nanobodies (V_{H}H) are functional recombinant single domain antibodies derived from the variable domains from heavy chain-only antibodies (HCAbs) found in camelids. The nanobodies present many known attractive characteristics, such as small size, high affinity, minimal cross-reactivity, and low immunogenicity.

Cimzia (certolizumab pegol) is another anti-TNFα biological drug and is used to treat rheumatoid arthritis and Crohn disease. The product takes advantage of the nanobodies characteristics mentioned above and comprised from humanized antigen-binding fragment (Fab′) of a monoclonal antibody. [Sandborn, William J., et al. s.l.: Clinical Gastroenterology and Hepatology, Vols. 8.8: 688-695 (2010)].

In light of the above, the inventors designed a product that targets gut bacteria to produce the Fab region of anti-TNFα antibody in the bacterial cells. The peptides are aimed to be secreted into the GI tract and act to prevent the suppression of the immune response at inflamed areas to reach local anti-inflammatory effect in the gut. The product of interest in the modulating component in this example is the pYF vector which contains the Fab region of anti-TNFα antibody (also denoted by SEQ ID NO: 21) under the expression of the J23113 promoter.

IL-10—this cytokine has anti-inflammatory properties and is pivotal for immune regulation [Iyer S S, and Cheng G. Crit. Rev. Immunol., Vol. 32(1), pp. 23-63 (2012)] and it is considered as a target for IBD therapy [Giuffrida, Paolo, et al. s.l.: Cells, Vol. 8.5: 397 (2019)]. Absence of IL-10 is associated with severe IBD in children who are born with a mutation in IL-10 receptor. Furthermore, one of the most common models for IBD in mice is based on impairing IL-10 production. Systemic use of IL-10 in clinical trials has been associated with adverse events. On the other hand, preclinical studies using it locally has been promising [Giuffrida, Paolo, ibid.]. The inventors design a product that targets gut bacteria to produce mature human IL-10 that is secreted into the gut to treat IBD with superior safety profile.

IL-2—was found to be essentially required for the growth and survival of regulatory T cells (Treg), highlighting the fundamental importance of IL-2 in Treg biology [TR., Malek. s.l.: Annu Rev Immunol., Vol. 26:453-79 (2008)]. Tregs are an immune modulating subset of CD4+ lymphocytes that antagonize the activation and effector function of multiple immune cell types and promote tolerance to self-antigens. The use of IL-2, in the setting of immune-mediated and inflammatory diseases, aims to expand the Treg population, thereby counteracting pathogenic autoimmune responses and re-establishing immune tolerance [Setoguchi R, et al., J. Exp. Med. Vol. March 7; 201(5):723-35 (2005); Malek T R, et al., Immunity. Vol. August; 17(2):167-78 (2002); Sadlack B. et al., Eur. J. Immunol. Vol. November; 25(11):305 (1995)]. A product that produces IL-2 in gut bacteria to treat IBD patients, is therefore prepared.

Therefore, three microbiome modulating components containing therapeutic targets aiming to treat IBD, specifically, IL-2, IL-10 and anti-TNFα antibody, were designed as specified above and developed.

Detection of IL-2 (Cat no. #ab229441, Abcam) and IL-10 (Cat no. #DY217B, R&D systems) in the bacterial culture was performed by ELISA assay according to manufacture instructions.

More specifically, as shown by FIG. 8, the inventors successfully designed and constructed microbiome modulating components for these two targets, IL-2 (FIG. 8A) and IL-10 (FIG. 8B, also referred to herein as the 323113 hIL-10 mod construct), and showed that these therapeutic proteins were produced and secreted from target bacteria.

The inventors developed the anti-TNFα antibody, as a nanobody (e.g., a nanobody encoded by a sequence comprising the nucleic acid sequence as denoted by SEQ ID NO: 21, or any variants and derivative thereof. Nanobodies (V_{H}H) are functional recombinant single domain antibodies derived from the variable domains from heavy chain-only antibodies (HCAbs) found in camelids [S., Muyldermans. Annu Rev Biochem., Vol. 2013; 82:775-97 (2013)]. The present invention takes advantage of the many attractive characteristics the nanobodies present (such as small size, high affinity, minimal cross-reactivity and low immunogenicity) to produce and secret anti-TNFα nanobody [S., Muyldermans. Annu. Rev. Biochem., Vol. 2013; 82:775-97 (2013); Helma J, et al., J. Cell Biol. Vol. June 8; 209(5):633-44 (2015); Steeland S, et al., Drug Discov Today. Vol. July; 21(7):1076-113 (2016)]. Moreover, once established, the inventors leverage the production of nanobodies to develop additional therapeutic targets to treat IBD in humans.

### Example 8

Establishment of a hIL-10 Production System

To further optimize the production and secretion of IL-10 by the disclosed system, various constructs of IL-10 were designed as described in the Experimental procedures. Detection and determination of the secreted hIL-10 concentration from bacteria expressing each of the six variants was next performed.

More specifically, bacteria harboring the hIL-10-encoding vector were grown over-night. The culture was centrifugated and the supernatant was taken and sterile filtered. Serial dilutions of the supernatant sample and standards were loaded to ELISA 96-well plate. Measurement of hIL-10 was performed according to manufacture instructions (#DY217B, R&D Systems). As illustrated in FIG. 9, the six constructs detailed in Example 3 were all express and secrete the hIL-10 from the E. coli host. The secretion levels of the hIL-10_CUT construct were shown to be significantly higher (up to 40-fold change) than the secretion of the remaining constructs.

Altering the hIL-10 upstream promoter had different effect on the secretion levels, and was shown to depend on the modified construct. This alteration resulted in 6-fold improvement in the case of the hIL-10_mod construct, but of an 8-fold decrease in the case of the hIL-10_CUT construct.

Although mutations in the hIL-10 proteins did not affect the secretion levels of the hIL-10_mod construct, they decreased these levels in the hIL-10_CUT construct by 20-fold.

hIL-10 Bioassay

The biological function of the three variants of hIL-10 was validated using a HEK-Blue™ IL-10 system (#hkb-il10, InvivoGen), which is a cellular system that provides measurable signal in response to binding of hIL-10 to its cellular receptor. To detect the biological activity of the recombinant hIL-10, each of the three variants and standards were incubated with HEK293 cells engineered to express IL-10 receptor. Activation of the IL-10 receptor results with measurable signal.

As illustrated in FIG. 10, three variants of the hIL-10 proteins were shown to be biologically functional according to the cellular signaling system (hIL-10_mod, the plasmid is illustrated in FIG. 4B), hIL-10_mut_mod the plasmid is illustrated in FIG. 4F) and hIL-10_mut_CUT. the plasmid is illustrated in FIG. 4E)). Interestingly, even though the hIL-10_CUT construct (the plasmid is illustrated in FIG. 4A), secretes the highest concentrations of the protein (out of the six constructs tested), it did not activate the human IL-10 receptor. On the other hand, its cognate mutant, the hIL-10_mut_CUT construct, was found to be bioactive, demonstrating the positive effect of the mutations on the ability of the protein to bind and activate its target cellular receptor. The hIL-10_mod construct and its cognate mutant hIL-10_mut_mod both shown to secrete biological functional hIL-10 proteins.

These results establish the feasibility of optimized effective expression of a biologically active IL-10.

### Example 9

In-Vitro Assay to Modify and Enrich Bacterial Population with hIL-10-Producing Bacteria

To demonstrate sustained conversion of the bacterial population to enrich the culture with IL-10-producing bacterial population in-vitro, the system of the present disclosure has been utilized to produce and secrete IL-10 in target bacteria and to enrich the bacterial culture with the desired IL-10-producing population.

More specifically, the platform (also referred to herein as a system) of the present disclosure is composed of two components provided in two transducing particles (e.g., phages), the modulating components (also referred to herein as the “IL-10-CRISPR”, or “CRISPR”), and the selective component (also referred to herein as “Selective”), which are used sequentially (FIG. 11A). In a first step, the “CRISPR” phages transduce the IL-10 payload, which express and secrete IL-10 from bacteria and also express the CRISPR system. Thereafter, the “Selective” component phages are used to kill the remaining bacteria that were not transduced with the IL-10 payload. The CRISPR system in the modulatory component together with the IL-10 payload, renders the successfully transduced bacteria protected against the “Selective” phages. The system results with bacterial population enriched with IL-10-producing bacteria (also referred to herein as the converted population).

The proof of concept (POC) of this system is demonstrated in FIG. 11B. Mid-exponential BW25113 bacterial culture, was incubated with the modulating component encoding IL-10, specifically, the pYF57_hIL-10_mod_YebF (FIG. 4B) and the CRISPR system (also referred to herein as “CRISPR” phages) at timepoint 0 hr. Two hours later, at time point 2 hr, approximately 0.1% of the bacteria in the culture contained the IL-10 payload and secrete IL-10. At timepoint 2 hr the “Selective” phages were used to selectively kill the remaining non-secreting bacteria that were not transduced by the CRISPR phages. The selective killing by the “Selective” phages enriched the bacterial culture with the desired IL-10 producing bacterial population. As shown in FIG. 11B, at the end of the study (time point 48 hr) the majority (80%) of the bacterial population was composed of IL-10 producing bacteria.

It should be noted that in spite the fact that only 0.1% of the bacterial population initially transduced by the modulating component of the invention, the “CRISPR” phages, the disclosed platform successfully selected and enriched the desired IL-10-producing bacterial population. Transduction of the “CRISPR” phages to the majority of the of the bacterial population, and the repeated use of the “Selective” phages, thus maximizes efficacy.

To determine the effect of the conversion of the bacterial population on the IL-10 concentrations that was secreted into the media, the culture was tested before and after the conversion. As shown in FIG. 11C, no IL-10 was detected in the culture before using the conversion system, whereas 24 hours after the conversion significant concentration of IL-10 was measured due to the enrichment of the IL-10 producing bacteria which secreted the protein to the media.

These results demonstrate effective conversion of bacterial population and the successful production and secretion of recombinant human protein in transduced bacterial strains, thereby modulating the composition and function of microbiome populations.

### Example 10

### In-Vivo Assay to Evaluate the Effect of IL-10 Expressing Bacteria on Colon Inflammation

The efficacy of the hIL-10-producing bacteria to attenuate the immune response in gut inflammation disorders, such as IBS, colitis etc., is demonstrated in the well-established Dextran sulfate sodium (DSS) murine model.

In this model, DSS-treated animals are also receiving the CRISPR and the Selective phages, aiming to enrich animal's gut with IL-10 producing E. coli and alleviate inflammatory responses, in comparison to non-treated animals.

More specifically, female C57BL/6 mice (8 weeks old) are used in six groups of 12 animals each (n=12) (see table below). Dysbiosis is induced by treating all animals with antibiotic, followed by oral inoculation of target E. coli using a gavage (108 CFU). The target bacteria are allowed to establish colonization in the animal's gut for 2 days, and then the drinking water of the animals are supplemented with (2.5%) DSS to induce inflammation. Two days later, the animals are orally treated with the CRISPR phages (10¹⁰ PFU) to introduce the hIL-10 construct into the E. coli target bacteria, followed by oral treatment of the Selective phages 24-hours later. Throughout the study, clinical signs are taken, body weight is recorded, and fecal samples are collected once a day. GI tissues samples are also collected and homogenized at the study termination.

TABLE 1

| Group | Bacteria | phages | DSS |
|---|---|---|---|
| 1 | − | − | − |
| 2 | + | − | − |
| 3 | + | − | + |
| 4 | + | + | − |
| 5 | + | + (concentration 1) | (2.5% in drinking water) |
| 6 | + | + (concentration 2) | (2.5% in drinking water) |

The study endpoints are:
   a) DAI score: a combined score for weight loss, stool consistency and rectal bleeding to evaluate clinical manifest.
   b) Histopathology score to GI tissue (epithelial surface structure and length of intestines) to evaluate intestinal structure.
   c) Determination of the inflammation response intensity using a set of 12 different inflammation biomarkers (ELISA).

### Example 11

### Formulation of the Selective and the Modulating Components

The product master banks (both cells and phages) are manufactured by CRO according to current GMP guidelines.

### Master Cell Banks (MCB)

The bacterial host MCB used to manufacture the clinical product have a fully documented tracking that including the following details: Storage conditions, origin of the cells, genomic manipulation, and tracking of shipment and transportation.

The bacterial host MCB are also be used as a working cell bank (WCB).

The bacterial host MCB are fully characterized for the parameters below and bank qualification guidelines:
Genomic sequencing of prophage and phage-like elements incorporated into the bacterial host genome. BLAST and sequences analysis;
Phenotypic and genomic antibiogram profiling;
Viability; determination of concentration (CFU/ml), determination of conversion from OD 600 nm to CFU/ml, tracking of viability over time (stability);
Identification; full genome and plasmidome sequencing, 16S RNA, Genotyping;
Purity;
Plating: absence of specific phage;
Official reference for stock origin (Source);
Volume and number of cryovials, buffer and storage condition;

The MCBs are stored in the CMO facility under GMP requirements and CMO monitoring and control.

### Master Phage Banks (MPB)

The MPB used to manufacture the clinical product will have full document tracking includes the following details: Storage conditions, number of infections and tracking of shipment and transportation.

The phage banks MPB will also be used as a working phage bank (WPB).

The MPB will be fully characterized according to the specifications below and bank qualification guidelines:
Volume and number of cryovials, buffer and storage condition;
Official reference for stock origin (source);
Sterility;
Identification; host range and full genome sequencing;
TEM evaluation;
Infectivity; determination of titer by plaque assay (PFU/ml), tracking of infectivity over time;
Generalized transduction;

The MPBs are stored in the CMO facility under GMP requirements and CMO monitoring and control.

### Upstream Process Development

In order to reach the required scale to produce material for FIM (first in man) studies, the following upstream process development stages are performed. The following description refers to the development of one component (either the modulating component or the Selective component, presenting one tail type, as the host recognition element). However, it should be understood that this process is used as a platform for the process of additional tail targeting various target cells and phage types. Briefly, the inventors optimize and evaluate the molecular infrastructure of the technology and perform initial production development in small scale. Tech transfer is followed by setting and scaling up to IL bioreactor to reach similar potency as in small scale. Process settings is determined during optimization to evaluate parameters effect on the production process. Confirmatory runs are produced to evaluate the process (reproducibility, analytical specification, risk/s). Further scale up to 5 L and then to 30 L to reach required scale. Prior to producing first GMP batch, an engineering run are produced and evaluated (reproducibility and analytical specification).

### Upstream Process

The following description refers to the development of one component (either the modulating component or the Selective component, presenting one tail type, as the host recognition element). However, it should be understood that this process is used as a platform for the process of additional tail targeting various target cells and phage types. Briefly, an appropriate bacterial host strain from MCB is used to inoculate small scale and grown in chemically-defined media (animal-derived material) until reaching optimal growth phase. The small-scale culture is used to inoculate fed-batched bioreactor and grown until reaching optimal growth phase. An appropriate phage from MPB is used to inoculate the bioreactor culture to allow propagation. Phage particles are harvested at the end of propagation followed by clarification steps.

### Downstream Process Development

In order to reach required scale to produce material for FIM studies, the following downstream process development stages are performed. The following description refers to the development of one component (either the modulating component or the Selective component, presenting one tail type, as the host recognition element). However, it should be understood that this process is used as a platform for the process of additional tail targeting various target cells and phage types. Briefly, for each component comprising the flow (Chromatography, TFF, formulation), setup studies are performed. According to analytical results and process performance, the process parameters are determined. For each component comprising the flow, the parameters are adjusted to optimize analytical results and process performance. The components are assembled to give a flow comprising minimal steps and maximal performance. Confirmatory runs are produced to evaluate the process (reproducibility, analytical specification, risk/s). Scaling up to reach suitable scale, which are determined according to each component flow performance and the USP product scale. Prior to producing first GMP batch, an engineering run are produced and evaluated (reproducibility and analytical specification).

### Downstream Process

The following description refers to the development of one component (either the modulating component or the Selective component, presenting one tail type, as the host recognition element). However, it should be understood that this process is used as a platform for the process of additional tail targeting various target cells and phage types. Briefly, harvested clarified product is purified using multiple chromatography steps, concentrated and buffer exchanged to formulation buffer using TFF (tangential flow filtration). Each DS (either the modulating component or the Selective component, presenting one tail type, as the host recognition element) is formulated separately in aqueous solution. Different DS of either the modulating or the selective components, that differ only in their tail, is combined to a DS cocktail of the same element (i.e. separate DS cocktails of modulating or the selective components). The cocktails DS is gelatinized and encapsulated to DP (i.e. separate DP of modulating or the selective components).

### Characterization of the Drug Substance

A DS CoA is issued separately for each element of the clinical product (modulating or the selective components). Since each element is composed of a cocktail of different phages, for each of these particles, a DS CoA will be issued separately. For instance, if the clinical product is composed of a cocktail of three different components (e.g., phages) for each of its two types of components (modulating or the selective components), six different DS CoA are issued. The CoA includes the following parameters:
Identity (by Next generation sequencing);
Potency (titer) (by Transducing formation assay);
Potency (activity) (e.g., by IL-10 receptor activation);
Residual host DNA (by qPCR);
Residual host-cell protein (by ELISA);
Endotoxin (USP<85>/EP2.6.14);
Total microbial Bioburden (USP<61>);
pH (USP<791>/EP2.2.3);
Appearance (by Visual inspection).

### Characterization of the Drug Product

A Drug product CoA is issued separately for each phage types of the product (Cargo or Selective). The CoA will include the following parameters:
Identity (by Next generation sequencing);
Potency (titer) (by Transducing formation assay);
Potency (activity) (by IL-10 receptor activation);
Residual host DNA (by qPCR);
Residual host-cell protein (by ELISA);
Endotoxin (USP<85>/EP2.6.14);
Total microbial Bioburden (USP<61>);
Specific microbial Bioburden (USP<62>);
Appearance and opalescence (EP2.2.1/EP2.2.2);
Pharmaceutical Form (by Visual inspection);
Capsule size (by Visual inspection);
Capsule weight (by Visual inspection);
Average Fill Weight (by Visual inspection);
Disintegration time (<USP701>);
pH (USP<791>/EP2.2.3);
Appearance (by Visual inspection).

### Formulation and Capsulation

The investors is formulate each of the DS and use the Capsugel® (Lonza) for DP encapsulation according to the development plan that includes all aspects of the DP preparation (formulation, capsule size and type, packaging etc.).

### Example 12

Use of the Modulatory System Producing IL-10 as a Product of Interest for Treating Patients Treated with Immune Checkpoint Inhibitors (ICIs)—a Clinical Study

This study evaluates the safety and efficacy of oral administration of the disclosed system designed for expressing IL-10 as the product of interest, to subjects treated with Immune Checkpoint Inhibitors (ICIs) that are either treated with a combination of ICIs or have a pre-existing autoimmune disorder. The study evaluates the effect of the system of the present disclosure in the incidence and severity of ICI-IC of the patients.

The study is performed following IND approval by the FDA.

More specifically, the IL-10-modulatory system formulated in an orally administrated enteric-coated capsules containing a cocktail of components (e.g., phages) that target the E. coli strains in the gut. The aim of the study is to reduce the incidence and severity of ICI-IC in treated patients.

The following clinical trials is performed as outlined below:

### 1.1. Phase 1/2a Study

### Study Outline:

Dose escalation of various doses and dosing regimens given to study patients for the duration of the ICI treatment.

Intended patient number: TBD based on pre-clinical in-vivo studies, initial estimate of 60-84 patients in study cohort based on 10-12 patients per study group and 6-7 study groups including placebo control.

### Objectives:

Safety and initial efficacy of oral administration of the IL-10-modulatory system at various doses and dosing regimens in oncology patients treated with ICIs or placebo.

### 1.2. Phase 2b Study

### Study Outline:

Treatment of study patients with dose and dosing regimen selected following phase 1/2a.

Intended patient number: TBD based phase 1/2a data.

### Objectives:

a) Reduction in incidence of IC as measured by clinical manifest of colitis (all grades) in patients treated with the IL-10-modulatory system or placebo.
b) Reduction in severity of IC as measured by MMS grade scale in patients treated with the IL-10-modulatory system or placebo.
c) Safety (AEs and SAEs) of the selected dose and dosage regimen of the IL-10-modulatory system in study patients.
d) Pharmacokinetic objectives
a. Systemic exposure analysis of human IL 10, as measured by levels of human IL10 and anti-human IL10 antibodies (immunogenicity) in the plasma of study patients.
b. Immunological changes (such as pro inflammatory cytokines) associated with IL10 activity in the gut.

These experiments establish the feasibility of using the IL-10-modulatory system for treating subjects treated with ICIs (e.g., anti-CTLA-4, and anti PD1-L).

1-70. (canceled)

71. A system for modifying target bacterial cell/s and/or target bacterial cell population to produce a product of interest, comprising:
(a) at least one microbiome modulating component comprising:
(i) at least one nucleic acid sequence encoding and/or modulating said at least one product of interest and/or at least one element participating in the production and/or metabolism of said at least one product of interest; and
(ii) at least one cas gene and at least one Clustered, Regularly Interspaced Short Palindromic Repeat (CRISPR) array, comprising at least one spacer; and
(b) at least one selective component comprising at least one protospacer, wherein said at least one protospacer is targeted by at least one spacer of the CRISPR array of (a), thereby inactivating said selective component.

72. The system according to claim 71, wherein at least one of:
(a) said selective component comprises at least one protospacer targeted by at least one spacer of said microbiome modulating component, such that said selective component is specifically inactivated by the CRISPR array of said microbiome modulating component, and wherein said selective component is comprised within at least one delivery vehicle;
(b) said microbiome modulating component is comprised within at least one delivery vehicle, said delivery vehicle specifically targets said target cell/s and/or said cells in a bacterial cell population;
(c) said delivery vehicle is, or comprises at least one genetic element, said genetic element is at least one of: at least one transducing particle, at least one bacteriophage or any fragments or parts thereof, any bacteriophage-like transducing particle, or any mixture or cocktail thereof;
(d) said selective component is comprised within at least one delivery vehicle that is, or comprises at least one genetic element, said genetic element is at least one of: at least one transducing particle, at least one lytic bacteriophage or any fragments or parts thereof, at least one plasmid that comprises and/or encodes at least one toxic element or factor that kill bacterial cells or affects, disrupt, attenuate, and/or inhibit bacterial cell growth;
(e) said bacterial cell/s and/or cell population is, or comprised within gut microbiome cell population, said cell population comprises at least one bacterium of the Proteobacteria, Firmicutes, Bacteroidetes, Actinobacteria, and phyla, or any mutant, variant or isolate or any combination thereof;
(f) said microbiome metabolism modulating component affects and/or modulates at least one of the expression, production, activity, bioavailability, clearance, stability, toxicity, and absorption of said product of interest.

73. The system according to claim 71, wherein at least one of:
(a) said product of interest is at least one of: a product affecting and/or modulating immune-related processes and/or conditions, a metabolic product and/or a product affecting and/or modulating a metabolic process or condition/s, a product affecting and/or modulating neurological or neuronal and/or neuro-behavioral processes or conditions, a therapeutic product, a cosmetic product and a nutritional product; and
(b) said product of interest is a product affecting and/or modulating immune-related processes and/or conditions, and wherein said product is a protein or polypeptide product encoded by a sequence comprised within at least one nucleic acid sequence of said microbiome modulating component of (a)(i).

74. The system according to claim 73, wherein at least one of:
(a) said product of interest is a product affecting and/or modulating at least one immune-related condition, said immune-related condition is an inflammatory condition;
(b) said product of interest is a product affecting and/or modulating at least one immune-related condition, said immune-related condition is an inflammatory condition, and wherein said product of interest is at least one cytokine;
(c) wherein said cytokine is at least one of Interleukine-10 (IL-10), Interleukine-2 (IL-2), and Interleukine-17 (IL-17);
(d) said cytokine is IL-10; and
(e) said inflammatory condition is any one of Crohn's disease, colitis, rheumatoid arthritis (RA) and psoriasis.

75. The system according to claim 73, wherein at least one of:
(a) said product of interest is a product affecting and/or modulating at least one immune-related condition, said product of interest is at least one Gliadin degrading enzyme, and wherein said immune-related condition is at least one Gluten-related disorder, wherein said at least one Gliadin degrading enzyme is at least one of: at least one prolyl endopeptidase, at least one cysteine protease and at least one subtilisin; and
(b) said product of interest is at least one agonist of the guanylyl cyclase receptor guanylate cyclase 2C (GC-C), said immune-related condition is irritable bowel syndrome, wherein said GC-C agonist is at least one peptide of the Guanylin peptides (GPs) family, or any mimetics thereof.

76. The system according to claim 73, wherein at least one of:
(a) said product of interest is a metabolic product and/or a product affecting and/or modulating a metabolic process or condition/s, and wherein said product is a protein or polypeptide product encoded by at least one nucleic acid sequence of said microbiome modulating component of (a)(i);
(b) said protein or polypeptide product increases insulin secretion and/or production, and wherein said metabolic condition/s is at least one of diabetes and metabolic syndrome and associated conditions; and
(c) said protein or polypeptide product is glucagon-like peptide-1 (GLP-1) or any mimetics and derivatives thereof.

77. The system according to claim 73, wherein at least one of:
(A) said product of interest is a product affecting and/or modulating neurological and/or neuro-behavioral processes or conditions, said product is a protein or polypeptide product encoded by at least one nucleic acid sequence of said microbiome modulating component of (a)(i), or a product produced directly or indirectly by an element encoded by the nucleic acid sequence of said microbiome modulating component of (a)(i); and
(B) said product of interest is a hormone peptide encoded by at least one nucleic acid sequence of said microbiome modulating component of (a)(i), and wherein said neuro-behavioral processes or condition is at least one of bonding and dyadic relations, and stress-related conditions; optionally wherein:
(I) said hormone peptide is Oxytocin; or
(II) said product of interest is at least one hormone produced by an element encoded by the nucleic acid sequence of said microbiome modulating component of (a)(i), and wherein said element is the tryptophan hydroxylase, and wherein said neuronal processes is at least one of neuronal differentiation and migration, axonal outgrowth, myelination and synapse formation, wherein said product is Serotonin.

78. A method for modifying target bacterial cell/s and/or target bacterial cell population to produce a product of interest, the method comprising at least one of:
a) contacting said cell/s or any cell population comprising said cell/s with at least one microbiome modulating component comprising:
(i) at least one nucleic acid sequence encoding and/or modulating at least one said product of interest and/or at least one element participating in the production and/or metabolism of said at least one product of interest; and
(ii) at least one cas gene and at least one CRISPR array comprising at least one spacer; and
(b) contacting said cells or any cell population comprising said cell/s with at least one selective component comprising at least one protospacer, wherein said at least one protospacer is targeted by at least one spacer of the CRISPR array of (a), such that said selective component is inactivated;
thereby modifying target bacterial cell/s and/or target bacterial cell population to produce said product of interest.

79. The method according to claim 78, wherein at least one of:
(a) said selective component comprises at least one protospacer targeted by at least one spacer of said microbiome modulating component, such that said selective component is specifically inactivated by the CRISPR array of said microbiome modulating component, and wherein said selective component is comprised within at least one delivery vehicle;
(b) said microbiome modulating component is comprised within at least one delivery vehicle, said delivery vehicle specifically targets said target cell/s and/or said cells in a bacterial cell population;
(c) said delivery vehicle is, or comprises at least one genetic element, said genetic element is at least one of: at least one transducing particle, at least one bacteriophage or any fragments or parts thereof, any bacteriophage-like transducing particle, or any mixture or cocktail thereof;
(d) said selective component is comprised within at least one delivery vehicle that is, or comprises at least one genetic element, said genetic element is at least one of: at least one transducing particle, at least one lytic bacteriophage or any fragments or parts thereof, at least one plasmid that comprises and/or encodes at least one toxic element or factor that kill bacterial cells or affects, disrupt, attenuate, and/or inhibit bacterial cell growth;
(e) said bacterial cell/s and/or cell population is, or comprised within gut microbiome cell population, said cell population comprises at least one bacterium of the Proteobacteria, Firmicutes, Bacteroidetes, Actinobacteria, and phyla, or any mutant, variant or isolate or any combination thereof; and
(f) said microbiome metabolism modulating component affects and/or modulates at least one of the expression, production, activity, bioavailability, clearance, stability, toxicity, and absorption of said product of interest.

80. The method according to claim 78, wherein at least one of:
(a) said product of interest is at least one of: a product affecting and/or modulating immune-related processes and/or conditions, a metabolic product and/or a product affecting and/or modulating a metabolic process or condition/s, a product affecting and/or modulating neurological, neuronal and/or neuro-behavioral processes or conditions, a therapeutic product, a cosmetic product and a nutritional product; and
(b) said product of interest is a product affecting and/or modulating immune-related processes and/or conditions, and wherein said product is a protein or polypeptide product encoded by a sequence comprised within at least one nucleic acid sequence of said microbiome modulating component of (a)(i).

81. The method according to claim 80, wherein at least one of:
(a) said product of interest is a product affecting and/or modulating at least one immune-related condition, said immune-related condition is an inflammatory condition;
(b) said product of interest is at least one cytokine;
(c) said cytokine is at least one of IL-10, IL-2, and IL-17; and
(d) said inflammatory condition is any one of Crohn's disease, colitis, RA and psoriasis.

82. The method according to claim 80, wherein at least one of:
(a) said product of interest is a product affecting and/or modulating at least one immune-related condition, said product of interest is at least one Gliadin degrading enzyme, and wherein said immune-related condition is at least one Gluten-related disorder, wherein said at least one Gliadin degrading enzyme is at least one of at least one prolyl endopeptidase, at least one cysteine protease and at least one subtilisin; and
b) said product of interest is at least one agonist of the guanylyl cyclase receptor guanylate cyclase 2C (GC-C), said immune-related condition is an inflammatory condition, said inflammatory condition is irritable bowel syndrome, wherein said GC-C agonist is at least one peptide of the Guanylin peptides (GPs) family, or any mimetics thereof.

83. The method according to claim 80, wherein at least one of:
(A) said product of interest is a metabolic product and/or a product affecting and/or modulating a metabolic process or condition/s, and wherein said product is a protein or polypeptide product encoded by at least one nucleic acid sequence of said microbiome modulating component of (a)(i); optionally, (I) said protein or polypeptide product increases insulin secretion and/or production, and wherein said metabolic condition/s is at least one of diabetes and metabolic syndrome and associated conditions; and/or (II) said protein or polypeptide product is glucagon-like peptide-1 (GLP-1) or any mimetics and derivatives thereof; and
(B) said product of interest is a product affecting and/or modulating neurological and/or neuro-behavioral processes or conditions, said product is a protein or polypeptide product encoded by at least one nucleic acid sequence of said microbiome modulating component of (a)(i), or a product produced directly or indirectly by an element encoded by the nucleic acid sequence of said microbiome modulating component of (a)(i); optionally, (I) said product of interest is a hormone peptide encoded by at least one nucleic acid sequence of said microbiome modulating component of (a)(i), and wherein said neuro-behavioral processes or condition is at least one of bonding and dyadic relations, and stress-related conditions, wherein said hormone peptide is Oxytocin; and/or (II)
said product of interest is at least one hormone produced by an element encoded by the nucleic acid sequence of said microbiome modulating component of (a)(i), and wherein said element is the tryptophan hydroxylase, and wherein said neuronal processes is at least one of neuronal differentiation and migration, axonal outgrowth, myelination and synapse formation, wherein said product is Serotonin.

84. At least one bacterial cell and/or a population of said bacterial cells, or any product thereof, producing at least one product of interest, wherein said cell and/or cell population is prepared by a method using the system according to claim 71, the method comprising at least one of:
(a) contacting said cell/s or any cell population comprising said cell/s with at least one microbiome modulating component comprising:
(i) at least one nucleic acid sequence encoding and/or modulating at least one said product of interest and/or at least one element participating in the production and/or metabolism of said at least one product of interest; and
(ii) at least one cas gene and at least one CRISPR array comprising at least one spacer; and
(b) contacting said cells or any cell population comprising said cell/s with at least one selective component comprising at least one protospacer, wherein said at least one protospacer is targeted by at least one spacer of the CRISPR array of (a), such that said selective component is inactivated;
thereby modifying target bacterial cell/s and/or target bacterial cell population to produce said product of interest.

85. The method according to claim 78, for modifying target bacterial cell/s and/or target bacterial cell population to produce a product of interest in a subject in need thereof, the method comprising the step of administering to said subject an effective amount of at least one of:
(a) at least one microbiome modulating component comprising:
(i) at least one nucleic acid sequence encoding and/or modulating at least one said product of interest and/or at least one element participating in the production and/or metabolism of said at least one product of interest; and
(ii) at least one cas gene and at least one Clustered, Regularly Interspaced Short Palindromic Repeat (CRISPR) array, comprising at least one spacer; and
(b) at least one selective component comprising at least one protospacer, wherein said at least one protospacer is targeted by at least one spacer of the CRISPR array of (a), such that said selective component is inactivated;
(c) at least one composition, kit or system comprising at least one of (a) and (b); and
(d) any combination of said at least one of (a), (b) and (c) with said at least one drug; optionally, wherein said subject is a subject suffering from an immune-related condition, a metabolic condition and a neurological condition and/or a subject undergoing/experiencing an immune-related process, a metabolic process and a neuronal and/or neuro-behavioral processes.

86. A method for treating, preventing, ameliorating, reducing or delaying the onset of at least one pathologic disorder in a subject in need thereof, the method comprising the step of administering to said subject an effective amount of at least one of:
(I) (a) at least one microbiome modulating component comprising:
(i) at least one nucleic acid sequence encoding and/or modulating at least one said product of interest and/or at least one element participating in the production and/or metabolism of said at least one product of interest; and
(ii) at least one cas gene and at least one CRISPR array, comprising at least one spacer; and
(b) at least one selective component comprising at least one protospacer, wherein said at least one protospacer is targeted by at least one spacer of the CRISPR array of (a), thereby inactivating said selective component;
(II) at least one bacterial cell and/or a population of said bacterial cells, or any composition or product thereof, producing said product of interest;
(III) at least one composition, kit or system comprising at least one of (I) and (II); and
(IV) any combination of said at least one of (I), (II) and (III) with at least one therapeutic agent.

87. The method according to claim 86, wherein at least one of:
(a) said pathologic disorder is at least one of an inflammatory disorder, a metabolic disorder, a neuronal condition, an autoimmune disorder and a neoplastic disorder;
(b) said inflammatory condition wherein said inflammatory condition is any one of Crohn's disease, colitis, rheumatoid arthritis (RA), and psoriasis;
(c) said product of interest is at least one cytokine; and
(d) said cytokine is IL-10.

88. A method for modulating at least one of an immune-related process, a metabolic process and a neuronal and/or neuro-behavioral processes in a subject in need thereof, the method comprising the step of administering to said subject an effective amount of at least one of:
I) a system as defined in claim 71, wherein the system comprising:
(a) at least one microbiome modulating component comprising:
(i) at least one nucleic acid sequence encoding and/or modulating at least one said product of interest and/or at least one element participating in the production and/or metabolism of said at least one product of interest; and
(ii) at least one cas gene and at least one CRISPR array, comprising at least one spacer; and
(b) at least one selective component comprising at least one protospacer, wherein said at least one protospacer is targeted by at least one spacer of the CRISPR array of (a), thereby inactivating said selective component;
(II) at least one bacterial cell and/or a population of said bacterial cells, or any composition or product thereof, producing said product of interest, said cells prepared using said system;
(III) at least one composition, kit or system comprising at least one of (I) and (II); and
(IV) any combination of said at least one of (I), (II) and (III) with at least one therapeutic agent.

89. The method according to claim 88, wherein at least one of:
(a) said inflammatory condition wherein said inflammatory condition is any one of Crohn's disease, colitis, rheumatoid arthritis (RA), and psoriasis;
(b) said product of interest is at least one cytokine; and
(c) said cytokine is IL-10.

90. A pharmaceutical composition or kit comprising at least one of:
(I) (a) at least one microbiome modulating component comprising:
(i) at least one nucleic acid sequence encoding and/or modulating said at least one product of interest and/or at least one element participating in the production and/or metabolism of said at least one product of interest; and
(ii) at least one cas gene and at least one CRISPR array, comprising at least one spacer; and
(b) at least one selective component comprising at least one protospacer, wherein said at least one protospacer is targeted by at least one spacer of the CRISPR array of (a), thereby inactivating said selective component;
(II) at least one bacterial cell and/or a population of said bacterial cells producing said product of interest, or any product thereof; and
(III) any combination of said at least one of (I) and (II), optionally, with at least one therapeutic agent, said composition or kit optionally further comprises at least one of pharmaceutically acceptable carrier/s, diluent/s, excipient/s and additive/s.


SEQUENCE LISTING (36 sequences)

SEQ ID NO: 1 - DNA, 10990 bp, synthetic construct
  source 1..10990: mol_type = other DNA; note = pYF57
atggaaccttttaaatatatatgccattactggggaaaatcctcaaaaagcttgacgaaaggaaatgatattcatctgttaatttatcattgccttgatgttgctgctgttgcagattgctggtgggatcaatcagtcgtactgcaaaatactttttgccgaaatgaaatgctatcaaaacagagggtgaaggcctggctgttatttttcattgctcttcatgatattggaaagtttgatatacgattccaatataaatcagcagaaagttggctgaaattaaatcctgcaacgccatcacttaatggtccatcaacacaaatgtgccgtaaatttaatcatggtgcagccggtctgtattggtttaaccaggattcactttcagagcaatctctcggggattttttcagtttttttgatgccgctcctcatccttatgagtcctggtttccatgggtagaggccgttacaggacatcatggttttatattacattcccaggatcaagataagtcgcgttgggaaatgccagcttctctggcatcttatgctgcgcaagataaacaggctcgtgaggagtggatatctgtactggaagcattatttttaacgccagcggggttatctataaacgatataccacctgattgttcatcactgttagcaggtttttgctcgcttgctgactggttaggctcctggactacaacgaatacctttctgtttaatgaggatgcgccttccgacataaatgctctgagaacgtatttccaggaccgacagcaggatgcgagccgggtattggagttgagtggacttgtatcaaataagcgatgttatgaaggtgttcatgcactactggacaatggctatcaacccagacaattacaggtgttagttgatgctcttccagtagctcccgggctgacggtaatagaggcacctacaggctccggtaaaacggaaacagcgctggcctatgcttggaaacttattgatcaacaaattgcggatagtgttatttttgccctcccaacacaagctaccgcgaatgctatgcttacgagaatggaagcgagcgcgagccacttattttcatccccaaatcttattcttgctcatggcaattcacggtttaaccacctctttcaatcaataaaatcacgcgcgattactgaacaggggcaagaagaagcgtgggttcagtgttgtcagtggttgtcacaaagcaataagaaagtgtttcttgggcaaatcggcgtttgcacgattgatcaggtgttgatatcggtattgccagttaaacaccgctttatccgtggtttgggaattggtcgaagtgttttaattgttgatgaagttcatgcttacgacacctatatgaacggcttgctggaggcagtgctcaaggctcaggctgatgttggagggagtgttattcttctttccgcaaccctaccaatgaaacaaaaacagaaacttctggatacttatggtctgcatacagatccagtggaaaataactccgcatatccactcattaactggcgaggtgtgaatggtgcgcaacgttttgatctgctagctcatccagaacaactcccgccccgcttttcgattcagccagaacctatttgtttagctgacatgttacctgaccttacgatgttagagcgaatgatcgcagcggcaaacgcgggtgcacaggtctgtcttatttgcaatttggttgacgttgcacaagtatgctaccaacggctaaaggagctaaataacacgcaagtagatatagatttgtttcatgcgcgctttacgctgaacgatcgtcgtgaaaaagagaatcgagttattagcaatttcggcaaaaatgggaagcgaaatgttggacggatacttgtcgcaacccaggtcgtggaacaatcactcgacgttgattttgattggttaattactcagcattgtcctgcagatttgcttttccaacgattgggccgtttacatcgccatcatcgcaaatatcgtcccgctggttttgagattcctgttgccaccattttgctgcctgatggcgagggttacggacgacatgagcatatttatagcaacgttagagtcatgtggcggacgcagcaacatattgaggagcttaatggagcatccttatttttccctgatgcttaccggcaatggctggatagcatttacgatgatgcggaaatggatgagccagaatgggtcggcaatggcatggataaatttgaaagcgccgagtgtgaaaaaaggttcaaggctcgcaaggtcctgcagtgggctgaagaatatagcttgcaggataacgatgaaaccattcttgcggtaacgagggatggggaaatgagcctgccattattgccttatgtacaaacgtcttcaggtaaacaactgctcgatggccaggtctacgaggacctaagtcatgaacagcagtatgaggcgcttgcacttaatcgcgtcaatgtacccttcacctggaaacgtagtttttctgaagtagtagatgaagatgggttactttggctggaagggaaacagaatctggatggatgggtctggcagggtaacagtattgttattacctatacaggggatgaagggatgaccagagtcatccctgcaaatcccaaataagcactcgagtgtagaaataattttgtttaactttaataaggagatataccatggctaatttgcttattgataactggatccctgtacgcccgcgaaacggggggaaagtccaaatcataaatctgcaatcgctatactgcagtagagatcagtggcgattaagtttgccccgtgacgatatggaactggccgctttagcactgctggtttgcattgggcaaattatcgccccggcaaaagatgacgttgaatttcgacatcgcataatgaatccgctcactgaagatgagtttcaacaactcatcgcgccgtggatagatatgttctaccttaatcacgcagaacatccctttatgcagaccaaaggtgtcaaagcaaatgatgtgactccaatggaaaaactgttggctggggtaagcggcgcgacgaattgtgcatttgtcaatcaaccggggcagggtgaagcattatgtggtggatgcactgcgattgcgttattcaaccaggcgaatcaggcaccaggttttggtggtggttttaaaagcggtttacgtggaggaacacctgtaacaacgttcgtacgtgggatcgatcttcgttcaacggtgttactcaatgtcctcacattacctcgtcttcaaaaacaatttcctaatgaatcacatacggaaaaccaacctacctggattaaacctatcaagtccaatgagtctatacctgcttcgtcaattgggtttgtccgtggtctattctggcaaccagcgcatattgaattatgcgatcccattgggattggtaaatgttcttgctgtggacaggaaagcaatttgcgttataccggttttcttaaggaaaaatttacctttacagttaatgggctatggccccatccgcattccccttgtctggtaacagtcaagaaaggggaggttgaggaaaaatttcttgctttcaccacctccgcaccatcatggacacaaatcagccgagttgtggtagataagattattcaaaatgaaaatggaaatcgcgtggcggcggttgtgaatcaattcagaaatattgcgccgcaaagtcctcttgaattgattatggggggatatcgtaataatcaagcatctattcttgaacggcgtcatgatgtgttgatgtttaatcaggggtggcaacaatacggcaatgtgataaacgaaatagtgactgttggtttgggatataaaacagccttacgcaaggcgttatatacctttgcagaagggtttaaaaataaagacttcaaaggggccggagtctctgttcatgagactgcagaaaggcatttctatcgacagagtgaattattaattcccgatgtactggcgaatgttaatttttcccaggctgatgaggtaatagctgatttacgagacaaacttcatcaattgtgtgaaatgctatttaatcaatctgtagctccctatgcacatcatcctaaattaataagcacattagcgcttgcccgcgccacgctatacaaacatttacgggagttaaaaccgcaaggagggccatcaaatggctgatgaaattgatgcaatggctttatatcgagcctggcaacaactggataatggatcatgtgcgcaaattagacgtgtttcagaacctgatgaattacgcgatatccctgcgttttataggctggtgcaaccttttggttgggaaaacccacgtcaccagcaggctcttttgcgcatggtgttttgcctgagcgcaggaaagaatgtcatccgacatcaggacaaaaaatcggagcaaacaacaggtatctcgttgggaagagctttagccaatagtggaagaattaacgagcgccgtatctttcaattaattcgggctgacagaacagccgatatggtccagttacgtcgattacttactcacgccgaacccgtacttgactggccattaatggccaggatgttgacctggtggggaaagcacgaacgccagcaacttctggaagattttgtattgaccacaaacaaaaatgcgtaaggaaacctttctatgtctaactttatcaatattcatgttctgatctctcacagcccttcatgtctgaaccgcgacgatatgaacatgcagaaagacgctattttcggcggcaaaagacgagtaagaatttcaagtcaaagccttaaacgtgcgatgcgtaaaagtggttattacgcacaaaatattggtgaatccagtctcagaaccattcatcttgcacaattacgtgatgttcttcggcaaaaacttggtgaacgttttgaccaaaaaatcatcgataagacattagcgctgctctccggtaaatcagttgatgaagccgaaaagatttctgccgatgcggttactccctgggttgtgggagaaatagcctggttctgtgagcaggttgcaaaagcagaggctgataatctggatgataaaaagctgctcaaagttcttaaggaagatattgccgccatacgtgtgaatttacagcagggtgttgatattgcgcttagtggaagaatggcaaccagcggcatgatgactgagttgggaaaagttgatggtgcaatgtccattgcgcatgcgatcactactcatcaggttgattctgatattgactggttcaccgctgtagatgatttacaggaacaaggttctgcacatctgggaactcaggaattttcatcgggtgttttttatcgttatgccaacattaacctcgctcaacttcaggaaaatttaggtggtgcctccagggagcaggctctggaaattgcaacccatgttgttcatatgctggcaacagaggtccctggagcaaaacagcgtacttatgccgcttttaaccctgcggatatggtaatggttaatttctccgatatgccactttctatggcaaatgcttttgaaaaagcggttaaagcgaaagatggctttttgcaaccgtctatacaggcgtttaatcaatattgggatcgcgttgccaatggatatggtctgaacggagctgctgcgcaattcagcttatctgatgtagacccaattactgctcaagttaaacaaatgcctactttagaacagttaaaatcctgggttcgtaataatggcgaggcgtgaacatgagatcttatttgatcttgcggcttgctgggccaatgcaagcctgggggcagccgacctttgaaggaacgcgacctaccggaagatttccgacccgaagcgggttattagggctactcggggcttgtcttgggatccaacgtgatgatacttcttcattacaggcgttatcagagagtgtgcaatttgcagtgcgctgcgatgaactcattcttgacgatcgtcgtgtgtctgtaacggggttgcgtgattaccatacagtccttggagcgcgagaagattaccgtggtttgaaaagtcatgaaacgattcaaacatggcgcgaatatttatgtgatgcctcctttaccgtcgctctctggttaacaccccatgcaacgatggttatctcagaacttgaaaaagcagtattaaagcctcggtatacaccttacctggggcggagaagttgcccactaacacacccgctttttttggggacatgtcaggcatcggatcctcagaaggcgctattaaattatgagcccgttggcggcgatatatatagtgaggaatcagttacagggcatcatttaaaatttacggcgcgcgacgaaccgatgatcaccttgcctcgacaatttgcttcccgagaatggtatgtgattaaaggaggtatggatgtatctcagtaaagtcatcattgccagggcctggagcagggatctttaccaacttcaccagggattatggcatttatttccaaacagaccggatgctgctcgtgattttctttttcatgttgagaagcgaaacacaccagaaggctgtcatgttttattgcagtcagcgcaaatgcctgtttcaactgccgttgcgacagtcattaaaactaaacaggttgaatttcaacttcaggttggtgttccactctattttcggcttcgggcaaatccgatcaaaactattctcgacaatcaaaagcgcctggacagtaaagggaatattaaacgctgtcgggttccgttaataaaagaagcagaacaaatcgcgtggttgcaacgtaaattgggcaatgcggcgcgcgttgaagatgtgcatcccatatcggaacggccacagtatttttctggtgatggtaaaagtggaaagatccaaacggtttgctttgaaggtgtgctcaccatcaacgacgcgccagcgttaatagatcttgtacagcaaggtgttgggccagctaaatcgatgggatgtggcttgctatctttggctccactgtgagcggcctcttcccaacgtcctgactggtataatgagccagttcttaaaatcgcataataatactagagccaggcatcaaataaaacgaaaggctcagtcgaaagactgggcctttcgttttatctgttgtttgtcggtgaacgctctctactagagtcacactggctcaccttcgggtgggcctttctgcgtttataaggagacacaagcttaatttagtacatgtaggtctagataggaggtgactttaggaggatactttaggagactgttacagatagggacacagagagacactcaaggtaacacccaaagacaaacccaagggtcatccccaacccttagggaaccctcaaggtcaaccgaaggttgactttaggtgattggctgggctttaggtaccccctatgggggaactttaggtccgtacactgtgagatagtcactcagatttttatggtatattttaaaggaccctataggaactccataggtcctctctaagaccctctttaggtgacactgataggatggcctatctttaggtatagaccttaggatagaccattagaggctctttatgggtataccttaggacttgactctatagggacgaagtggtgtgatgcaatcataccgctgaatccctcggtgagccatcagtcggtcaggaagaccctaatcgctacaagtgagtatagagcaaacgacagactccagtgaactgaggtctccagtaagagatgcgtacccaagggcagcacaaagtaccacaagaaatcggtaggtgactctatgccgcagtaggttcaacaataagcggaacattagtctcctccctttatgttggtccttaagttaaggtgatactaatacaatataatcaccattcttagaggtcactctaggtacaccattatgcatactttatgcactctataagtattcattcagtatctatatagtaacctttcgtccctcctctccctatagtgagtcgtattaatttatcgtgtcttttcagtaacttaaagaactgaccatccgtggtctatagtgcatccctatgcagtcctaatgcataaatcaccactcaatgaaagacgtaccgtaaccctcatcgtcctcagagtacacatcaactcctcccacagacatctcaatgatatgcgtagcagcaaccgtaggacgcatcatgtgttcctcaaggaagtcagcaagtacttcaccctcgacctacgcgtcattatcctgtatcacatattctgctgacgcaccggtgcagccttttttctcctgccacatgaagcacttcactgacaccctcatcagtgccaacatagtaaggaattctactacttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacgactagtctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaataccagagctctaagtgaaatcgcgcgtccatccggattgacagctagctcagtcctaggtataatgctagcgtcgacagacgtacaggtcggacgtcgagttccccgcgccagcggggataaaccgctgagcaccgcattagccgctgcattgatgctgagttccccgcgccagcggggataaaccgcaggagatcaacctgccggtcgcgctggcggtgagttccccgcgccagcggggataaaccgctgattgctcacgttggcggcccggctagcgtgagttccccgcgccagcggggataaaccgtgtgccgctgtatgcgcaaacggcggacgtacgagttccccgcgccagcggggataaaccggatttaccaataatcttaaacgggcgaaccaatgagttccccgcgccagcggggataaaccgctgtatcacaataagttacacgtatcggagcggagttccccgcgccagcggggataaaccgttaccgcgctgaggagcgcttcccactgtgcagagttccccgcgccagcggggataaaccgcttacaaaaactgacactgggctctgcactgggagttccccgcgccagcggggataaaccggcgccgagtcattgtgtgccagatctccggaactccgctagcgctgaggtcgactgcggccgctgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttatactagttggtgcccttaatttcagataaaaaaaatccttagctttcgctaaggatgatttcaacgccctcggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgttctgcagtggtgctaggctaggtgtacgtagaagttccccttgacggctagctcagtcctaggtacagtgctagctttctccatacccgtttttttgggctagcaataaggagatatacc

SEQ ID NO: 2 - DNA, 357 bp, synthetic construct
  source 1..357: mol_type = other DNA; note = YebF
atgaaaaaaagaggggcgtttttagggctgttgttggtttctgcctgcgcatcagttttcgctgccaataatgaaaccagcaagtcggtcactttcccaaagtgtgaagatctggatgctgccggaattgccgcgagcgtaaaacgtgattatcaacaaaatcgcgtggcgcgttgggcagatgatcaaaaaattgtcggtcaggccgatcccgtggcttgggtcagtttgcaggacattcagggtaaagatgataaatggtcagtaccgctaaccgtgcgtggtaaaagtgccgatattcattaccaggtcagcgtggactgcaaagcgggaatggcggaatatcagcggcgttaa

SEQ ID NO: 3 - DNA, 5256 bp, Homo sapiens
  source 1..5256: mol_type = genomic DNA; note = Human Interleukin 2
tttatatttatcaaatttattaaatagttttactaaccaatctacatagatactatatttaacattcaacataataataaatattttgggataaataagtgaaaccattttagagcccctagggcttacaaaaagaatcataaaagatccatatttatagttttaagattaagaataatagttacaataggtagcaaaccatacattcaacaataaatataaaatttaaatatttaaataaatagaaggcctgatatgttttaagtgggaagcacttaattatcaagtcagtgttgagatgatgctttgacaaaaggtaatccatctgttcagaaattctacaatggttgctgtctcatcagcatattcacacatgaatgttgtttcagatccctataaaagaaaaatgttaattttttaaagtacagagtagtttaccttatatacagttattcccaattgaagtcttataggcctgttgcctttattttcaagcttaccaaacatattaattattcacattttcttgaaatgatcaaaatgaatgccaaaattacatattttgttatgataccaaatgatagtaacacagaagctgagattttcctcatttgttaattcagtcaatgaacacaaaaattttttccctaaattgggtgcaaataaagatttaaataaaaatagatctagacaaatattaaatgctggatgtgaaataaacttgatcgcttttcctctgaatgtacacctatatttgtgtaagttaaatgtatgaattgatatgcacattatactttcttcttttctaaatcttgattagagtctcctatttttctacttaagtgaatctccttatattttccccaaagcagaacagaactacactagagtaagctaggacatgcttctgtttgggataaatttgtgtgaagaacatcttcataaatacttacacctatccctaccccatcatagtatcaatgcaggtgaatttgccaacttttaaaagtttaaacattagaaattttgttctgtggactggctttttagaaattaggcttctctgctgaaaggagctatttaaacgcttcctctagttgataagctaaggaggaaatattttgatatatagtgagatttaaaagagagcttttcaaatttgaagtgattcctaccctgggaacaaaagcaggtgagatagagtgttccctttaaataagtattctcttttaaaaaactgttgcagtgttctgtatcctcttaagcataattgtttaaatattgtcatatcttgggatttttttattttaaaaagtgtttggcttttgaaacacagttttcagatttttgcatggttttgtcttaaatttgtatcaagagttaagaatgagttttctgatgttaattattttagagtaaaactcttgccatagataaggaactgaatctgtcaatgtctagatgacattgatctcaatgtatttttttaattcaactgaaacagtccactaataagtgattcatttggacacaactggagttacttttaggaatgtcttttactactatacttatgctgcttatttaggatacctattaactcatgaattttataccttaggagacgggatataaaaagacaaaagtgaatctcttttacatcacctgagtcccttgcatattttatgcttcctgtgcatttaaaaatacttaagattctgaaagtggggattgcctctaaaaagattgttgtttgtttagtccactggccattttaatggctccagtttttttttttaagacaaatctcttatgggtagtccttccatcaaatgataaggaaacaccatttgaaaagcctttcaggtaatcagtattggctattccagtaaatgatctgaaggaattgaaaaatatatagtaaaatctgcctgctttctgtgaaactcaactcagtaccttctctcactcccagttttgtgttaatatgaaaggtgatggagtttgggatgaggcttgtccattagtagatggtaggtgcaaactcagtagcttatactccccagttaatcatttaacattagaaatgattcatgttcacagtgtacttaaaatgtaggctaattacatgcatgggtactttacaaataccaaaaatgttatgtcactttaaaatgtggtacttttccccctacttttttttttttatttccaggagagcaaataaagtaatgccttacctttagttccagaactattacgttgatattgctgattaagtccctgggtcttaagtgaaagtttttgctttgagctaaatttagcacttcctccagaggtttgagttcttcttctagacactgaagatgtttcagttctgtggcctagaataataattatcatcagctcagtttacatagaggtcagaatcagaaattaatttccaatttattttatttggagtagcagtatgtagtttttaccattctctctgttctcaatgtcactgtaaagatggaccagtagagtttacattataaaactggaaaaactagggctcaagtacctatgactagcgttaagtgatagttgactcactggttttagcttaatttggctttgcaaaatgttagactttctgcatctaaatgcctattacagatgtaggctgattacttttccaatctgcatagaaaatcattagaaccagatgagttagaatgtggaagcataaaaatcacattctatgattataatatttgatagtctaagagttaagaatcacagaatgttgagagagaaggaactcttggatgccatctatcacaatcccaacattttacactaggtaacaataatcatgtagtagtggctctcaatatagccttgagcaagacacatttttaaatatgtcttaaatttttacttttctacaatcactacattaagatgcattttaaaaatgtttgtcccagtgaaataaaaaattataatactttgggaggacttttggacaggcctcttgattctatattatatataatatttcatatttctatggtatattacaatttaaaagaattttctccaaaataggcccatttacttctcacagtcatcttttgaagtaggcagtgaagtatcatctccactttataaataagcacactaaatctcagaatttgaccacataagtaggtagagccagaattagatttcatttctttctcattatagcattaacatagcatccttggaatcaaatatttttattagtttctaaaatcttttgtaaatgtgcaatgagggttaaatgaacaaatgaatagagtttatcattacttttggtttcaaacttagatttccatgaattctaccaccccctaaattatgcacataatctcatctttctaacactctttagtgggaaaggtaggtcaagacaataccaatcccctgatatgtccatctattgcgctttcaattcaccactacaaattctacaaattcgggtttaagtttttggcatgaacaatgtggagaaattttcttcagaaaacagcatctagtatctttatgttgctggagttgcattatgtggctctgattaaactagagtaacatctgtaagctgtctcctttacatctttcagaagggtagttagaagcagatttctcttgaattaatagaatttgagtcaatgggattgcatctgtgtccccttaaatcctattattttctgggcttggattcaccctggcacaagctctttctggcaccagattttgttcattctctcttaactggcacagctactaggtagctttacacacctaatgtagcctaggagcttggatgattatgcttatgctctatctgaatccaaaaacaacctaatgaagcttttaatcttggaaaataagtcatatagcattataaatctcaagttctattggtaaaaagataaagtccaaaattttactttgtatttctcagtgaaaattttatctttaatactactaataatagctaatgcttgtatggtatttaccttatgccagacacggttctgagtgctttacatttacttactaattctcacaaagccatattaagtatatacagttattcatttaagtagacatgaaactgaatcacaaacagagtaggtaatttgccaaagatcatgaaagctaggacatggcagagctgagatttcacttcagatatctggccccagactctgtgctattagtgttatgctgcctctcacttagaaacctttcctaaaacaagaaaagatttttaaaagtgggctccttggaaatagattttggtattttacatactaaaatgacataatcaaatattttagagttttacatttttaaatgaaaccataaatgaacatttttgtttagaattgtcttatcgataaagtgagaatattatttttatgttttcttcccacccccaccaacacacaaatatatattaaaaatataggtaaaactgttttaatagaggcttcattatcaaacttgggttttcaaaaaaacaaattaaagttactctttacctcagatgagctgctattagtcccatctgtgcaaattttcatattactttgaattttattaaaacagaaattgaacataaaatattgtacttaccttcttgggcatgtaaaacttaaatgtgagcatcctggtgagtttgggattcttgtaattctaagaaagtataatgcattgttattaagaaatgatctccagctagattactaaatgtaataattttagtaagaaaggaaatatacttacattaattccattcaaaatcatctgtaaatccagcagtaaatgctccagttgtagctgtgttttctttgtagaacttgaagtaggtgcactgtttgtgacaagtgcaagacttagtgcaatgcaagacaggagttgcatcctgtacattgtggcaggagttgaggttactgtgagtagtgattaaagagagtgatagggaactcttgaacaagagatgcaatttatactgttaattctggaaaaatattatgggggtgtcaaaatgttttacatattacacatattttcaaagactttacctgtctgaaaaaacattaccttcatttttcctcttctgatgactctttggaatttctttaaacccccaaagactgactgaatggatgtaggtgaaatccctctttgttacattagcccacacttaggtgatag

SEQ ID NO: 4 - DNA, 6940 bp, Homo sapiens
  source 1..6940: mol_type = genomic DNA; note = Human Interleukin 10
tgatgtgaataagatacatttatttattcaaaattaaaatctgctatgaagacagacaaacaatgtaacattcccagaggaattgaataaattctagttaaaactgagttctattagaaccaaatttatttttatttttattttttgagacagagtcttgctctgtcacccaggctggagtacaggggcatgatatcagctcactgcaacttccatctcctgggttcaagcaattctcttgcctcagcctcccaagtagctgggattacaggtgcgcgccaccatgcccggctaatttttgtatttttagtagagacggggtttcaccatgttgaccaggctggttaggaactcctgacctcaagtgatccacccgcctcagcctcccaaagtgctgggattacaggcgtgagccaccgcgcccggcctagaaccaaatttaggttgttataaataacaagctggccacagctttcaagaatgaagtggttggggaatgaggttaggggaatccctccgagacactggaaggtgaattaatcatcaaaggggctccctggtttctcttcctaagagtatttgtagcagttaggaagccccaagcccagagacaagataaattagagggaggtcagggaaaacagctcaacagctagaaagcgtggtcaggcttggaatggaagcttctgttggctccccaaagaacattttttttcctcccttatgtaacttataatttatcttaaaacactcaaataccatagtgtgtcaccctatggaaacagcttaaaaacaggtgaaaataataaatattgaaaaaaattataatattgggcttctttctaaatcgttcacagagaagctcagtaaataaatagaaatgggggttgaggtatcagaggtaataaatattctataagagaggtacaataaggtttctcaaggggctgggtcagctatcccagagccccagatccgattttggagacctctaatttatgtcctagagtctatagagtcgccaccctgatgtctcagtttcgtatcttcattgtcatgtaggcttctatgtagttgatgaagatgtcaaactcactcatggctttgtagatgcctttctcttggagctgtgcagagagataagaaacatacagttaaaatcctttctggggactaaataggctcccctccctcatgctcatggatgggcatgaccttgagtgcagaggttgcttgttctccctcacgctgggaagtaaaccagaggataggcctagctgccttgggaaaggctgtgcattgaccttcatctcctccaggcctccccctagcaaggggagcattcattttgcaaagcactgtcttcaaaatgatgccttttcaacaattttaaaggtcacgtattcacatgatgattatgcagagtttgatgaaaagacattagaggaagcgcttcgaaagcaagacggtgagaggagaggagggagccttttaaatgtctgtgttctcccaccaacacactgagcaggccctgccctgccgccctgacctactatgagaaattcacaccccctgatgttggcactctgtgcagtgtctgtccccagcccacgaggaggacataggccgcacggtttctgggaaatcagactgaaaaagttcacgcacagttggaagtgtgaaaccccatttcgtttgggaactgacattctcaggcacctccgcaggtgcgaagggcagatggaaagcgggcttgaaccaactccaggttaagtgagaatccagcttcctgctcattctgagttctggggagtctgggctgaacccccaggtgtatttgtatgtgcacatttatgtgccagcttcctgcacgtgtgggttcagcctagattcccaacaagctactttctgtactttctgtttcctttgtcactctccccagcatgcagctcactgtgttcacaggctggccggccagcctaacccgcaagcctgcaaaggcagcgagcagtcatttagaaacccccaaagacacttaacagaaaacaaatacaatcaaggcagcagctcctcactggctgagcaggtcataccatctgtcaggttcccacactctctccaatgaagaatggggcctattgagtccccaccaccttccatgctttggggttggggagtgggcatgggttgtgctctgcagaccctctctgccaccatccaagctcaccttattaaaggcattcttcacctgctccacggccttgctcttgttttcacagggaagaaatcgatgctgtggaagaaaagagaaagtgttggtgatcctggcttccagctccatgtccatcacacttaggggacaagctggtggcatcatgaggaggccagatttatccaaatgccttgcctcacagctcccagagagaactgagccctgcttccatggccctgggagttaaatgtatttctccaggactcactccaagatgtttgcaaggcttggatgacaacccaggtcttgggattccttcctcccccagaactctacagtatctcctgcagtgctgagcgaggcattcttcagcctgtccttttctgcctctctctcctgttcctccccgtcagtcacagccaccgaggaacacttctaagcatctgaggagggaagctactgggaccaaacccttcacatagccttgtcctacaaatagacaccaactgattttccaatattggtgacagaacaggggaaaccattagtagtgagggcatcatttcagttggagaaataaattacctggaggaattactcatagacactagcctggggatgaatagcccccttgtcccttctaaaatgccaggggaaatcctgtcagtcaattgggcccttcttagagctttgctttcaaaacattgagattaaaagaggtgaatgatgtttcctctgcttgggaaccttagctccccgcccagggtctagttacaaagaggcagtggaggcctgactgaagctctgggctccttttatgaagagagaggggagtagttaataacccacaaatgactcacaaataacaggaaagctgtttgcaaatttcacatgtaaatgcctagcagccacccccaacgcctgctcaaagagaaatgagcaagagatctgactccaggagtctttcctcatttacagctagctctgccagtctgtgtctttgctgtgtctgtggatgtgagtgtccctgctggtctgtaggagatggtattttgggggcagctgcaagggaaaaaactgatctgctacttacacagcgccgtagcctcagcctgagggtcttcaggttctcccccagggagttcacatgcgccttgatgtctgggtcttggttctcagcttggggcatcacctcctccaggtaaaactggatcatctcagacaaggcttggcaacccaggtaaccctaagggcaggagccaaaggtgagtgagagattggcggaggtggctgggaggaggggctgctgcaactagcttaagaggacagcttggttctagcgatcctccttcaccagaagcctccccgaagggactgagccctttgtaaaccctctggctgctggatgtgctgagttaacatcttcccacttctccttttcaaagcgaaggaaacaaacccaattccctgcaatcaggaagcagagtctcccttcccttaatcatgctgcacactcccccagcaccccgcccctgctctcaccttaaagtcctccagcaaggactcctttaacaacaagttgtccagctgatccttcatttgctgcaggaagaacaaaaggagaatgaacttgaggtttggggaaataactgaaatgcggtctttttgatgccctttcatttagagatttttttttttaaataaaattggctctggcccaaaaaaatcaaaaggggagttttaaaaacagaggattcaacagtgatgggaccatcacttaaatcaggtcctcctcctcagagtcaagttatttaaaaaatctggccgattttgaagggaaagcagaagcttgactcagtcctggtcttctttctgcggagctacattcggctttaagggacacctatgtcaacccttcggggccttgagcccccctcatgtcctgttctctgcttttctttctccccactgtagacatccagtttagcttggaaagatcccagggattaagaagctccttctaatgcaggtttccctcatgtagagtgcttccctaaaccactggtctttcaggtctggaaacgctctaagcagaggcaatttaaatttccctgctgcaaggcatggggagcatcttcacctcgaatcagacatttctttacttctcctagccaataaagtatagagcgccagcaggatcttataagtttctcagcacgagagagaacgcatttactttataaggcaggtttcctgcacatttactgtatcatctggcttagcttaactcaccaaacccatggcttgatctaggattctcttaagaatttaagtttgggggaataggtgttggggatggaggtggaggcgcaggaggagggttcttatagttccaggagaatgtctagttcaggcagtcccaggaagagagaaaggacaggaaggaatcatactcacaaagaaagtcttcactctgctgaaggcatctcggagatctcgaagcatgttaggcaggttgcctgggaagtgggtgcagctgttctcagactgggtgccctggcctgggctggccctcaccccagtcaggaggaccaggcaacagagcagtgctgagctgtgcatgccttcttttgcaagtctgtcttgtggtttggttttgcaagagcaagcccctgatgtgtagaccttcacctctctgtcccccttttatattgtaagctcagggaggcctcttcattcattaaaaagccacaatcaaggtttcccggcacaggattttttctgcttagagctcctccttctctaacctctctaataaacttagttttcaatttttgcatcgtaagcaaaaatgattggttgaacatgaacttctgcattacagctatttttaggatgggctacctctcttagaataattttttagcttctcaattaaaaaaagttgatttcctggggagaacagctgttctgtgcgcagaggccctcagctgtgggttctcattcgcgtgttcctaggtcacagtgacgtggacaaattgcccattccagaatacaatgggattgagaaataattgggtccccccaacctgggatgaatacccaagacttctccttgctaacttaggcagtcaccttaggtctctgggccttagtttccccaagtaaaaatgagggggtgggctaaatatcctcaaagttcccaagcagcccttccattttactttccagagactggcttcctacagtacaggcggggtcacaggatgtgttccaggctcctttaccccgatttcattaggattctcaggcacatgtttccacctcttcagctgtcccccaccccaactgtgcttgggggaagtgggtaagagtagtctgcacttgctgaaagcttcttatatgctagtcaggtagtgctcaccatgacccctaccgtctctattttatagtgagcaaactgaggcacagagatattacatcacctgtacaagggtacaccagtgccaactgagaatttggtttcctcaccctactgtacaccatctccagcacatagaatgaaaccttggattaaattggccttagagtttcttttagttgtaagcttctgtggctggagtctaaagtttaaaagatggggtggaagaagttgaaataacaaggaaaagaagtcaggattccatggaggctggataggaggtcccttactttcctcttacctatccctacttccccttcccaaagaagccttagtagtgttgtcttggatttgtgtgtgtgtgtgtgtgtgtgtgtgtgtgtgtgtgtgtgtgtgtagaaggtaaggggagccagttgggttgcggcgaggagtgttgctctacctggggaaggaccggggcagtgacatcaggacttcttcagatatctggaaagtctctggggatcctttgattgagccttcaccttccccttccacaggggctttgcctgccattccagtttagactgtaactgggaggaacactgacctaggtgcaggaggcctgggctctgccttggccctgccactctatagtcagggcaaatcactcatcctccctgtgtctgtttttcatctgtcccaacagggcaatagcacctgtcctcctgcctgccagggagcattgagcttaagtagccatggacccagatggagggcatttccagtgttctgctagttcttcacctttctgcatcagcaatgggacaactccagtggcagcaggttctgagcttgctgcactagagaaggtcactcactgcaaggggtgcaaacaccttggggcccagccagcgtgaagtgagtgggagggagggatgagagggcccacgcccaccctgtggatgtccctgctgtgtgacctatggatcagggtcaggggactgtggtgtctcttcccaactcttcagggagtgtcaatgtagaaacctaagaatcaggaagtgagaaagagcctgctagaaactagtatggagctaactcagaacttactggtcctgccagacttagactggcttaagggttctgtctgagttctgctgccaggaagcagagagaaggggaccaggacctgggtttccccttcttgcacctttctccttccccctcggctcctgggttttctgggctgtgtctgtgttagcctgcaggtctaaccccctctctgagctgggtgttccagaagtggactcagcatgggcacc

SEQ ID NO: 5 - DNA, 7159 bp, synthetic construct
  source 1..7159: mol_type = other DNA; note = Tail plasmid-TC1
gccggcgcggccgcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatcgcggaacccctatttgtttatttttctaaatacattcaaatatgtatccgctcatgagacaataaccctgataaatgcttcaataatattgaaaaaggaagagtcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaagatgcccagctgtctttcgctgctgagggtgacgatcccgcggatcccgggcccgtcgactgcagaggcctgcatgcaagcttggcgtaatcggcgaacgtggtccggataatacgactcactatagggcgaattgggcccgacgtcgcatgctcccggccgccatggccgcgggatataaggtcgacaagaaggagatatacatatggctaacgtaattaaaaccgttttgacttaccagttagatggctccaatcgtgattttaatatcccgtttgagtatctagcccgtaagttcgtagtggtaactcttattggtgtagaccgaaaggtccttacgattaatacagactatcgctttgctacacgtactactatctctctgacaaaggcttggggtccagccgatggctacacgaccatcgagttacgtcgagtaacctccactaccgaccgattggttgactttacggatggttcaatcctccgcgcgtatgaccttaacgtcgctcagattcaaacgatgcacgtagcggaagaggcccgtgacctcactacggatactatcggtgtcaataacgatggtcacttggatgctcgtggtcgtcgaattgtgaacctagcgaacgccgtggatgaccgcgatgctgttccgtttggtcaactaaagaccatgaaccagaactcatggcaagcacgtaatgaagccttacagttccgtaatgaggctgagactttcagaaaccaagcggagggctttaagaacgagtccagtaccaacgctacgaacacaaagcagtggcgcgatgagaccaagggtttccgagacgaagccaagcggttcaagaatacggctggtcaatacgctacatctgctgggaactctgcttccgctgcgcatcaatctgaggtaaacgctgagaactctgccacagcatccgctaactctgctcatttggcagaacagcaagcagaccgtgcggaacgtgaggcagacaagctggaaaattacaatggattggctggtgcaattgataaggtagatggaaccaatgtgtactggaaaggaaatattcacgctaacgggcgcctttacatgaccacaaacggttttgactgtggccagtatcaacagttctttggtggtgtcactaatcgttactctgtcatggagtggggagatgagaacggatggctgatgtatgttcaacgtagagagtggacaacagcgataggcggtaacatccagttagtagtaaacggacagatcatcacccaaggtggagccatgaccggtcagctaaaattgcagaatgggcatgttcttcaattagagtccgcatccgacaaggcgcactatattctatctaaagatggtaacaggaataactggtacattggtagagggtcagataacaacaatgactgtaccttccactcctatgtacatggtacgaccttaacactcaagcaggactatgcagtagttaacaaacacttccacgtaggtcaggccgttgtggccactgatggtaatattcaaggtactaagtggggaggtaaatggctggatgcttacctacgtgacagcttcgttgcgaagtccaaggcgtggactcaggtgtggtctggtagtgctggcggtggggtaagtgtgactgtttcacaggatctccgcttccgcaatatctggattaagtgtgccaacaactcttggaacttcttccgtactggccccgatggaatctacttcatagcctctgatggtggatggttacgattccaaatacactccaacggtctcggattcaagaatattgcagacagtcgttcagtacctaatgcaatcatggtggagaacgagtaagtcgacactagttaatacgactcactatagggagattgaggggttttttgctgaaaggaggaactatatgcgctcatacgatatgaacgttgagactgccgctgagttatcagctgtgaacgacattctggcgtctatcggtgaacctccggtatcaacgctggaaggtgacgctaacgcagatgcagcgaacgctcggcgtattctcaacaagattaaccgacagattcaatctcgtggatggacgttcaacattgaggaaggcataacgctactacctgatgtttactccaacctgattgtatacagtgacgactatttatccctaatgtctacttccggtcaatccatctacgttaaccgaggtggctatgtgtatgaccgaacgagtcaatcagaccgctttgactctggtattactgtgaacattattcgtctccgcgactacgatgagatgcctgagtgcttccgttactggattgtcaccaaggcttcccgtcagttcaacaaccgattctttggggcaccggaagtagagggtgtactccaagaagaggaagatgaggctagacgtctctgcatggagtatgagatggactacggtgggtacaatatgctggatggagatgcgttcacttctggtctactgactcgctaacattaataaataaggaggctctaatggcactcattagccaatcaatcaagaacttgaagggtggtatcagccaacagcctgacatccttcgttatccagaccaagggtcacgccaagttaacggttggtcttcggagaccgagggcctccaaaagcgtccacctcttgttttcttaaatacacttggagacaacggtgcgttaggtcaagctccgtacatccacctgattaaccgagatgagcacgaacagtattacgctgtgttcactggtagcggaatccgagtgttcgacctttctggtaacgagaagcaagttaggtatcctaacggttccaactacatcaagaccgctaatccacgtaacgacctgcgaatggttactgtagcagactatacgttcatcgttaaccgtaacgttgttgcacagaagaacacaaagtctgtcaacttaccgaattacaaccctaatcaagacggattgattaacgttcgtggtggtcagtatggtagggaactaattgtacacattaacggtaaagacgttgcgaagtataagataccagatggtagtcaacctgaacacgtaaacaatacggatgcccaatggttagctgaagagttagccaagcagatgcgcactaacttgtctgattggactgtaaatgtagggcaagggttcatccatgtgaccgcacctagtggtcaacagattgactccttcacgactaaagatggctacgcagaccagttgattaaccctgtgacccactacgctcagtcgttctctaagctgccacctaatgctcctaacggctacatggtgaaaatcgtaggggacgcctctaagtctgccgaccagtattacgttcggtatgacgctgagcggaaagtttggactgagactttaggttggaacactgaggaccaagttctatgggaaaccatgccacacgctcttgtgcgagccgctgacggtaatttcgacttcaagtggcttgagtggtctcctaagtcttgtggtgacgttgacaccaacccttggccttcttttgttggttcaagtattaacgatgtgttcttcttccgtaaccgcttaggattccttagtggggagaacatcatattgagtcgtacagccaaatacttcaacttctaccctgcgtccattgcgaaccttagtgatgacgaccctatagacgtagctgtgagtaccaaccgaatagcaatccttaagtacgccgttccgttctcagaagagttactcatctggtccgatgaagcacaattcgtcctgactgcctcgggtactctcacatctaagtcggttgagttgaacctaacgacccagtttgacgtacaggaccgagcgagaccttttgggattgggcgtaatgtctactttgctagtccgaggtccagcttcacgtccatccacaggtactacgctgtgcaggatgtcagttccgttaagaatgctgaggacattacatcacacgttcctaactacatccctaatggtgtgttcagtatttgcggaagtggtacggaaaacttctgttcggtactatctcacggggaccctagtaaaatcttcatgtacaaattcctgtacctgaacgaagagttaaggcaacagtcgtggtctcattgggactttggggaaaacgtacaggttctagcttgtcagagtatcagctcagatatgtatgtgattcttcgcaatgagttcaatacgttcctagctagaatctctttcactaagaacgccattgacttacagggagaaccctatcgtgcctttatggacatgaagattcgatacacgattcctagtggaacatacaacgatgacacattcactacctctattcatattccaacaatttatggtgcaaacttcgggaggggcaaaatcactgtattggagcctgatggtaagataaccgtgtttgagcaacctacggctgggtggaatagcgacccttggctgagactcagcggtaacttggagggacgcatggtgtacattgggttcaacattaacttcgtatatgagttctctaagttcctcatcaagcagactgccgacgacgggtctacctccacggaagacattgggcgcttacagttacgccgagcgtgggttaactacgagaactctggtacgtttgacatttatgttgagaaccaatcgtctaactggaagtacacaatggctggtgcccgattaggctctaacactctgagggctgggagactgaacttagggaccggacaatatcgattccctgtggttggtaacgccaagttcaacactgtatacatcttgtcagatgagactacccctctgaacatcattgggtgtggctgggaaggtaactacttacggagaagttccggtatttaactcgaggatatcgctagc

SEQ ID NO: 6 - DNA, 7172 bp, synthetic construct
  source 1..7172: mol_type = other DNA; note = Tail plasmid-TC20
gccggcgcggccgcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaagatgcccagctgtctttcgctgctgagggtgacgatcccgcggatcccgggcccgtcgactgcagaggcctgcatgcaagcttggcgtaatcggcgaacgtggtccggtaatacgactcactatagggcgaattgggcccgacgtcgcatgctcccggccgccatggccgcgggattaaggtcgacaagaaggagatatacatatggctaacgtaattaaaaccgttttgacttaccagttagatggctccaatcgtgattttaatatcccgtttgagtatctagcccgtaagttcgtagtggtaactcttattggtgtagaccgaaaggtccttacgattaatacagactatcgctttgctacacgtactactatctctctgacaaaggcttggggtccagccgatggctacacgaccatcgagttacgtcgagtaacctccactaccgaccgattggttgactttacggatggttcaatcctccgcgcgtatgaccttaacgtcgctcagattcaaacgatgcacgtagcggaagaggcccgtgacctcactacggatactatcggtgtcaataacgatggtcacttggatgctcgtggtcgtcgaattgtgaacctagcgaacgccgtggatgaccgcgatgctgttccgtttggtcagttaaagacaatgaaccagaatagctggcaagcccgcaatgaagcactgcagttccgcaacgaagccgagacatttcgcaaccagaccgaagtgtttaaaaacgagagcggcaccaacgccaccaatacaaaacagtggcgcgatgaagccaacggcagtcgtgatgaagccgaacaattcaagaacaccgccggccagtatgccaccagtgccggtaatagcgcaaccgcagcccatcagagcgaagttaacgccgagaatagcgccaccgcagccgccaatagtgcaaatctggcagagcagcatgccgatcgtgccgagcgcgaagccgataaactgggcaactttaacggtctggccggcgcaattgatcgcgtggatggcaccaacgtgtactggaaaggtggcatccatgcaaatggtcgtttatatctgaccagcgatggctttgattgcggccagtaccagcagttctttggtggcagtgccggtcgttatagcgttatggaatggggcgatgaaaacggctggttaatgcatgtgcagcgccgtgaatggaccacagccattggcgacaacatccagctggtggtgaatggccacatcattgcccaaggtggcgatatgaccggtcctctgaaactgcagaacggccacgctttatatttagaaagcgccagcgataaggcccagtatatcttaagcaaagatggtaatcgcaataactggtatatcggtcgcggcagcgataacaacaatgattgcaccttccacagttacgtgtacggcaccaatttaacactgaagccggattatgccgtggtgaacaaacgctttcatgtgggtcaagctgtggtggccacagatggcaacatccaaggtaccaaatggggcggtaagtggctggacgcctatctgaacgatacctatgttaagaaaactatggcttggacacaagtttgggcagcagcaagcggtagctatatgggtggtggtagccagaccgacactttaccgcaagatttacgctttcgcaatatctggattaaaacccgtaataactactggaatttctttcgcactggtccggatggcatttactatttaagcgcagagggcggttggctgaaattccagatccacagcaacggccgtgtgttcaagaatattgccgaccgtgacgcaccgccgacagcaattgcagttgaagatgtttaagtcgacactagttaatacgactcactatagggagattgaggggttttttgctgaaaggaggaactatatgcgctcatacgatatgaacgttgagactgccgctgagttatcagctgtgaacgacattctggcgtctatcggtgaacctccggtatcaacgctggaaggtgacgctaacgcagatgcagcgaacgctcggcgtattctcaacaagattaaccgacagattcaatctcgtggatggacgttcaacattgaggaaggcataacgctactacctgatgtttactccaacctgattgtatacagtgacgactatttatccctaatgtctacttccggtcaatccatctacgttaaccgaggtggctatgtgtatgaccgaacgagtcaatcagaccgctttgactctggtattactgtgaacattattcgtctccgcgactacgatgagatgcctgagtgcttccgttactggattgtcaccaaggcttcccgtcagttcaacaaccgattctttggggcaccggaagtagagggtgtactccaagaagaggaagatgaggctagacgtctctgcatggagtatgagatggactacggtgggtacaatatgctggatggagatgcgttcacttctggtctactgactcgctaacattaataaataaggaggctctaatggcactcattagccaatcaatcaagaacttgaagggtggtatcagccaacagcctgacatccttcgttatccagaccaagggtcacgccaagttaacggttggtcttcggagaccgagggcctccaaaagcgtccacctcttgttttcttaaatacacttggagacaacggtgcgttaggtcaagctccgtacatccacctgattaaccgagatgagcacgaacagtattacgctgtgttcactggtagcggaatccgagtgttcgacctttctggtaacgagaagcaagttaggtatcctaacggttccaactacatcaagaccgctaatccacgtaacgacctgcgaatggttactgtagcagactatacgttcatcgttaaccgtaacgttgttgcacagaagaacacaaagtctgtcaacttaccgaattacaaccctaatcaagacggattgattaacgttcgtggtggtcagtatggtagggaactaattgtacacattaacggtaaagacgttgcgaagtataagataccagatggtagtcaacctgaacacgtaaacaatacggatgcccaatggttagctgaagagttagccaagcagatgcgcactaacttgtctgattggactgtaaatgtagggcaagggttcatccatgtgaccgcacctagtggtcaacagattgactccttcacgactaaagatggctacgcagaccagttgattaaccctgtgacccactacgctcagtcgttctctaagctgccacctaatgctcctaacggctacatggtgaaaatcgtaggggacgcctctaagtctgccgaccagtattacgttcggtatgacgctgagcggaaagtttggactgagactttaggttggaacactgaggaccaagttctatgggaaaccatgccacacgctcttgtgcgagccgctgacggtaatttcgacttcaagtggcttgagtggtctcctaagtcttgtggtgacgttgacaccaacccttggccttcttttgttggttcaagtattaacgatgtgttcttcttccgtaaccgcttaggattccttagtggggagaacatcatattgagtcgtacagccaaatacttcaacttctaccctgcgtccattgcgaaccttagtgatgacgaccctatagacgtagctgtgagtaccaaccgaatagcaatccttaagtacgccgttccgttctcagaagagttactcatctggtccgatgaagcacaattcgtcctgactgcctcgggtactctcacatctaagtcggttgagttgaacctaacgacccagtttgacgtacaggaccgagcgagaccttttgggattgggcgtaatgtctactttgctagtccgaggtccagcttcacgtccatccacaggtactacgctgtgcaggatgtcagttccgttaagaatgctgaggacattacatcacacgttcctaactacatccctaatggtgtgttcagtatttgcggaagtggtacggaaaacttctgttcggtactatctcacggggaccctagtaaaatcttcatgtacaaattcctgtacctgaacgaagagttaaggcaacagtcgtggtctcattgggactttggggaaaacgtacaggttctagcttgtcagagtatcagctcagatatgtatgtgattcttcgcaatgagttcaatacgttcctagctagaatctctttcactaagaacgccattgacttacagggagaaccctatcgtgcctttatggacatgaagattcgatacacgattcctagtggaacatacaacgatgacacattcactacctctattcatattccaacaatttatggtgcaaacttcgggaggggcaaaatcactgtattggagcctgatggtaagataaccgtgtttgagcaacctacggctgggtggaatagcgacccttggctgagactcagcggtaacttggagggacgcatggtgtacattgggttcaacattaacttcgtatatgagttctctaagttcctcatcaagcagactgccgacgacgggtctacctccacggaagacattgggcgcttacagttacgccgagcgtgggttaactacgagaactctggtacgtttgacatttatgttgagaaccaatcgtctaactggaagtacacaatggctggtgcccgattaggctctaacactctgagggctgggagactgaacttagggaccggacaatatcgattccctgtggttggtaacgccaagttcaacactgtatacatcttgtcagatgagactacccctctgaacatcattgggtgtggctgggaaggtaactacttacggagaagttccggtatttaactcgaggatatcgctagc

SEQ ID NO: 7 - DNA, 4325 bp, synthetic construct
  source 1..4325: mol_type = other DNA; note = Selective vector
aacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcgaggatccagtgccaacatagtaagccagtagagctcgccggcaagctgagcaccgcattagccgctgcattgatgctgggccgagcgattaagcgtgacaaagtgtgccgctgtatgcgcaaacggcggacgtacagggccgcgcagagttagttcataagaaccattcgctaaactcgaacaggactttggcggggactttaggtcgagcacagtaaggtctatgatacgaccatgctccaaaatgccctaggccatggtaagtacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgatagtacatgtaggtcgagggtgaagtacttgctgacttccttgaggaacacatgatgcgtcctacggttgctgctacgcatatcattgagatgtctgtgggaggagttgatgtgtactctgaggacgatgagggttacggtacgtctttcattgagtggtgatttatgcattaggactgcatagggatgcactatagaccacggatggtcagttctttaagttactgaaaagacacgataaattaatacgactcactatagggagaggagggacgaaaggttactatatagatactgaatgaatacttatagagtgcataaagtatgcataatggtgtacctagagtgacctctaagaatggtgattatattgtattagtatcaccttaacttaaggcgggatcgtcaccctcagcagcgaaagacagctgtcggtcagagcgtcattgcgaagctgagtgtgatcgatgccatcagcgaagggcccaaactccgagcgattaagcgtttgctggctgtcacgcctgcctgttgcttgcttggacttgcgatgtacgtgctcagctgtctttcgctgctgagggtgacgatcccgcgagggcctatggagttcctatagggtcctttaaaatataccataaaaatctgagtgactatctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccctatctgttacagtctcctaaagtatcctcctaaagtcacctcctaacgacatgtactacgatcaaaggatcttcttgagatcctttttttctgcgcgtaatcttttgccctgtaaacgaaaaaaccacctggggaggtggtttgatcgaaggttaagtcagttggggaactgcttaaccgtggtaactggctttcgcagagcacagcaaccaaatctgtccttccagtgtagccggactttggcgcacacttcaagagcaaccgcgtgtttagctaaacaaatcctctgcgaactcccagttaccaatggctgctgccagtggcgttttaccgtgcttttccgggttggactcaagtgaacagttaccggataaggcgcagcagtcgggctgaacggggagttcttgcttacagcccagcttggagcgaacgacctacaccgagccgagataccagtgtgtgagctatgagaaagcgccacacttcccgtaagggagaaaggcggaacaggtatccggtaaacggcagggtcggaacaggagagcgcaagagggagcgacccgccggaaacggtggggatctttaagtcctgtcgggtttcgcccgtactgtcagattcatggttgagcctcacggctcccacagatgcaccggaaaagcgtctgtttatgtgaactctggcaggagggcggagcctatggaaaaacgccaccggcgcggccctgctgttttgcctcacatgttagtcccctgcttatccacggaatctgtgggtaactttgtatgtgtccgcagcgcaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtattgacagctagctcagtcctagggactatgctagctttctccatacccgtttttttgggctagcgaattcgagctcggtacccggggatcctctagagaggaggatgaagagtaatgtctactaccaacgtgcaatacggtctgaccgctcaaactgtacttttctatagcgacatggtgcgctgtggctttaactggtcactcgcaatggcacagctcaaagaactgtacgaaaacaacaaggcaatagctttagaatctgctgagtgatagactcaaggtcgctcctagcgagtggcctttatgattatcacgtcgacctgtaggttcctactttaccgcttgcatcatcataggaatcatcaaaggggcactacgcaaatgatgaagcactacgttatgccaatccacacgtccaacggggcaaccgtatgtacacctgatgggttcgcaatgaaacaacgaatcgaacgccttaagcgtgaactccgcattaaccgcaagattaacaagataggttccggctatgacagaacgcactgactgcaggcatgcaagcttggctgttttggcggatgagagaagattttcagcctgatacagattaaatcag

SEQ ID NO: 8 - DNA, 35422 bp, unidentified
  source 1..35422: mol_type = other DNA; note = Enterobacteria phage
tctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccctatctgttacagtctcctaaagtatcctcctaaagtcacctcctaacgtccatcctaaagccaacacctaaagcctacacctaaagacccatcaagtcaacgcctatcttaaagtttaaacataaagaccagacctaaagaccagacctaaagacactacataaagaccagacctaaagacgccttgttgttagccataaagtgataacctttaatcattgtctttattaatacaactcactataaggagagacaacttaaagagacttaaaagattaatttaaaatttatcaaaaagagtattgacttaaagtctaacctataggatacttacagccatcgagagggacacggcgaatagccatcccaatcgacaccggggtcaaccggataagtagacagcctgataagtcgcacgaaaaacaggtattgacaacatgaagtaacatgcagtaagatacaaatcgctaggtaacactagcagcgtcaaccgggcgcacagtgccttctaggtgacttaagcgcaccacggcacataaggtgaaacaaaacggttgacaacatgaagtaaacacggtacgatgtaccacatgaaacgacagtgagtcaccacactgaaaggtgatgcggtctaacgaaacctgacctaagacgctctttaacaatctggtaaatagctcttgagtgcatgactagcggataactcaagggtatcgcaaggtgccctttatgatattcactaataactgcacgaggtaacacaagatggctatgtctaacatgacttacaacaacgttttcgaccacgcttacgaaatgctgaaagaaaacatccgttatgatgacatccgtgacactgatgacctgcacgatgctattcacatggctgccgataatgcagttccgcactactacgctgacatctttagcgtaatggcaagtgagggcattgaccttgagttcgaagactctggtctgatgcctgacaccaaggacgtaatccgcatcctgcaagcgcgtatctatgagcaattaacgattgacctctgggaagacgcagaagacttgctcaatgaatacttggaggaagtcgaggagtacgaggaggatgaagagtaatgtctactaccaacgtgcaatacggtctgaccgctcaaactgtacttttctatagcgacatggtgcgctgtggctttaactggtcactcgcaatggcacagctcaaagaactgtacgaaaacaacaaggcaatagctttagaatctgctgagtgatagactcaaggtcgctcctagcgagtggcctttatgattatcactttacttatgagggagtaatgtatatgcttactatcggtctactcaccgctctaggtctagctgtaggtgcatcctttgggaaggctttaggtgtagctgtaggttcctactttaccgcttgcatcatcataggaatcatcaaaggggcactacgcaaatgatgaagcactacgttatgccaatccacacgtccaacggggcaaccgtatgtacacctgatgggttcgcaatgaaacaacgaatcgaacgccttaagcgtgaactccgcattaaccgcaagattaacaagataggttccggctatgacagaacgcactgatggcttaaagaaaggttatatgcccaatggcacactatacgctgcaaatcggcgaatagtgagaacttggcgagagaacaacctcgaacgccgcaaggacaagagagggcggcgtggcatagacgaaaggaaaaggttaaagccaagaaactcgccgcacttgaacaggcactagccaacacactgaacgctatctcataacgaacataaaggacacaatgcaatgaacattaccgacatcatgaacgctatcgacgcaatcaaagcactgccaatctgtgaacttgacaagcgtcaaggtatgcttatcgacttactggtcgagatggtcaacagcgagacgtgtgatggcgagctaaccgaactaaatcaggcacttgagcatcaagattggtggactaccttgaagtgtctcacggctgacgcagggttcaagatgctcggtaatggtcacttctcggctgcttatagtcacccgctgctacctaacagagtgattaaggtgggctttaagaaagaggattcaggcgcagcctataccgcattctgccgcatgtatcagggtcgtcctggtatccctaacgtctacgatgtacagcgccacgctggatgctatacggtggtacttgacgcacttaaggattgcgagcgtttcaacaatgatgcccattataaatacgctgagattgcaagcgacatcattgattgcaattcggatgagcatgatgagttaactggatgggatggtgagtttgttgaaacttgtaaactaatccgcaagttctttgagggcatcgcctcattcgacatgcatagcgggaacatcatgttctcaaatggagacgtaccatacatcaccgacccggtatcattctcgcagaagaaagacggtggcgcattcagcatcgaccctgaggaactcatcaaggaagtcgaggaagtcgcacgacagaaagaaattgaccgcgctaaggcccgtaaagaacgtcacgaggggcgcttagaggcacgcagattcaaacgtcgcaaccgcaaggcacgtaaagcacacaaagctaagcgcgaaagaatgcttgctgcgtggcgatgggctgaacgtcaagaacggcgtaaccatgaggtagctgtagatgtactaggaagaaccaataacgctatgctctgggtcaacatgttctctggggactttaaggcgcttgaggaacgaatcgcgctgcactggcgtaatgctgaccggatggctatcgctaatggtcttacgctcaacattgataagcaacttgacgcaatgttaatgggctgatagtcttatcttacaggtcatctgcgggtggcctgaataggtacgatttactaactggaagaggcactaaatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaacgccaaatcaatacgactcactatagagggacaaactcaaggtcattcgcaagagtggcctttatgattgaccttcttccggttaatacgactcactataggagaaccttaaggtttaactttaagacccttaagtgttaattagagatttaaattaaagaattactaagagaggactttaagtatgcgtaacttcgaaaagatgaccaaacgttctaaccgtaatgctcgtgacttcgaggcaaccaaaggtcgcaagttgaataagactaagcgtgaccgctctcacaagcgtagctgggagggtcagtaagatgggacgtttatatagtggtaatctggcagcattcaaggcagcaacaaacaagctgttccagttagacttagcggtcatttatgatgactggtatgatgcctatacaagaaaagattgcatacggttacgtattgaggacaggagtggaaacctgattgatactagcaccttctaccaccacgacgaggacgttctgttcaatatgtgtactgattggttgaaccatatgtatgaccagttgaaggactggaagtaatacgactcagtatagggacaatgcttaaggtcgctctctaggagtggccttagtcatttaaccaataggagataaacattatgatgaacattaagactaacccgtttaaagccgtgtctttcgtagagtctgccattaagaaggctctggataacgctgggtatcttatcgctgaaatcaagtacgatggtgtacgcgggaacatctgcgtagacaatactgctaacagttactggctctctcgtgtatctaaaacgattccggcactggagcacttaaacgggtttgatgttcgctggaagcgtctactgaacgatgaccgttgcttctacaaagatggctttatgcttgatggggaactcatggtcaagggcgtagactttaacacagggtccggcctactgcgtaccaaatggactgacacgaagaaccaagagttccatgaagagttattcgttgaaccaatccgtaagaaagataaagttccctttaagctgcacactggacaccttcacataaaactgtacgctatcctcccgctgcacatcgtggagtctggagaagactgtgatgtcatgacgttgctcatgcaggaacacgttaagaacatgctgcctctgctacaggaatacttccctgaaatcgaatggcaagcggctgaatcttacgaggtctacgatatggtagaactacagcaactgtacgagcagaagcgagcagaaggccatgagggtctcattgtgaaagacccgatgtgtatctataagcgcggtaagaaatctggctggtggaaaatgaaacctgagaacgaagctgacggtatcattcagggtctggtatggggtacaaaaggtctggctaatgaaggtaaagtgattggttttgaggtgcttcttgagagtggtcgtttagttaacgccacgaatatctctcgcgccttaatggatgagttcactgagacagtaaaagaggccaccctaagtcaatggggattctttagcccatacggtattggcgacaacgatgcttgtactattaacccttacgatggctgggcgtgtcaaattagctacatggaggaaacacctgatggctctttgcggcacccatcgttcgtaatgttccgtggcaccgaggacaaccctcaagagaaaatgtaatcacactggctcaccttcgggtgggcctttctgcgtttataaggagacactttatgtttaagaaggttggtaaattccttgcggctttggcagctatcctgacgcttgcgtatattcttgcggtataccctcaagtagcactagtagtagttggcgcttgttacttagcggcagtgtgtgcttgcgtgtggagtatagttaactggtaatacgactcactaaaggaggtacacaccatgatgtacttaatgccattactcatcgtcattgtaggatgccttgcgctccactgtagcgatgatgatatgccagatggtcacgcttaatacgactcactaaaggagacactatatgtttcgacttcattacaacaaaagcgttaagaatttcacggttcgccgtgctgaccgttcaatcgtatgtgcgagcgagcgccgagctaagatacctcttattggtaacacagttcctttggcaccgagcgtccacatcattatcacccgtggtgactttgagaaagcaatagacaagaaacgtccggttcttagtgtggcagtgacccgcttcccgttcgtccgtctgttactcaaacgaatcaaggaggtgttctgatgggactgttagatggtgaagcctgggaaaaagaaaacccgccagtacaagcaactgggtgtatagcttgcttagagaaagatgaccgttatccacacacctgtaacaaaggagctaacgatatgaccgaacgtgaacaagagatgatcattaagttgatagacaataatgaaggtcgcccagatgatttgaatggctgcggtattctctgctccaatgtcccttgccacctctgccccgcaaataacgatcaaaagataaccttaggtgaaatccgagcgatggacccacgtaaaccacatctgaataaacctgaggtaactcctacagatgaccagccttccgctgagacaatcgaaggtgtcactaagccttcccactacatgctgtttgacgacattgaggctatcgaagtgattgctcgttcaatgaccgttgagcagttcaagggatactgcttcggtaacatcttaaagtacagactacgtgctggtaagaagtcagagttagcgtacttagagaaagacctagcgaaagcagacttctataaagaactctttgagaaacataaggataaatgttatgcataacttcaagtcaaccccacctgccgacagcctatctgatgacttcacatcttgctcagagtggtgccgaaagatgtgggaagagacattcgacgatgcgtacatcaagctgtatgaactttggaaatcgagaggtcaatgactatgtcaaacgtaaatacaggttcacttagtgtggacaataagaagttttgggctaccgtagagtcctcggagcattccttcgaggttccaatctacgctgagaccctagacgaagctctggagttagccgaatggcaatacgttccggctggctttgaggttactcgtgtgcgtccttgtgtagcaccgaagtaatacgactcactattagggaagactccctctgagaaaccaaacgaaacctaaaggagattaacattatggctaagaagattttcacctctgcgctgggtaccgctgaaccttacgcttacatcgccaagccggactacggcaacgaagagcgtggctttgggaaccctcgtggtgtctataaagttgacctgactattcccaacaaagacccgcgctgccagcgtatggtcgatgaaatcgtgaagtgtcacgaagaggcttatgctgctgccgttgaggaatacgaagctaatccacctgctgtagctcgtggtaagaaaccgctgaaaccgtatgagggtgacatgccgttcttcgataacggtgacggtacgactacctttaagttcaaatgctacgcgtctttccaagacaagaagaccaaagagaccaagcacatcaatctggttgtggttgactcaaaaggtaagaagatggaagacgttccgattatcggtggtggctctaagctgaaagttaaatattctctggttccatacaagtggaacactgctgtaggtgcgagcgttaagctgcaactggaatccgtgatgctggtcgaactggctacctttggtggcggtgaagacgattgggctgacgaagttgaagagaacggctatgttgcctctggttctgccaaagcgagcaaaccacgcgacgaagaaagctgggacgaagacgacgaagagtccgaggaagcagacgaagacggagacttctaagtggaactgcgggagaaaatccttgagcgaatcaaggtgacttcctctgggtgttgggagtggcagggcgctacgaacaataaagggtacgggcaggtgtggtgcagcaataccggaaaggttgtctactgtcatcgcgtaatgtctaatgctccgaaaggttctaccgtcctgcactcctgtgataatccattatgttgtaaccctgaacacctatccataggaactccaaaagagaactccactgacatggtaaataagggtcgctcacacaaggggtataaactttcagacgaagacgtaatggcaatcatggagtccagcgagtccaatgtatccttagctcgcacctatggtgtctcccaacagactatttgtgatatacgcaaagggaggcgacatggcaggttacggcgctaaaggaatccgaaaggttggagcgtttcgctctggcctagaggacaaggtttcaaagcagttggaatcaaaaggtattaaattcgagtatgaagagtggaaagtgccttatgtaattccggcgagcaatcacacttacactccagacttcttacttccaaacggtatattcgttgagacaaagggtctgtgggaaagcgatgatagaaagaagcacttattaattagggagcagcaccccgagctagacatccgtattgtcttctcaagctcacgtactaagttatacaaaggttctccaacgtcttatggagagttctgcgaaaagcatggtattaagttcgctgataaactgatacctgctgagtggataaaggaacccaagaaggaggtcccctttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctgtgcttcgcgcccatcatgaggtggcgccgaaggcttgcccttcgttcgaccttaagcgttggtgggagaagaacgaactggtcacttctgaccgtggataattaattgaactcactaaagggagaccacagcggtttccctttgttcgcattggaggtcaaataatgcgcaagtcttataaacaattctataaggctccgaggaggcatatccaagtgtgggaggcagccaatgggcctataccaaaaggttattatatagaccacattgacggcaatccactcaacgacgccttagacaatctccgtctggctctcccaaaagaaaactcatggaacatgaagactccaaagagcaatacctcaggactaaagggactgagttggagcaaggaaagggagatgtggagaggcactgtaacagctgagggtaaacagcataactttcgtagtagagatctattggaagtcgttgcgtggatttatagaactaggagggaattgcatggacaattcgcacgattccgatagtgtatttctttaccacattccttgtgacaactgtgggagtagtgatgggaactcgctgttctctgacggacacacgttctgctacgtatgcgagaagtggactgctggtaatgaagacactaaagagagggcttcaaaacggaaaccctcaggaggtaaaccaatgacttacaacgtgtggaacttcggggaatccaatggacgctactccgcgttaactgcgagaggaatctccaaggaaacctgtcagaaggctggctactggattgccaaagtagacggtgtgatgtaccaagtggctgactatcgggaccagaacggcaacattgtgagtcagaaggttcgagataaagataagaactttaagaccactggtagtcacaagagtgacgctctgttcgggaagcacttgtggaatggtggtaagaagattgtcgttacagaaggtgaaatcgacatgcttaccgtgatggaacttcaagactgtaagtatcctgtagtgtcgttgggtcacggtgcctctgccgctaagaagacatgcgctgccaactacgaatactttgaccagttcgaacagattatcttaatgttcgatatggacgaagcagggcgcaaagcagtcgaagaggctgcacaggttctacctgctggtaaggtacgagtggcagttcttccgtgtaaggatgcaaacgagtgtcacctaaatggtcacgaccgtgaaatcatggagcaagtgtggaatgctggtccttggattcctgatggtgtggtatcggctctttcgttacgtgaacgaatccgtgagcacctatcgtccgaggaatcagtaggtttacttttcagtggctgcactggtatcaacgataagaccttaggtgcccgtggtggtgaagtcattatggtcacttccggttccggtatgggtaagtcaacgttcgtccgtcaacaagctctacaatggggcacagcgatgggcaagaaggtaggcttagcgatgcttgaggagtccgttgaggagaccgctgaggaccttataggtctacacaaccgtgtccgactgagacaatccgactcactaaagagagagattattgagaacggtaagttcgaccaatggttcgatgaactgttcggcaacgatacgttccatctatatgactcattcgccgaggctgagacggatagactgctcgctaagctggcctacatgcgctcaggcttgggctgtgacgtaatcattctagaccacatctcaatcgtcgtatccgcttctggtgaatccgatgagcgtaagatgattgacaacctgatgaccaagctcaaagggttcgctaagtcaactggggtggtgctggtcgtaatttgtcaccttaagaacccagacaaaggtaaagcacatgaggaaggtcgccccgtttctattactgacctacgtggttctggcgcactacgccaactatctgatactattattgcccttgagcgtaatcagcaaggcgatatgcctaaccttgtcctcgttcgtattctcaagtgccgctttactggtgatactggtatcgctggctacatggaatacaacaaggaaaccggatggcttgaaccatcaagttactcaggggaagaagagtcacactcagagtcaacagactggtccaacgacactgacttctgacaggattcttgatgactttccagacgactacgagaagtttcgctggagagtcccattctaatacgactcactaaaggagacacaccatgttcaaactgattaagaagttaggccaactgctggttcgtatgtacaacgtggaagccaagcgactgaacgatgaggctcgtaaagaggccacacagtcacgcgctctggcgattcgctccaacgaactggctgacagtgcatccactaaagttaccgaggctgcccgtgtggcaaaccaagctcaacagctttccaaattctttgagtaatcaaacaggagaaaccattatgtctaacgtagctgaaactatccgtctatccgatacagctgaccagtggaaccgtcgagtccacatcaacgttcgcaacggtaaggcgactatggtttaccgctggaaggactctaagtcctctaagaatcacactcagcgtatgacgttgacagatgagcaagcactgcgtctggtcaatgcgcttaccaaagctgccgtgacagcaattcatgaagctggtcgcgtcaatgaagctatggctatcctcgacaagattgataactaagagtggtatcctcaaggtcgccaaagtggtggccttcatgaatactattcgactcactataggagatattaccatgcgtgaccctaaagttatccaagcagaaatcgctaaactggaagctgaactggaggacgttaagtaccatgaagctaagactcgctccgctgttcacatcttgaagaacttaggctggacttggacaagacagactggctggaagaaaccagaagttaccaagctgagtcataaggtgttcgataaggacactatgacccacatcaaggctggtgattgggttaaggttgacatgggagttgttggtggatacggctacgtccgctcagttagtggcaaatatgcacaagtgtcatacatcacaggtgttactccacgcggtgcaatcgttgccgataagaccaacatgattcacacaggtttcttgacagttgtttcatatgaagagattgttaagtcacgataatcaataggagaaatcaatatgatcgtttctgacatcgaagctaacgccctcttagagagcgtcactaagttccactgcggggttatctacgactactccaccgctgagtacgtaagctaccgtccgagtgacttcggtgcgtatctggatgcgctggaagccgaggttgcacgaggcggtcttattgtgttccacaacggtcacaagtatgacgttcctgcattgaccaaactggcaaagttgcaattgaaccgagagttccaccttcctcgtgagaactgtattgacacccttgtgttgtcacgtttgattcattccaacctcaaggacaccgatatgggtcttctgcgttccggcaagttgcccggaaaacgctttgggtctcacgctttggaggcgtggggttatcgcttaggcgagatgaagggtgaatacaaagacgactttaagcgtatgcttgaagagcagggtgaagaatacgttgacggaatggagtggtggaacttcaacgaagagatgatggactataacgttcaggacgttgtggtaactaaagctctccttgagaagctactctctgacaaacattacttccctcctgagattgactttacggacgtaggatacactacgttctggtcagaatcccttgaggccgttgacattgaacatcgtgctgcatggctgctcgctaaacaagagcgcaacgggttcccgtttgacacaaaagcaatcgaagagttgtacgtagagttagctgctcgccgctctgagttgctccgtaaattgaccgaaacgttcggctcgtggtatcagcctaaaggtggcactgagatgttctgccatccgcgaacaggtaagccactacctaaataccctcgcattaagacacctaaagttggtggtatctttaagaagcctaagaacaaggcacagcgagaaggccgtgagccttgcgaacttgatacccgcgagtacgttgctggtgctccttacaccccagttgaacatgttgtgtttaacccttcgtctcgtgaccacattcagaagaaactccaagaggctgggtgggtcccgaccaagtacaccgataagggtgctcctgtggtggacgatgaggtactcgaaggagtacgtgtagatgaccctgagaagcaagccgctatcgacctcattaaagagtacttgatgattcagaagcgaatcggacagtctgctgagggagacaaagcatggcttcgttatgttgctgaggatggtaagattcatggttctgttaaccctaatggagcagttacgggtcgtgcgacccatgcgttcccaaaccttgcgcaaattccgggtgtacgttctccttatggagagcagtgtcgcgctgcttttggcgctgagcaccatttggatgggataactggtaagccttgggttcaggctggcatcgacgcatccggtcttgagctacgctgcttggctcacttcatggctcgctttgataacggcgagtacgctcacgagattcttaacggcgacatccacactaagaaccagatagctgctgaactacctacccgagataacgctaagacgttcatctatgggttcctctatggtgctggtgatgagaagattggacagattgttggtgctggtaaagagcgcggtaaggaactcaagaagaaattccttgagaacacccccgcgattgcagcactccgcgagtctatccaacagacacttgtcgagtcctctcaatgggtagctggtgagcaacaagtcaagtggaaacgccgctggattaaaggtctggatggtcgtaaggtacacgttcgtagtcctcacgctgccttgaataccctactgcaatctgctggtgctctcatctgcaaactgtggattatcaagaccgaagagatgctcgtagagaaaggcttgaagcatggctgggatggggactttgcgtacatggcatgggtacatgatgaaatccaagtaggctgccgtaccgaagagattgctcaggtggtcattgagaccgcacaagaagcgatgcgctgggttggagaccactggaacttccggtgtcttctggataccgaaggtaagatgggtcctaattgggcgatttgccactgatacaggaggctactcatgaacgaaagacacttaacaggtgctgcttctgaaatgctagtagcctacaaatttaccaaagctgggtacactgtctattaccctatgctgactcagagtaaagaggacttggttgtatgtaaggatggtaaatttagtaaggttcaggttaaaacagccacaacggttcaaaccaacacaggagatgccaagcaggttaggctaggtggatgcggtaggtccgaatataaggatggagactttgacattcttgcggttgtggttgacgaagatgtgcttattttcacatgggacgaagtaaaaggtaagacatccatgtgtgtcggcaagagaaacaaaggcataaaactataggagaaattattatggctatgacaaagaaatttaaagtgtccttcgacgttaccgcaaagatgtcgtctgacgttcaggcaatcttagagaaagatatgctgcatctatgtaagcaggtcggctcaggtgcgattgtccccaatggtaaacagaaggaaatgattgtccagttcctgacacacggtatggaaggattgatgacattcgtagtacgtacatcatttcgtgaggccattaaggacatgcacgaagagtatgcagataaggactctttcaaacaatctcctgcaacagtacgggaggtgttctgatgtctgactacctgaaagtgctgcaagcaatcaaaagttgccctaagactttccagtccaactatgtacggaacaatgcgagcctcgtagcggaggccgcttcccgtggtcacatctcgtgcctgactactagtggacgtaacggtggcgcttgggaaatcactgcttccggtactcgctttctgaaacgaatgggaggatgtgtctaatgtctcgtgaccttgtgactattccacgcgatgtgtggaacgatatacagggctacatcgactctctggaacgtgagaacgatagccttaagaatcaactaatggaagctgacgaatacgtagcggaactagaggagaaacttaatggcacttcttgaccttaaacaattctatgagttacgtgaaggctgcgacgacaagggtatccttgtgatggacggcgactggctggtcttccaagctatgagtgctgctgagtttgatgcctcttgggaggaagagatttggcaccgatgctgtgaccacgctaaggcccgtcagattcttgaggattccattaagtcctacgagacccgtaagaaggcttgggcaggtgctccaattgtccttgcgttcaccgatagtgttaactggcgtaaagaactggttgacccgaactataaggctaaccgtaaggccgtgaagaaacctgtagggtactttgagttccttgatgctctctttgagcgcgaagagttctattgcatccgtgagcctatgcttgagggtgatgacgttatgggagttattgcttccaatccgtctgccttcggtgctcgtaaggctgtaatcatctcttgcgataaggactttaagaccatccctaactgtgacttcctgtggtgtaccactggtaacatcctgactcagaccgaagagtccgctgactggtggcacctcttccagaccatcaagggtgacatcactgatggttactcagggattgctggatggggtgataccgccgaggacttcttgaataacccgttcataaccgagcctaaaacgtctgtgcttaagtccggtaagaacaaaggccaagaggttactaaatgggttaaacgcgaccctgagcctcatgagacgctttgggactgcattaagtccattggcgcgaaggctggtatgaccgaagaggatattatcaagcagggccaaatggctcgaatcctacggttcaacgagtacaactttattgacaaggagatttacctgtggagaccgtagcgtatattggtctgggtctttgtgttctcggagtgtgcctcatttcgtggggcctttgggacttagccagaataatcaagtcgttacacgacactaagtgataaactcaaggtccctaaattaatacgactcactatagggagataggggcctttacgattattactttaagatttaactctaagaggaatctttattatgttaacacctattaaccaattacttaagaaccctaacgatattccagatgtacctcgtgcaaccgctgagtatctacaggttcgattcaactatgcgtacctcgaagcgtctggtcatataggacttatgcgtgctaatggttgtagtgaggcccacatcttgggtttcattcagggcctacagtatgcctctaacgtcattgacgagattgagttacgcaaggaacaactaagagatgatggggaggattgacactatgtgtttctcaccgaaaattaaaactccgaagatggataccaatcagattcgagccgttgagccagcgcctctgacccaagaagtgtcaagcgtggagttcggtgggtcttctgatgagacggataccgagggcaccgaagtgtctggacgcaaaggcctcaaggtcgaacgtgatgattccgtagcgaagtctaaagccagcggcaatggctccgctcgtatgaaatcttccatccgtaagtccgcatttggaggtaagaagtgatgtctgagttcacatgtgtggaggctaagagtcgcttccgtgcaatccggtggactgtggaacaccttgggttgcctaaaggattcgaaggacactttgtgggctacagcctctacgtagacgaagtgatggacatgtctggttgccgtgaagagtacattctggactctaccggaaaacatgtagcgtacttcgcgtggtgcgtaagctgtgacattcaccacaaaggagacattctggatgtaacgtccgttgtcattaatcctgaggcagactctaagggcttacagcgattcctagcgaaacgctttaagtaccttgcggaactccacgattgcgattgggtgtctcgttgtaagcatgaaggcgagacaatgcgtgtatactttaaggaggtataagttatgggtaagaaagttaagaaggccgtgaagaaagtcaccaagtccgttaagaaagtcgttaaggaaggggctcgtccggttaaacaggttgctggcggtctagctggtctggctggtggtactggtgaagcacagatggtggaagtaccacaagctgccgcacagattgttgacgtacctgagaaagaggtttccactgaggacgaagcacagacagaaagcggacgcaagaaagctcgtgctggcggtaagaaatccttgagtgtagcccgtagctccggtggcggtatcaacatttaatcaggaggttatcgtggaagactgcattgaatggaccggaggtgtcaactctaagggttatggtcgtaagtgggttaatggtaaacttgtgactccacataggcacatctatgaggagacatatggtccagttccaacaggaattgtggtgatgcatatctgcgataaccctaggtgctataacataaagcaccttacgcttggaactccaaaggataattccgaggacatggttaccaaaggtagacaggctaaaggagaggaactaagcaagaaacttacagagtcagacgttctcgctatacgctcttcaaccttaagccaccgctccttaggagaactgtatggagtcagtcaatcaaccataacgcgaatactacagcgtaagacatggagacacatttaatggctgagaaacgaacaggacttgcggaggatggcgcaaagtctgtctatgagcgtttaaagaacgaccgtgctccctatgagacacgcgctcagaattgcgctcaatataccatcccatcattgttccctaaggactccgataacgcctctacagattatcaaactccgtggcaagccgtgggcgctcgtggtctgaacaatctagcctctaagctcatgctggctctattccctatgcagacttggatgcgacttactatatctgaatatgaagcaaagcagttactgagcgaccccgatggactcgctaaggtcgatgagggcctctcgatggtagagcgtatcatcatgaactacattgagtctaacagttaccgcgtgactctctttgaggctctcaaacagttagtcgtagctggtaacgtcctgctgtacctaccggaaccggaagggtcaaactataatcccatgaagctgtaccgattgtcttcttatgtggtccaacgagacgcattcggcaacgttctgcaaatggtgactcgtgaccagatagcttttggtgctctccctgaggacatccgtaaggctgtagaaggtcaaggtggtgagaagaaagctgatgagacaatcgacgtgtacactcacatctatctggatgaggactcaggtgaatacctccgatacgaagaggtcgagggtatggaagtccaaggctccgatgggacttatcctaaagaggcttgcccatacatcccgattcggatggtcagactagatggtgaatcctacggtcgttcgtacattgaggaatacttaggtgacttacggtcccttgaaaatctccaagaggctatcgtcaagatgtccatgattagctctaaggttatcggcttagtgaatcctgctggtatcacccagccacgccgactgaccaaagctcagactggtgacttcgttactggtcgtccagaagacatctcgttcctccaactggagaagcaagcagactttactgtagctaaagccgtaagtgacgctatcgaggctcgcctttcgtttgcctttatgttgaactctgcggttcagcgtacaggtgaacgtgtgaccgccgaagagattcggtatgtagcttctgaacttgaagatactttaggtggtgtctactctatcctttctcaagaattacaattgcctctggtacgagtgctcttgaagcaactacaagccacgcaacagattcctgagttacctaaggaagccgtagagccaaccattagtacaggtctggaagcaattggtcgaggacaagaccttgataagctggagcggtgtgtcactgcgtgggctgcactggcacctatgcgggacgaccctgatattaaccttgcgatgattaagttacgtattgccaacgctatcggtattgacacttctggtattctactcaccgaagaacagaagcaacagaagatggcccaacagtctatgcaaatgggtatggataatggtgctgctgcgctggctcaaggtatggctgcacaagctacagcttcacctgaggctatggctgctgccgctgattccgtaggtttacagccgggaatttaatacgactcactatagggagacctcatctttgaaatgagcgatgacaagaggttggagtcctcggtcttcctgtagttcaactttaaggagacaataataatggctgaatctaatgcagacgtatatgcatcttttggcgtgaactccgctgtgatgtctggtggttccgttgaggaacatgagcagaacatgctggctcttgatgttgctgcccgtgatggcgatgatgcaatcgagttagcgtcagacgaagtggaaacagaacgtgacctgtatgacaactctgacccgttcggtcaagaggatgacgaaggccgcattcaggttcgtatcggtgatggctctgagccgaccgatgtggacactggagaagaaggcgttgagggcaccgaaggttccgaagagtttaccccactgggcgagactccagaagaactggtagctgcctctgagcaacttggtgagcacgaagagggcttccaagagatgattaacattgctgctgagcgtggcatgagtgtcgagaccattgaggctatccagcgtgagtacgaggagaacgaagagttgtccgccgagtcctacgctaagctggctgaaattggctacacgaaggctttcattgactcgtatatccgtggtcaagaagctctggtggagcagtacgtaaacagtgtcattgagtacgctggtggtcgtgaacgttttgatgcactgtataaccaccttgagacgcacaaccctgaggctgcacagtcgctggataatgcgttgaccaatcgtgacttagcgaccgttaaggctatcatcaacttggctggtgagtctcgcgctaaggcgttcggtcgtaagccaactcgtagtgtgactaatcgtgctattccggctaaacctcaggctaccaagcgtgaaggctttgcggaccgtagcgagatgattaaagctatgagtgaccctcggtatcgcacagatgccaactatcgtcgtcaagtcgaacagaaagtaatcgattcgaacttctgatagacttcgaaattaatacgactcactatagggagaccacaacggtttccctctagaaataattttgtttaactttaagaaggagatatacatatggctagcatgactggtggacagcaaatgggtactaaccaaggtaaaggtgtagttgctgctggagataaactggcgttgttcttgaaggtatttggcggtgaagtcctgactgcgttcgctcgtacctccgtgaccacttctcgccacatggtacgttccatctccagcggtaaatccgctcagttccctgttctgggtcgcactcaggcagcgtatctggctccgggcgagaacctcgacgataaacgtaaggacatcaaacacaccgagaaggtaatcaccattgacggtctcctgacggctgacgttctgatttatgatattgaggacgcgatgaaccactacgacgttcgctctgagtatacctctcagttgggtgaatctctggcgatggctgcggatggtgcggttctggctgagattgccggtctgtgtaacgtggaaagcaaatataatgagaacatcgagggcttaggtactgctaccgtaattgagaccactcagaacaaggccgcacttaccgaccaagttgcgctgggtaaggagattattgcggctctgactaaggctcgtgcggctctgaccaagaactatgttccggctgctgaccgtgtgttctactgtgacccagatagctactctgcgattctggcagcactgatgccgaacgcagcaaactacgctgctctgattgaccctgagaagggttctatccgcaacgttatgggctttgaggttgtagaagttccgcacctcaccgctggtggtgctggtaccgctcgtgagggcactactggtcagaagcacgtcttccctgccaataaaggtgagggtaatgtcaaggttgctaaggacaacgttatcggcctgttcatgcaccgctctgcggtaggtactgttaagctgcgtgacttggctctggagcgcgctcgccgtgctaacttccaagcggaccagattatcgctaagtacgcaatgggccacggtggtcttcgcccagaagctgctggtgcagtggttttcaaagtggagtaatgctgggggtggcctcaacggtcgctgctagtcccgaagaggcgagtgttacttcaacagaagaaaccttaacgccagcacaggaggccgcacgcacccgcgctgctaacaaagcccgaaaggaagctgagttggctgctgccaccgctgagcaataactagcataaccccttggggcctctaaacgggtcttgaggggttttttgctgaaaggaggaactatgatccgtcagcctgcagttcgaagttcctatactttctagagaataggaacttcgaagcagctccagcctacattaaatattctccctgtggtggctcgaaattaatacgactcactatagggagaacaatacgactacgggagggttttcttatgatgactataagacctactaaaagtacagactttgaggtattcactccggctcaccatgacattcttgaagctaaggctgctggtattgagccgagtttccctgatgcttccgagtgtgtcacgttgagcctctatgggttccctctagctatcggtggtaactgcggggaccagtgctggttcgttacgagcgaccaagtgtggcgacttagtggaaaggctaagcgaaagttccgtaagttaatcatggagtatcgcgataagatgcttgagaagtatgatactctttggaattacgtatgggtaggcaatacgtcccacattcgtttcctcaagactatcggtgcggtattccatgaagagtacacacgagatggtcaatttcagttatttacaatcacgaaaggaggataaccatatgtgttgggcagccgcaatacctatcgctatatctggcgctcaggctatcagtggtcagaacgctcaggccaaaatgattgccgctcagaccgctgctggtcgtcgtcaagctatggaaatcatgaggcagacgaacatccagaatgctgacctatcgttgcaagctcgaagtaaacttgaggaagcgtccgccgagttgacctcacagaacatgcagaaggtccaagctattgggtctatccgagcggctatcggagagagtatgcttgaaggttcctcaatggaccgcattaagcgagtcacagaaggacagttcattcgggaagccaatatggtaactgagaactatcgccgtgactaccaagcaatcttcgcacagcaacttggtggtactcaaagtgctgcaagtcagattgacgaaatctataagagcgaacagaaacagaagagtaagctacagatggttctggacccactggctatcatggggtcttccgctgcgagtgcttacgcatccggtgcgttcgactctaagtccacaactaaggcacctattgttgccgctaaaggaaccaagacggggaggtaatgagctatgagtaaaattgaatctgcccttcaagcggcacaaccgggactctctcggttacgtggtggtgctggaggtatgggctatcgtgcagcaaccactcaggccgaacagccaaggtcaagcctattggacaccattggtcggttcgctaaggctggtgccgatatgtataccgctaaggaacaacgagcacgagacctagctgatgaacgctctaacgagattatccgtaagctgacccctgagcaacgtcgagaagctctcaacaacgggacccttctgtatcaggatgacccatacgctatggaagcactccgagtcaagactggtcgtaacgctgcgtatcttgtggacgatgacgttatgcagaagataaaagagggtgtcttccgtactcgcgaagagatggaagagtatcgccatagtcgccttcaagagggcgctaaggtatacgctgagcagttcggcatcgaccctgaggacgttgattatcagcgtggtttcaacggggacattaccgagcgtaacatctcgctgtatggtgcgcatgataacttcttgagccagcaagctcagaagggcgctatcatgaacagccgagtggaactcaacggtgtccttcaagaccctgatatgctgcgtcgtccagactctgctgacttctttgagaagtatatcgacaacggtctggttactggcgcaatcccatctgatgctcaagccacacagcttataagccaagcgttcagtgacgcttctagccgtgctggtggtgctgacttcctgatgcgagtcggtgacaagaaggtaacacttaacggagccactacgacttaccgagagttgattggtgaggaacagtggaacgctctcatggtcacagcacaacgttctcagtttgagactgacgcgaagctgaacgagcagtatcgcttgaagattaactctgcgctgaaccaagaggacccaaggacagcttgggagatgcttcaaggtatcaaggctgaactagataaggtccaacctgatgagcagatgacaccacaacgtgagtggctaatctccgcacaggaacaagttcagaatcagatgaacgcatggacgaaagctcaggccaaggctctggacgattccatgaagtcaatgaacaaacttgacgtaatcgacaagcaattccagaagcgaatcaacggtgagtgggtctcaacggattttaaggatatgccagtcaacgagaacactggtgagttcaagcatagcgatatggttaactacgccaataagaagctcgctgagattgacagtatggacattccagacggtgccaaggatgctatgaagttgaagtaccttcaagcggactctaaggacggagcattccgtacagccatcggaaccatggtcactgacgctggtcaagagtggtctgccgctgtgattaacggtaagttaccagaacgaaccccagctatggatgctctgcgcagaatccgcaatgctgaccctcagttgattgctgcgctatacccagaccaagctgagctattcctgacgatggacatgatggacaagcagggtattgaccctcaggttattcttgatgccgaccgactgactgttaagcggtccaaagagcaacgctttgaggatgataaagcattcgagtctgcactgaatgcatctaaggctcctgagattgcccgtatgccagcgtcactgcgcgaatctgcacgtaagatttatgactccgttaagtatcgctcggggaacgaaagcatggctatggagcagatgaccaagttccttaaggaatctacctacacgttcactggtgatgatgttgacggtgataccgttggtgtgattcctaagaatatgatgcaggttaactctgacccgaaatcatgggagcaaggtcgggatattctggaggaagcacgtaagggaatcattgcgagcaacccttggataaccaataagcaactgaccatgtattctcaaggtgactccatttaccttatggacaccacaggtcaagtcagagtccgatacgacaaagagttactctcgaaggtctggagtgagaaccagaagaaactcgaagagaaagctcgtgagaaggctctggctgatgtgaacaagcgagcacctatagttgccgctacgaaggcccgtgaagctgctgctaaacgagtccgagagaaacgtaaacagactcctaagttcatctacggacgtaaggagtaactaaaggctacataaggaggccctaaatggataagtacgataagaacgtaccaagtgattatgatggtctgttccaaaaggctgctgatgccaacggggtctcttatgaccttttacgtaaagtcgcttggacagaatcacgatttgtgcctacagcaaaatctaagactggaccattaggcatgatgcaatttaccaaggcaaccgctaaggccctcggtctgcgagttaccgatggtccagacgacgaccgactgaaccctgagttagctattaatgctgccgctaagcaacttgcaggtctggtagggaagtttgatggcgatgaactcaaagctgcccttgcgtacaaccaaggcgagggacgcttgggtaatccacaacttgaggcgtactctaagggagacttcgcatcaatctctgaggagggacgtaactacatgcgtaaccttctggatgttgctaagtcacctatggctggacagttggaaacttttggtggcataaccccaaagggtaaaggcattccggctgaggtaggattggctggaattggtcacaagcagaaagtaacacaggaacttcctgagtccacaagttttgacgttaagggtatcgaacaggaggctacggcgaaaccattcgccaaggacttttgggagacccacggagaaacacttgacgagtacaacagtcgttcaaccttcttcggattcaaaaatgctgccgaagctgaactctccaactcagtcgctgggatggctttccgtgctggtcgtctcgataatggttttgatgtgtttaaagacaccattacgccgactcgctggaactctcacatctggactccagaggagttagagaagattcgaacagaggttaagaaccctgcgtacatcaacgttgtaactggtggttcccctgagaacctcgatgacctcattaaattggctaacgagaactttgagaatgactcccgcgctgccgaggctggcctaggtgccaaactgagtgctggtattattggtgctggtgtggacccgcttagctatgttcctatggtcggtgtcactggtaagggctttaagttaatcaataaggctcttgtagttggtgccgaaagtgctgctctgaacgttgcatccgaaggtctccgtacctccgtagctggtggtgacgcagactatgcgggtgctgccttaggtggctttgtgtttggcgcaggcatgtctgcaatcagtgacgctgtagctgctggactgaaacgcagtaaaccagaagctgagttcgacaatgagttcatcggtcctatgatgcgattggaagcccgtgagacagcacgaaacgccaactctgcggacctctctcggatgaacactgagaacatgaagtttgaaggtgaacataatggtgtcccttatgaggacttaccaacagagagaggtgccgtggtgttacatgatggctccgttctaagtgcaagcaacccaatcaaccctaagactctaaaagagttctccgaggttgaccctgagaaggctgcgcgaggaatcaaactggctgggttcaccgagattggcttgaagaccttggggtctgacgatgctgacatccgtagagtggctatcgacctcgttcgctctcctactggtatgcagtctggtgcctcaggtaagttcggtgcaacagcttctgacatccatgagagacttcatggtactgaccagcgtacttataatgacttgtacaaagcaatgtctgacgctatgaaagaccctgagttctctactggcggcgctaagatgtcccgtgaagaaactcgatacactatctaccgtagagcggcactagctattgagcgtccagaactacagaaggcactcactccgtctgagagaatcgttatggacatcattaagcgtcactttgacaccaagcgtgaacttatggaaaacccagcaatattcggtaacacaaaggctgtgagtatcttccctgagagtcgccacaaaggtacttacgttcctcacgtatatgaccgtcatgccaaggcgctgatgattcaacgctacggtgccgaaggtttgcaggaagggattgcccgctcatggatgaacagctacgtctccagacctgaggtcaaggccagagtcgatgagatgcttaaggaattacacggggtgaaggaagtaacaccagagatggtagagaagtacgctatggataaggcttatggtatctcccactcagaccagttcaccaacagttccataatagaagagaacattgagggcttagtaggtatcgagaataactcattccttgaggcacgtaacttgtttgattcggacctatccatcactatgccagacggacagcaattctcagtgaatgacctaagggacttcgatatgttccgcatcatgccagcgtatgaccgccgtgtcaatggtgacatcgccatcatggggtctactggtaaaaccactaaggaacttaaggatgagattttggctctcaaagcgaaagctgagggagacggtaagaagactggcgaggtacatgctttaatggataccgttaagattcttactggtcgtgctagacgcaatcaggacactgtgtgggaaacctcactgcgtgccatcaatgacctagggttcttcgctaagaacgcctacatgggtgctcagaacattacggagattgctgggatgattgtcactggtaacgttcgtgctctagggcatggtatcccaattctgcgtgatacactctacaagtctaaaccagtttcagctaaggaactcaaggaactccatgcgtctctgttcgggaaggaggtggaccagttgattcggcctaaacgtgctgacattgtgcagcgcctaagggaagcaactgataccggacctgccgtggcgaacatcgtagggaccttgaagtattcaacacaggaactggctgctcgctctccgtggactaagctactgaacggaaccactaactaccttctggatgctgcgcgtcaaggtatgcttggggatgttattagtgccaccctaacaggtaagactacccgctgggagaaagaaggcttccttcgtggtgcctccgtaactcctgagcagatggctggcatcaagtctctcatcaaggaacatatggtacgcggtgaggacgggaagtttaccgttaaggacaagcaagcgttctctatggacccacgggctatggacttatggagactggctgacaaggtagctgatgaggcaatgctgcgtccacataaggtgtccttacaggattcccatgcgttcggagcactaggtaagatggttatgcagtttaagtctttcactatcaagtcccttaactctaagttcctgcgaaccttctatgatggatacaagaacaaccgagcgattgacgctgcgctgagcatcatcacctctatgggtctcgctggtggtttctatgctatggctgcacacgtcaaagcatacgctctgcctaaggagaaacgtaaggagtacttggagcgtgcactggacccaaccatgattgcccacgctgcgttatctcgtagttctcaattgggtgctcctttggctatggttgacctagttggtggtgttttagggttcgagtcctccaagatggctcgctctacgattctacctaaggacaccgtgaaggaacgtgacccaaacaaaccgtacacctctagagaggtaatgggcgctatgggttcaaaccttctggaacagatgccttcggctggctttgtggctaacgtaggggctaccttaatgaatgctgctggcgtggtcaactcacctaataaagcaaccgagcaggacttcatgactggtcttatgaactccacaaaagagttagtaccgaacgacccattgactcaacagcttgtgttgaagatttatgaggcgaacggtgttaacttgagggagcgtaggaaataatacgactcactatagggagaggcgaaataatcttctccctgtagtctcttagatttactttaaggaggtcaagatccgtcagcctgcagttcgaagttcctatactttctagagaataggaacttcgaagcagctccagcctacattggtaaatcacaaggaaagacgtgtagtccacggatggactctcaaggaggtacaaggtgctatcattagactttaacaacgaattgattaaggctgctccaattgttgggacgggtgtagcagatgttagtgctcgactgttctttgggttaagccttaacgaatggttctacgttgctgctatcgcctacacagtggttcagattggtgccaaggtagtcgataagatgattgactggaagaaagccaataaggagtgatatgtatggaaaaggataagagccttattacattcttagagatgttggacactgcgatggctcagcgtatgcttgcggacctttcggaccatgagcgtcgctctccgcaactctataatgctattaacaaactgttagaccgccacaagttccagattggtaagttgcagccggatgttcacatcttaggtggccttgctggtgctcttgaagagtacaaagagaaagtcggtgataacggtcttacggatgatgatatttacacattacagtgatatactcaaggccactacagatagtggtctttatggatgtcattgtctatacgagatgctcctacgtgaaatctgaaagttaacgggaggcattatgctagaatttttacgtaagctaatcccttgggttctcgctgggatgctattcgggttaggatggcatctagggtcagactcaatggacgctaaatggaaacaggaggtacacaatgagtacgttaagagagttgaggctgcgaagagcactcaaagagcaatcgatgcggtatctgctaagtatcaagaagaccttgccgcgctggaagggagcactgataggattatttctgatttgcgtagcgacaataagcggttgcgcgtcagagtcaaaactaccggaacctccgatggtcagtgtggattcgagcctgatggtcgagccgaacttgacgaccgagatgctaaacgtattctcgcagtgacccagaagggtgacgcatggattcgtgcgttacaggatactattcgtgaactgcaacgtaagtaggaaatcaagtaaggaggcaatgtgtctactcaatccaatcgtaatgcgctcgtagtggcgcaactgaaaggagacttcgtggcgttcctattcgtcttatggaaggcgctaaacctaccggtgcccactaagtgtcagattgacatggctaaggtgctggcgaatggagacaacaagaagttcatcttacaggctttccgtggtatcggtaagtcgttcatcacatgtgcgttcgttgtgtggtccttatggagagaccctcagttgaagatacttatcgtatcagcctctaaggagcgtgcagacgctaactccatctttattaagaacatcattgacctgctgccattcctatctgagttaaagccaagacccggacagcgtgactcggtaatcagctttgatgtaggcccagccaatcctgaccactctcctagtgtgaaatcagtaggtatcactggtcagttaactggtagccgtgctgacattatcattgcggatgacgttgagattccgtctaacagcgcaactatgggtgcccgtgagaagctatggactctggttcaggagttcgctgcgttacttaaaccgctgccttcctctcgcgttatctaccttggtacacctcagacagagatgactctctataaggaacttgaggataaccgtgggtacacaaccattatctggcctgctctgtacccaaggacacgtgaagagaacctctattactcacagcgtcttgctcctatgttacgcgctgagtacgatgagaaccctgaggcacttgctgggactccaacagacccagtgcgctttgaccgtgatgacctgcgcgagcgtgagttggaatacggtaaggctggctttacgctacagttcatgcttaaccctaaccttagtgatgccgagaagtacccgctgaggcttcgtgacgctatcgtagcggccttagacttagagaaggccccaatgcattaccagtggcttccgaaccgtcagaacatcattgaggaccttcctaacgttggccttaagggtgatgacctgcatacgtaccacgattgttccaacaactcaggtcagtaccaacagaagattctggtcattgaccctagtggtcgcggtaaggacgaaacaggttacgctgtgctgtacacactgaacggttacatctaccttatggaagctggaggtttccgtgatggctactccgataagacccttgagttactcgctaagaaggcaaagcaatggggagtccagacggttgtctacgagagtaacttcggtgacggtatgttcggtaaggtattcagtcctatccttcttaaacaccacaactgtgcgatggaagagattcgtgcccgtggtatgaaagagatgcgtatttgcgatacccttgagccagtcatgcagactcaccgccttgtaattcgtgatgaggtcattagggccgactaccagtccgctcgtgacgtagacggtaagcatgacgttaagtactcgttgttctaccagatgacccgtatcactcgtgagaaaggcgctctggctcatgatgaccgattggatgcccttgcgttaggcattgagtatctccgtgagtccatgcagttggattccgttaaggtcgagggtgaagtacttgctgacttccttgaggaacacatgatgcgtcctacggttgctgctacgcatatcattgagatgtctgtgggaggagttgatgtgtactctgaggacgatgagggttacggtacgtctttcattgagtggtgatttatgcattaggactgcatagggatgcactatagaccacggatggtcagttctttaagttactgaaaagacacgataaattaatacgactcactatagggagaggagggacgaaaggttactatatagatactgaatgaatacttatagagtgcataaagtatgcataatggtgtacctagagtgacctctaagaatggtgattatattgtattagtatcaccttaacttaaggaccaacataaagggaggagactcatgttccgcttattgttgaacctactgcggcatagagtcacctaccgatttcttgtggtactttgtgctgcccttgggtacgcatctcttactggagacctcagttcactggagtctgtcgtttgctctatactcacttgtagcgattagggtcttcctgaccgactgatggctcaccgagggattcagcggtatgattgcatcacaccacttcatccctatagagtcaagtcctaaggtatacccataaagagcctctaatggtctatcctaaggtctatacctaaagataggccatcctatcagtgtcacctaaagagggtcttagagagggcctatggagttcctatagggtcctttaaaatataccataaaaatctgagtgactatctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccct

SEQ ID NO: 9 - DNA, 2070 bp, Myxococcus xanthus
  source 1..2070: mol_type = genomic DNA; note = prolyl endopeptidases(Myxococcus xanthus)
atgtcctacccggcgacccgggcggagcaggttgtcgacacgttgcacggcgtccaggtagcggacccgtatcgctggctcgaggacgagaaggcccccgaggtccagacgtggatgacggcgcagaacgcgcacgcccgcgaagcgctggcgaagttccccggccgtgaggccctggccgcgcgcttcaaggagctgttctacaccgactccgtctccaccccgtcgcgccgcaacgggcgcttcttctacgtccgcacccacaaggacaaggagaaggccatcctctactggcgccagggggagagcgggcaggagaaggtgctgttggatccgaacggctggagcaaggacggcaccgtgtccctggggacgtgggccgtgtcctgggacggcaagaaggtggccttcgcccagaagcccaacgccgcggatgaggcggtgctgcacgtcatcgacgtggactctggcgagtggtccaaggtggacgtcatcgagggcggcaagtacgccacgcccaagtggacgcccgacagcaagggcttctattacgagtggctgcccacggacccgtccatcaaggtggacgagcgccccggctacaccaccatccgctaccacacgctgggcacggagccgtcgaaggacaccgtggtgcacgagcgcaccggcgacccgacgacgttcctccagagcgacctgagccgcgacggcaagtacctgttcgtctacatcctccgcggctggagcgagaacgacgtctactggaagcggccgggtgagaaggacttccgcctgctggtgaagggcgtgggcgccaagtacgaggtgcacgcctggaaggaccgcttctacgtcctcaccgacgagggcgccccgcgccagcgcgtcttcgaggtggatccggcgaagccggcccgcgcgtcgtggaaggagattgtccccgaggactcgtccgcgtccctgctgtccgtcagcatcgtcggcgggcacctgtcgctggagtacctcaaggacgcgacgtccgaggtgcgcgtggccacgctgaagggcaagccggtgcgcacggtgcagctgccgggcgtgggcgcggcgtccaacctgatggggctggaggacctggatgacgcctactacgtcttcacgtccttcaccacgccccgtcaaatctacaagacgtccgtcagcaccgggaagtctgagctctgggccaaggtggacgtgcccatgaacccggagcagtaccaggtcgagcaggtcttctacgcgtccaaggacgggacgaaggtgcccatgttcgtggtgcaccgcaaggacctgaagcgtgacggcaacgcgcccacgctgctctacggctacggcggcttcaacgtgaacatggaggccaacttccgctcgagcatcctgccctggctggacgcgggcggcgtgtacgcggtggccaacctgcgcggcggcggcgagtacggcaaggcctggcacgacgccggccgcctggacaagaagcagaacgtcttcgacgacttccacgcggcggccgagtacttggtccagcagaagtacacgcagcccaagcggctggccatctacggcggcagcaacggcggcctgctggtgggcgcggccatgacgcagcggccggagctgtacggcgcggtggtgtgcgcggtgcccctgctggacatggtgcgctaccacctcttcggcagcggccggacctggattccggagtacggcacggcggagaagcccgaggacttcaagacgctgcacgcctactcgccctaccaccacgtgcggccggacgtgcgctaccccgcgctgctgatgatggcggcggaccacgacgaccgggtggaccccatgcacgcccgcaagttcgtggcggcggtgcagaactcgcccggaaacccggcgacggccctgctgcgcatcgaggccaacgcgggccacggtggcgcggatcaggtggccaaggccattgagtccagcgtggacctgtattcgttcctgttccaagtcctggatgtccagggggcacagggtggggtggcggcgcagggccgctga

SEQ ID NO: 10 - DNA, 1122 bp, Hordeum vulgare
  source 1..1122: mol_type = genomic DNA; note = cysteine proteinase(Hordeum vulgare)
atggggctgcttagcaagaagcttctggtggcatccatggtggctgccgtgctggccgtggcggccgtggagctctgcagcgccatcccgatggaggacaaggacctggaatcggaggaggcgctatgggacctctacgagcggtggcagtcggcgcaccgcgtgcgccgacaccacgccgagaagcaccgccgcttcggcaccttcaagtccaacgcccacttcatccactcccacaacaagcgcggcgaccacccctatcgcctccacctcaaccgcttcggcgacatggaccaggccgagttccgcgccaccttcgtcggcgacctccgccgcgacaccccgtccaagccgccgtccgtccccggattcatgtacgccgccctgaacgtgtccgacctgccgccgtccgtggactggcggcagaagggcgccgtgacgggcgtcaaggaccagggcaagtgcggcagctgctgggcattctccacggtggtgtccgtggaaggcatcaacgccatccgcacgggcagcctcgtgtccctctcggagcaggagctcatcgactgcgacacggcggacaacgacgggtgccagggcgggctcatggacaacgccttcgagtacatcaagaacaacggcgggctaatcaccgaggccgcctacccgtaccgcgccgccaggggcacctgcaacgtcgcgagagcggcccagaactcgcccgtggtggtgcatatcgacgggcaccaggacgtgccggccaacagcgaggaggacctggccagggcggtggcgaaccagcccgtgtccgtggccgtcgaggccagcgggaaggcgttcatgttctactccgagggggtgttcaccggtgaatgtggaacagagctggaccacggcgtggcggtggtcgggtacggggtggcagaggacggcaaggcgtactggacggtgaagaactcgtgggggccgtcgtggggggagcagggctacatcagggtggagaaggattctggtgcctcgggggggctctgcggcatcgccatggaggcgtcctatcccgtcaagacctacagcaagcccaagcccacgcccaggcgcgcccttggagccagggagtcactgtga

SEQ ID NO: 11 - DNA, 3957 bp, Rothia mucilaginosa
  source 1..3957: mol_type = genomic DNA; note = Subtilisins(Rothia mucilaginosa)
atgaaggctgtcggtgcgaccggtttgagtgcagctcttgcccttactctcggcgttcccgccactttctctgcagctcacgctcagagcccccagcaggtcgaaggaagcactgcgagcgcatccggtgacgctgcctcgcgtatcagcccgggcctgcagaaggctgaaggccaaatcaccgtgtatgtgcagttcaagggcaagggcgcgtatgagcagacccagtccgcggctgttctggcacgtaaggaggcgccggcgaaccgtcaggcgcaggtgcaggctattgcagctcaggtgcagtcgcaggctcagtctgtggcggctgcttctggcgcgaagctgatgtacaccacgcataatgcgatgcgtggtgcggcgattactggtgatgctgcgcagattcgtgcgttggcggagcgcccggatgtggagcgtatttcgccgattatcgcgaaggagcgtatgaactccggtagcgagattgatacgaagactcttgccacgtggactcgtgagaatacggggtacacgggtaagggcgtgaagattgcggttgttgactccggtgttgactatacgcacgctgattttggtggccccggtacggtggattcgtacctgaaggctaaggcgatgacggagcttccgtccgctgattcgggtctgattgatcgtaataagttcattggcggcattgacttggtcggtgatgactacaatgcgtcggtggctgagaagtcgactccgcagccggataataacccgctggattgccgcccggatggtttcggttctggcggtcacggcacgcacgtggcgggtactgccgccggttatggtgtgacggcgaacggtacgacttaccgtggcgattacaagaacctgacggaggagcagctgaagggcatgtctattggccccggtactgctcctgatgctcagattttggcgattcgtgtgttcggttgctacggcaattcgagtgttgtgatgaaggcgctcgataccgtgatggacccgaatggtgacggtgatttctctgaccgcgccgacattgtgaacctgtcgctcggtggcgagttcgccccggctgacgaccctgagtcgtacatgattaatacgatggcgcgccagggtgtgttcactgtggctgctgccggtaacgccaataactacaacggtgtgggtgacacctactccgattcgggtagcccggcgaatgctgctgcggcgctgtcggtggcgaacgcctacggttcgactcagccgattgaccgtgcccgcgtgaccacgaagactggtttggagtggttgcagggtgattattcggtgaacttcgattattcgaaggcatccgctgatcagctgcgcggcgaggttgttgccgcgccgaagcgtaaccgttacgcttgcgaggctttcaccgctgaagaggcgaaggctttgaagggtaagtgggtttacttcgactgggatcaggacgatttgacgttcccgtgcggttcgaaggtgcgtttcgataacgttcaggctgccggcggtgttggcgtggtgatggccggtaaggctgagcgctacacgatcggcattggtggtaatgcgaccattccgggtctgcgtctgactgcttcttcaacgaaggacctggagaaggctctggctgccggtccggtgaccgttgagatgaacctggactacaaggcttcgggccgcggtcctcactcgcacgctttcgatttgaattcctcgagcgctcgcggtcagcacggttctgatggcttcattaagcctgaccttgcggcaccgggtactgagattgtctctgccgctgtgggcacgggtaataagggtgtttccttcaccggtacctccatggcgaccccgcatgttgccggtgttgccgctctggtgatgcaggcgcatcaggattacaacccgcagatgattaaggctgcgctgatgaacggcgcgtccaccccgatcaagaatgagcagggtgcgcagtacgcggtggatcgtgtgggtacgggcatggtgaatgctcgcgccgccgtggacgcgaaggttatcgcgtacgatgcgaagacccccgagcgtgtttcgaccgcgtttggtgtgctggagtacacccccgattcgggtattcagactgttcagcgtgaaatcgtgctggacaacaccgattcgcaggctcacacctacactctgagctacgaggcaagcacgaccatcccgggtgtggagtactcctacccgcagcaggtgagcgtgggtgccggtgagcgtaagaacgtgaccgtgaccgtgcgtattgacccgtccaagctggagaagaccatggacccggcgatgagcgccgaccaggttgctcaggattggacgaccggtaagactctggccgccggtaagcgccagtacattgcgagcgcttctggtcgcctgattttctctgagaatggtcgcgaggcgatccgccagtcgattcacgtggcgccgaagcctgtctcgaagatgcgtgttgatgcgtcccgtattgattacaagggcatttccgataaggagtccacggtgactctgcgcggtacgaccctgaaccagggcggttaccgttcgctgctgggtgcgtttgagctgggtgcggtcagtgaccgcattccgtccggtcagctgaagctgccgtcgaaccagtctgttgacctgcagtatgtgggtgccgcctcggacgcaccggcgttgaaggctgccggcaagaacccgaacgatggttctctgttcttcggtatttcgacctggggcacctgggacagcatgcactggggccgtcaggtacaggttcagattgatacgaacaacgactccaccgcagattacgttctggaggtgacccgcgagaagggtctggactacccgctggtgaaggtctggtccatctccggtaacgcatccaccgtggtggctcgctacccgctgaacagcgcctggggcgacaccgacaccaacatcatggacaccaacaccatgattctgggtgtgccgctgaaggacctgggtctgaccgcggagaaggctcagagcatcaagtacacggtgcagactgacacctggcacaacgagggtaactcctacgtggacaccacctcgactatcgagtactcccccttcaaccccggtgtgtggttcaccggtgaggagtcgggtgtgcccggcctgtttgtggatcgtgacggcggtcagctgaccgttcaccgcaagaacaacaacaaggagcgtcaggctctgttcctgcacatgcacaacgcgaccggtgacctgagcggccgtaagaccgctaacggtgttgccgctggcgaccgtgcgcaggtcgtgaaggtggctcgtaccatccacgatgcacgcttcaccgacgttcctgcggataaccagttctaccgtgaaatcacgtggattgctgcccgtcagattgaccgcggctaccaggacggcaccttccgtccgctgaacaacatggaccgcgcaaccatggctgcgtacttctaccgcatgagcggctcgccgcagtacaccgcaccgtccaccccgagcttctcggatgttccgctcaaccacccctactacaaggagattgagtggatgaaggctcagggcatcacgaccggctggcccgatggcacctaccgcccggagggttcggtgaaccgtgacgcgatggctgcgttcttctaccgttacgcaggttcgcccgagtacactgcccccgcgcaggctcgtttcacggacgttccgaccgacaagcagttctaccgcgagatctcgtggctggctgagcagggcgtcaccaccggctggcccgatggttcgttccgccccgtagagccggtgcaccgcgacgctatggcggcgttcgtgtaccgctacagcacgggcgtgctgaaggaaagccctgaaatctag

SEQ ID NO: 12 - DNA, 944 bp, synthetic construct
  source 1..944: mol_type = other DNA; note = hIL-10_CUT
tgaacgtctagactgatggctagctcagtcctagggattatgctagctttctccatacccgtttttttgggctagcaataaggagatataccatgaaaaaaagaggggcgtttttagggctgttgttggtttctgcctgcgcatcagttttcgctgccaataatgaaaccagcaagtcggtcactttcccaaagtgtgaagatctggatgctgccggaattgccgcgagcgtaaaacgtgattatcaacaaaatcgcgtggcgcgttgggcagatgatcaaaaaattgtcggtcaggccgatcccgtggcttgggtcagtttgcaggacattcagggtaaagatgataaatggtcagtaccgctaaccgtgcgtggtaaaagtgccgatattcattaccaggtcagcgtggactgcaaagcgggaatggcggaatatcagcggcgtctgtctccgggtcaaggtacgcaaagtgaaaattcatgtactcattttcctggaaatcttccgaatatgttacgtgacctgcgtgacgcgttttctcgtgtcaaaaccttcttccagatgaaagaccaattagataatcttctgctgaaagaatcactgctcgaagatttcaaagggtatttagggtgtcaggctcttagcgaaatgattcaattctatcttgaagaagtaatgccgcaggccgaaaatcaggatccggatattaaagcccacgtaaatagccttggagaaaatctgaaaacactgcgtttacgtctgcgtcgatgccaccgtttcctgccgtgcgagaataagtcaaaagctgttgaacaagtgaagaacgcattcaacaagctgcaggaaaagggtatctataaggcgatgtccgagttcgatatattcattaattatatcgaggcttatatgaccatgaagattcggaattaaagtactgctaac

SEQ ID NO: 13 - DNA, 956 bp, synthetic construct
  source 1..956: mol_type = other DNA; note = hIL-10_mod
tgaacgtctagactgatggctagctcagtcctagggattatgctagctttctccatacccgtttttttgggctagcaataaggagatataccatgaaaaaaagaggggcgtttttagggctgttgttggtttctgcctgcgcatcagttttcgctgccaataatgaaaccagcaagtcggtcactttcccaaagtgtgaagatctggatgctgccggaattgccgcgagcgtaaaacgtgattatcaacaaaatcgcgtggcgcgttgggcagatgatcaaaaaattgtcggtcaggccgatcccgtggcttgggtcagtttgcaggacattcagggtaaagatgataaatggtcagtaccgctaaccgtgcgtggtaaaagtgccgatattcattaccaggtcagcgtggactgcaaagcgggaatggcggaatatcagcggcgtctgagcccgggacagggaacacagagtgaaaacagctgtacacattttcctggcaatttaccgaatatgctacgcgatttacgcgatgctttttccagagtaaaaacattcttccagatgaaagatcaactggataacctgctgttaaaagagtctttactggaagattttaaaggttatcttggctgccaggcgttaagtgagatgatccagttttatctggaagaggtaatgccgcaggcagaaaatcaggatccggatattaaggcacatgtcaactcactcggtgaaaatttaaaaacgctgcgtctgcgtctgcgtcgctgccaccgttttcttccttgtgaaaacaaaagcaaagcggtggagcaagtgaaaaatgccttcaataaactgcaagaaaaaggcatttataaagccatgagcgagtttgatattttcattaattatattgaagcctacatgacaatgaaaataaggaattgaagtactgctaacagtactgctaac

SEQ ID NO: 14 - DNA, 962 bp, synthetic construct
  source 1..962: mol_type = other DNA; note = hIL-10_LPP_CUT
tgaacgtctagatgcatgcatccatcaaaaaaatattctcaacataaaaaactttgtgtaatacttttctccatacccgtttttttgggctagcaataaggagatataccatgaaaaaaagaggggcgtttttagggctgttgttggtttctgcctgcgcatcagttttcgctgccaataatgaaaccagcaagtcggtcactttcccaaagtgtgaagatctggatgctgccggaattgccgcgagcgtaaaacgtgattatcaacaaaatcgcgtggcgcgttgggcagatgatcaaaaaattgtcggtcaggccgatcccgtggcttgggtcagtttgcaggacattcagggtaaagatgataaatggtcagtaccgctaaccgtgcgtggtaaaagtgccgatattcattaccaggtcagcgtggactgcaaagcgggaatggcggaatatcagcggcgtctgagtccggggcaagggacgcaaagcgaaaattcctgtactcattttccgggaaatcttccgaatatgctccgtgacttgcgggacgcattttcccgggttaaaacgttcttccagatgaaagaccaattggataatctcctgttgaaagaatctctgttagaagatttcaaaggctatcttgggtgtcaggcgttatccgaaatgattcaattctatctcgaagaagttatgccacaggccgaaaatcaggatccggatataaaagcacacgttaattcgttaggtgaaaatttaaagacactgagactccggctgcgccgttgccaccgtttcttgccatgcgagaataaatcgaaagcggttgaacaagtaaagaacgcattcaacaaacttcaggaaaaggggatctataaggcaatgtcagagttcgatatcttcataaattatattgaggcttacatgaccatgaaaatccggaattaaagtactgctaac

SEQ ID NO: 15 - DNA, 962 bp, synthetic construct; identical to SEQ ID NO: 14
  source 1..962: mol_type = other DNA; note = hIL-10_LPP_mod

SEQ ID NO: 16 - DNA, 944 bp, synthetic construct
  source 1..944: mol_type = other DNA; note = hIL-10_mut_CUT
tgaacgtctagactgatggctagctcagtcctagggattatgctagctttctccatacccgtttttttgggctagcaataaggagatataccatgaaaaaaagaggggcgtttttagggctgttgttggtttctgcctgcgcatcagttttcgctgccaataatgaaaccagcaagtcggtcactttcccaaagtgtgaagatctggatgctgccggaattgccgcgagcgtaaaacgtgattatcaacaaaatcgcgtggcgcgttgggcagatgatcaaaaaattgtcggtcaggccgatcccgtggcttgggtcagtttgcaggacattcagggtaaagatgataaatggtcagtaccgctaaccgtgcgtggtaaaagtgccgatattcattaccaggtcagcgtggactgcaaagcgggaatggcggaatatcagcggcgtctgtcacccgggcaaggtacccaatcagagaattcatgcacgcacttcccgggtatactgcccaacatgcttcgagaccttcgggacgcgttctcacgggtcaagacgttctttcaaatgaaggaccagcttgacaatttgttactgaaggaatcgctgctcgaggacttcaagggctacctgggttgtcaagctctctccgaaatgatacaattctacttggaagaagtgatgccacaagcggagaaccaagaccccgacatcaaagcccacgtgataagcctgggagagaacttgaacacattacgcttacggttgcgcagatgtcatcggctgctgccatgcgagaataagtcgaaggctgtcgaacaggtcaagaacgcgtttaacaagcttcaggagaagggtatatacaaggcgatgagtgaattcgacatctttataaactacatcgaggcttatatgactatgaagattagaaactaaagtactgctaac

SEQ ID NO: 17 - DNA, 944 bp, synthetic construct
  source 1..944: mol_type = other DNA; note = hIL-10_mut_mod
tgaacgtctagactgatggctagctcagtcctagggattatgctagctttctccatacccgtttttttgggctagcaataaggagatataccatgaaaaaaagaggggcgtttttagggctgttgttggtttctgcctgcgcatcagttttcgctgccaataatgaaaccagcaagtcggtcactttcccaaagtgtgaagatctggatgctgccggaattgccgcgagcgtaaaacgtgattatcaacaaaatcgcgtggcgcgttgggcagatgatcaaaaaattgtcggtcaggccgatcccgtggcttgggtcagtttgcaggacattcagggtaaagatgataaatggtcagtaccgctaaccgtgcgtggtaaaagtgccgatattcattaccaggtcagcgtggactgcaaagcgggaatggcggaatatcagcggcgtctgagcccgggacagggaacacagagtgaaaacagctgtacacattttcctggcattttaccgaatatgctacgcgatttacgcgatgctttttccagagtaaaaacattcttccagatgaaagatcaactggataacctgctgttaaaagagtctttactggaagattttaaaggttatcttggctgccaggcgttaagtgagatgatccagttttatctggaagaggtaatgccgcaggcagaaaatcaggatccggatattaaggcacatgtcatttcactcggtgaaaatttaaatacgctgcgtctgcgtctgcgtcgctgccaccgtcttcttccttgtgaaaacaaaagcaaagcggtggagcaagtgaaaaatgccttcaataaactgcaagaaaaaggcatttataaagccatgagcgagtttgatattttcattaattatattgaagcctacatgacaatgaaaataaggaattagagtactgctaac

SEQ ID NO: 18 - DNA, 35 bp, synthetic construct
  source 1..35: mol_type = other DNA; note = J23113 promoter
ctgatggctagctcagtcctagggattatgctagc

SEQ ID NO: 19 - DNA, 49 bp, synthetic construct
  source 1..49: mol_type = other DNA; note = pLPP
atcaaaaaaatattctcaacataaaaaactttgtgtaatacttttctcc

SEQ ID NO: 20 - DNA, 373 bp, Homo sapiens
  source 1..373: mol_type = other DNA; note = GLP-1
atgcacgatgagtttgaaaggcatgctgaaggcaccttcaccagcgatgtcagttcatatctggaaggtcaggccgccaaggaatttattgcttggctggtgaaaggcagaggttaacgttgtaacctgattaactgagactgacggcaacgccaaattgcctgatgcgctgcgcttatcaggcctacaagttgaattgcaatttattgaatttgcacatttttgtaggccggataaggcgtttacgccgcatccggcaacataaagcgcaatttgtcagcaacgtgcttccccgccaccggcggggtttttttctgcctggaatttacctgtaacccccaaataacccctcatttcacccactaatttataa

SEQ ID NO: 21 - DNA, 390 bp, synthetic construct
  source 1..390: mol_type = other DNA; note = Anti-TNFalfa
gctcaagtacaattacaagagagtggtggcgggcttgttcaacctggtgggtcccttcgtctctcctgtgctgcttctggacgtacattctcggaccactcaggttacacatacacaatcggatggttccggcaagctccagggaaggagcgggagttcgtagctcgtatctactggtcgtctggaaatacgtactacgccgactccgtaaaggggcgtttcgccatatcacgggacatcgcaaagaatacagttgacttgacaatgaataatttagagcctgaggacacagctgtttactactgtgctgcccgtgacgggatccctacaagtcggtccgtagagagttacaattactggggacaaggtacgcaagtcacagtctcttcg

SEQ ID NO: 22 - DNA, 39937 bp, unidentified
  source 1..39937: mol_type = other DNA; note = Enterobacteria phage
tctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccctatctgttacagtctcctaaagtatcctcctaaagtcacctcctaacgtccatcctaaagccaacacctaaagcctacacctaaagacccatcaagtcaacgcctatcttaaagtttaaacataaagaccagacctaaagaccagacctaaagacactacataaagaccagacctaaagacgccttgttgttagccataaagtgataacctttaatcattgtctttattaatacaactcactataaggagagacaacttaaagagacttaaaagattaatttaaaatttatcaaaaagagtattgacttaaagtctaacctataggatacttacagccatcgagagggacacggcgaatagccatcccaatcgacaccggggtcaaccggataagtagacagcctgataagtcgcacgaaaaacaggtattgacaacatgaagtaacatgcagtaagatacaaatcgctaggtaacactagcagcgtcaaccgggcgcacagtgccttctaggtgacttaagcgcaccacggcacataaggtgaaacaaaacggttgacaacatgaagtaaacacggtacgatgtaccacatgaaacgacagtgagtcaccacactgaaaggtgatgcggtctaacgaaacctgacctaagacgctctttaacaatctggtaaatagctcttgagtgcatgactagcggataactcaagggtatcgcaaggtgccctttatgatattcactaataactgcacgaggtaacacaagatggctatgtctaacatgacttacaacaacgttttcgaccacgcttacgaaatgctgaaagaaaacatccgttatgatgacatccgtgacactgatgacctgcacgatgctattcacatggctgccgataatgcagttccgcactactacgctgacatctttagcgtaatggcaagtgagggcattgaccttgagttcgaagactctggtctgatgcctgacaccaaggacgtaatccgcatcctgcaagcgcgtatctatgagcaattaacgattgacctctgggaagacgcagaagacttgctcaatgaatacttggaggaagtcgaggagtacgaggaggatgaagagtaatgtctactaccaacgtgcaatacggtctgaccgctcaaactgtacttttctatagcgacatggtgcgctgtggctttaactggtcactcgcaatggcacagctcaaagaactgtacgaaaacaacaaggcaatagctttagaatctgctgagtgatagactcaaggtcgctcctagcgagtggcctttatgattatcactttacttatgagggagtaatgtatatgcttactatcggtctactcaccgctctaggtctagctgtaggtgcatcctttgggaaggctttaggtgtagctgtaggttcctactttaccgcttgcatcatcataggaatcatcaaaggggcactacgcaaatgatgaagcactacgttatgccaatccacacgtccaacggggcaaccgtatgtacacctgatgggttcgcaatgaaacaacgaatcgaacgccttaagcgtgaactccgcattaaccgcaagattaacaagataggttccggctatgacagaacgcactgatggcttaaagaaaggttatatgcccaatggcacactatacgctgcaaatcggcgaatagtgagaacttggcgagagaacaacctcgaacgccgcaaggacaagagagggcggcgtggcatagacgaaaggaaaaggttaaagccaagaaactcgccgcacttgaacaggcactagccaacacactgaacgctatctcataacgaacataaaggacacaatgcaatgaacattaccgacatcatgaacgctatcgacgcaatcaaagcactgccaatctgtgaacttgacaagcgtcaaggtatgcttatcgacttactggtcgagatggtcaacagcgagacgtgtgatggcgagctaaccgaactaaatcaggcacttgagcatcaagattggtggactaccttgaagtgtctcacggctgacgcagggttcaagatgctcggtaatggtcacttctcggctgcttatagtcacccgctgctacctaacagagtgattaaggtgggctttaagaaagaggattcaggcgcagcctataccgcattctgccgcatgtatcagggtcgtcctggtatccctaacgtctacgatgtacagcgccacgctggatgctatacggtggtacttgacgcacttaaggattgcgagcgtttcaacaatgatgcccattataaatacgctgagattgcaagcgacatcattgattgcaattcggatgagcatgatgagttaactggatgggatggtgagtttgttgaaacttgtaaactaatccgcaagttctttgagggcatcgcctcattcgacatgcatagcgggaacatcatgttctcaaatggagacgtaccatacatcaccgacccggtatcattctcgcagaagaaagacggtggcgcattcagcatcgaccctgaggaactcatcaaggaagtcgaggaagtcgcacgacagaaagaaattgaccgcgctaaggcccgtaaagaacgtcacgaggggcgcttagaggcacgcagattcaaacgtcgcaaccgcaaggcacgtaaagcacacaaagctaagcgcgaaagaatgcttgctgcgtggcgatgggctgaacgtcaagaacggcgtaaccatgaggtagctgtagatgtactaggaagaaccaataacgctatgctctgggtcaacatgttctctggggactttaaggcgcttgaggaacgaatcgcgctgcactggcgtaatgctgaccggatggctatcgctaatggtcttacgctcaacattgataagcaacttgacgcaatgttaatgggctgatagtcttatcttacaggtcatctgcgggtggcctgaataggtacgatttactaactggaagaggcactaaatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaacgccaaatcaatacgactcactatagagggacaaactcaaggtcattcgcaagagtggcctttatgattgaccttcttccggttaatacgactcactataggagaaccttaaggtttaactttaagacccttaagtgttaattagagatttaaattaaagaattactaagagaggactttaagtatgcgtaacttcgaaaagatgaccaaacgttctaaccgtaatgctcgtgacttcgaggcaaccaaaggtcgcaagttgaataagactaagcgtgaccgctctcacaagcgtagctgggagggtcagtaagatgggacgtttatatagtggtaatctggcagcattcaaggcagcaacaaacaagctgttccagttagacttagcggtcatttatgatgactggtatgatgcctatacaagaaaagattgcatacggttacgtattgaggacaggagtggaaacctgattgatactagcaccttctaccaccacgacgaggacgttctgttcaatatgtgtactgattggttgaaccatatgtatgaccagttgaaggactggaagtaatacgactcagtatagggacaatgcttaaggtcgctctctaggagtggccttagtcatttaaccaataggagataaacattatgatgaacattaagactaacccgtttaaagccgtgtctttcgtagagtctgccattaagaaggctctggataacgctgggtatcttatcgctgaaatcaagtacgatggtgtacgcgggaacatctgcgtagacaatactgctaacagttactggctctctcgtgtatctaaaacgattccggcactggagcacttaaacgggtttgatgttcgctggaagcgtctactgaacgatgaccgttgcttctacaaagatggctttatgcttgatggggaactcatggtcaagggcgtagactttaacacagggtccggcctactgcgtaccaaatggactgacacgaagaaccaagagttccatgaagagttattcgttgaaccaatccgtaagaaagataaagttccctttaagctgcacactggacaccttcacataaaactgtacgctatcctcccgctgcacatcgtggagtctggagaagactgtgatgtcatgacgttgctcatgcaggaacacgttaagaacatgctgcctctgctacaggaatacttccctgaaatcgaatggcaagcggctgaatcttacgaggtctacgatatggtagaactacagcaactgtacgagcagaagcgagcagaaggccatgagggtctcattgtgaaagacccgatgtgtatctataagcgcggtaagaaatctggctggtggaaaatgaaacctgagaacgaagctgacggtatcattcagggtctggtatggggtacaaaaggtctggctaatgaaggtaaagtgattggttttgaggtgcttcttgagagtggtcgtttagttaacgccacgaatatctctcgcgccttaatggatgagttcactgagacagtaaaagaggccaccctaagtcaatggggattctttagcccatacggtattggcgacaacgatgcttgtactattaacccttacgatggctgggcgtgtcaaattagctacatggaggaaacacctgatggctctttgcggcacccatcgttcgtaatgttccgtggcaccgaggacaaccctcaagagaaaatgtaatcacactggctcaccttcgggtgggcctttctgcgtttataaggagacactttatgtttaagaaggttggtaaattccttgcggctttggcagctatcctgacgcttgcgtatattcttgcggtataccctcaagtagcactagtagtagttggcgcttgttacttagcggcagtgtgtgcttgcgtgtggagtatagttaactggtaatacgactcactaaaggaggtacacaccatgatgtacttaatgccattactcatcgtcattgtaggatgccttgcgctccactgtagcgatgatgatatgccagatggtcacgcttaatacgactcactaaaggagacactatatgtttcgacttcattacaacaaaagcgttaagaatttcacggttcgccgtgctgaccgttcaatcgtatgtgcgagcgagcgccgagctaagatacctcttattggtaacacagttcctttggcaccgagcgtccacatcattatcacccgtggtgactttgagaaagcaatagacaagaaacgtccggttcttagtgtggcagtgacccgcttcccgttcgtccgtctgttactcaaacgaatcaaggaggtgttctgatgggactgttagatggtgaagcctgggaaaaagaaaacccgccagtacaagcaactgggtgtatagcttgcttagagaaagatgaccgttatccacacacctgtaacaaaggagctaacgatatgaccgaacgtgaacaagagatgatcattaagttgatagacaataatgaaggtcgcccagatgatttgaatggctgcggtattctctgctccaatgtcccttgccacctctgccccgcaaataacgatcaaaagataaccttaggtgaaatccgagcgatggacccacgtaaaccacatctgaataaacctgaggtaactcctacagatgaccagccttccgctgagacaatcgaaggtgtcactaagccttcccactacatgctgtttgacgacattgaggctatcgaagtgattgctcgttcaatgaccgttgagcagttcaagggatactgcttcggtaacatcttaaagtacagactacgtgctggtaagaagtcagagttagcgtacttagagaaagacctagcgaaagcagacttctataaagaactctttgagaaacataaggataaatgttatgcataacttcaagtcaaccccacctgccgacagcctatctgatgacttcacatcttgctcagagtggtgccgaaagatgtgggaagagacattcgacgatgcgtacatcaagctgtatgaactttggaaatcgagaggtcaatgactatgtcaaacgtaaatacaggttcacttagtgtggacaataagaagttttgggctaccgtagagtcctcggagcattccttcgaggttccaatctacgctgagaccctagacgaagctctggagttagccgaatggcaatacgttccggctggctttgaggttactcgtgtgcgtccttgtgtagcaccgaagtaatacgactcactattagggaagactccctctgagaaaccaaacgaaacctaaaggagattaacattatggctaagaagattttcacctctgcgctgggtaccgctgaaccttacgcttacatcgccaagccggactacggcaacgaagagcgtggctttgggaaccctcgtggtgtctataaagttgacctgactattcccaacaaagacccgcgctgccagcgtatggtcgatgaaatcgtgaagtgtcacgaagaggcttatgctgctgccgttgaggaatacgaagctaatccacctgctgtagctcgtggtaagaaaccgctgaaaccgtatgagggtgacatgccgttcttcgataacggtgacggtacgactacctttaagttcaaatgctacgcgtctttccaagacaagaagaccaaagagaccaagcacatcaatctggttgtggttgactcaaaaggtaagaagatggaagacgttccgattatcggtggtggctctaagctgaaagttaaatattctctggttccatacaagtggaacactgctgtaggtgcgagcgttaagctgcaactggaatccgtgatgctggtcgaactggctacctttggtggcggtgaagacgattgggctgacgaagttgaagagaacggctatgttgcctctggttctgccaaagcgagcaaaccacgcgacgaagaaagctgggacgaagacgacgaagagtccgaggaagcagacgaagacggagacttctaagtggaactgcgggagaaaatccttgagcgaatcaaggtgacttcctctgggtgttgggagtggcagggcgctacgaacaataaagggtacgggcaggtgtggtgcagcaataccggaaaggttgtctactgtcatcgcgtaatgtctaatgctccgaaaggttctaccgtcctgcactcctgtgataatccattatgttgtaaccctgaacacctatccataggaactccaaaagagaactccactgacatggtaaataagggtcgctcacacaaggggtataaactttcagacgaagacgtaatggcaatcatggagtccagcgagtccaatgtatccttagctcgcacctatggtgtctcccaacagactatttgtgatatacgcaaagggaggcgacatggcaggttacggcgctaaaggaatccgaaaggttggagcgtttcgctctggcctagaggacaaggtttcaaagcagttggaatcaaaaggtattaaattcgagtatgaagagtggaaagtgccttatgtaattccggcgagcaatcacacttacactccagacttcttacttccaaacggtatattcgttgagacaaagggtctgtgggaaagcgatgatagaaagaagcacttattaattagggagcagcaccccgagctagacatccgtattgtcttctcaagctcacgtactaagttatacaaaggttctccaacgtcttatggagagttctgcgaaaagcatggtattaagttcgctgataaactgatacctgctgagtggataaaggaacccaagaaggaggtcccctttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctgtgcttcgcgcccatcatgaggtggcgccgaaggcttgcccttcgttcgaccttaagcgttggtgggagaagaacgaactggtcacttctgaccgtggataattaattgaactcactaaagggagaccacagcggtttccctttgttcgcattggaggtcaaataatgcgcaagtcttataaacaattctataaggctccgaggaggcatatccaagtgtgggaggcagccaatgggcctataccaaaaggttattatatagaccacattgacggcaatccactcaacgacgccttagacaatctccgtctggctctcccaaaagaaaactcatggaacatgaagactccaaagagcaatacctcaggactaaagggactgagttggagcaaggaaagggagatgtggagaggcactgtaacagctgagggtaaacagcataactttcgtagtagagatctattggaagtcgttgcgtggatttatagaactaggagggaattgcatggacaattcgcacgattccgatagtgtatttctttaccacattccttgtgacaactgtgggagtagtgatgggaactcgctgttctctgacggacacacgttctgctacgtatgcgagaagtggactgctggtaatgaagacactaaagagagggcttcaaaacggaaaccctcaggaggtaaaccaatgacttacaacgtgtggaacttcggggaatccaatggacgctactccgcgttaactgcgagaggaatctccaaggaaacctgtcagaaggctggctactggattgccaaagtagacggtgtgatgtaccaagtggctgactatcgggaccagaacggcaacattgtgagtcagaaggttcgagataaagataagaactttaagaccactggtagtcacaagagtgacgctctgttcgggaagcacttgtggaatggtggtaagaagattgtcgttacagaaggtgaaatcgacatgcttaccgtgatggaacttcaagactgtaagtatcctgtagtgtcgttgggtcacggtgcctctgccgctaagaagacatgcgctgccaactacgaatactttgaccagttcgaacagattatcttaatgttcgatatggacgaagcagggcgcaaagcagtcgaagaggctgcacaggttctacctgctggtaaggtacgagtggcagttcttccgtgtaaggatgcaaacgagtgtcacctaaatggtcacgaccgtgaaatcatggagcaagtgtggaatgctggtccttggattcctgatggtgtggtatcggctctttcgttacgtgaacgaatccgtgagcacctatcgtccgaggaatcagtaggtttacttttcagtggctgcactggtatcaacgataagaccttaggtgcccgtggtggtgaagtcattatggtcacttccggttccggtatgggtaagtcaacgttcgtccgtcaacaagctctacaatggggcacagcgatgggcaagaaggtaggcttagcgatgcttgaggagtccgttgaggagaccgctgaggaccttataggtctacacaaccgtgtccgactgagacaatccgactcactaaagagagagattattgagaacggtaagttcgaccaatggttcgatgaactgttcggcaacgatacgttccatctatatgactcattcgccgaggctgagacggatagactgctcgctaagctggcctacatgcgctcaggcttgggctgtgacgtaatcattctagaccacatctcaatcgtcgtatccgcttctggtgaatccgatgagcgtaagatgattgacaacctgatgaccaagctcaaagggttcgctaagtcaactggggtggtgctggtcgtaatttgtcaccttaagaacccagacaaaggtaaagcacatgaggaaggtcgccccgtttctattactgacctacgtggttctggcgcactacgccaactatctgatactattattgcccttgagcgtaatcagcaaggcgatatgcctaaccttgtcctcgttcgtattctcaagtgccgctttactggtgatactggtatcgctggctacatggaatacaacaaggaaaccggatggcttgaaccatcaagttactcaggggaagaagagtcacactcagagtcaacagactggtccaacgacactgacttctgacaggattcttgatgactttccagacgactacgagaagtttcgctggagagtcccattctaatacgactcactaaaggagacacaccatgttcaaactgattaagaagttaggccaactgctggttcgtatgtacaacgtggaagccaagcgactgaacgatgaggctcgtaaagaggccacacagtcacgcgctctggcgattcgctccaacgaactggctgacagtgcatccactaaagttaccgaggctgcccgtgtggcaaaccaagctcaacagctttccaaattctttgagtaatcaaacaggagaaaccattatgtctaacgtagctgaaactatccgtctatccgatacagctgaccagtggaaccgtcgagtccacatcaacgttcgcaacggtaaggcgactatggtttaccgctggaaggactctaagtcctctaagaatcacactcagcgtatgacgttgacagatgagcaagcactgcgtctggtcaatgcgcttaccaaagctgccgtgacagcaattcatgaagctggtcgcgtcaatgaagctatggctatcctcgacaagattgataactaagagtggtatcctcaaggtcgccaaagtggtggccttcatgaatactattcgactcactataggagatattaccatgcgtgaccctaaagttatccaagcagaaatcgctaaactggaagctgaactggaggacgttaagtaccatgaagctaagactcgctccgctgttcacatcttgaagaacttaggctggacttggacaagacagactggctggaagaaaccagaagttaccaagctgagtcataaggtgttcgataaggacactatgacccacatcaaggctggtgattgggttaaggttgacatgggagttgttggtggatacggctacgtccgctcagttagtggcaaatatgcacaagtgtcatacatcacaggtgttactccacgcggtgcaatcgttgccgataagaccaacatgattcacacaggtttcttgacagttgtttcatatgaagagattgttaagtcacgataatcaataggagaaatcaatatgatcgtttctgacatcgaagctaacgccctcttagagagcgtcactaagttccactgcggggttatctacgactactccaccgctgagtacgtaagctaccgtccgagtgacttcggtgcgtatctggatgcgctggaagccgaggttgcacgaggcggtcttattgtgttccacaacggtcacaagtatgacgttcctgcattgaccaaactggcaaagttgcaattgaaccgagagttccaccttcctcgtgagaactgtattgacacccttgtgttgtcacgtttgattcattccaacctcaaggacaccgatatgggtcttctgcgttccggcaagttgcccggaaaacgctttgggtctcacgctttggaggcgtggggttatcgcttaggcgagatgaagggtgaatacaaagacgactttaagcgtatgcttgaagagcagggtgaagaatacgttgacggaatggagtggtggaacttcaacgaagagatgatggactataacgttcaggacgttgtggtaactaaagctctccttgagaagctactctctgacaaacattacttccctcctgagattgactttacggacgtaggatacactacgttctggtcagaatcccttgaggccgttgacattgaacatcgtgctgcatggctgctcgctaaacaagagcgcaacgggttcccgtttgacacaaaagcaatcgaagagttgtacgtagagttagctgctcgccgctctgagttgctccgtaaattgaccgaaacgttcggctcgtggtatcagcctaaaggtggcactgagatgttctgccatccgcgaacaggtaagccactacctaaataccctcgcattaagacacctaaagttggtggtatctttaagaagcctaagaacaaggcacagcgagaaggccgtgagccttgcgaacttgatacccgcgagtacgttgctggtgctccttacaccccagttgaacatgttgtgtttaacccttcgtctcgtgaccacattcagaagaaactccaagaggctgggtgggtcccgaccaagtacaccgataagggtgctcctgtggtggacgatgaggtactcgaaggagtacgtgtagatgaccctgagaagcaagccgctatcgacctcattaaagagtacttgatgattcagaagcgaatcggacagtctgctgagggagacaaagcatggcttcgttatgttgctgaggatggtaagattcatggttctgttaaccctaatggagcagttacgggtcgtgcgacccatgcgttcccaaaccttgcgcaaattccgggtgtacgttctccttatggagagcagtgtcgcgctgcttttggcgctgagcaccatttggatgggataactggtaagccttgggttcaggctggcatcgacgcatccggtcttgagctacgctgcttggctcacttcatggctcgctttgataacggcgagtacgctcacgagattcttaacggcgacatccacactaagaaccagatagctgctgaactacctacccgagataacgctaagacgttcatctatgggttcctctatggtgctggtgatgagaagattggacagattgttggtgctggtaaagagcgcggtaaggaactcaagaagaaattccttgagaacacccccgcgattgcagcactccgcgagtctatccaacagacacttgtcgagtcctctcaatgggtagctggtgagcaacaagtcaagtggaaacgccgctggattaaaggtctggatggtcgtaaggtacacgttcgtagtcctcacgctgccttgaataccctactgcaatctgctggtgctctcatctgcaaactgtggattatcaagaccgaagagatgctcgtagagaaaggcttgaagcatggctgggatggggactttgcgtacatggcatgggtacatgatgaaatccaagtaggctgccgtaccgaagagattgctcaggtggtcattgagaccgcacaagaagcgatgcgctgggttggagaccactggaacttccggtgtcttctggataccgaaggtaagatgggtcctaattgggcgatttgccactgatacaggaggctactcatgaacgaaagacacttaacaggtgctgcttctgaaatgctagtagcctacaaatttaccaaagctgggtacactgtctattaccctatgctgactcagagtaaagaggacttggttgtatgtaaggatggtaaatttagtaaggttcaggttaaaacagccacaacggttcaaaccaacacaggagatgccaagcaggttaggctaggtggatgcggtaggtccgaatataaggatggagactttgacattcttgcggttgtggttgacgaagatgtgcttattttcacatgggacgaagtaaaaggtaagacatccatgtgtgtcggcaagagaaacaaaggcataaaactataggagaaattattatggctatgacaaagaaatttaaagtgtccttcgacgttaccgcaaagatgtcgtctgacgttcaggcaatcttagagaaagatatgctgcatctatgtaagcaggtcggctcaggtgcgattgtccccaatggtaaacagaaggaaatgattgtccagttcctgacacacggtatggaaggattgatgacattcgtagtacgtacatcatttcgtgaggccattaaggacatgcacgaagagtatgcagataaggactctttcaaacaatctcctgcaacagtacgggaggtgttctgatgtctgactacctgaaagtgctgcaagcaatcaaaagttgccctaagactttccagtccaactatgtacggaacaatgcgagcctcgtagcggaggccgcttcccgtggtcacatctcgtgcctgactactagtggacgtaacggtggcgcttgggaaatcactgcttccggtactcgctttctgaaacgaatgggaggatgtgtctaatgtctcgtgaccttgtgactattccacgcgatgtgtggaacgatatacagggctacatcgactctctggaacgtgagaacgatagccttaagaatcaactaatggaagctgacgaatacgtagcggaactagaggagaaacttaatggcacttcttgaccttaaacaattctatgagttacgtgaaggctgcgacgacaagggtatccttgtgatggacggcgactggctggtcttccaagctatgagtgctgctgagtttgatgcctcttgggaggaagagatttggcaccgatgctgtgaccacgctaaggcccgtcagattcttgaggattccattaagtcctacgagacccgtaagaaggcttgggcaggtgctccaattgtccttgcgttcaccgatagtgttaactggcgtaaagaactggttgacccgaactataaggctaaccgtaaggccgtgaagaaacctgtagggtactttgagttccttgatgctctctttgagcgcgaagagttctattgcatccgtgagcctatgcttgagggtgatgacgttatgggagttattgcttccaatccgtctgccttcggtgctcgtaaggctgtaatcatctcttgcgataaggactttaagaccatccctaactgtgacttcctgtggtgtaccactggtaacatcctgactcagaccgaagagtccgctgactggtggcacctcttccagaccatcaagggtgacatcactgatggttactcagggattgctggatggggtgataccgccgaggacttcttgaataacccgttcataaccgagcctaaaacgtctgtgcttaagtccggtaagaacaaaggccaagaggttactaaatgggttaaacgcgaccctgagcctcatgagacgctttgggactgcattaagtccattggcgcgaaggctggtatgaccgaagaggatattatcaagcagggccaaatggctcgaatcctacggttcaacgagtacaactttattgacaaggagatttacctgtggagaccgtagcgtatattggtctgggtctttgtgttctcggagtgtgcctcatttcgtggggcctttgggacttagccagaataatcaagtcgttacacgacactaagtgataaactcaaggtccctaaattaatacgactcactatagggagataggggcctttacgattattactttaagatttaactctaagaggaatctttattatgttaacacctattaaccaattacttaagaaccctaacgatattccagatgtacctcgtgcaaccgctgagtatctacaggttcgattcaactatgcgtacctcgaagcgtctggtcatataggacttatgcgtgctaatggttgtagtgaggcccacatcttgggtttcattcagggcctacagtatgcctctaacgtcattgacgagattgagttacgcaaggaacaactaagagatgatggggaggattgacactatgtgtttctcaccgaaaattaaaactccgaagatggataccaatcagattcgagccgttgagccagcgcctctgacccaagaagtgtcaagcgtggagttcggtgggtcttctgatgagacggataccgagggcaccgaagtgtctggacgcaaaggcctcaaggtcgaacgtgatgattccgtagcgaagtctaaagccagcggcaatggctccgctcgtatgaaatcttccatccgtaagtccgcatttggaggtaagaagtgatgtctgagttcacatgtgtggaggctaagagtcgcttccgtgcaatccggtggactgtggaacaccttgggttgcctaaaggattcgaaggacactttgtgggctacagcctctacgtagacgaagtgatggacatgtctggttgccgtgaagagtacattctggactctaccggaaaacatgtagcgtacttcgcgtggtgcgtaagctgtgacattcaccacaaaggagacattctggatgtaacgtccgttgtcattaatcctgaggcagactctaagggcttacagcgattcctagcgaaacgctttaagtaccttgcggaactccacgattgcgattgggtgtctcgttgtaagcatgaaggcgagacaatgcgtgtatactttaaggaggtataagttatgggtaagaaagttaagaaggccgtgaagaaagtcaccaagtccgttaagaaagtcgttaaggaaggggctcgtccggttaaacaggttgctggcggtctagctggtctggctggtggtactggtgaagcacagatggtggaagtaccacaagctgccgcacagattgttgacgtacctgagaaagaggtttccactgaggacgaagcacagacagaaagcggacgcaagaaagctcgtgctggcggtaagaaatccttgagtgtagcccgtagctccggtggcggtatcaacatttaatcaggaggttatcgtggaagactgcattgaatggaccggaggtgtcaactctaagggttatggtcgtaagtgggttaatggtaaacttgtgactccacataggcacatctatgaggagacatatggtccagttccaacaggaattgtggtgatgcatatctgcgataaccctaggtgctataacataaagcaccttacgcttggaactccaaaggataattccgaggacatggttaccaaaggtagacaggctaaaggagaggaactaagcaagaaacttacagagtcagacgttctcgctatacgctcttcaaccttaagccaccgctccttaggagaactgtatggagtcagtcaatcaaccataacgcgaatactacagcgtaagacatggagacacatttaatggctgagaaacgaacaggacttgcggaggatggcgcaaagtctgtctatgagcgtttaaagaacgaccgtgctccctatgagacacgcgctcagaattgcgctcaatataccatcccatcattgttccctaaggactccgataacgcctctacagattatcaaactccgtggcaagccgtgggcgctcgtggtctgaacaatctagcctctaagctcatgctggctctattccctatgcagacttggatgcgacttactatatctgaatatgaagcaaagcagttactgagcgaccccgatggactcgctaaggtcgatgagggcctctcgatggtagagcgtatcatcatgaactacattgagtctaacagttaccgcgtgactctctttgaggctctcaaacagttagtcgtagctggtaacgtcctgctgtacctaccggaaccggaagggtcaaactataatcccatgaagctgtaccgattgtcttcttatgtggtccaacgagacgcattcggcaacgttctgcaaatggtgactcgtgaccagatagcttttggtgctctccctgaggacatccgtaaggctgtagaaggtcaaggtggtgagaagaaagctgatgagacaatcgacgtgtacactcacatctatctggatgaggactcaggtgaatacctccgatacgaagaggtcgagggtatggaagtccaaggctccgatgggacttatcctaaagaggcttgcccatacatcccgattcggatggtcagactagatggtgaatcctacggtcgttcgtacattgaggaatacttaggtgacttacggtcccttgaaaatctccaagaggctatcgtcaagatgtccatgattagctctaaggttatcggcttagtgaatcctgctggtatcacccagccacgccgactgaccaaagctcagactggtgacttcgttactggtcgtccagaagacatctcgttcctccaactggagaagcaagcagactttactgtagctaaagccgtaagtgacgctatcgaggctcgcctttcgtttgcctttatgttgaactctgcggttcagcgtacaggtgaacgtgtgaccgccgaagagattcggtatgtagcttctgaacttgaagatactttaggtggtgtctactctatcctttctcaagaattacaattgcctctggtacgagtgctcttgaagcaactacaagccacgcaacagattcctgagttacctaaggaagccgtagagccaaccattagtacaggtctggaagcaattggtcgaggacaagaccttgataagctggagcggtgtgtcactgcgtgggctgcactggcacctatgcgggacgaccctgatattaaccttgcgatgattaagttacgtattgccaacgctatcggtattgacacttctggtattctactcaccgaagaacagaagcaacagaagatggcccaacagtctatgcaaatgggtatggataatggtgctgctgcgctggctcaaggtatggctgcacaagctacagcttcacctgaggctatggctgctgccgctgattccgtaggtttacagccgggaatttaatacgactcactatagggagacctcatctttgaaatgagcgatgacaagaggttggagtcctcggtcttcctgtagttcaactttaaggagacaataataatggctgaatctaatgcagacgtatatgcatcttttggcgtgaactccgctgtgatgtctggtggttccgttgaggaacatgagcagaacatgctggctcttgatgttgctgcccgtgatggcgatgatgcaatcgagttagcgtcagacgaagtggaaacagaacgtgacctgtatgacaactctgacccgttcggtcaagaggatgacgaaggccgcattcaggttcgtatcggtgatggctctgagccgaccgatgtggacactggagaagaaggcgttgagggcaccgaaggttccgaagagtttaccccactgggcgagactccagaagaactggtagctgcctctgagcaacttggtgagcacgaagagggcttccaagagatgattaacattgctgctgagcgtggcatgagtgtcgagaccattgaggctatccagcgtgagtacgaggagaacgaagagttgtccgccgagtcctacgctaagctggctgaaattggctacacgaaggctttcattgactcgtatatccgtggtcaagaagctctggtggagcagtacgtaaacagtgtcattgagtacgctggtggtcgtgaacgttttgatgcactgtataaccaccttgagacgcacaaccctgaggctgcacagtcgctggataatgcgttgaccaatcgtgacttagcgaccgttaaggctatcatcaacttggctggtgagtctcgcgctaaggcgttcggtcgtaagccaactcgtagtgtgactaatcgtgctattccggctaaacctcaggctaccaagcgtgaaggctttgcggaccgtagcgagatgattaaagctatgagtgaccctcggtatcgcacagatgccaactatcgtcgtcaagtcgaacagaaagtaatcgattcgaacttctgatagacttcgaaattaatacgactcactatagggagaccacaacggtttccctctagaaataattttgtttaactttaagaaggagatatacatatggctagcatgactggtggacagcaaatgggtactaaccaaggtaaaggtgtagttgctgctggagataaactggcgttgttcttgaaggtatttggcggtgaagtcctgactgcgttcgctcgtacctccgtgaccacttctcgccacatggtacgttccatctccagcggtaaatccgctcagttccctgttctgggtcgcactcaggcagcgtatctggctccgggcgagaacctcgacgataaacgtaaggacatcaaacacaccgagaaggtaatcaccattgacggtctcctgacggctgacgttctgatttatgatattgaggacgcgatgaaccactacgacgttcgctctgagtatacctctcagttgggtgaatctctggcgatggctgcggatggtgcggttctggctgagattgccggtctgtgtaacgtggaaagcaaatataatgagaacatcgagggcttaggtactgctaccgtaattgagaccactcagaacaaggccgcacttaccgaccaagttgcgctgggtaaggagattattgcggctctgactaaggctcgtgcggctctgaccaagaactatgttccggctgctgaccgtgtgttctactgtgacccagatagctactctgcgattctggcagcactgatgccgaacgcagcaaactacgctgctctgattgaccctgagaagggttctatccgcaacgttatgggctttgaggttgtagaagttccgcacctcaccgctggtggtgctggtaccgctcgtgagggcactactggtcagaagcacgtcttccctgccaataaaggtgagggtaatgtcaaggttgctaaggacaacgttatcggcctgttcatgcaccgctctgcggtaggtactgttaagctgcgtgacttggctctggagcgcgctcgccgtgctaacttccaagcggaccagattatcgctaagtacgcaatgggccacggtggtcttcgcccagaagctgctggtgcagtggttttcaaagtggagtaatgctgggggtggcctcaacggtcgctgctagtcccgaagaggcgagtgttacttcaacagaagaaaccttaacgccagcacaggaggccgcacgcacccgcgctgctaacaaagcccgaaaggaagctgagttggctgctgccaccgctgagcaataactagcataaccccttggggcctctaaacgggtcttgaggggttttttgctgaaaggaggaactatatgcgctcatacgatatgaacgttgagactgccgctgagttatcagctgtgaacgacattctggcgtctatcggtgaacctccggtatcaacgctggaaggtgacgctaacgcagatgcagcgaacgctcggcgtattctcaacaagattaaccgacagattcaatctcgtggatggacgttcaacattgaggaaggcataacgctactacctgatgtttactccaacctgattgtatacagtgacgactatttatccctaatgtctacttccggtcaatccatctacgttaaccgaggtggctatgtgtatgaccgaacgagtcaatcagaccgctttgactctggtattactgtgaacattattcgtctccgcgactacgatgagatgcctgagtgcttccgttactggattgtcaccaaggcttcccgtcagttcaacaaccgattctttggggcaccggaagtagagggtgtactccaagaagaggaagatgaggctagacgtctctgcatggagtatgagatggactacggtgggtacaatatgctggatggagatgcgttcacttctggtctactgactcgctaacattaataaataaggaggctctaatggcactcattagccaatcaatcaagaacttgaagggtggtatcagccaacagcctgacatccttcgttatccagaccaagggtcacgccaagttaacggttggtcttcggagaccgagggcctccaaaagcgtccacctcttgttttcttaaatacacttggagacaacggtgcgttaggtcaagctccgtacatccacctgattaaccgagatgagcacgaacagtattacgctgtgttcactggtagcggaatccgagtgttcgacctttctggtaacgagaagcaagttaggtatcctaacggttccaactacatcaagaccgctaatccacgtaacgacctgcgaatggttactgtagcagactatacgttcatcgttaaccgtaacgttgttgcacagaagaacacaaagtctgtcaacttaccgaattacaaccctaatcaagacggattgattaacgttcgtggtggtcagtatggtagggaactaattgtacacattaacggtaaagacgttgcgaagtataagataccagatggtagtcaacctgaacacgtaaacaatacggatgcccaatggttagctgaagagttagccaagcagatgcgcactaacttgtctgattggactgtaaatgtagggcaagggttcatccatgtgaccgcacctagtggtcaacagattgactccttcacgactaaagatggctacgcagaccagttgattaaccctgtgacccactacgctcagtcgttctctaagctgccacctaatgctcctaacggctacatggtgaaaatcgtaggggacgcctctaagtctgccgaccagtattacgttcggtatgacgctgagcggaaagtttggactgagactttaggttggaacactgaggaccaagttctatgggaaaccatgccacacgctcttgtgcgagccgctgacggtaatttcgacttcaagtggcttgagtggtctcctaagtcttgtggtgacgttgacaccaacccttggccttcttttgttggttcaagtattaacgatgtgttcttcttccgtaaccgcttaggattccttagtggggagaacatcatattgagtcgtacagccaaatacttcaacttctaccctgcgtccattgcgaaccttagtgatgacgaccctatagacgtagctgtgagtaccaaccgaatagcaatccttaagtacgccgttccgttctcagaagagttactcatctggtccgatgaagcacaattcgtcctgactgcctcgggtactctcacatctaagtcggttgagttgaacctaacgacccagtttgacgtacaggaccgagcgagaccttttgggattgggcgtaatgtctactttgctagtccgaggtccagcttcacgtccatccacaggtactacgctgtgcaggatgtcagttccgttaagaatgctgaggacattacatcacacgttcctaactacatccctaatggtgtgttcagtatttgcggaagtggtacggaaaacttctgttcggtactatctcacggggaccctagtaaaatcttcatgtacaaattcctgtacctgaacgaagagttaaggcaacagtcgtggtctcattgggactttggggaaaacgtacaggttctagcttgtcagagtatcagctcagatatgtatgtgattcttcgcaatgagttcaatacgttcctagctagaatctctttcactaagaacgccattgacttacagggagaaccctatcgtgcctttatggacatgaagattcgatacacgattcctagtggaacatacaacgatgacacattcactacctctattcatattccaacaatttatggtgcaaacttcgggaggggcaaaatcactgtattggagcctgatggtaagataaccgtgtttgagcaacctacggctgggtggaatagcgacccttggctgagactcagcggtaacttggagggacgcatggtgtacattgggttcaacattaacttcgtatatgagttctctaagttcctcatcaagcagactgccgacgacgggtctacctccacggaagacattgggcgcttacagttacgccgagcgtgggttaactacgagaactctggtacgtttgacatttatgttgagaaccaatcgtctaactggaagtacacaatggctggtgcccgattaggctctaacactctgagggctgggagactgaacttagggaccggacaatatcgattccctgtggttggtaacgccaagttcaacactgtatacatcttgtcagatgagactacccctctgaacatcattgggtgtggctgggaaggtaactacttacggagaagttccggtatttaattaaatattctccctgtggtggctcgaaattaatacgactcactatagggagaacaatacgactacgggagggttttcttatgatgactataagacctactaaaagtacagactttgaggtattcactccggctcaccatgacattcttgaagctaaggctgctggtattgagccgagtttccctgatgcttccgagtgtgtcacgttgagcctctatgggttccctctagctatcggtggtaactgcggggaccagtgctggttcgttacgagcgaccaagtgtggcgacttagtggaaaggctaagcgaaagttccgtaagttaatcatggagtatcgcgataagatgcttgagaagtatgatactctttggaattacgtatgggtaggcaatacgtcccacattcgtttcctcaagactatcggtgcggtattccatgaagagtacacacgagatggtcaatttcagttatttacaatcacgaaaggaggataaccatatgtgttgggcagccgcaatacctatcgctatatctggcgctcaggctatcagtggtcagaacgctcaggccaaaatgattgccgctcagaccgctgctggtcgtcgtcaagctatggaaatcatgaggcagacgaacatccagaatgctgacctatcgttgcaagctcgaagtaaacttgaggaagcgtccgccgagttgacctcacagaacatgcagaaggtccaagctattgggtctatccgagcggctatcggagagagtatgcttgaaggttcctcaatggaccgcattaagcgagtcacagaaggacagttcattcgggaagccaatatggtaactgagaactatcgccgtgactaccaagcaatcttcgcacagcaacttggtggtactcaaagtgctgcaagtcagattgacgaaatctataagagcgaacagaaacagaagagtaagctacagatggttctggacccactggctatcatggggtcttccgctgcgagtgcttacgcatccggtgcgttcgactctaagtccacaactaaggcacctattgttgccgctaaaggaaccaagacggggaggtaatgagctatgagtaaaattgaatctgcccttcaagcggcacaaccgggactctctcggttacgtggtggtgctggaggtatgggctatcgtgcagcaaccactcaggccgaacagccaaggtcaagcctattggacaccattggtcggttcgctaaggctggtgccgatatgtataccgctaaggaacaacgagcacgagacctagctgatgaacgctctaacgagattatccgtaagctgacccctgagcaacgtcgagaagctctcaacaacgggacccttctgtatcaggatgacccatacgctatggaagcactccgagtcaagactggtcgtaacgctgcgtatcttgtggacgatgacgttatgcagaagataaaagagggtgtcttccgtactcgcgaagagatggaagagtatcgccatagtcgccttcaagagggcgctaaggtatacgctgagcagttcggcatcgaccctgaggacgttgattatcagcgtggtttcaacggggacattaccgagcgtaacatctcgctgtatggtgcgcatgataacttcttgagccagcaagctcagaagggcgctatcatgaacagccgagtggaactcaacggtgtccttcaagaccctgatatgctgcgtcgtccagactctgctgacttctttgagaagtatatcgacaacggtctggttactggcgcaatcccatctgatgctcaagccacacagcttataagccaagcgttcagtgacgcttctagccgtgctggtggtgctgacttcctgatgcgagtcggtgacaagaaggtaacacttaacggagccactacgacttaccgagagttgattggtgaggaacagtggaacgctctcatggtcacagcacaacgttctcagtttgagactgacgcgaagctgaacgagcagtatcgcttgaagattaactctgcgctgaaccaagaggacccaaggacagcttgggagatgcttcaaggtatcaaggctgaactagataaggtccaacctgatgagcagatgacaccacaacgtgagtggctaatctccgcacaggaacaagttcagaatcagatgaacgcatggacgaaagctcaggccaaggctctggacgattccatgaagtcaatgaacaaacttgacgtaatcgacaagcaattccagaagcgaatcaacggtgagtgggtctcaacggattttaaggatatgccagtcaacgagaacactggtgagttcaagcatagcgatatggttaactacgccaataagaagctcgctgagattgacagtatggacattccagacggtgccaaggatgctatgaagttgaagtaccttcaagcggactctaaggacggagcattccgtacagccatcggaaccatggtcactgacgctggtcaagagtggtctgccgctgtgattaacggtaagttaccagaacgaaccccagctatggatgctctgcgcagaatccgcaatgctgaccctcagttgattgctgcgctatacccagaccaagctgagctattcctgacgatggacatgatggacaagcagggtattgaccctcaggttattcttgatgccgaccgactgactgttaagcggtccaaagagcaacgctttgaggatgataaagcattcgagtctgcactgaatgcatctaaggctcctgagattgcccgtatgccagcgtcactgcgcgaatctgcacgtaagatttatgactccgttaagtatcgctcggggaacgaaagcatggctatggagcagatgaccaagttccttaaggaatctacctacacgttcactggtgatgatgttgacggtgataccgttggtgtgattcctaagaatatgatgcaggttaactctgacccgaaatcatgggagcaaggtcgggatattctggaggaagcacgtaagggaatcattgcgagcaacccttggataaccaataagcaactgaccatgtattctcaaggtgactccatttaccttatggacaccacaggtcaagtcagagtccgatacgacaaagagttactctcgaaggtctggagtgagaaccagaagaaactcgaagagaaagctcgtgagaaggctctggctgatgtgaacaagcgagcacctatagttgccgctacgaaggcccgtgaagctgctgctaaacgagtccgagagaaacgtaaacagactcctaagttcatctacggacgtaaggagtaactaaaggctacataaggaggccctaaatggataagtacgataagaacgtaccaagtgattatgatggtctgttccaaaaggctgctgatgccaacggggtctcttatgaccttttacgtaaagtcgcttggacagaatcacgatttgtgcctacagcaaaatctaagactggaccattaggcatgatgcaatttaccaaggcaaccgctaaggccctcggtctgcgagttaccgatggtccagacgacgaccgactgaaccctgagttagctattaatgctgccgctaagcaacttgcaggtctggtagggaagtttgatggcgatgaactcaaagctgcccttgcgtacaaccaaggcgagggacgcttgggtaatccacaacttgaggcgtactctaagggagacttcgcatcaatctctgaggagggacgtaactacatgcgtaaccttctggatgttgctaagtcacctatggctggacagttggaaacttttggtggcataaccccaaagggtaaaggcattccggctgaggtaggattggctggaattggtcacaagcagaaagtaacacaggaacttcctgagtccacaagttttgacgttaagggtatcgaacaggaggctacggcgaaaccattcgccaaggacttttgggagacccacggagaaacacttgacgagtacaacagtcgttcaaccttcttcggattcaaaaatgctgccgaagctgaactctccaactcagtcgctgggatggctttccgtgctggtcgtctcgataatggttttgatgtgtttaaagacaccattacgccgactcgctggaactctcacatctggactccagaggagttagagaagattcgaacagaggttaagaaccctgcgtacatcaacgttgtaactggtggttcccctgagaacctcgatgacctcattaaattggctaacgagaactttgagaatgactcccgcgctgccgaggctggcctaggtgccaaactgagtgctggtattattggtgctggtgtggacccgcttagctatgttcctatggtcggtgtcactggtaagggctttaagttaatcaataaggctcttgtagttggtgccgaaagtgctgctctgaacgttgcatccgaaggtctccgtacctccgtagctggtggtgacgcagactatgcgggtgctgccttaggtggctttgtgtttggcgcaggcatgtctgcaatcagtgacgctgtagctgctggactgaaacgcagtaaaccagaagctgagttcgacaatgagttcatcggtcctatgatgcgattggaagcccgtgagacagcacgaaacgccaactctgcggacctctctcggatgaacactgagaacatgaagtttgaaggtgaacataatggtgtcccttatgaggacttaccaacagagagaggtgccgtggtgttacatgatggctccgttctaagtgcaagcaacccaatcaaccctaagactctaaaagagttctccgaggttgaccctgagaaggctgcgcgaggaatcaaactggctgggttcaccgagattggcttgaagaccttggggtctgacgatgctgacatccgtagagtggctatcgacctcgttcgctctcctactggtatgcagtctggtgcctcaggtaagttcggtgcaacagcttctgacatccatgagagacttcatggtactgaccagcgtacttataatgacttgtacaaagcaatgtctgacgctatgaaagaccctgagttctctactggcggcgctaagatgtcccgtgaagaaactcgatacactatctaccgtagagcggcactagctattgagcgtccagaactacagaaggcactcactccgtctgagagaatcgttatggacatcattaagcgtcactttgacaccaagcgtgaacttatggaaaacccagcaatattcggtaacacaaaggctgtgagtatcttccctgagagtcgccacaaaggtacttacgttcctcacgtatatgaccgtcatgccaaggcgctgatgattcaacgctacggtgccgaaggtttgcaggaagggattgcccgctcatggatgaacagctacgtctccagacctgaggtcaaggccagagtcgatgagatgcttaaggaattacacggggtgaaggaagtaacaccagagatggtagagaagtacgctatggataaggcttatggtatctcccactcagaccagttcaccaacagttccataatagaagagaacattgagggcttagtaggtatcgagaataactcattccttgaggcacgtaacttgtttgattcggacctatccatcactatgccagacggacagcaattctcagtgaatgacctaagggacttcgatatgttccgcatcatgccagcgtatgaccgccgtgtcaatggtgacatcgccatcatggggtctactggtaaaaccactaaggaacttaaggatgagattttggctctcaaagcgaaagctgagggagacggtaagaagactggcgaggtacatgctttaatggataccgttaagattcttactggtcgtgctagacgcaatcaggacactgtgtgggaaacctcactgcgtgccatcaatgacctagggttcttcgctaagaacgcctacatgggtgctcagaacattacggagattgctgggatgattgtcactggtaacgttcgtgctctagggcatggtatcccaattctgcgtgatacactctacaagtctaaaccagtttcagctaaggaactcaaggaactccatgcgtctctgttcgggaaggaggtggaccagttgattcggcctaaacgtgctgacattgtgcagcgcctaagggaagcaactgataccggacctgccgtggcgaacatcgtagggaccttgaagtattcaacacaggaactggctgctcgctctccgtggactaagctactgaacggaaccactaactaccttctggatgctgcgcgtcaaggtatgcttggggatgttattagtgccaccctaacaggtaagactacccgctgggagaaagaaggcttccttcgtggtgcctccgtaactcctgagcagatggctggcatcaagtctctcatcaaggaacatatggtacgcggtgaggacgggaagtttaccgttaaggacaagcaagcgttctctatggacccacgggctatggacttatggagactggctgacaaggtagctgatgaggcaatgctgcgtccacataaggtgtccttacaggattcccatgcgttcggagcactaggtaagatggttatgcagtttaagtctttcactatcaagtcccttaactctaagttcctgcgaaccttctatgatggatacaagaacaaccgagcgattgacgctgcgctgagcatcatcacctctatgggtctcgctggtggtttctatgctatggctgcacacgtcaaagcatacgctctgcctaaggagaaacgtaaggagtacttggagcgtgcactggacccaaccatgattgcccacgctgcgttatctcgtagttctcaattgggtgctcctttggctatggttgacctagttggtggtgttttagggttcgagtcctccaagatggctcgctctacgattctacctaaggacaccgtgaaggaacgtgacccaaacaaaccgtacacctctagagaggtaatgggcgctatgggttcaaaccttctggaacagatgccttcggctggctttgtggctaacgtaggggctaccttaatgaatgctgctggcgtggtcaactcacctaataaagcaaccgagcaggacttcatgactggtcttatgaactccacaaaagagttagtaccgaacgacccattgactcaacagcttgtgttgaagatttatgaggcgaacggtgttaacttgagggagcgtaggaaataatacgactcactatagggagaggcgaaataatcttctccctgtagtctcttagatttactttaaggaggtcaaatggctaacgtaattaaaaccgttttgacttaccagttagatggctccaatcgtgattttaatatcccgtttgagtatctagcccgtaagttcgtagtggtaactcttattggtgtagaccgaaaggtccttacgattaatacagactatcgctttgctacacgtactactatctctctgacaaaggcttggggtccagccgatggctacacgaccatcgagttacgtcgagtaacctccactaccgaccgattggttgactttacggatggttcaatcctccgcgcgtatgaccttaacgtcgctcagattcaaacgatgcacgtagcggaagaggcccgtgacctcactacggatactatcggtgtcaataacgatggtcacttggatgctcgtggtcgtcgaattgtgaacctagcgaacgccgtggatgaccgcgatgctgttccgtttggtcaactaaagaccatgaaccagaactcatggcaagcacgtaatgaagccttacagttccgtaatgaggctgagactttcagaaaccaagcggagggctttaagaacgagtccagtaccaacgctacgaacacaaagcagtggcgcgatgagaccaagggtttccgagacgaagccaagcggttcaagaatacggctggtcaatacgctacatctgctgggaactctgcttccgctgcgcatcaatctgaggtaaacgctgagaactctgccacagcatccgctaactctgctcatttggcagaacagcaagcagaccgtgcggaacgtgaggcagacaagctggaaaattacaatggattggctggtgcaattgataaggtagatggaaccaatgtgtactggaaaggaaatattcacgctaacgggcgcctttacatgaccacaaacggttttgactgtggccagtatcaacagttctttggtggtgtcactaatcgttactctgtcatggagtggggagatgagaacggatggctgatgtatgttcaacgtagagagtggacaacagcgataggcggtaacatccagttagtagtaaacggacagatcatcacccaaggtggagccatgaccggtcagctaaaattgcagaatgggcatgttcttcaattagagtccgcatccgacaaggcgcactatattctatctaaagatggtaacaggaataactggtacattggtagagggtcagataacaacaatgactgtaccttccactcctatgtacatggtacgaccttaacactcaagcaggactatgcagtagttaacaaacacttccacgtaggtcaggccgttgtggccactgatggtaatattcaaggtactaagtggggaggtaaatggctggatgcttacctacgtgacagcttcgttgcgaagtccaaggcgtggactcaggtgtggtctggtagtgctggcggtggggtaagtgtgactgtttcacaggatctccgcttccgcaatatctggattaagtgtgccaacaactcttggaacttcttccgtactggccccgatggaatctacttcatagcctctgatggtggatggttacgattccaaatacactccaacggtctcggattcaagaatattgcagacagtcgttcagtacctaatgcaatcatggtggagaacgagtaattggtaaatcacaaggaaagacgtgtagtccacggatggactctcaaggaggtacaaggtgctatcattagactttaacaacgaattgattaaggctgctccaattgttgggacgggtgtagcagatgttagtgctcgactgttctttgggttaagccttaacgaatggttctacgttgctgctatcgcctacacagtggttcagattggtgccaaggtagtcgataagatgattgactggaagaaagccaataaggagtgatatgtatggaaaaggataagagccttattacattcttagagatgttggacactgcgatggctcagcgtatgcttgcggacctttcggaccatgagcgtcgctctccgcaactctataatgctattaacaaactgttagaccgccacaagttccagattggtaagttgcagccggatgttcacatcttaggtggccttgctggtgctcttgaagagtacaaagagaaagtcggtgataacggtcttacggatgatgatatttacacattacagtgatatactcaaggccactacagatagtggtctttatggatgtcattgtctatacgagatgctcctacgtgaaatctgaaagttaacgggaggcattatgctagaatttttacgtaagctaatcccttgggttctcgctgggatgctattcgggttaggatggcatctagggtcagactcaatggacgctaaatggaaacaggaggtacacaatgagtacgttaagagagttgaggctgcgaagagcactcaaagagcaatcgatgcggtatctgctaagtatcaagaagaccttgccgcgctggaagggagcactgataggattatttctgatttgcgtagcgacaataagcggttgcgcgtcagagtcaaaactaccggaacctccgatggtcagtgtggattcgagcctgatggtcgagccgaacttgacgaccgagatgctaaacgtattctcgcagtgacccagaagggtgacgcatggattcgtgcgttacaggatactattcgtgaactgcaacgtaagtaggaaatcaagtaaggaggcaatgtgtctactcaatccaatcgtaatgcgctcgtagtggcgcaactgaaaggagacttcgtggcgttcctattcgtcttatggaaggcgctaaacctaccggtgcccactaagtgtcagattgacatggctaaggtgctggcgaatggagacaacaagaagttcatcttacaggctttccgtggtatcggtaagtcgttcatcacatgtgcgttcgttgtgtggtccttatggagagaccctcagttgaagatacttatcgtatcagcctctaaggagcgtgcagacgctaactccatctttattaagaacatcattgacctgctgccattcctatctgagttaaagccaagacccggacagcgtgactcggtaatcagctttgatgtaggcccagccaatcctgaccactctcctagtgtgaaatcagtaggtatcactggtcagttaactggtagccgtgctgacattatcattgcggatgacgttgagattccgtctaacagcgcaactatgggtgcccgtgagaagctatggactctggttcaggagttcgctgcgttacttaaaccgctgccttcctctcgcgttatctaccttggtacacctcagacagagatgactctctataaggaacttgaggataaccgtgggtacacaaccattatctggcctgctctgtacccaaggacacgtgaagagaacctctattactcacagcgtcttgctcctatgttacgcgctgagtacgatgagaaccctgaggcacttgctgggactccaacagacccagtgcgctttgaccgtgatgacctgcgcgagcgtgagttggaatacggtaaggctggctttacgctacagttcatgcttaaccctaaccttagtgatgccgagaagtacccgctgaggcttcgtgacgctatcgtagcggccttagacttagagaaggccccaatgcattaccagtggcttccgaaccgtcagaacatcattgaggaccttcctaacgttggccttaagggtgatgacctgcatacgtaccacgattgttccaacaactcaggtcagtaccaacagaagattctggtcattgaccctagtggtcgcggtaaggacgaaacaggttacgctgtgctgtacacactgaacggttacatctaccttatggaagctggaggtttccgtgatggctactccgataagacccttgagttactcgctaagaaggcaaagcaatggggagtccagacggttgtctacgagagtaacttcggtgacggtatgttcggtaaggtattcagtcctatccttcttaaacaccacaactgtgcgatggaagagattcgtgcccgtggtatgaaagagatgcgtatttgcgatacccttgagccagtcatgcagactcaccgccttgtaattcgtgatgaggtcattagggccgactaccagtccgctcgtgacgtagacggtaagcatgacgttaagtactcgttgttctaccagatgacccgtatcactcgtgagaaaggcgctctggctcatgatgaccgattggatgcccttgcgttaggcattgagtatctccgtgagtccatgcagttggattccgttaaggtcgagggtgaagtacttgctgacttccttgaggaacacatgatgcgtcctacggttgctgctacgcatatcattgagatgtctgtgggaggagttgatgtgtactctgaggacgatgagggttacggtacgtctttcattgagtggtgatttatgcattaggactgcatagggatgcactatagaccacggatggtcagttctttaagttactgaaaagacacgataaattaatacgactcactatagggagaggagggacgaaaggttactatatagatactgaatgaatacttatagagtgcataaagtatgcataatggtgtacctagagtgacctctaagaatggtgattatattgtattagtatcaccttaacttaaggaccaacataaagggaggagactcatgttccgcttattgttgaacctactgcggcatagagtcacctaccgatttcttgtggtactttgtgctgcccttgggtacgcatctcttactggagacctcagttcactggagtctgtcgtttgctctatactcacttgtagcgattagggtcttcctgaccgactgatggctcaccgagggattcagcggtatgattgcatcacaccacttcatccctatagagtcaagtcctaaggtatacccataaagagcctctaatggtctatcctaaggtctatacctaaagataggccatcctatcagtgtcacctaaagagggtcttagagagggcctatggagttcctatagggtcctttaaaatataccataaaaatctgagtgactatctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccct

SEQ ID NO: 23 - DNA, 156 bp, synthetic construct
  source 1..156: mol_type = other DNA; note = Gp0.4
atgtctactaccaacgtgcaatacggtctgaccgctcaaactgtacttttctatagcgacatggtgcgctgtggctttaactggtcactcgcaatggcacagctcaaagaactgtacgaaaacaacaaggcaatagctttagaatctgctgagtga

SEQ ID NO: 24 - DNA, 162 bp, synthetic construct
  source 1..162: mol_type = other DNA; note = Gp0.6
atgatgaagcactacgttatgccaatccacacgtccaacggggcaaccgtatgtacacctgatgggttcgcaatgaaacaacgaatcgaacgccttaagcgtgaactccgcattaaccgcaagattaacaagataggttccggctatgacagaacgcactga

SEQ ID NO: 25 - DNA, 483 bp, synthetic construct
  source 1..483: mol_type = other DNA; note = IL-10 CUT
tctccgggtcaaggtacgcaaagtgaaaattcatgtactcattttcctggaaatcttccgaatatgttacgtgacctgcgtgacgcgttttctcgtgtcaaaaccttcttccagatgaaagaccaattagataatcttctgctgaaagaatcactgctcgaagatttcaaagggtatttagggtgtcaggctcttagcgaaatgattcaattctatcttgaagaagtaatgccgcaggccgaaaatcaggatccggatattaaagcccacgtaaatagccttggagaaaatctgaaaacactgcgtttacgtctgcgtcgatgccaccgtttcctgccgtgcgagaataagtcaaaagctgttgaacaagtgaagaacgcattcaacaagctgcaggaaaagggtatctataaggcgatgtccgagttcgatatattcattaattatatcgaggcttatatgaccatgaagattcggaattaa

SEQ ID NO: 26 - DNA, 495 bp, synthetic construct
  source 1..495: mol_type = other DNA; note = IL-10 mod
agcccgggacagggaacacagagtgaaaacagctgtacacattttcctggcaatttaccgaatatgctacgcgatttacgcgatgctttttccagagtaaaaacattcttccagatgaaagatcaactggataacctgctgttaaaagagtctttactggaagattttaaaggttatcttggctgccaggcgttaagtgagatgatccagttttatctggaagaggtaatgccgcaggcagaaaatcaggatccggatattaaggcacatgtcaactcactcggtgaaaatttaaaaacgctgcgtctgcgtctgcgtcgctgccaccgttttcttccttgtgaaaacaaaagcaaagcggtggagcaagtgaaaaatgccttcaataaactgcaagaaaaaggcatttataaagccatgagcgagtttgatattttcattaattatattgaagcctacatgacaatgaaaataaggaattgaagtactgctaac

SEQ ID NO: 27 - DNA, 483 bp, synthetic construct
  source 1..483: mol_type = other DNA; note = IL-10 LPP_CUT
agtccggggcaagggacgcaaagcgaaaattcctgtactcattttccgggaaatcttccgaatatgctccgtgacttgcgggacgcattttcccgggttaaaacgttcttccagatgaaagaccaattggataatctcctgttgaaagaatctctgttagaagatttcaaaggctatcttgggtgtcaggcgttatccgaaatgattcaattctatctcgaagaagttatgccacaggccgaaaatcaggatccggatataaaagcacacgttaattcgttaggtgaaaatttaaagacactgagactccggctgcgccgttgccaccgtttcttgccatgcgagaataaatcgaaagcggttgaacaagtaaagaacgcattcaacaaacttcaggaaaaggggatctataaggcaatgtcagagttcgatatcttcataaattatattgaggcttacatgaccatgaaaatccggaattaa

SEQ ID NO: 28 - DNA, 482 bp, synthetic construct
  source 1..482: mol_type = other DNA; note = IL-10 LPP-mod
agtccggggcaagggacgcaaagcgaaaattcctgtactcattttccgggaaatcttccgaatatgctccgtgacttgcgggacgcattttcccgggttaaaacgttcttccagatgaaagaccaattggataatctcctgttgaaagaatctctgttagaagatttcaaaggctatcttgggtgtcaggcgttatccgaaatgattcaattctatctcgaagaagttatgccacaggccgaaaatcaggatccggatataaaagcacacgttaattcgttaggtgaaaatttaaagacactgagactccggctgcgccgttgccaccgtttcttgccatgcgagaataaatcgaaagcggttgaacaagtaaagaacgcattcaacaaacttcaggaaaaggggatctataaggcaatgtcagagttcgatatcttcataaattatattgaggcttacatgaccatgaaaatccggaatta

SEQ ID NO: 29 - DNA, 483 bp, synthetic construct
  source 1..483: mol_type = other DNA; note = IL-10 Mut_CUT
tcacccgggcaaggtacccaatcagagaattcatgcacgcacttcccgggtatactgcccaacatgcttcgagaccttcgggacgcgttctcacgggtcaagacgttctttcaaatgaaggaccagcttgacaatttgttactgaaggaatcgctgctcgaggacttcaagggctacctgggttgtcaagctctctccgaaatgatacaattctacttggaagaagtgatgccacaagcggagaaccaagaccccgacatcaaagcccacgtgataagcctgggagagaacttgaacacattacgcttacggttgcgcagatgtcatcggctgctgccatgcgagaataagtcgaaggctgtcgaacaggtcaagaacgcgtttaacaagcttcaggagaagggtatatacaaggcgatgagtgaattcgacatctttataaactacatcgaggcttatatgactatgaagattagaaactaa

SEQ ID NO: 30 - DNA, 483 bp, synthetic construct
  source 1..483: mol_type = other DNA; note = IL-10 Mut_mod
agcccgggacagggaacacagagtgaaaacagctgtacacattttcctggcattttaccgaatatgctacgcgatttacgcgatgctttttccagagtaaaaacattcttccagatgaaagatcaactggataacctgctgttaaaagagtctttactggaagattttaaaggttatcttggctgccaggcgttaagtgagatgatccagttttatctggaagaggtaatgccgcaggcagaaaatcaggatccggatattaaggcacatgtcatttcactcggtgaaaatttaaatacgctgcgtctgcgtctgcgtcgctgccaccgtcttcttccttgtgaaaacaaaagcaaagcggtggagcaagtgaaaaatgccttcaataaactgcaagaaaaaggcatttataaagccatgagcgagtttgatattttcattaattatattgaagcctacatgacaatgaaaataaggaattag

SEQ ID NO: 31 - DNA, 2667 bp, Escherichia coli
  source 1..2667: mol_type = genomic DNA; note = CAS3
atggaaccttttaaatatatatgccattactggggaaaatcctcaaaaagcttgacgaaaggaaatgatattcatctgttaatttatcattgccttgatgttgctgctgttgcagattgctggtgggatcaatcagtcgtactgcaaaatactttttgccgaaatgaaatgctatcaaaacagagggtgaaggcctggctgttatttttcattgctcttcatgatattggaaagtttgatatacgattccaatataaatcagcagaaagttggctgaaattaaatcctgcaacgccatcacttaatggtccatcaacacaaatgtgccgtaaatttaatcatggtgcagccggtctgtattggtttaaccaggattcactttcagagcaatctctcggggattttttcagtttttttgatgccgctcctcatccttatgagtcctggtttccatgggtagaggccgttacaggacatcatggttttatattacattcccaggatcaagataagtcgcgttgggaaatgccagcttctctggcatcttatgctgcgcaagataaacaggctcgtgaggagtggatatctgtactggaagcattatttttaacgccagcggggttatctataaacgatataccacctgattgttcatcactgttagcaggtttttgctcgcttgctgactggttaggctcctggactacaacgaatacctttctgtttaatgaggatgcgccttccgacataaatgctctgagaacgtatttccaggaccgacagcaggatgcgagccgggtattggagttgagtggacttgtatcaaataagcgatgttatgaaggtgttcatgcactactggacaatggctatcaacccagacaattacaggtgttagttgatgctcttccagtagctcccgggctgacggtaatagaggcacctacaggctccggtaaaacggaaacagcgctggcctatgcttggaaacttattgatcaacaaattgcggatagtgttatttttgccctcccaacacaagctaccgcgaatgctatgcttacgagaatggaagcgagcgcgagccacttattttcatccccaaatcttattcttgctcatggcaattcacggtttaaccacctctttcaatcaataaaatcacgcgcgattactgaacaggggcaagaagaagcgtgggttcagtgttgtcagtggttgtcacaaagcaataagaaagtgtttcttgggcaaatcggcgtttgcacgattgatcaggtgttgatatcggtattgccagttaaacaccgctttatccgtggtttgggaattggtcgaagtgttttaattgttgatgaagttcatgcttacgacacctatatgaacggcttgctggaggcagtgctcaaggctcaggctgatgttggagggagtgttattcttctttccgcaaccctaccaatgaaacaaaaacagaaacttctggatacttatggtctgcatacagatccagtggaaaataactccgcatatccactcattaactggcgaggtgtgaatggtgcgcaacgttttgatctgctagctcatccagaacaactcccgccccgcttttcgattcagccagaacctatttgtttagctgacatgttacctgaccttacgatgttagagcgaatgatcgcagcggcaaacgcgggtgcacaggtctgtcttatttgcaatttggttgacgttgcacaagtatgctaccaacggctaaaggagctaaataacacgcaagtagatatagatttgtttcatgcgcgctttacgctgaacgatcgtcgtgaaaaagagaatcgagttattagcaatttcggcaaaaatgggaagcgaaatgttggacggatacttgtcgcaacccaggtcgtggaacaatcactcgacgttgattttgattggttaattactcagcattgtcctgcagatttgcttttccaacgattgggccgtttacatcgccatcatcgcaaatatcgtcccgctggttttgagattcctgttgccaccattttgctgcctgatggcgagggttacggacgacatgagcatatttatagcaacgttagagtcatgtggcggacgcagcaacatattgaggagcttaatggagcatccttatttttccctgatgcttaccggcaatggctggatagcatttacgatgatgcggaaatggatgagccagaatgggtcggcaatggcatggataaatttgaaagcgccgagtgtgaaaaaaggttcaaggctcgcaaggtcctgcagtgggctgaagaatatagcttgcaggataacgatgaaaccattcttgcggtaacgagggatggggaaatgagcctgccattattgccttatgtacaaacgtcttcaggtaaacaactgctcgatggccaggtctacgaggacctaagtcatgaacagcagtatgaggcgcttgcacttaatcgcgtcaatgtacccttcacctggaaacgtagtttttctgaagtagtagatgaagatgggttactttggctggaagggaaacagaatctggatggatgggtctggcagggtaacagtattgttattacctatacaggggatgaagggatgaccagagtcatccctgcaaatcccaaataa

SEQ ID NO: 32 - DNA, 1512 bp, Escherichia coli
  source 1..1512: mol_type = genomic DNA; note = CASA
atggctaatttgcttattgataactggatccctgtacgcccgcgaaacggggggaaagtccaaatcataaatctgcaatcgctatactgcagtagagatcagtggcgattaagtttgccccgtgacgatatggaactggccgctttagcactgctggtttgcattgggcaaattatcgccccggcaaaagatgacgttgaatttcgacatcgcataatgaatccgctcactgaagatgagtttcaacaactcatcgcgccgtggatagatatgttctaccttaatcacgcagaacatccctttatgcagaccaaaggtgtcaaagcaaatgatgtgactccaatggaaaaactgttggctggggtaagcggcgcgacgaattgtgcatttgtcaatcaaccggggcagggtgaagcattatgtggtggatgcactgcgattgcgttattcaaccaggcgaatcaggcaccaggttttggtggtggttttaaaagcggtttacgtggaggaacacctgtaacaacgttcgtacgtgggatcgatcttcgttcaacggtgttactcaatgtcctcacattacctcgtcttcaaaaacaatttcctaatgaatcacatacggaaaaccaacctacctggattaaacctatcaagtccaatgagtctatacctgcttcgtcaattgggtttgtccgtggtctattctggcaaccagcgcatattgaattatgcgatcccattgggattggtaaatgttcttgctgtggacaggaaagcaatttgcgttataccggttttcttaaggaaaaatttacctttacagttaatgggctatggccccatccgcattccccttgtctggtaacagtcaagaaaggggaggttgaggaaaaatttcttgctttcaccacctccgcaccatcatggacacaaatcagccgagttgtggtagataagattattcaaaatgaaaatggaaatcgcgtggcggcggttgtgaatcaattcagaaatattgcgccgcaaagtcctcttgaattgattatggggggatatcgtaataatcaagcatctattcttgaacggcgtcatgatgtgttgatgtttaatcaggggtggcaacaatacggcaatgtgataaacgaaatagtgactgttggtttgggatataaaacagccttacgcaaggcgttatatacctttgcagaagggtttaaaaataaagacttcaaaggggccggagtctctgttcatgagactgcagaaaggcatttctatcgacagagtgaattattaattcccgatgtactggcgaatgttaatttttcccaggctgatgaggtaatagctgatttacgagacaaacttcatcaattgtgtgaaatgctatttaatcaatctgtagctccctatgcacatcatcctaaattaataagcacattagcgcttgcccgcgccacgctatacaaacatttacgggagttaaaaccgcaaggagggccatcaaatggctga

SEQ ID NO: 33 - DNA, 483 bp, Escherichia coli
  source 1..483: mol_type = genomic DNA; note = CASB(OVERLAPS WITH A):
atggctgatgaaattgatgcaatggctttatatcgagcctggcaacaactggataatggatcatgtgcgcaaattagacgtgtttcagaacctgatgaattacgcgatatccctgcgttttataggctggtgcaaccttttggttgggaaaacccacgtcaccagcaggctcttttgcgcatggtgttttgcctgagcgcaggaaagaatgtcatccgacatcaggacaaaaaatcggagcaaacaacaggtatctcgttgggaagagctttagccaatagtggaagaattaacgagcgccgtatctttcaattaattcgggctgacagaacagccgatatggtccagttacgtcgattacttactcacgccgaacccgtacttgactggccattaatggccaggatgttgacctggtggggaaagcacgaacgccagcaacttctggaagattttgtattgaccacaaacaaaaatgcgtaa

SEQ ID NO: 34 - DNA, 1092 bp, Escherichia coli
  source 1..1092: mol_type = genomic DNA; note = CASC
atgtctaactttatcaatattcatgttctgatctctcacagcccttcatgtctgaaccgcgacgatatgaacatgcagaaagacgctattttcggcggcaaaagacgagtaagaatttcaagtcaaagccttaaacgtgcgatgcgtaaaagtggttattacgcacaaaatattggtgaatccagtctcagaaccattcatcttgcacaattacgtgatgttcttcggcaaaaacttggtgaacgttttgaccaaaaaatcatcgataagacattagcgctgctctccggtaaatcagttgatgaagccgaaaagatttctgccgatgcggttactccctgggttgtgggagaaatagcctggttctgtgagcaggttgcaaaagcagaggctgataatctggatgataaaaagctgctcaaagttcttaaggaagatattgccgccatacgtgtgaatttacagcagggtgttgatattgcgcttagtggaagaatggcaaccagcggcatgatgactgagttgggaaaagttgatggtgcaatgtccattgcgcatgcgatcactactcatcaggttgattctgatattgactggttcaccgctgtagatgatttacaggaacaaggttctgcacatctgggaactcaggaattttcatcgggtgttttttatcgttatgccaacattaacctcgctcaacttcaggaaaatttaggtggtgcctccagggagcaggctctggaaattgcaacccatgttgttcatatgctggcaacagaggtccctggagcaaaacagcgtacttatgccgcttttaaccctgcggatatggtaatggttaatttctccgatatgccactttctatggcaaatgcttttgaaaaagcggttaaagcgaaagatggctttttgcaaccgtctatacaggcgtttaatcaatattgggatcgcgttgccaatggatatggtctgaacggagctgctgcgcaattcagcttatctgatgtagacccaattactgctcaagttaaacaaatgcctactttagaacagttaaaatcctgggttcgtaataatggcgaggcgtga

SEQ ID NO: 35 - DNA, 672 bp, Escherichia coli
  source 1..672: mol_type = genomic DNA; note = CASD
atgagatcttatttgatcttgcggcttgctgggccaatgcaagcctgggggcagccgacctttgaaggaacgcgacctaccggaagatttccgacccgaagcgggttattagggctactcggggcttgtcttgggatccaacgtgatgatacttcttcattacaggcgttatcagagagtgtgcaatttgcagtgcgctgcgatgaactcattcttgacgatcgtcgtgtgtctgtaacggggttgcgtgattaccatacagtccttggagcgcgagaagattaccgtggtttgaaaagtcatgaaacgattcaaacatggcgcgaatatttatgtgatgcctcctttaccgtcgctctctggttaacaccccatgcaacgatggttatctcagaacttgaaaaagcagtattaaagcctcggtatacaccttacctggggcggagaagttgcccactaacacacccgctttttttggggacatgtcaggcatcggatcctcagaaggcgctattaaattatgagcccgttggcggcgatatatatagtgaggaatcagttacagggcatcatttaaaatttacggcgcgcgacgaaccgatgatcaccttgcctcgacaatttgcttcccgagaatggtatgtgattaaaggaggtatggatgtatctcag

SEQ ID NO: 36 - DNA, 600 bp, Escherichia coli
  source 1..600: mol_type = genomic DNA; note = CASE(OVERLAPS WITH D)
atgtatctcagtaaagtcatcattgccagggcctggagcagggatctttaccaacttcaccagggattatggcatttatttccaaacagaccggatgctgctcgtgattttctttttcatgttgagaagcgaaacacaccagaaggctgtcatgttttattgcagtcagcgcaaatgcctgtttcaactgccgttgcgacagtcattaaaactaaacaggttgaatttcaacttcaggttggtgttccactctattttcggcttcgggcaaatccgatcaaaactattctcgacaatcaaaagcgcctggacagtaaagggaatattaaacgctgtcgggttccgttaataaaagaagcagaacaaatcgcgtggttgcaacgtaaattgggcaatgcggcgcgcgttgaagatgtgcatcccatatcggaacggccacagtatttttctggtgatggtaaaagtggaaagatccaaacggtttgctttgaaggtgtgctcaccatcaacgacgcgccagcgttaatagatcttgtacagcaaggtgttgggccagctaaatcgatgggatgtggcttgctatctttggctccactgtga